(19) United States
(12) Patent Application Publication
(10) Pub. No.: US 2024/0239771 A1
(43) Pub. Date: Jul. 18, 2024

(54) BACTERIAL EFFLUX PUMP INHIBITORS

(71) Applicant: TAXIS PHARMACEUTICALS, INC., Monmouth Junction, NJ (US)

(72) Inventors: Ajit K. PARHI, Monmouth Junction, NJ (US); Yi YUAN, Monmouth Junction, NJ (US); Yongzheng ZHANG, Monmouth Junction, NJ (US); Jesus ROSADO, Monmouth Junction, NJ (US); Lu JUN, Monmouth Junction, NJ (US)

(21) Appl. No.: 17/927,645

(22) PCT Filed: May 28, 2021
(86) PCT No.: PCT/US2021/034963

§ 371 Date: Nov. 23, 2022

Related U.S. Application Data
(60) Provisional application No. 63/032,160, filed on May 29, 2020.

(51) Int. Cl. C07D 403/12; A61K 31/4045; A61P 31/04; C07D 209/42

(52) U.S. Cl. CPC C07D 403/12; A61K 31/4045; A61P 31/04; C07D 209/42

(57) ABSTRACT

Disclosed herein are compounds of formula I: (formula I) and salts thereof. Also disclosed are compositions comprising compounds of formula I and methods using compounds of formula I.

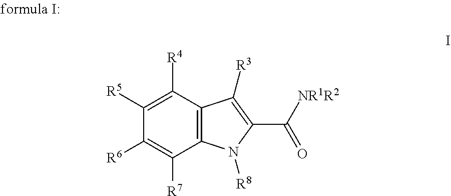

### PRIORITY OF INVENTION

This application claims priority from U.S. Provisional Patent Application No. 63/032,160 filed May 29, 2020, which is hereby incorporated by reference in its entirety.

### BACKGROUND OF THE INVENTION

Antibiotics have been effective tools in the treatment of infectious diseases. However, bacteria have developed several different mechanisms to overcome the action of antibiotics.

These mechanisms of resistance can be specific such as for a molecule or a family of antibiotics, or the mechanisms can be non-specific. Several mechanisms of resistance can exist in a single bacterial strain, and those mechanisms may act independently or they may act synergistically to overcome the action of an antibiotic or a combination of antibiotics. Specific mechanisms include, for example, degradation of the drug, inactivation of the drug by enzymatic modification, and alteration of the drug target. Additional mechanisms of drug resistance include mechanisms in which access of the antibiotic to the target is prevented or reduced by decreasing the transport of the antibiotic into the cell or by increasing the efflux of the drug from the cell to the outside medium. Both of these mechanisms can lower the concentration of drug at the target site and allow bacterial survival in the presence of one or more antibiotics that would otherwise inhibit or kill the bacterial cells. Some bacteria utilize both mechanisms, combining low permeability of the cell wall (including membranes) with an active efflux of antibiotics. It has been shown that efflux of antibiotics can be mediated by more than one pump in a single organism and that almost all antibiotics are subject to resistance by this mechanism.

These multiple resistance mechanisms have become widespread and threaten the clinical utility of antibacterial therapy. The increase in antibiotic resistant strains has been particularly noted in major hospitals and care centers. The consequences of the increase in resistant strains include, for example higher morbidity and mortality, longer patient hospitalization, and an increase in treatment costs. Accordingly, there is a need for agents and methods for inhibiting one or more of these mechanisms of bacterial resistance.

### SUMMARY OF THE INVENTION

Compounds disclose herein, when tested in combination with an antibiotic, lower the minimum inhibitory concentration of the antibiotic to inhibit bacterial cell growth. Not to be bound by theory the compounds are believed to exert this effect by the inhibition of a bacterial efflux pump(s).

Accordingly, one embodiment provides a compound of formula I:

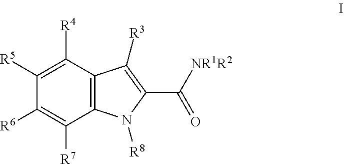

wherein:
R¹ is:
   (a) (C₁-C₁₄)alkyl wherein the (C₁-C₁₄)alkyl is substituted with one or more (e.g., 1, 2, or 3) —NR^{a1}R^{b1} groups, and wherein the (C₁-C₁₄)alkyl is substituted with one or more (e.g., 1, 2, or 3) groups independently selected from the group consisting of —OH, —OR^{c}, —CN, NO₂, B(OH)₂, —SO₂R^{d}, —CO₂R^{e}, —OC(═O)—NR^{a2}R^{b2}, and —O(C═O)—(C₁-C₆)alkyl-NR^{a2}R^{b2}, and wherein the (C₁-C₁₄)alkyl is optionally substituted independently with one or more (e.g., 1, 2, 3, 4, or 5) halo, (C₁-C₄)alkyl or (C₃-C₇)carbocyclyl; or
   (b) 4-7 membered monocyclic heterocyclyl-(C₁-C₆)alkyl- wherein the 4-7 membered monocyclic heterocyclyl-(C₁-C₆)alkyl- is substituted independently with one or more (e.g., 1, 2, or 3) Z¹ or Z², and wherein the 4-7 membered monocyclic heterocyclyl-(C₁-C₆)alkyl- is substituted independently with one or more (e.g., 1, 2, or 3) oxo, Z³, or Z⁴, and wherein the 4-7 membered monocyclic heterocyclyl-(C₁-C₆)alkyl- is optionally substituted independently with one or more (e.g., 1, 2, 3, 4, or 5) halo, (C₁-C₄)alkyl, or (C₃-C₇)carbocyclyl;
R² is hydrogen, (C₁-C₄)alkyl, or (C₃-C₇)carbocyclyl;
R³ is hydrogen, halo, (C₁-C₄)alkyl, (C₁-C₄)haloalkyl, (C₁-C₄)alkoxy, (C₁-C₄)haloalkoxy, OH, —X—(C₃-C₇)carbocyclyl, —X-aryl, —X-heterocyclyl, or —X-heteroaryl, wherein the —X—(C₃-C₇)carbocyclyl, —X-aryl, —X-heterocyclyl, or —X-heteroaryl is independently optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁴ is hydrogen, halo, (C₁-C₄)alkyl, (C₁-C₄)haloalkyl, (C₁-C₄)alkoxy, (C₁-C₄)haloalkoxy, OH, X-aryl, —X-heterocyclyl, or —X-heteroaryl, wherein the —X—(C₃-C₇)carbocyclyl, —X-aryl, —X— heterocyclyl, or —X-heteroaryl is independently optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁵ is hydrogen, halo, (C₁-C₄)alkyl, (C₁-C₄)haloalkyl, (C₁-C₄)alkoxy, (C₁-C₄)haloalkoxy, —OH, X-aryl, —X-heterocyclyl, or —X-heteroaryl, wherein the —X—(C₃-C₇)carbocyclyl, —X-aryl, —X— heterocyclyl, or —X-heteroaryl is independently optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁶ is hydrogen, halo, (C₁-C₄)alkyl, (C₁-C₄)haloalkyl, (C₁-C₄)alkoxy, (C₁-C₄)haloalkoxy, OH, X-aryl, —X-heterocyclyl, or —X-heteroaryl, wherein the —X—(C₃-C₇)carbocyclyl, —X-aryl, —X— heterocyclyl, or —X-heteroaryl is independently optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁷ is hydrogen, halo, (C₁-C₄)alkyl, (C₁-C₄)haloalkyl, (C₁-C₄)alkoxy, (C₁-C₄)haloalkoxy, OH, X-aryl, —X-heterocyclyl, or —X-heteroaryl, wherein the —X—(C₃-C₇)carbocyclyl, —X-aryl, —X— heterocyclyl, or —X-heteroaryl is independently optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁸ is hydrogen, (C₁-C₄)alkyl, or (C₃-C₇)carbocyclyl;
each X is independently absent, —(C₁-C₆)alkyl-, —O—, —S—, —S(═O)—, —S(═O)₂—, or —NR—;
Z¹ is —NR^{a1}R^{b1};
each Z² is independently —(C₁-C₆)alkyl substituted with one or more (e.g., 1, 2, or 3) Z¹, wherein the —(C₁-C₆)alkyl is optionally substituted independently with one or more (e.g., 1, 2, 3, 4, or 5) halo or (C₃-C₇)carbocyclyl;
each Z³ is independently —OH, —OR^{c}, —CN, NO₂, —B(OH)₂, —SO₂R^{d}, —CO₂R^{e}, —OC(═O)—NR^{a2}R^{b2} and —O(C═O)—(C₁-C₆)alkyl-NR^{a2}R^{b2},
each Z⁴ is independently —(C₁-C₆)alkyl substituted with one or more (e.g., 1, 2, or 3) Z³, wherein the —(C₁-C₆)alkyl is optionally substituted independently with one or more (e.g., 1, 2, 3, 4, or 5) halo or (C₃-C₇)carbocyclyl;
each R^{a1} and R^{b1} is independently hydrogen, (C₁-C₆)alkyl or (C₃-C₇)carbocyclyl;
each R^{a2} and R^{b2} is independently hydrogen, (C₁-C₆)alkyl, or (C₃-C₇)carbocyclyl; and
each R^{a3} and R^{b3} is independently hydrogen, (C₁-C₆)alkyl, or (C₃-C₇)carbocyclyl;
each R^{c} is independently (C₁-C₆)alkyl, wherein the (C₁-C₆)alkyl is optionally substituted independently with one or more (e.g., 1, 2, 3, 4, or 5) —NR^{a3}R^{b3} OH, (C₁-C₆)alkoxy, or (C₁-C₆)haloalkoxy;
each R^{d} is independently (C₁-C₆)alkyl or (C₃-C₇)carbocyclyl;
each R^{e} is independently hydrogen, (C₁-C₆)alkyl, or (C₃-C₇)carbocyclyl; and
each R^{f} is independently hydrogen, (C₁-C₆)alkyl, or (C₃-C₇)carbocyclyl;
or a salt thereof.

One embodiment provides a compound of formula I:

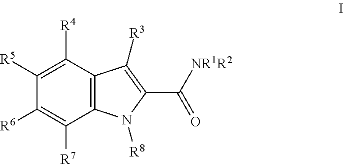

wherein:
R¹ is:
   (a) (C₁-C₁₄)alkyl wherein the (C₁-C₁₄)alkyl is substituted with one or more (e.g., 1, 2, or 3) —NR^{a1}R^{b1} groups, and wherein the (C₁-C₁₄)alkyl is substituted with one or more (e.g., 1, 2, or 3) groups independently selected from the group consisting of —OH, —OR^{c}, —CN, —B(OH)₂, —SO₂R^{d}, —CO₂R, —OC(═O)—NR^{a2}R^{b2}, and —O(C═O)—(C₁-C₆)alkyl-NR^{a2}R^{b2}, and wherein the (C₁-C₁₄)alkyl is optionally substituted independently with one or more (e.g., 1, 2, 3, 4, or 5) halo, (C₁-C₄)alkyl or (C₃-C₇)carbocyclyl; or
   (b) 4-7 membered monocyclic heterocyclyl-(C₁-C₆)alkyl- wherein the 4-7 membered monocyclic heterocyclyl-(C₁-C₆)alkyl- is substituted independently with one or more (e.g., 1, 2, or 3) Z¹ or Z², and wherein the 4-7 membered monocyclic heterocyclyl-(C₁-C₆)alkyl- is substituted independently with one or more (e.g., 1, 2, or 3) oxo, Z³, or Z⁴, and wherein the 4-7 membered monocyclic heterocyclyl-(C₁-C₆)alkyl- is optionally substituted independently with one or more (e.g., 1, 2, 3, 4, or 5) halo, (C₁-C₄)alkyl, or (C₃-C₇)carbocyclyl;
R² is hydrogen, (C₁-C₄)alkyl, or (C₃-C₇)carbocyclyl;
R³ is —X—(C₃-C₇)carbocyclyl, —X-aryl, —X-heterocyclyl, or —X-heteroaryl wherein the —X—(C₃-C₇)carbocyclyl, —X-aryl, —X-heterocyclyl, or —X-heteroaryl is independently optionally substituted with one or more (e.g., 1, 2, 3, 4, or 5) groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁴ is hydrogen, halo, (C₁-C₄)alkyl, (C₁-C₄)haloalkyl, (C₁-C₄)alkoxy, (C₁-C₄)haloalkoxy, or —OH;
R⁵ is hydrogen, halo, (C₁-C₄)alkyl, (C₁-C₄)haloalkyl, (C₁-C₄)alkoxy, (C₁-C₄)haloalkoxy, or —OH;
R⁶ is hydrogen, halo, (C₁-C₄)alkyl, (C₁-C₄)haloalkyl, (C₁-C₄)alkoxy, (C₁-C₄)haloalkoxy, or —OH;
R⁷ is hydrogen, halo, (C₁-C₄)alkyl, (C₁-C₄)haloalkyl, (C₁-C₄)alkoxy, (C₁-C₄)haloalkoxy, or —OH;
R⁸ is hydrogen, (C₁-C₄)alkyl, or (C₃-C₇)carbocyclyl;
each X is independently absent, —(C₁-C₆)alkyl-, —O—, —S—, —S(═O)—, —S(═O)₂—, or —NR^{f}—;
Z¹ is —NR^{a1}R^{b1};
each Z² is independently —(C₁-C₆)alkyl substituted with one or more (e.g., 1, 2, or 3) Z¹, wherein the —(C₁-C₆)alkyl is optionally substituted independently with one or more (e.g., 1, 2, 3, 4, or 5) halo or (C₃-C₇)carbocyclyl;
each Z³ is independently —OH, —OR^{c}, —CN, —B(OH)₂, —SO₂R^{d}, —CO₂R^{e}, —OC(═O)—NR^{a2}R^{b2} and —O(C═O)—(C₁-C₆)alkyl-NR^{a2}R^{b2},
each Z⁴ is independently —(C₁-C₆)alkyl substituted with one or more (e.g., 1, 2, or 3) Z³, wherein the —(C₁-C₆)alkyl is optionally substituted independently with one or more (e.g., 1, 2, 3, 4, or 5) halo or (C₃-C₇)carbocyclyl;
each R^{a1} and R^{b1} is independently hydrogen, (C₁-C₆)alkyl or (C₃-C₇)carbocyclyl;
each R^{a2} and R^{b2} is independently hydrogen, (C₁-C₆)alkyl, or (C₃-C₇)carbocyclyl; and
each R^{a3} and R^{b3} is independently hydrogen, (C₁-C₆)alkyl, or (C₃-C₇)carbocyclyl;
each R^{c} is independently (C₁-C₆)alkyl, wherein the (C₁-C₆)alkyl is optionally substituted independently with one or more (e.g., 1, 2, 3, 4, or 5) —NR^{a3}R^{b3} OH, (C₁-C₆)alkoxy, or (C₁-C₆)haloalkoxy;
each R^{d} is independently (C₁-C₆)alkyl or (C₃-C₇)carbocyclyl;
each R^{e} is independently hydrogen, (C₁-C₆)alkyl, or (C₃-C₇)carbocyclyl; and
each R^{f} is independently hydrogen, (C₁-C₆)alkyl, or (C₃-C₇)carbocyclyl;
or a salt thereof.

One embodiment provides a pharmaceutical composition comprising a compound of formula I or a pharmaceutically acceptable salt thereof as described herein, and a pharmaceutically acceptable vehicle.

One embodiment provides pharmaceutical composition comprising a compound of formula I or a pharmaceutically acceptable salt thereof as described herein, one or more antibacterial agents and a pharmaceutically acceptable vehicle.

One embodiment provides a method of inhibiting a bacterial efflux pump in an animal (e.g., a mammal such as a human) comprising administering to the animal a compound of formula I or a pharmaceutically acceptable salt thereof as described herein.

One embodiment provides a method of inhibiting a bacterial efflux pump in an animal (e.g., a mammal such as a human) comprising administering to the animal in need thereof a compound of formula I or a pharmaceutically acceptable salt thereof as described herein.

One embodiment provides a method of treating or preventing a bacterial infection in an animal (e.g., a mammal such as a human) comprising co-administering to the animal a compound of formula I or a pharmaceutically acceptable salt thereof as described herein and one or more antibacterial agents.

One embodiment provides a method of treating or preventing a bacterial infection in an animal (e.g., a mammal such as a human) comprising co-administering to the animal in need thereof a compound of formula I or a pharmaceutically acceptable salt thereof as described herein and one or more antibacterial agents.

One embodiment provides a method of inhibiting a bacterial efflux pump in an animal (e.g., a mammal such as a human) with a bacterial infection comprising administering to the animal a compound of formula I or a pharmaceutically acceptable salt thereof as described herein.

One embodiment provides a method of treating or preventing a bacterial infection in an animal (e.g., a mammal such as a human) infected with bacteria comprising co-administering to the animal a compound of formula I or a pharmaceutically acceptable salt thereof as described herein and one or more antibacterial agents.

One embodiment provides a compound of formula I or a pharmaceutically acceptable salt thereof as described herein for use in medical treatment.

One embodiment provides a compound of formula I or a pharmaceutically acceptable salt thereof as described herein for the prophylactic or therapeutic inhibition of a bacterial efflux pump for the treatment of a bacterial infection.

One embodiment provides a compound of formula I or a pharmaceutically acceptable salt thereof as described herein which is used in combination with one or more antibacterial agents for the prophylactic or therapeutic treatment of a bacterial infection.

One embodiment provides the use of a compound of formula I or a pharmaceutically acceptable salt thereof as described herein for the preparation of a medicament for inhibiting a bacterial efflux pump.

One embodiment provides the use of a compound of formula I or a pharmaceutically acceptable salt thereof as described herein for the preparation of a medicament for treating a bacterial infection in an animal (e.g., a mammal such as a human).

One embodiment provides the use of a compound of formula I or a pharmaceutically acceptable salt thereof as described herein for the preparation of a medicament which is used in combination with one or more antibacterial agents for treating a bacterial infection in an animal (e.g., a mammal such as a human).

One embodiment provides processes and intermediates disclosed herein that are useful for preparing compounds of formula I or salts thereof.

### DETAILED DESCRIPTION

The following definitions are used, unless otherwise described: halo or halogen is fluoro, chloro, bromo, or iodo. Alkyl and alkoxy, etc. denote both straight and branched groups but reference to an individual radical such as propyl embraces only the straight chain radical (a branched chain isomer such as isopropyl being specifically referred to).

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

The term “aryl” as used herein refers to a single aromatic ring or a multiple condensed ring system wherein the ring atoms are carbon. For example, an aryl group can have 6 to 10 carbon atoms, or 6 to 12 carbon atoms. Aryl includes a phenyl radical. Aryl also includes multiple condensed ring systems (e.g., ring systems comprising 2 rings) having about 9 to 12 carbon atoms or 9 to 10 carbon atoms in which at least one ring is aromatic. Such multiple condensed ring systems may be optionally substituted with one or more (e.g., 1 or 2) oxo groups on any cycloalkyl portion of the multiple condensed ring system. It is to be understood that the point of attachment of a multiple condensed ring system, as defined above, can be at any position of the ring system including an aryl or a cycloalkyl portion of the ring. Typical aryl groups include, but are not limited to, phenyl, indenyl, naphthyl, 1, 2, 3, 4-tetrahydronaphthyl, anthracenyl, and the like.

The term “heteroaryl” as used herein refers to a single aromatic ring or a multiple condensed ring system. The term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term also includes multiple condensed ring systems (e.g. ring systems comprising 2 rings) wherein a heteroaryl group, as defined above, can be condensed with one or more heteroaryls (e.g., naphthyridinyl), heterocycles, (e.g., 1, 2, 3, 4-tetrahydronaphthyridinyl), cycloalkyls (e.g., 5,6,7,8-tetrahydroquinolyl) or aryls (e.g. indazolyl) to form a multiple condensed ring system. Such multiple condensed ring systems may be optionally substituted with one or more (e.g., 1 or 2) oxo groups on the cycloalkyl or heterocycle portions of the condensed ring. In one embodiment a monocyclic or bicyclic heteroaryl has 5 to 10 ring atoms comprising 1 to 9 carbon atoms and 1 to 4 heteroatoms. It is to be understood that the point of attachment of a multiple condensed ring system (as defined above for a heteroaryl) can be at any position of the multiple condensed ring system including a heteroaryl, heterocycle, aryl or cycloalkyl portion of the multiple condensed ring system and at any suitable atom of the multiple condensed ring system including a carbon atom and heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl and thianaphthenyl.

The term “heterocyclyl” or “heterocycle” as used herein refers to a single saturated or partially unsaturated ring or a multiple condensed ring system. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl or piperidinyl. It is to be understood that the point of attachment for a heterocycle can be at any suitable atom of the heterocycle Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl and tetrahydrothiopyranyl.

The term “haloalkyl” includes an alkyl group as defined herein that is substituted with one or more (e.g., 1, 2, 3, or 4) halo groups. One specific halo alkyl is a “(C₁-C₆)haloalkyl”.

The term cycloalkyl, carbocycle, or carbocyclyl includes saturated and partially unsaturated carbocyclic ring systems. In one embodiment the cycloalkyl is a monocyclic carbocyclic ring. Such cycloalkyls include “(C₃-C₇)carbocyclyl” and “(C₃-C₈)cycloalkyl”

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₃-C₈)cycloalkyl can be cyclopropyl, cyclobutyl, cyclopentyl, or cyclohexyl; (C₁-C₆)haloalkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; aryl can be phenyl, indenyl, or naphthyl; and heteroaryl can be fury, imidazolyl, triazolyl, triazinyl, oxazolyl, isoxazolyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

It is understood that the embodiments provided below are for compounds of formula I and all sub-formulas thereof (e.g., formulas Ia, Ib). It is to be understood the two or more embodiments may be combined.

In one embodiment R² is hydrogen.

In one embodiment R⁸ is hydrogen.

In one embodiment a compound of formula I is a compound of formula Ia:

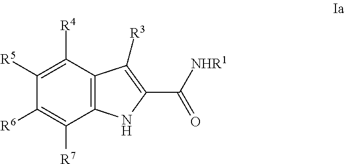

or a salt thereof.

In one embodiment R⁴ is hydrogen or halo.

In one embodiment R⁴ is hydrogen.

In one embodiment R⁴ is hydrogen, halo, or aryl, wherein the aryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R⁴ is hydrogen, halo or phenyl, wherein the phenyl is substituted with halo.

In one embodiment R⁴ is hydrogen or 4-fluorophenyl.

In one embodiment R⁶ is hydrogen or halo.

In one embodiment R⁶ is hydrogen.

In one embodiment R⁶ is hydrogen, halo, or aryl wherein the aryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

In one embodiment R⁶ is hydrogen, halo, or phenyl, wherein the phenyl is substituted with halo.

In one embodiment R⁶ is hydrogen, phenyl, or 4-fluorophenyl.

In one embodiment a compound of formula I is a compound of formula Ib:

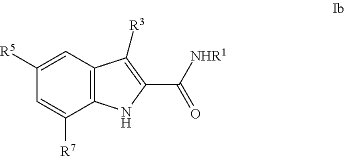

or a salt thereof.

In one embodiment R⁵ is hydrogen or halo.

In one embodiment R⁵ is hydrogen or fluoro.

In one embodiment R⁷ is hydrogen or halo.

In one embodiment R⁷ is hydrogen or fluoro.

In one embodiment R⁷ is hydrogen, halo, or aryl wherein the aryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R⁷ is hydrogen, halo, or phenyl, wherein the phenyl is substituted with halo.

In one embodiment R⁷ is hydrogen, fluoro, or 4-fluorophenyl.

In one embodiment R³ is —X—(C₃-C₇)carbocyclyl or —X-aryl, wherein the —X—(C₃-C₇)carbocyclyl or —X-aryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R³ is (C₃-C₇)carbocyclyl or aryl, wherein the (C₃-C₇)carbocyclyl or aryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R³ is (C₃-C₇)carbocyclyl or phenyl, wherein the (C₃-C₇)carbocyclyl or phenyl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R³ is (C₄-C₇)carbocyclyl or phenyl, wherein the (C₃-C₇)carbocyclyl or phenyl is independently optionally substituted with one or more groups independently selected from the group consisting of halo and —CN.

In one embodiment R³ is:

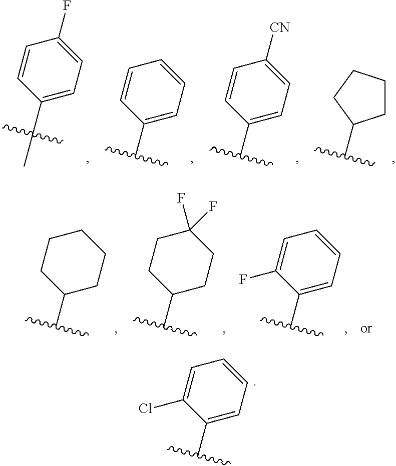

In one embodiment R³ is hydrogen, (C₁-C₄)alkyl, —X—(C₃-C₇)carbocyclyl or —X-aryl, wherein the —X—(C₃-C₇)carbocyclyl or —X-aryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R³ is hydrogen, (C₁-C₄)alkyl, (C₃-C₇)carbocyclyl or —X-aryl, wherein the or —X-aryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment X is absent or —S—.

In one embodiment R³ is hydrogen, (C₁-C₄)alkyl, (C₃-C₇)carbocyclyl or aryl, wherein the (C₃-C₇)carbocyclyl or aryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R³ is hydrogen, (C₁-C₄)alkyl, (C₃-C₇)carbocyclyl or phenyl, wherein the (C₃-C₇)carbocyclyl or phenyl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R³ is hydrogen, (C₁-C₄)alkyl, (C₄-C₇)carbocyclyl or phenyl, wherein the (C₃-C₇)carbocyclyl or phenyl is independently optionally substituted with one or more groups independently selected from the group consisting of halo and —CN.

In one embodiment R³ is: hydrogen, (C₁-C₄)alkyl,

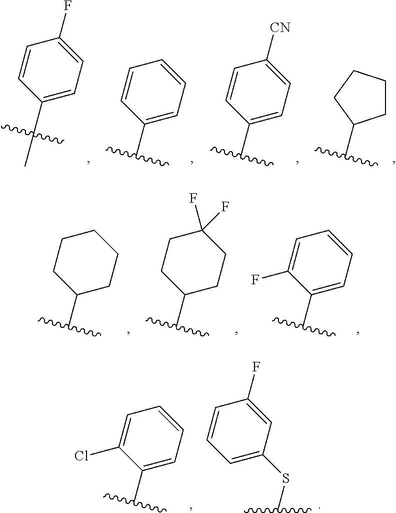

In one embodiment R¹ is:
(a) (C₂-C₇)alkyl wherein the (C₂-C₇)alkyl is substituted with one or more —NR^{a1}R^{b1} groups, and wherein the (C₂-C₇)alkyl is substituted with one or more groups independently selected from the group consisting of —OH, —OR^{c}, —CN, —B(OH)₂, —SO₂R^{d}, —CO₂R^{e}, —OC(═O)—NR^{a2}R^{b2}, and —O(C═O)—(C₁-C₆)alkyl-NR^{a2}R^{b2}, and wherein the (C₂-C₇)alkyl is optionally substituted independently with one or more halo, (C₁-C₄)alkyl or (C₃-C₇)carbocyclyl;
or
(b) 4-7 membered monocyclic heterocyclyl-(C₁-C₂)alkyl- wherein the 4-7 membered monocyclic heterocyclyl-(C₁-C₂)alkyl- is substituted independently with one or more Z¹ or Z², and wherein the 4-7 membered monocyclic heterocyclyl-(C₁-C₁)alkyl- is substituted independently with one or more oxo, Z³, or Z⁴, and wherein the 4-7 membered monocyclic heterocyclyl-(C₁-C₆)alkyl- is optionally substituted independently with one or more halo, (C₁-C₄)alkyl, or (C₃-C₇)carbocyclyl.

In one embodiment R¹ is:
(a) (C₂-C₇)alkyl wherein the (C₂-C₇)alkyl is substituted with one or more —NR^{a1}R^{b1} groups, and wherein the (C₂-C₇)alkyl is substituted with one or more groups independently selected from the group consisting of —OH and —OR^{c} and wherein the (C₂-C₇)alkyl is optionally substituted independently with one or more halo, (C₁-C₄)alkyl or (C₃-C₇)carbocyclyl; or
(b) 4-7 membered monocyclic heterocyclyl-(C₁-C₂)alkyl- wherein the 4-7 membered monocyclic heterocyclyl-(C₁-C₂)alkyl- is substituted independently with one or more —NR^{a1}R^{b1} or —(C₁-C₆)alkyl substituted with one or more —NR^{a1}R^{b1} and wherein the 4-7 membered monocyclic heterocyclyl-(C₁-C₂)alkyl- is substituted independently with one or more oxo or —OH, and wherein the 4-7 membered monocyclic heterocyclyl-(C₁-C₆)alkyl- is optionally substituted independently with one or more halo, (C₁-C₄)alkyl, or (C₃-C₇)carbocyclyl.

In one embodiment R¹ is:
(a) (C₂-C₇)alkyl wherein the (C₂-C₇)alkyl is substituted with one or more —NR^{a1}R^{b1} groups, and wherein the (C₂-C₇)alkyl is substituted with one or more groups independently selected from the group consisting of —OH and —OR^{c}; or
(b) 4-7 membered monocyclic heterocyclyl-(C₁-C₂)alkyl- wherein the 4-7 membered monocyclic heterocyclyl-(C₁-C₂)alkyl- is substituted independently with one or more —NR^{a1}R^{b1} or —(C₁-C₆)alkyl-NR^{a1}R^{b1} and wherein the 4-7 membered monocyclic heterocyclyl-(C₁-C₂)alkyl- is substituted independently with one or more oxo or —OH.

In one embodiment R¹ is:
(a) (C₂-C₇)alkyl wherein the (C₂-C₇)alkyl is substituted with one or more —NH₂, and wherein the (C₂-C₇)alkyl is substituted with one or more groups independently selected from the group consisting of —OH and —O(C₁-C₆)alkyl-NH₂; or
(b) 4-7 membered monocyclic heterocyclyl-(C₁-C₂)alkyl- wherein the 4-7 membered monocyclic heterocyclyl-(C₁-C₂)alkyl- is substituted independently with one or more —(C₁-C₆)alkyl-NH₂, and wherein the 4-7 membered monocyclic heterocyclyl-(C₁-C₂)alkyl- is substituted independently with one or more oxo or —OH.

In one embodiment R¹ is (C₂-C₇)alkyl wherein the (C₂-C₇)alkyl is substituted with one or more —NR^{a1}R^{b1} groups, and wherein the (C₂-C₇)alkyl is substituted with one or more groups independently selected from the group consisting of —OH, —OR^{c}, —CN, —B(OH)₂, —SO₂R^{d}, —CO₂R^{e}, —OC(═O)—NR^{a2}R^{b2}, and —O(C═O)—(C₁-C₆)alkyl-NR^{a2}R^{b2}, and wherein the (C₂-C₇)alkyl is optionally substituted independently with one or more halo, (C₁-C₄)alkyl or (C₃-C₇)carbocyclyl.

In one embodiment R¹ is (C₂-C₇)alkyl wherein the (C₂-C₇)alkyl is substituted with one or more —NR^{a1}R^{b1} groups, and wherein the (C₂-C₇)alkyl is substituted with one or more groups independently selected from the group consisting of —OH and —OR^{c} and wherein the (C₂-C₇)alkyl is optionally substituted independently with one or more halo, (C₁-C₄)alkyl or (C₃-C₇)carbocyclyl.

In one embodiment R¹ is (C₂-C₇)alkyl wherein the (C₂-C₇)alkyl is substituted with one or more —NR^{a1}R^{b1} groups, and wherein the (C₂-C₇)alkyl is substituted with one or more groups independently selected from the group consisting of —OH and —OR^{c}.

In one embodiment R¹ is (C₂-C₇)alkyl wherein the (C₂-C₇)alkyl is substituted with one or more —NH₂, and wherein the (C₂-C₇)alkyl is substituted with one or more groups independently selected from the group consisting of —OH and —O(C₁-C₆)alkyl-NH₂.

In one embodiment R¹ is 4-7 membered monocyclic heterocyclyl-(C₁-C₂)alkyl- wherein the 4-7 membered monocyclic heterocyclyl-(C₁-C₂)alkyl- is substituted independently with one or more Z¹ or Z², and wherein the 4-7 membered monocyclic heterocyclyl-(C₁-C₁)alkyl- is substituted independently with one or more oxo, Z³, or Z⁴, and wherein the 4-7 membered monocyclic heterocyclyl-(C₁-C₆)alkyl- is optionally substituted independently with one or more halo, (C₁-C₄)alkyl, or (C₃-C₇)carbocyclyl.

In one embodiment R¹ is 4-7 membered monocyclic heterocyclyl-(C₁-C₂)alkyl- wherein the 4-7 membered monocyclic heterocyclyl-(C₁-C₂)alkyl- is substituted independently with one or more —NR^{a1}R^{b1} or —(C₁-C₆)alkyl substituted with one or more —NR^{a1}R^{b1} and wherein the 4-7 membered monocyclic heterocyclyl-(C₁-C₂)alkyl- is substituted independently with one or more oxo or —OH, and wherein the 4-7 membered monocyclic heterocyclyl-(C₁-C₆)alkyl- is optionally substituted independently with one or more halo, (C₁-C₄)alkyl, or (C₃-C₇)carbocyclyl.

In one embodiment R¹ is 4-7 membered monocyclic heterocyclyl-(C₁-C₂)alkyl- wherein the 4-7 membered monocyclic heterocyclyl-(C₁-C₂)alkyl- is substituted independently with one or more —NR^{a1}R^{b1} or —(C₁-C₆)alkyl-NR^{a1}R^{b1} and wherein the 4-7 membered monocyclic heterocyclyl-(C₁-C₂)alkyl- is substituted independently with one or more oxo or —OH.

In one embodiment R¹ is 4-7 membered monocyclic heterocyclyl-(C₁-C₂)alkyl- wherein the 4-7 membered monocyclic heterocyclyl-(C₁-C₂)alkyl- is substituted independently with one or more —(C₁-C₆)alkyl-NH₂, and wherein the 4-7 membered monocyclic heterocyclyl-(C₁-C₂)alkyl- is substituted independently with one or more oxo or —OH.

In one embodiment R¹ is:
(a) (C₂-C₇)alkyl wherein the (C₂-C₇)alkyl is substituted with two or more —NR^{a1}R^{b1} groups, and wherein the (C₂-C₇)alkyl is substituted with one or more groups independently selected from the group consisting of —OH, —OR^{c}, —CN, —B(OH)₂, —SO₂R^{d}, —CO₂R^{e}, —OC(═O)—NR^{a2}R^{b2}, and —O(C═O)—(C₁-C₆)alkyl-NR^{a2}R^{b2}, and wherein the (C₂-C₇)alkyl is optionally substituted independently with one or more halo, (C₁-C₄)alkyl or (C₃-C₇)carbocyclyl;
or
(b) 4-7 membered monocyclic heterocyclyl-(C₁-C₂)alkyl- wherein the 4-7 membered monocyclic heterocyclyl-(C₁-C₂)alkyl- is substituted independently with one or more Z¹ or Z², and wherein the 4-7 membered monocyclic heterocyclyl-(C₁-C₁)alkyl- is substituted independently with one or more oxo, Z³, or Z⁴, and wherein the 4-7 membered monocyclic heterocyclyl-(C₁-C₆)alkyl- is optionally substituted independently with one or more halo, (C₁-C₄)alkyl, or (C₃-C₇)carbocyclyl.

In one embodiment R¹ is:
(a) (C₂-C₇)alkyl wherein the (C₂-C₇)alkyl is substituted with two or more —NR^{a1}R^{b1} groups, and wherein the (C₂-C₇)alkyl is substituted with one or more groups independently selected from the group consisting of —OH and —OR^{c} and wherein the (C₂-C₇)alkyl is optionally substituted independently with one or more halo, (C₁-C₄)alkyl or (C₃-C₇)carbocyclyl; or
(b) 4-7 membered monocyclic heterocyclyl-(C₁-C₂)alkyl- wherein the 4-7 membered monocyclic heterocyclyl-(C₁-C₂)alkyl- is substituted independently with one or more —NR^{a1}R^{b1} or —(C₁-C₆)alkyl substituted with one or more —NR^{a1}R^{b1} and wherein the 4-7 membered monocyclic heterocyclyl-(C₁-C₂)alkyl- is substituted independently with one or more oxo or —OH, and wherein the 4-7 membered monocyclic heterocyclyl-(C₁-C₆)alkyl- is optionally substituted independently with one or more halo, (C₁-C₄)alkyl, or (C₃-C₇)carbocyclyl.

In one embodiment R¹ is:
(a) (C₂-C₇)alkyl wherein the (C₂-C₇)alkyl is substituted with two or more —NR^{a1}R^{b1} groups, and wherein the (C₂-C₇)alkyl is substituted with one or more groups independently selected from the group consisting of —OH and —OR^{c}; or
(b) 4-7 membered monocyclic heterocyclyl-(C₁-C₂)alkyl- wherein the 4-7 membered monocyclic heterocyclyl-(C₁-C₂)alkyl- is substituted independently with one or more —NR^{a1}R^{b1} or —(C₁-C₆)alkyl-NR^{a1}R^{b1} and wherein the 4-7 membered monocyclic heterocyclyl-(C₁-C₂)alkyl- is substituted independently with one or more oxo or —OH.

In one embodiment R¹ is:
(a) (C₂-C₇)alkyl wherein the (C₂-C₇)alkyl is substituted with two or more —NH₂, and wherein the (C₂-C₇)alkyl is substituted with one or more groups independently selected from the group consisting of —OH and —O(C₁-C₆)alkyl-NH₂; or
(b) 4-7 membered monocyclic heterocyclyl-(C₁-C₂)alkyl- wherein the 4-7 membered monocyclic heterocyclyl-(C₁-C₂)alkyl- is substituted independently with one or more —(C₁-C₆)alkyl-NH₂, and wherein the 4-7 membered monocyclic heterocyclyl-(C₁-C₂)alkyl- is substituted independently with one or more oxo or —OH.

In one embodiment R¹ is (C₂-C₇)alkyl wherein the (C₂-C₇)alkyl is substituted with two or more —NR^{a1}R^{b1} groups, and wherein the (C₂-C₇)alkyl is substituted with one or more groups independently selected from the group consisting of —OH, —OR^{c}, —CN, —B(OH)₂, —SO₂R^{d}, —CO₂R^{e}, —OC(═O)—NR^{a2}R^{b2}, and —O(C═O)—(C₁-C₆)alkyl-NR^{a2}R^{b2}, and wherein the (C₂-C₇)alkyl is optionally substituted independently with one or more halo, (C₁-C₄)alkyl or (C₃-C₇)carbocyclyl.

In one embodiment R¹ is (C₂-C₇)alkyl wherein the (C₂-C₇)alkyl is substituted with two or more —NR^{a1}R^{b1} groups, and wherein the (C₂-C₇)alkyl is substituted with one or more groups independently selected from the group consisting of —OH and —OR^{c} and wherein the (C₂-C₇)alkyl is optionally substituted independently with one or more halo, (C₁-C₄)alkyl or (C₃-C₇)carbocyclyl.

In one embodiment R¹ is (C₂-C₇)alkyl wherein the (C₂-C₇)alkyl is substituted with two or more —NR^{a1}R^{b1} groups, and wherein the (C₂-C₇)alkyl is substituted with one or more groups independently selected from the group consisting of —OH and —OR^{c}.

In one embodiment R¹ is (C₂-C₇)alkyl wherein the (C₂-C₇)alkyl is substituted with two or more —NH₂, and wherein the (C₂-C₇)alkyl is substituted with one or more groups independently selected from the group consisting of —OH and —O(C₁-C₆)alkyl-NH₂.

In one embodiment R¹ is 4-7 membered monocyclic heterocyclyl-(C₁-C₂)alkyl- wherein the 4-7 membered monocyclic heterocyclyl-(C₁-C₂)alkyl- is substituted independently with two or more Z¹ or Z², and wherein the 4-7 membered monocyclic heterocyclyl-(C₁-C₁)alkyl- is substituted independently with one or more oxo, Z³, or Z⁴, and wherein the 4-7 membered monocyclic heterocyclyl-(C₁-C₆)alkyl- is optionally substituted independently with one or more halo, (C₁-C₄)alkyl, or (C₃-C₇)carbocyclyl.

In one embodiment R¹ is 4-7 membered monocyclic heterocyclyl-(C₁-C₂)alkyl- wherein the 4-7 membered monocyclic heterocyclyl-(C₁-C₂)alkyl- is substituted independently with two or more —NR^{a1}R^{b1} or —(C₁-C₆)alkyl substituted with one or more —NR^{a1}R^{b1} and wherein the 4-7 membered monocyclic heterocyclyl-(C₁-C₂)alkyl- is substituted independently with one or more oxo or —OH, and wherein the 4-7 membered monocyclic heterocyclyl-(C₁-C₆)alkyl- is optionally substituted independently with one or more halo, (C₁-C₄)alkyl, or (C₃-C₇)carbocyclyl.

In one embodiment R¹ is 4-7 membered monocyclic heterocyclyl-(C₁-C₂)alkyl- wherein the 4-7 membered monocyclic heterocyclyl-(C₁-C₂)alkyl- is substituted independently with two or more —NR^{a1}R^{b1} or —(C₁-C₆)alkyl-NR^{a1}R^{b1} and wherein the 4-7 membered monocyclic heterocyclyl-(C₁-C₂)alkyl- is substituted independently with one or more oxo or —OH.

In one embodiment R¹ is 4-7 membered monocyclic heterocyclyl-(C₁-C₂)alkyl- wherein the 4-7 membered monocyclic heterocyclyl-(C₁-C₂)alkyl- is substituted independently with two or more —(C₁-C₆)alkyl-NH₂, and wherein the 4-7 membered monocyclic heterocyclyl-(C₁-C₂)alkyl- is substituted independently with one or more oxo or —OH.

In one embodiment R¹ is:

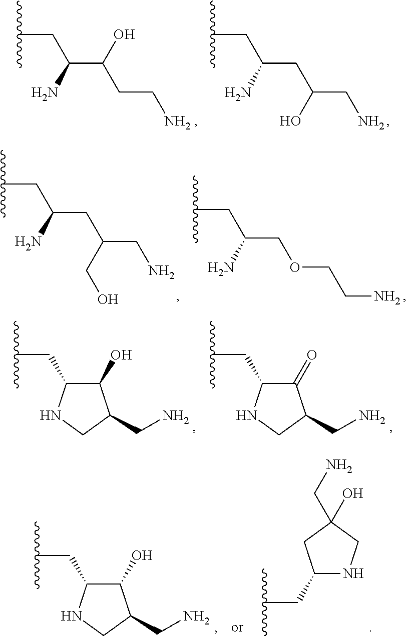

In one embodiment R¹ is:
(a) (C₂-C₇)alkyl wherein the (C₂-C₇)alkyl is substituted with one or more —NH₂, and wherein the (C₂-C₇)alkyl is substituted with one or more groups independently selected from the group consisting of —OH and —O(C₁-C₆)alkyl, wherein the —O(C₁-C₆)alkyl is substituted with one or two —NH₂; or
(b) 4-7 membered monocyclic heterocyclyl-(C₁-C₂)alkyl- wherein the 4-7 membered monocyclic heterocyclyl-(C₁-C₂)alkyl- is substituted independently with one or more —(C₁-C₆)alkyl-NH₂, and wherein the 4-7 membered monocyclic heterocyclyl-(C₁-C₂)alkyl- is substituted independently with one or more oxo or —OH.

In one embodiment R¹ is (C₂-C₇)alkyl wherein the (C₂-C₇)alkyl is substituted with one or more —NH₂, and wherein the (C₂-C₇)alkyl is substituted with one or more groups independently selected from the group consisting of —OH and —O(C₁-C₆)alkyl, wherein the —O(C₁-C₆)alkyl is substituted with one or two —NH₂.

In one embodiment R¹ is:

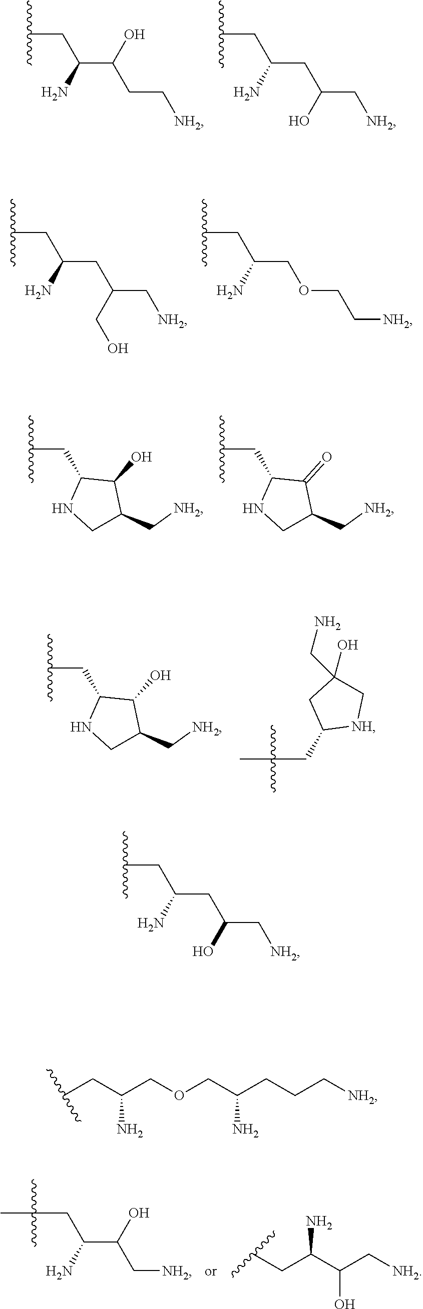

One embodiment provides a compound that is:

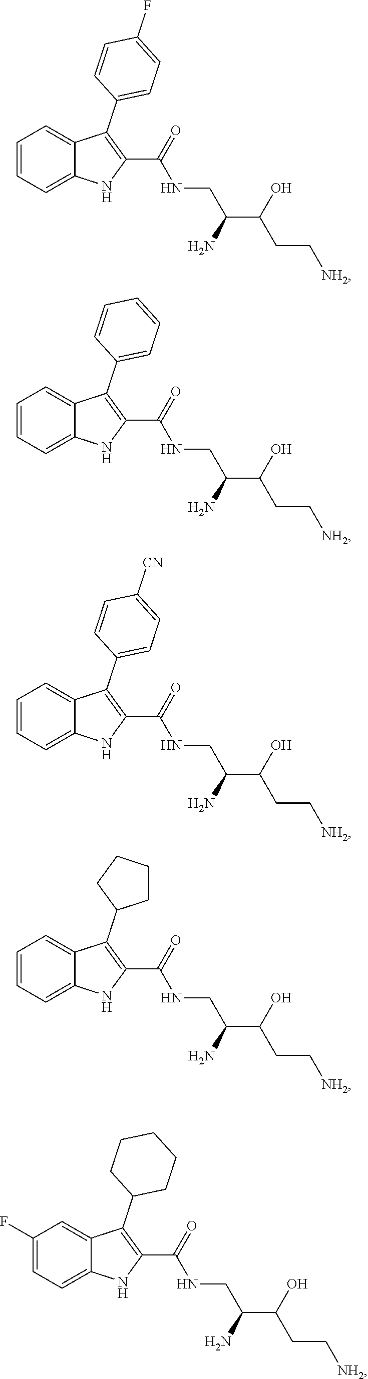 [SMILES: NCCC(O)[C@@H](N)CNC(=O)c1[nH]c2ccccc2c1-c1ccc(F)cc1 ; NCCC(O)[C@@H](N)CNC(=O)c1[nH]c2ccccc2c1-c1ccccc1 ; N#Cc1ccc(-c2c(C(=O)NC[C@H](N)C(O)CCN)[nH]c3ccccc23)cc1 ; NCCC(O)[C@@H](N)CNC(=O)c1[nH]c2ccccc2c1C1CCCC1 ; NCCC(O)[C@@H](N)CNC(=O)c1[nH]c2ccc(F)cc2c1C1CCCCC1]

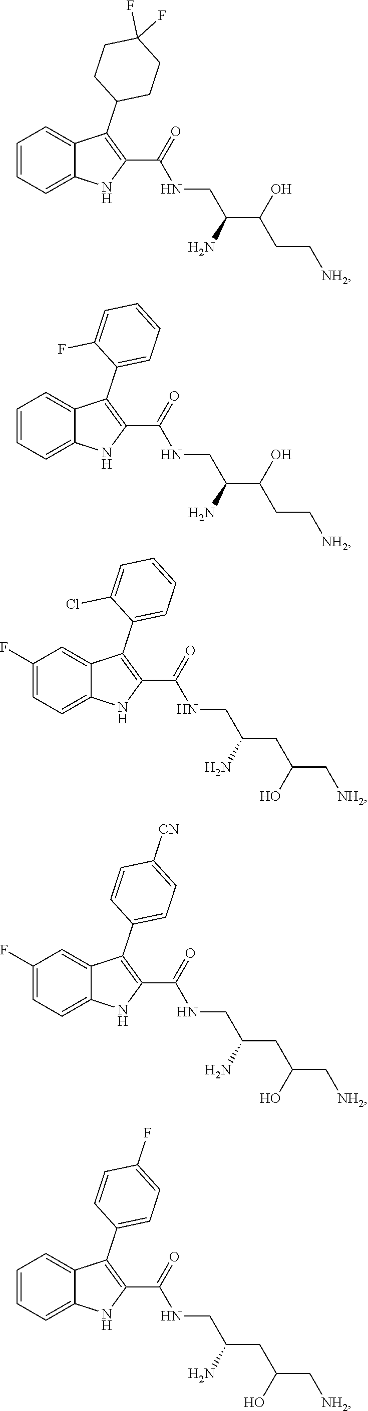 [SMILES: NCCC(O)[C@@H](N)CNC(=O)c1[nH]c2ccccc2c1C1CCC(F)(F)CC1 ; NCCC(O)[C@@H](N)CNC(=O)c1[nH]c2ccccc2c1-c1ccccc1F ; NCC(O)C[C@H](N)CNC(=O)c1[nH]c2ccc(F)cc2c1-c1ccccc1Cl ; N#Cc1ccc(-c2c(C(=O)NC[C@@H](N)CC(O)CN)[nH]c3ccc(F)cc23)cc1 ; NCC(O)C[C@H](N)CNC(=O)c1[nH]c2ccccc2c1-c1ccc(F)cc1]

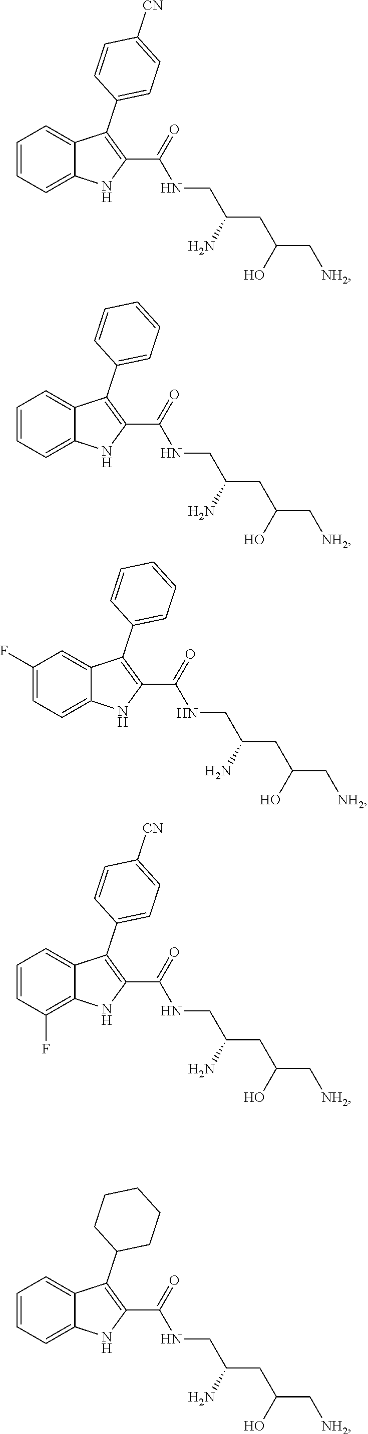 [SMILES: N#Cc1ccc(-c2c(C(=O)NC[C@@H](N)CC(O)CN)[nH]c3ccccc23)cc1 ; NCC(O)C[C@H](N)CNC(=O)c1[nH]c2ccccc2c1-c1ccccc1 ; NCC(O)C[C@H](N)CNC(=O)c1[nH]c2ccc(F)cc2c1-c1ccccc1 ; N#Cc1ccc(-c2c(C(=O)NC[C@@H](N)CC(O)CN)[nH]c3c(F)cccc23)cc1 ; NCC(O)C[C@H](N)CNC(=O)c1[nH]c2ccccc2c1C1CCCCC1]

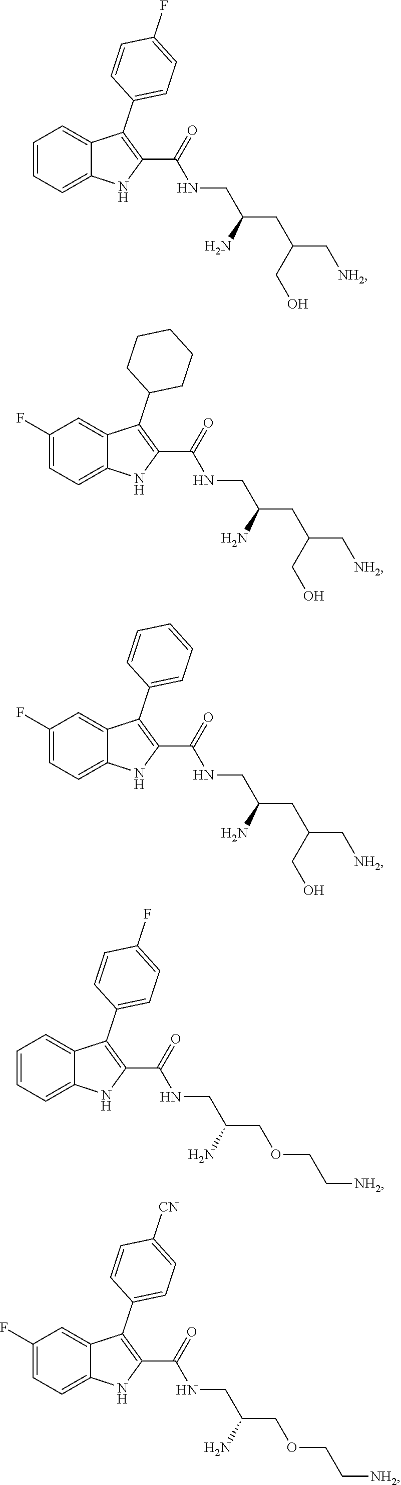 [SMILES: NCC(CO)C[C@@H](N)CNC(=O)c1[nH]c2ccccc2c1-c1ccc(F)cc1 ; NCC(CO)C[C@@H](N)CNC(=O)c1[nH]c2ccc(F)cc2c1C1CCCCC1 ; NCC(CO)C[C@@H](N)CNC(=O)c1[nH]c2ccc(F)cc2c1-c1ccccc1 ; NCCOC[C@H](N)CNC(=O)c1[nH]c2ccccc2c1-c1ccc(F)cc1 ; N#Cc1ccc(-c2c(C(=O)NC[C@@H](N)COCCN)[nH]c3ccc(F)cc23)cc1]

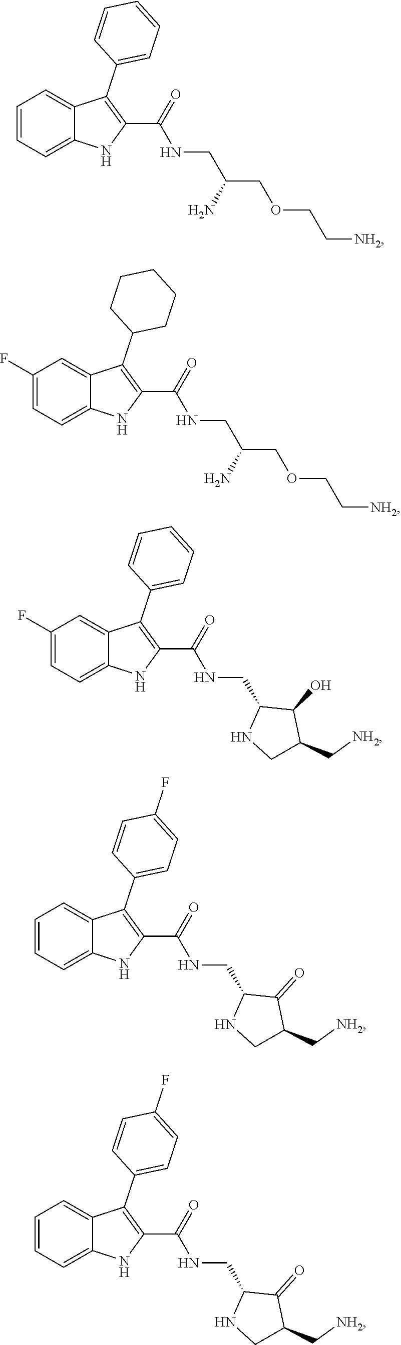 [SMILES: NCCOC[C@H](N)CNC(=O)c1[nH]c2ccccc2c1-c1ccccc1 ; NCCOC[C@H](N)CNC(=O)c1[nH]c2ccc(F)cc2c1C1CCCCC1 ; NC[C@H]1CN[C@H](CNC(=O)c2[nH]c3ccc(F)cc3c2-c2ccccc2)[C@H]1O ; NC[C@H]1CN[C@H](CNC(=O)c2[nH]c3ccccc3c2-c2ccc(F)cc2)C1=O ; NC[C@H]1CN[C@H](CNC(=O)c2[nH]c3ccccc3c2-c2ccc(F)cc2)C1=O]

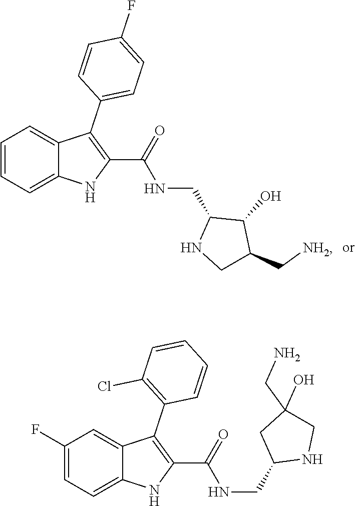 [SMILES: NC[C@H]1CN[C@H](CNC(=O)c2[nH]c3ccccc3c2-c2ccc(F)cc2)[C@@H]1O ; NCC1(O)CN[C@H](CNC(=O)c2[nH]c3ccc(F)cc3c2-c2ccccc2Cl)C1]

or a salt thereof.

One embodiment provides a compound that is:

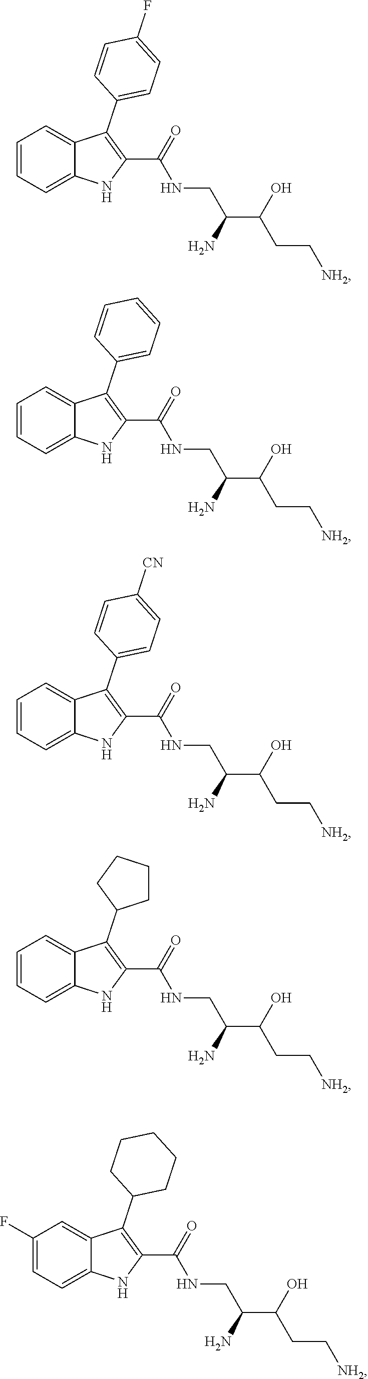 [SMILES: NCCC(O)[C@@H](N)CNC(=O)c1[nH]c2ccccc2c1-c1ccc(F)cc1 ; NCCC(O)[C@@H](N)CNC(=O)c1[nH]c2ccccc2c1-c1ccccc1 ; N#Cc1ccc(-c2c(C(=O)NC[C@H](N)C(O)CCN)[nH]c3ccccc23)cc1 ; NCCC(O)[C@@H](N)CNC(=O)c1[nH]c2ccccc2c1C1CCCC1 ; NCCC(O)[C@@H](N)CNC(=O)c1[nH]c2ccc(F)cc2c1C1CCCCC1]

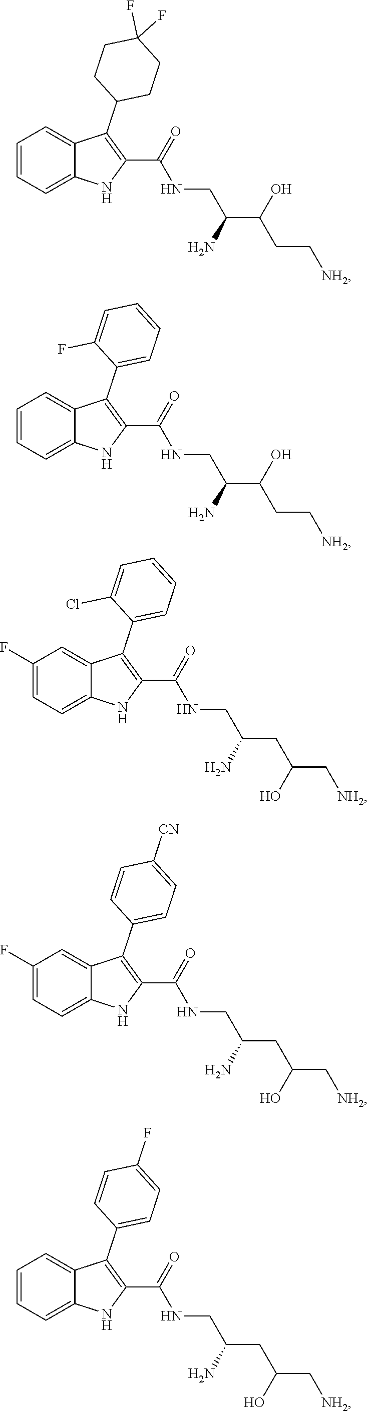 [SMILES: NCCC(O)[C@@H](N)CNC(=O)c1[nH]c2ccccc2c1C1CCC(F)(F)CC1 ; NCCC(O)[C@@H](N)CNC(=O)c1[nH]c2ccccc2c1-c1ccccc1F ; NCC(O)C[C@H](N)CNC(=O)c1[nH]c2ccc(F)cc2c1-c1ccccc1Cl ; N#Cc1ccc(-c2c(C(=O)NC[C@@H](N)CC(O)CN)[nH]c3ccc(F)cc23)cc1 ; NCC(O)C[C@H](N)CNC(=O)c1[nH]c2ccccc2c1-c1ccc(F)cc1]

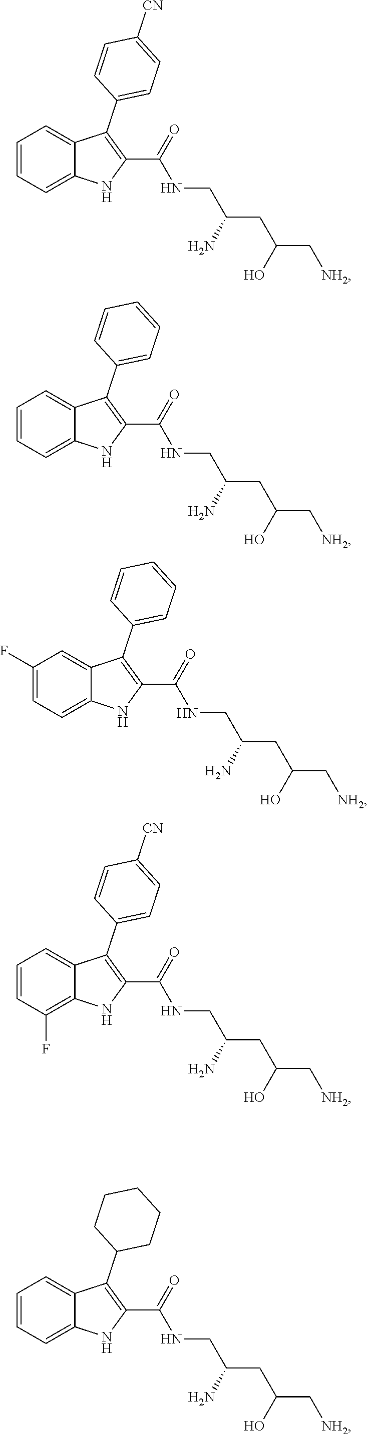 [SMILES: N#Cc1ccc(-c2c(C(=O)NC[C@@H](N)CC(O)CN)[nH]c3ccccc23)cc1 ; NCC(O)C[C@H](N)CNC(=O)c1[nH]c2ccccc2c1-c1ccccc1 ; NCC(O)C[C@H](N)CNC(=O)c1[nH]c2ccc(F)cc2c1-c1ccccc1 ; N#Cc1ccc(-c2c(C(=O)NC[C@@H](N)CC(O)CN)[nH]c3c(F)cccc23)cc1 ; NCC(O)C[C@H](N)CNC(=O)c1[nH]c2ccccc2c1C1CCCCC1]

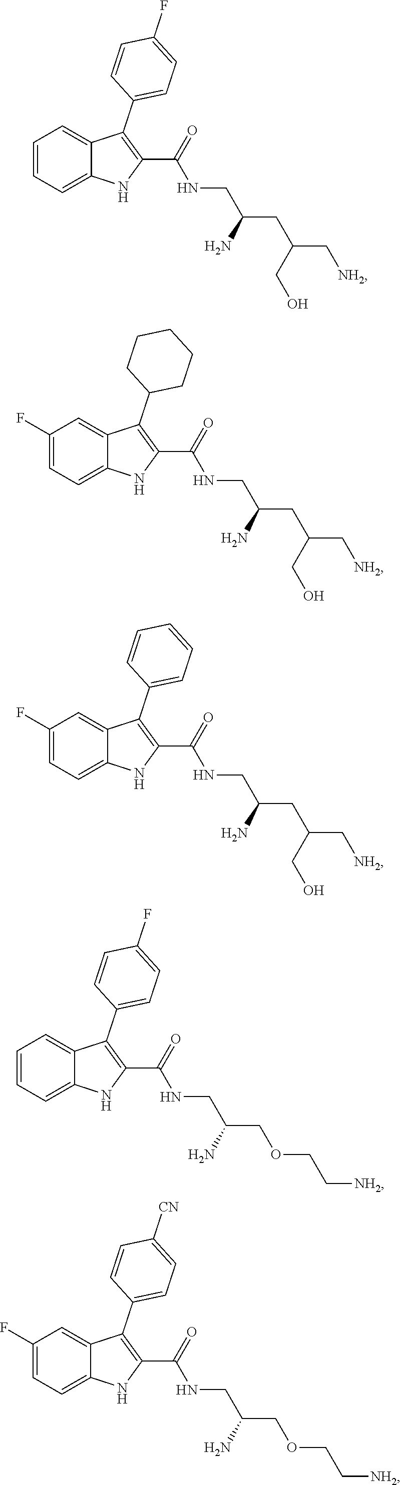 [SMILES: NCC(CO)C[C@@H](N)CNC(=O)c1[nH]c2ccccc2c1-c1ccc(F)cc1 ; NCC(CO)C[C@@H](N)CNC(=O)c1[nH]c2ccc(F)cc2c1C1CCCCC1 ; NCC(CO)C[C@@H](N)CNC(=O)c1[nH]c2ccc(F)cc2c1-c1ccccc1 ; NCCOC[C@H](N)CNC(=O)c1[nH]c2ccccc2c1-c1ccc(F)cc1 ; N#Cc1ccc(-c2c(C(=O)NC[C@@H](N)COCCN)[nH]c3ccc(F)cc23)cc1]

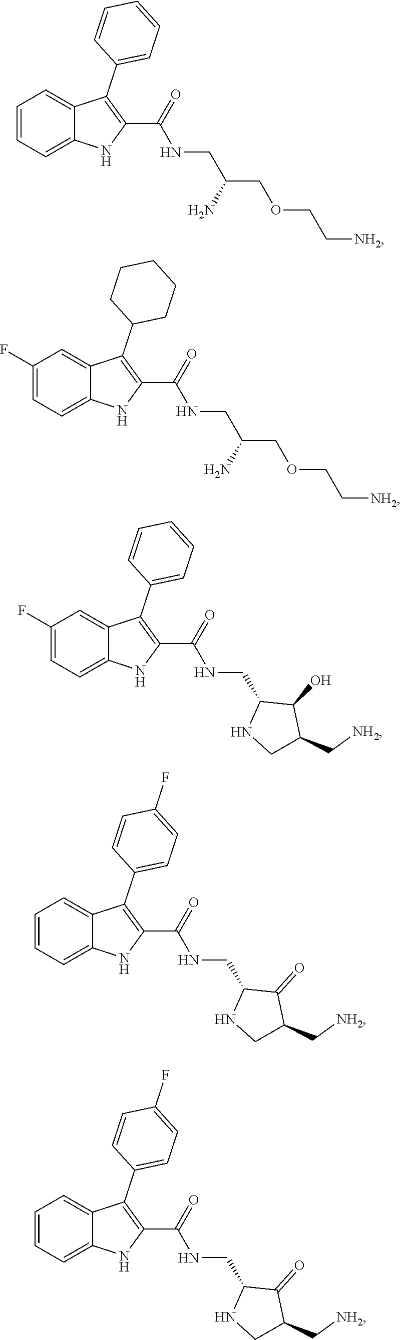 [SMILES: NCCOC[C@H](N)CNC(=O)c1[nH]c2ccccc2c1-c1ccccc1 ; NCCOC[C@H](N)CNC(=O)c1[nH]c2ccc(F)cc2c1C1CCCCC1 ; NC[C@H]1CN[C@H](CNC(=O)c2[nH]c3ccc(F)cc3c2-c2ccccc2)[C@H]1O ; NC[C@H]1CN[C@H](CNC(=O)c2[nH]c3ccccc3c2-c2ccc(F)cc2)C1=O ; NC[C@H]1CN[C@H](CNC(=O)c2[nH]c3ccccc3c2-c2ccc(F)cc2)C1=O]

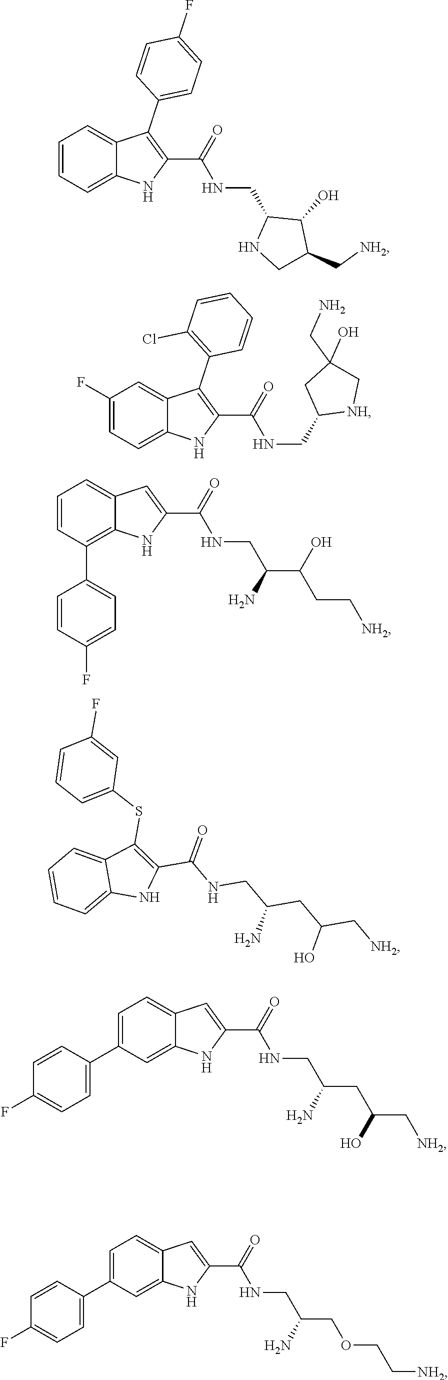

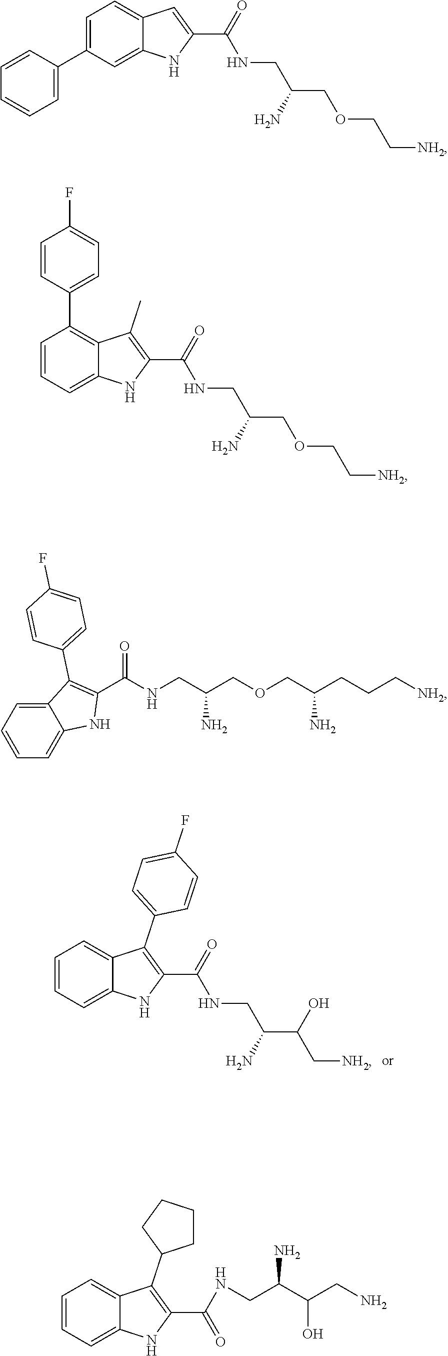 [SMILES: NCCOC[C@H](N)CNC(=O)c1cc2ccc(-c3ccccc3)cc2[nH]1 ; Cc1c(C(=O)NC[C@@H](N)COCCN)[nH]c2cccc(-c3ccc(F)cc3)c12 ; NCCC[C@H](N)COC[C@H](N)CNC(=O)c1[nH]c2ccccc2c1-c1ccc(F)cc1 ; NCC(O)[C@H](N)CNC(=O)c1[nH]c2ccccc2c1-c1ccc(F)cc1 ; NCC(O)[C@H](N)CNC(=O)c1[nH]c2ccccc2c1C1CCCC1]

or a salt thereof.

Generally, compounds of formula I as well as synthetic intermediates that can be used for preparing compounds of formula I can be prepared as illustrated in the following general schemes. It is understood that variable groups shown below (e.g., R¹, R², R³, R⁴, R⁵, R⁶. R⁷, R⁸) can represent the final corresponding groups present in a compound of formula I or that these groups can represent groups that can be converted to the final corresponding groups present in a compound of formula I at a convenient point in a synthetic sequence. For example, the variable groups can contain one or more protecting groups that can be removed at a convenient point in a synthetic sequence to provide the final corresponding groups in the compound of formula I. Schemes 1-5 illustrate general methods for the preparation of compounds of formula I.

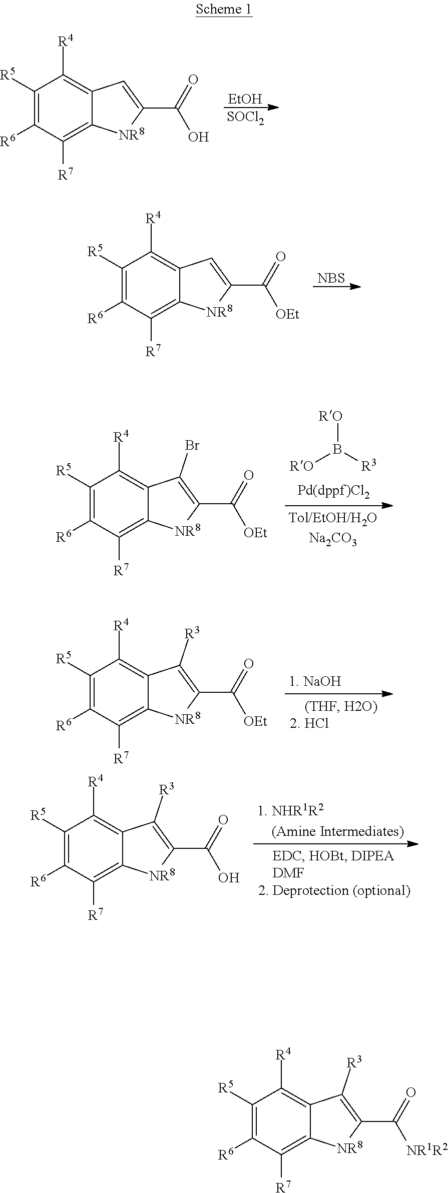

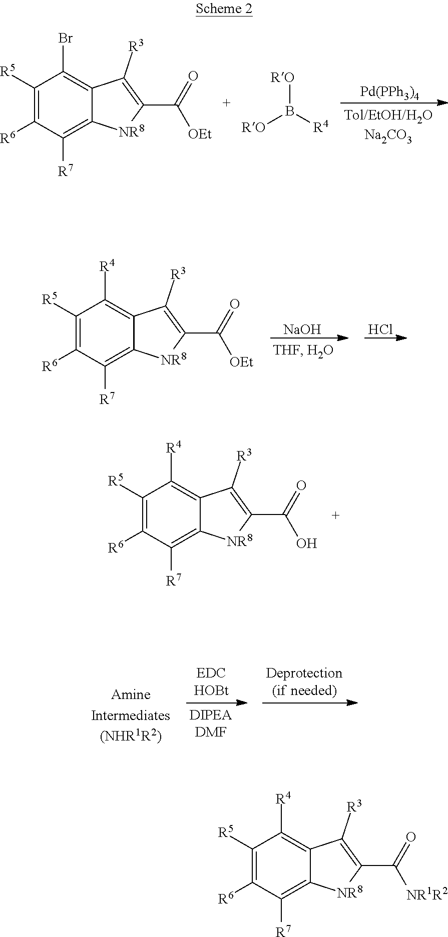

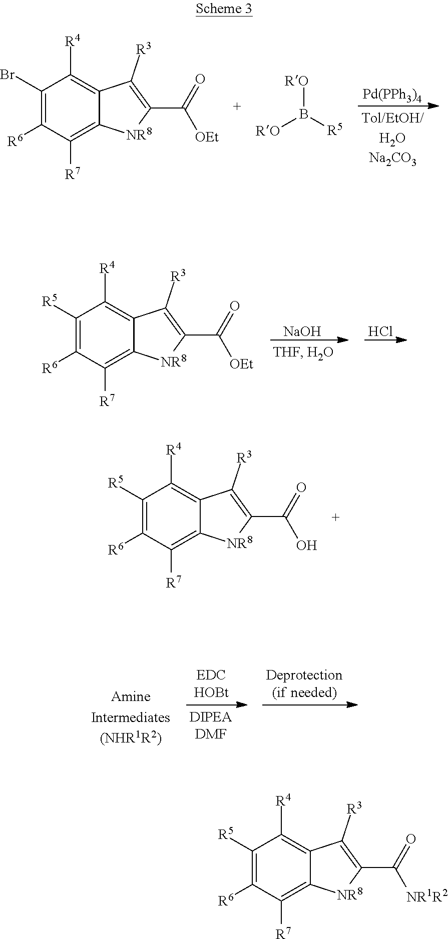

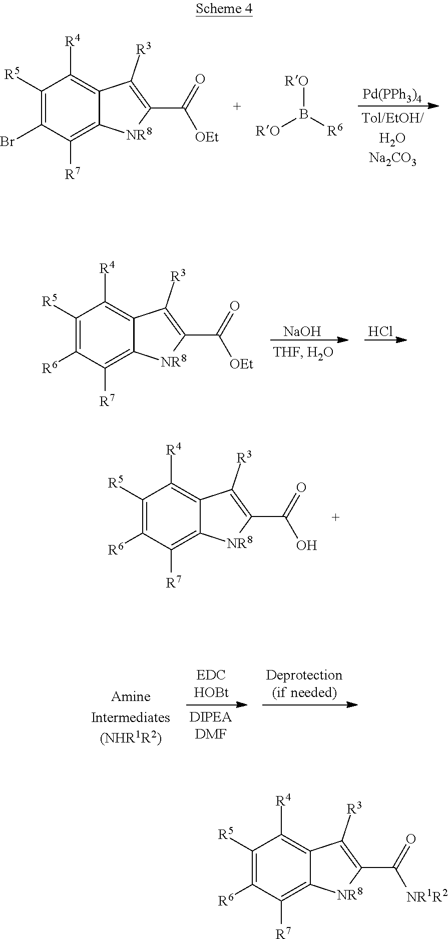

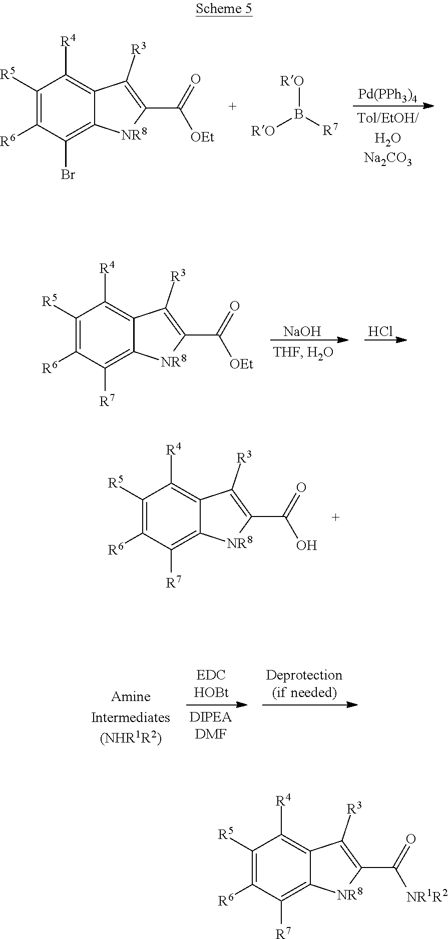

The compounds disclosed herein are bacterial efflux pump inhibitors. An efflux pump inhibitor is a compound that interferes with the ability of an efflux pump to export a substrate. The inhibitor may have intrinsic antibacterial properties of its own. The compounds disclosed herein may be useful for treating bacterial infections (e.g., gram negative and gram positive) when administered with an antibacterial agent.

In one embodiment the bacterial infection being treated is a Gram-negative bacterial strain infection. In one embodiment the Gram-negative bacterial strain is selected from the group consisting of Acinetobacter baumannii, Acinetobacter calcoaceticus, Acinetobacter haemolyticus, Acinetobacter lwoffi, Actinobacillus actinomycetemcomitans, Aeromonas hydrophilia, Aggregatibacter actinomycetemcomitans, Agrobacterium tumefaciens, Bacteroides distasonis, Bacteroides eggerthii, Bacteroides forsythus, Bacteroides fragilis, Bacteroides ovalus, Bacteroides splanchnicus, Bacteroides thetaiotaomicron, Bacteroides uniformis, Bacteroides vulgatus, Bordetella bronchiseptica, Bordetella parapertussis, Bordetella pertussis, Borrelia burgdorferi, Branhamella catarrhalis, Burkholderia cepacia, Campylobacter coli, Campylobacter fetus, Campylobacter jejuni, Caulobacter crescentus, Chlamydia trachomatis, Citrobacter diversus, Citrobacter freundii, Enterobacter aerogenes, Enterobacter asburiae, Enterobacter cloacae, Enterobacter sakazakii, Escherchia coli, Francisella tularensis, Fusobacterium nucleatum, Gardnerella vaginalis, Haemophilus ducreyi, Haemophilus haemolyticus, Haemophilus influenzae, Haemophilus parahaemolyticus, Haemophilus parainfluenzae, Helicobacter pylori, Kingella denitrificans, Kingella indologenes, Kingella kingae, Kingella oralis, Klebsiella oxytoca, Klebsiella pneumoniae, Klebsiella rhinoscleromatis, Legionella pneumophila, Listeria monocytogenes, Moraxella bovis, Moraxella catarrhalis, Moraxella lacunata, Morganella morganii, Neisseria gonorrhoeae, Neisseria meningitides, Pantoea agglomerans, Pasteurella canes, Pasteurella haemolytica, Pasteurella multocida, Pasteurella tularensis, Porphyromonas gingivalis, Proteus mirabilis, Proteus vu/gar/s, Providencia alcalifaciens, Providencia rettgeri, Providencia stuartii, Pseudomonas acidovorans, Pseudomonas aeruginosa, Pseudomonas alcaligenes, Pseudomonas fluorescens, Pseudomonas putida, Salmonella enteriditis, Salmonella paratyphi, Salmonella typhi, Salmonella typhimurium, Serratia marcescens, Shigella dysenteriae, Shigella jlexneri, Shigella sonnei, Stenotrophomonas maltophilla, Veillonella parvula, Vibrio cholerae, Vibrio parahaemolyticus, Yersinia enterocolitica, Yersinia intermedia, Yersinia pestis and Yersinia pseudotuberculosis.

In one embodiment the bacterial infection being treated is a Gram-positive bacterial strain infection. In one embodiment the Gram-positive bacterial strain is selected from the group consisting of Actinomyces naeslundii, Actinomyces viscosus, Bacillus anthraces, Bacillus cereus, Bacillus subtilis, Clostridium difficile, Corynebacterium diphtheriae, Corynebacterium ulcerans, Enterococcus faecalis, Enterococcus faecium, Micrococcus luteus, Mycobacterium avium, Mycobacterium intracellulare, Mycobacterium leprae, Mycobacterium tuberculosis, Propionibacterium acnes, Staphylococcus aureus, Staphylococcus epidermidis, Staphylococcus haemolyticus, Staphylococcus hominis, Staphylococcus hyicus, Staphylococcus intermedius, Staphylococcus saccharolyticus, Staphylococcus saprophyticus, Streptococcus agalactiae, Streptococcus mutans, Streptococcus pneumoniae, Streptococcus pyogenes, Streptococcus salivarius and Streptococcus sanguis.

The compositions can, if desired, also contain other active therapeutic agents, such as a narcotic, a non-steroid anti-inflammatory drug (NSAID), an analgesic, an anesthetic, a sedative, a local anesthetic, a neuromuscular blocker, an anti-cancer, an antimicrobial (for example, an aminoglycoside, an antifungal, an antiparasitic, an antiviral, a carbapenem, a cephalosporin (e.g., cefepime), a fluoroquinolone, a macrolide, a penicillin, a sulfonamide, a tetracycline, another antimicrobial), an anti-psoriatic, a corticosteriod, an anabolic steroid, a diabetes-related agent, a mineral, a nutritional, a thyroid agent, a vitamin, a calcium-related hormone, an antidiarrheal, an anti-tussive, an anti-emetic, an anti-ulcer, a laxative, an anticoagulant, an erythropoietin (for example, epoetin alpha), a filgrastim (for example, G-CSF, Neupogen), a sargramostim (GM-CSF, Leukine), an immunization, an immunoglobulin, an immunosuppressive (for example, basiliximab, cyclosporine, daclizumab), a growth hormone, a hormone replacement drug, an estrogen receptor modulator, a mydriatic, a cycloplegic, an alkylating agent, an anti-metabolite, a mitotic inhibitor, a radiopharmaceutical, an anti-depressant, an anti-manic agent, an anti-psychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog thereof, dornase alpha (Pulmozyme), a cytokine, or any combination thereof.

In one embodiment the antibacterial agent is selected from quinolones, tetracyclines, glycopeptides, aminoglycosides, β-lactams, rifamycins, macrolides, ketolides, oxazolidinones, coumermycins, and chloramphenicol.

It will be appreciated that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound (or composition thereof) may be at least 51% the absolute stereoisomer depicted. In another embodiment, the compound (or composition thereof) may be at least 60% the absolute stereoisomer depicted. In another embodiment, the compound (or composition thereof) may be at least 80% the absolute stereoisomer depicted. In another embodiment, the compound (or composition thereof) may be at least 90% the absolute stereoisomer depicted. In another embodiment, the compound (or composition thereof) may be at least 95 the absolute stereoisomer depicted. In another embodiment, the compound (or composition thereof) may be at least 99% the absolute stereoisomer depicted.

It will also be appreciated by those skilled in the art that certain compounds of the invention can exist in more than one tautomeric form. For example, a substituent of formula —NH—C(═O)H in a compound of formula (I) could exist in tautomeric form as —N═C(OH)H. The present invention encompasses all tautomeric forms of a compound of formula I as well as mixtures thereof that can exist in equilibrium with non-charged and charged entities depending upon pH, which possess the useful properties described herein

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, fumarate, benzoate, ascorbate, α-ketoglutarate, and α-glycerophosphate. Suitable inorganic salts may also be formed, including hydrochloride, sulfate, nitrate, bicarbonate, and carbonate salts. Salts may be obtained using standard procedures well known in the art, for example by reacting a sufficiently basic compound such as an amine with a suitable acid affording the corresponding anion. Alkali metal (for example, sodium, potassium or lithium) or alkaline earth metal (for example calcium) salts of carboxylic acids can also be made.

Pharmaceutically suitable counterions include pharmaceutically suitable cations and pharmaceutically suitable anions that are well known in the art. Examples of pharmaceutically suitable anions include, but are not limited to those described above (e.g. physiologically acceptable anions) including Cl⁻, Br⁻, I⁻, CH₃SO₃⁻, H₂PO₄⁻, CF₃SO₃⁻, p-CH₃C₆H₄SO₃⁻, citrate, tartrate, phosphate, malate, fumarate, formate, or acetate.

It will be appreciated by those skilled in the art that a compound of the invention comprising a counterion can be converted to a compound of the invention comprising a different counterion. Such a conversion can be accomplished using a variety of well-known techniques and materials including but not limited to ion exchange resins, ion exchange chromatography and selective crystallization.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes. For oral administration, the compounds can be formulated as a solid dosage form with or without an enteric coating.

Thus, the present compounds may be systemically administered, e.g., orally, in combination with a pharmaceutically acceptable vehicle such as an inert diluent, excipient or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 90% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations, particles, and devices.

The active compound may also be administered intravenously or intramuscularly by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina, nanoparticles, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 1 to about 500 mg/kg, e.g., from about 5 to about 400 mg/kg of body weight per day, such as 1 to about 250 mg per kilogram body weight of the recipient per day.

The compound is conveniently formulated in unit dosage form; for example, containing 5 to 500 mg, 10 to 400 mg, or 5 to 100 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Co-administration of a compound disclosed herein with one or more other active therapeutic agents (e.g., antibacterial agents) generally refers to simultaneous or sequential administration of a compound disclosed herein and one or more other active therapeutic agents, such that therapeutically effective amounts of disclosed herein and one or more other active therapeutic agents are both present in the body of the patient.

The ability of a compound disclosed herein to inhibit a bacterial efflux pump can be determined using a method as described in Example 29 and as shown in Table 1.

TABLE 1

| | | Intrinsic | | | |
|---|---|---|---|---|---|
| | | MIC | Enhanced Antibiotic's Activity | | |
| | | of | MIC (μg/mL) (fold increase) with compound at | | |
| | | Compound | 6.25 μg/mL | | |
| Example | Structure | (μg/mL) | Levofloxacin¹ | Ceftazidime² | Doxycycline³ |
| 1 | 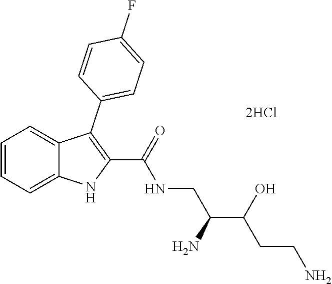 [SMILES: NCCC(O)[C@@H](N)CNC(=O)c1[nH]c2ccccc2c1-c1ccc(F)cc1] | >100 | 0.125 (8) | 2 (1) | 2 (16) |
| 2 | 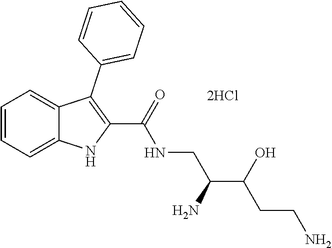 [SMILES: NCCC(O)[C@@H](N)CNC(=O)c1[nH]c2ccccc2c1-c1ccccc1] | >100 | 0.125 (8) | 1 (2) | 2 (16) |
| 2 | 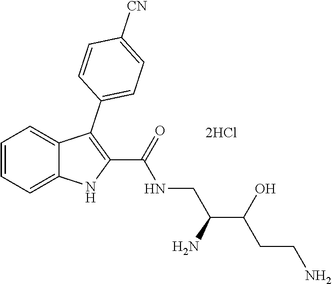 [SMILES: N#Cc1ccc(-c2c(C(=O)NC[C@H](N)C(O)CCN)[nH]c3ccccc23)cc1] | >100 | 0.125 (8) | 2 (1) | 2 (16) |
| 4 | 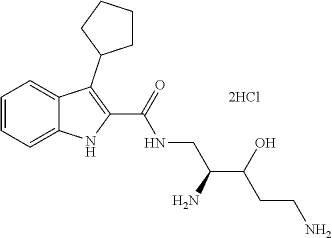 [SMILES: NCCC(O)[C@@H](N)CNC(=O)c1[nH]c2ccccc2c1C1CCCC1] | >100 | 0.063 (32) | 0.5 (4) | 1 (32) |
| 5 | 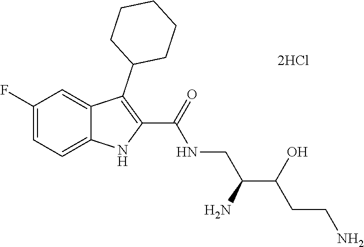 [SMILES: NCCC(O)[C@@H](N)CNC(=O)c1[nH]c2ccc(F)cc2c1C1CCCCC1] | 50 | 0.063 (32) | 0.125 (16) | 1 (32) |
| 6 | 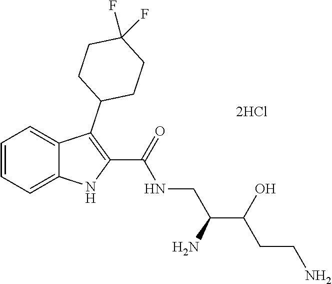 [SMILES: NCCC(O)[C@@H](N)CNC(=O)c1[nH]c2ccccc2c1C1CCC(F)(F)CC1] | 100 | 0.016 (64) | 0.5 (4) | 2 (16) |
| 7 | 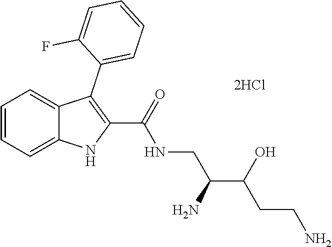 [SMILES: NCCC(O)[C@@H](N)CNC(=O)c1[nH]c2ccccc2c1-c1ccccc1F] | >100 | 0.5 (2) | 2 (1) | 8 (4) |
| 8 | 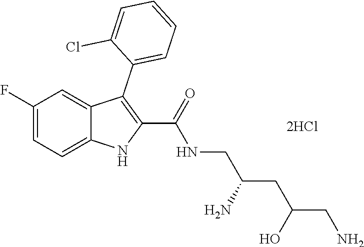 [SMILES: NCC(O)C[C@H](N)CNC(=O)c1[nH]c2ccc(F)cc2c1-c1ccccc1Cl] | >100 | 0.063 (16) | 1 (2) | 2 (16) |
| 9 | 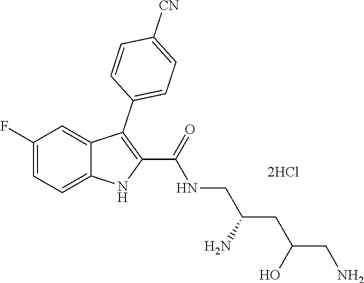 [SMILES: N#Cc1ccc(-c2c(C(=O)NC[C@@H](N)CC(O)CN)[nH]c3ccc(F)cc23)cc1] | >100 | 0.031 (32) | 1 (2) | 1 (32) |
| 10 | 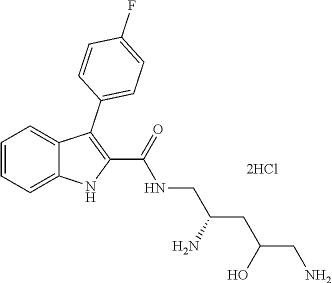 [SMILES: NCC(O)C[C@H](N)CNC(=O)c1[nH]c2ccccc2c1-c1ccc(F)cc1] | 100 | 0.063 (16) | 0.5 (4) | 1 (32) |
| 11 | 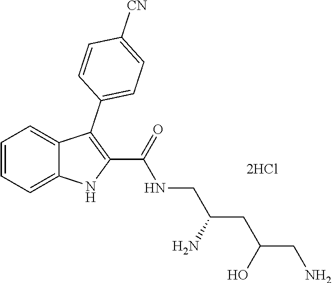 [SMILES: N#Cc1ccc(-c2c(C(=O)NC[C@@H](N)CC(O)CN)[nH]c3ccccc23)cc1] | >100 | 0.063 (16) | 2 (1) | 2 (16) |
| 12 | 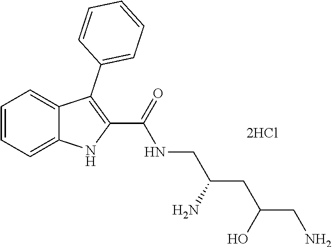 [SMILES: NCC(O)C[C@H](N)CNC(=O)c1[nH]c2ccccc2c1-c1ccccc1] | >100 | 0.063 (16) | 1 (2) | 1 (32) |
| 13 | 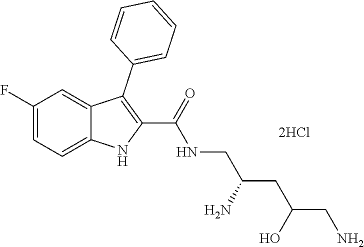 [SMILES: NCC(O)C[C@H](N)CNC(=O)c1[nH]c2ccc(F)cc2c1-c1ccccc1] | >100 | 0.031 (32) | 1 (2) | 1 (32) |
| 14 | 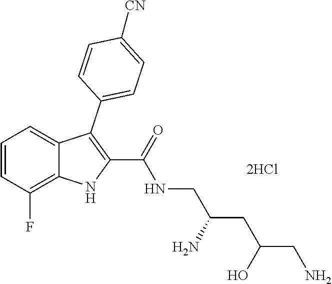 [SMILES: N#Cc1ccc(-c2c(C(=O)NC[C@@H](N)CC(O)CN)[nH]c3c(F)cccc23)cc1] | >100 | 0.031 (32) | 2 (1) | 2 (16) |
| 15 | 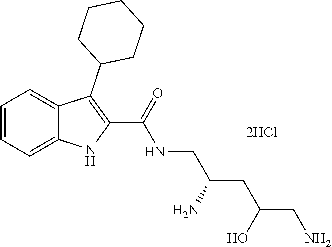 [SMILES: NCC(O)C[C@H](N)CNC(=O)c1[nH]c2ccccc2c1C1CCCCC1] | 50 | 0.031 (32) | 0.5 (4) | 1 (32) |
| 16 | 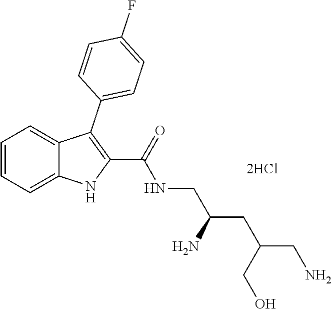 [SMILES: NCC(CO)C[C@@H](N)CNC(=O)c1[nH]c2ccccc2c1-c1ccc(F)cc1] | >100 | 0.063 (16) | 1 (2) | 1 (32) |
| 17 | 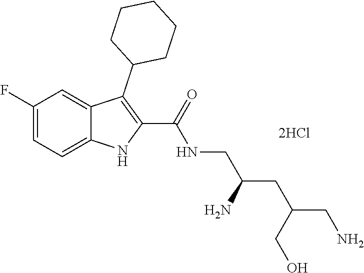 [SMILES: NCC(CO)C[C@@H](N)CNC(=O)c1[nH]c2ccc(F)cc2c1C1CCCCC1] | 25 | 0.063 (32) | 0.25 (8) | 1 (32) |
| 18 | 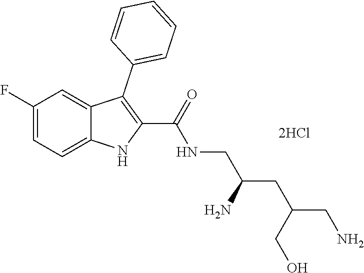 [SMILES: NCC(CO)C[C@@H](N)CNC(=O)c1[nH]c2ccc(F)cc2c1-c1ccccc1] | >100 | 0.063 (32) | ND | ND |
| 19 | 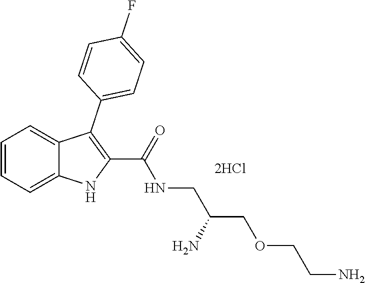 [SMILES: NCCOC[C@H](N)CNC(=O)c1[nH]c2ccccc2c1-c1ccc(F)cc1] | >100 | 0.063 (16) | 1 (2) | 2 (16) |
| 20 | 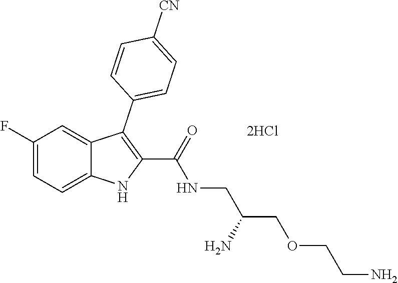 [SMILES: N#Cc1ccc(-c2c(C(=O)NC[C@@H](N)COCCN)[nH]c3ccc(F)cc23)cc1] | >100 | 0.063 (16) | 1 (2) | 2 (16) |
| 21 | 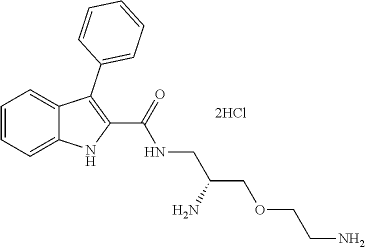 [SMILES: NCCOC[C@H](N)CNC(=O)c1[nH]c2ccccc2c1-c1ccccc1] | >100 | 0.25 (4) | 2 (1) | 2 (16) |
| 22 | 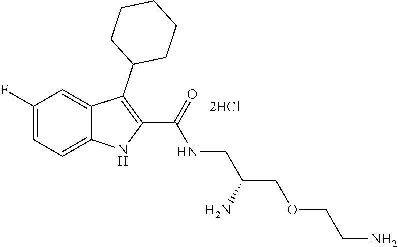 [SMILES: NCCOC[C@H](N)CNC(=O)c1[nH]c2ccc(F)cc2c1C1CCCCC1] | 25 | 0.031 (32) | ND | ND |
| 23 | 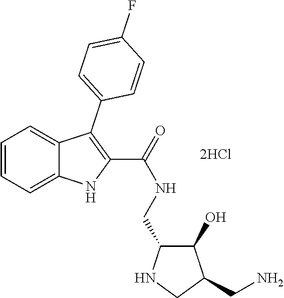 [SMILES: NC[C@H]1CN[C@H](CNC(=O)c2[nH]c3ccccc3c2-c2ccc(F)cc2)[C@H]1O] | >100 | 0.125 (8) | 1 (2) | 2 (16) |
| 24 | 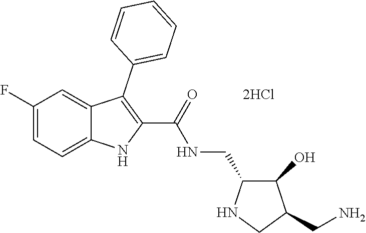 [SMILES: NC[C@H]1CN[C@H](CNC(=O)c2[nH]c3ccc(F)cc3c2-c2ccccc2)[C@H]1O] | >100 | 0.063 (16) | ND | ND |
| 25 | 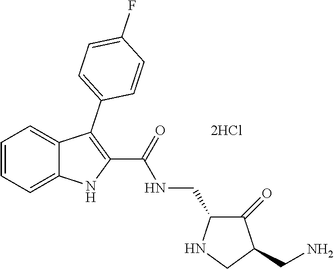 [SMILES: NC[C@H]1CN[C@H](CNC(=O)c2[nH]c3ccccc3c2-c2ccc(F)cc2)C1=O] | >100 | 1 (1) | 2 (1) | 32 (1) |
| 26 | 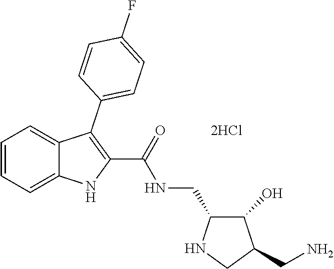 [SMILES: NC[C@H]1CN[C@H](CNC(=O)c2[nH]c3ccccc3c2-c2ccc(F)cc2)[C@@H]1O] | >100 | 0.063 (16) | 1 (2) | 1 (32) |
| 27 | 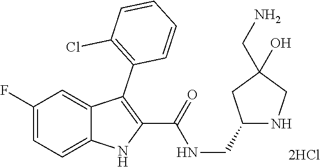 [SMILES: NCC1(O)CN[C@H](CNC(=O)c2[nH]c3ccc(F)cc3c2-c2ccccc2Cl)C1] | >100 | 1 (1) | ND | ND |
| 28 | 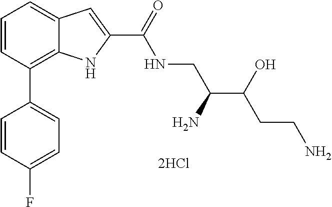 [SMILES: NCCC(O)[C@@H](N)CNC(=O)c1cc2cccc(-c3ccc(F)cc3)c2[nH]1] | 50 | 0.063 (16) | NA | NA |
| 29 | 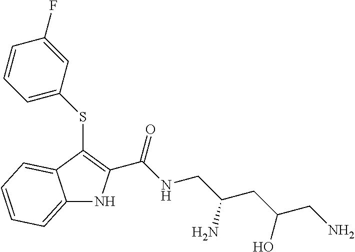 [SMILES: NCC(O)C[C@H](N)CNC(=O)c1[nH]c2ccccc2c1Sc1cccc(F)c1] | 200 | 0.031 (32) | 1 (2) | 0.5 (64) |
| 30 | 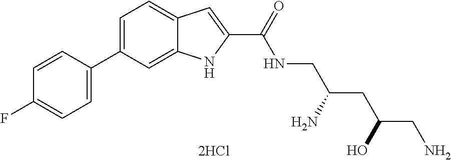 [SMILES: NC[C@@H](O)C[C@H](N)CNC(=O)c1cc2ccc(-c3ccc(F)cc3)cc2[nH]1] | 50 | 0.063 (16) | NA | NA |
| 31 | 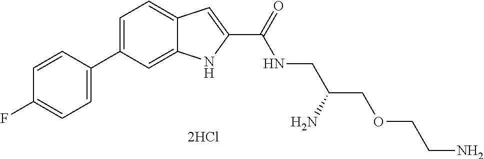 [SMILES: NCCOC[C@H](N)CNC(=O)c1cc2ccc(-c3ccc(F)cc3)cc2[nH]1] | >200 | 0.125 (8) | 0.5 (4) | 1 (32) |
| 32 | 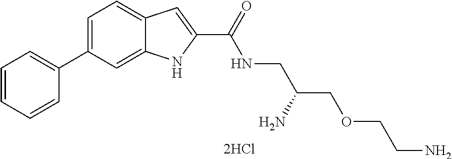 [SMILES: NCCOC[C@H](N)CNC(=O)c1cc2ccc(-c3ccccc3)cc2[nH]1] | >200 | 0.25 (4) | NA | NA |
| 33 | 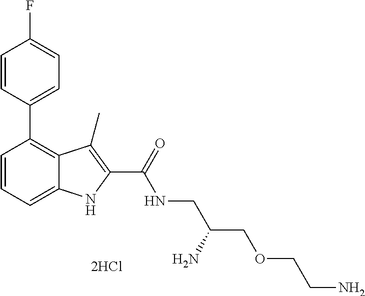 [SMILES: Cc1c(C(=O)NC[C@@H](N)COCCN)[nH]c2cccc(-c3ccc(F)cc3)c12] | 100 | 0.032 (32) | NA | 0.25 (128) |
| 34 | 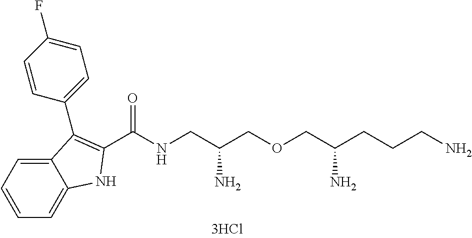 [SMILES: NCCC[C@H](N)COC[C@H](N)CNC(=O)c1[nH]c2ccccc2c1-c1ccc(F)cc1] | >200 | 0.125 (8) | NA | 1 (32) |
| 35 | 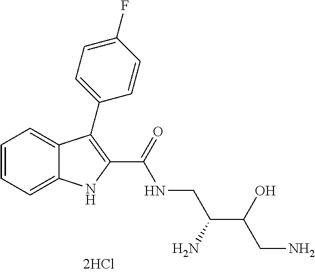 [SMILES: NCC(O)[C@H](N)CNC(=O)c1[nH]c2ccccc2c1-c1ccc(F)cc1] | 200 | 0.25 (4) | NA | NA |
| 36 | 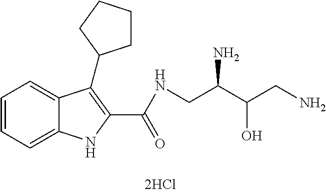 [SMILES: NCC(O)[C@H](N)CNC(=O)c1[nH]c2ccccc2c1C1CCCC1] | 100 | 0.125 (8) | 1 (2) | 1 (32) |

¹These data were generated using Levofloxacin as the antibiotic and the various EPIs against Pseudomonas aeruginosa ATCC 27853.
²These data were generated using Ceftazidime as the antibiotic and the various EPIs against Pseudomonas aeruginosa ATCC 27853.
³These data were generated using Doxycycline as the antibiotic and the various EPIs against Pseudomonas aeruginosa ATCC 27853.

The invention will now be illustrated by the following non-limiting examples.

### Preparation of Amine Intermediates A to I

Table 2 shows amine intermediates that were used to prepare compounds described herein.

TABLE 2 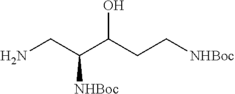 [SMILES: CC(C)(C)OC(=O)NCCC(O)[C@H](CN)NC(=O)OC(C)(C)C]
Intermediate A 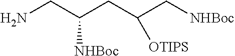
Intermediate B 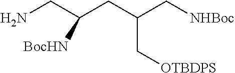
Intermediate C 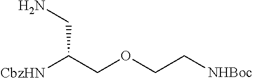 [SMILES: CC(C)(C)OC(=O)NCCOC[C@@H](CN)NC(=O)OCc1ccccc1]
Intermediate D 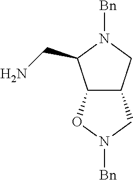 [SMILES: NC[C@@H]1[C@H]2ON(Cc3ccccc3)C[C@H]2CN1Cc1ccccc1]
Intermediate E 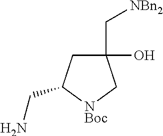
Intermediate F 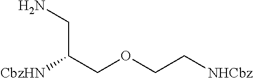 [SMILES: NC[C@H](COCCNC(=O)OCc1ccccc1)NC(=O)OCc1ccccc1]
Intermediate G 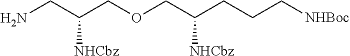 [SMILES: CC(C)(C)OC(=O)NCCC[C@@H](COC[C@@H](CN)NC(=O)OCc1ccccc1)NC(=O)OCc1ccccc1]
Intermediate H 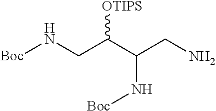
Intermediate I

### Preparation of amine Intermediate A (di-tert-butyl ((4S)-5-amino-3-hydroxypentane-1,4-diyl)dicarbamate)

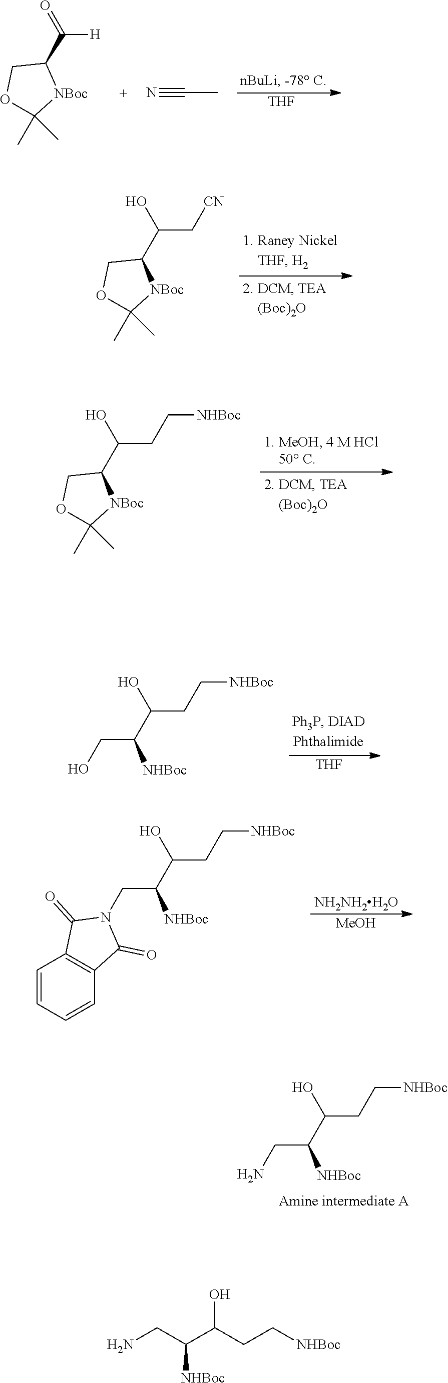

### di-tert-butyl ((4S)-5-amino-3-hydroxypentane-1,4-diyl)dicarbamate

To a solution of di-tert-butyl ((4S)-5-(1,3-dioxoisoindolin-2-yl)-3-hydroxypentane-1,4-diyl)dicarbamate (0.7 g, 1.51 mmol) in MeOH (10 mL) was added hydrazine monohydrate (0.35 mL, 4.53 mmol). The mixture was stirred at 50° C. for 1 h, then cooled to room temperature with stirring for additional 2 h. The reaction mixture was concentrated, and the residue was triturated with CH₂Cl₂. The solid was removed by filtration. The filtrate was diluted with CH₂Cl₂ and washed with brine. The organic layer was dried over Na₂SO₄, filtered and concentrated to afford the desired product (0.46 g, 91% yield) as a white solid. The crude product was used in next step without further purification. ¹H NMR (300 MHz, CDCl₃) δ 5.24 (br s, 1H), 5.03 (br s, 1H), 3.70 (m, 1H), 3.44 (m, 2H), 3.20 (m, 1H), 2.98 (m, 2H), 1.64 (m, 2H), 1.43 (m, 18H).

The requisite intermediates were prepared as shown in the following steps.

### Step 1)

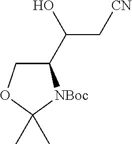

### tert-butyl (4S)-4-(-2-cyano-1-hydroxyethyl)-2,2-dimethyloxazolidine-3-carboxylate

To a solution of acetonitrile (2.08 mL, 40 mmol) in dry THE (50 mL) at −78° C. under N₂ was added n-BuLi (2.5 M in hexanes, 12 mL, 30 mmol) dropwise over 15 min. The reaction mixture was stirred at −78° C. for 1 h. A solution of tert-butyl (S′)-4-formyl-2,2-dimethyloxazolidine-3-carboxylate (5 g, 21.8 mmol) in THE (10 mL) was added dropwise over 10 min. The reaction mixture was stirred at −78° C. for 2 h, then quenched by addition of saturated NH₄Cl. The resulting mixture was stirred at room temperature for 30 min, diluted with EtOAc. The organic solution was washed with brine, dried over Na₂SO₄, filtered, and concentrated to give a crude product (5.5 g, 93% yield) as a light brown liquid. The crude product was used in next step without further purification. ¹H NMR (300 MHz, CDCl₃) δ 5.08 (d, J=4.5 Hz, 1H), 3.79-4.36 (m, 4H), 2.43-2.68 (m, 2H), 1.48-1.61 (m, 15H).

### Step 2)

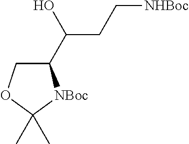

### tert-butyl (4S)-4-(3-((tert-butoxycarbonyl)amino)-1-hydroxypropyl)-2,2-dimethyloxazolidine-3-carboxylate

To a solution tert-butyl (S)-4-((R)-2-cyano-1-hydroxyethyl)-2,2-dimethyloxazolidine-3-carboxylate (3.0 g, 11.1 mmol) in THE (50 mL) was added Raney-Ni (3 g, H₂O was removed by washing with dry THF). The reaction mixture was hydrogenated under hydrogen (55 psi) at room temperature overnight. The solid was removed by passing through a Celite plug. The Celite was washed with CH₂Cl₂. To the filtrate was added TEA (3.1 mL, 22.2 mmol), (Boc)₂O (2.9 g, 13.3 mmol), stirred at room temperature for 3 h, then concentrated to afford a residue. The residue was diluted with EtOAc and washed with H₂O, saturated NaHCO₃, and brine. The organic solution was dried over Na₂SO₄, filtered, and concentrated to give a crude product. The crude product was purified by column chromatography on silica gel using 10-30% EtOAc/Hexanes to afford the desired product (2.9 g, 72% yield) as a white solid. ¹H NMR (300 MHz, CDCl₃) δ 5.12 (br s, 1H), 4.41 (br s, 1H), 4.05 (m, 1H), 3.77 (m, 2H), 3.46 (m, 1H), 3.21 (m, 1H), 1.43-1.58 (m, 26H).

### Step 3)

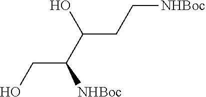 [SMILES: CC(C)(C)OC(=O)NCCC(O)[C@H](CO)NC(=O)OC(C)(C)C]

### di-tert-butyl ((4S)-3,5-dihydroxypentane-1,4-diyl)dicarbamate

To a solution of tert-butyl (4S)-4-(3-((tert-butoxycarbonyl)amino)-1-hydroxypropyl)-2,2-dimethyloxazolidine-3-carboxylate (2.27 g, 6.1 mmol) in MeOH (30 mL) was added HCl (4 M in dioxane, 7.6 mL, 30.4 mmol). The reaction mixture was stirred at 50° C. for 1 h, then concentrated to give a residue. The residue was dissolved in MeOH/CH₂Cl₂ (5 mL/50 mL) to which TEA (3.4 mL, 24.4 mmol) and (Boc)₂O (3.97 g, 18.2 mmol) were added. The reaction mixture was stirred at room temperature for 3 h, then concentrated to afford a residue. The residue was diluted with EtOAc and washed with H₂O, saturated NaHCO₃, and brine. The organic solution was dried over Na₂SO₄, filtered, and concentrated to give a crude product. The crude product was purified by column chromatography on silica gel using EtOAc to afford the desired product (1.88 g, 93% yield) as a white solid. ¹H NMR (300 MHz, CDCl₃) δ 5.49 (d, J=7.8 Hz, 1H), 4.98 (br s, 1H), 4.53 (br s, 1H), 3.09-3.40 (m, 6H), 1.63 (m, 2H), 1.43 (s, 9H), 1.42 (s, 9H).

### Step 4)

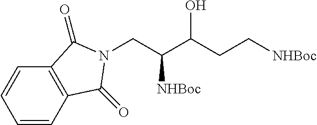 [SMILES: CC(C)(C)OC(=O)NCCC(O)[C@H](CN1C(=O)c2ccccc2C1=O)NC(=O)OC(C)(C)C]

### di-tert-butyl ((4S)-5-(1,3-dioxoisoindolin-2-yl)-3-hydroxypentane-1,4-diyl)dicarbamate

To a solution of di-tert-butyl ((4S)-3,5-dihydroxypentane-1,4-diyl)dicarbamate (1.88 g, 5.30 mmol), triphenylphosphine (1.47 g, 5.63 mmol) and phthalimide (0.83 g, 5.63 mmol) in THE (200 mL) was added DIAD (1.14 mL, 5.63 mmol) at 0° C. The reaction mixture was stirred at 0° C., then warmed to the room temperature overnight. The reaction mixture was concentrated and purified by column chromatography on silica gel using 20-50% EtOAc/hexanes to give the product (0.7 g, 27% yield) as a white solid. ¹H NMR (300 MHz, CDCl₃) δ 7.83 (m, 2H), 7.69 (m, 2H), 5.31 (br s, 1H), 4.92 (br s, 1H), 4.23 (br s, 1H), 3.89 (m, 3H), 3.68 (m, 1H), 3.52 (m, 1H), 3.14 (m, 1H), 1.72 (m, 2H), 1.42 (s, 9H), 1.23 (s, 9H).

### Preparation of amine Intermediate B (di-tert-butyl ((4S)-5-amino-2-Pic ((triisopropylsilyl)oxy)pentane-1,4-diyl)dicarbamate)

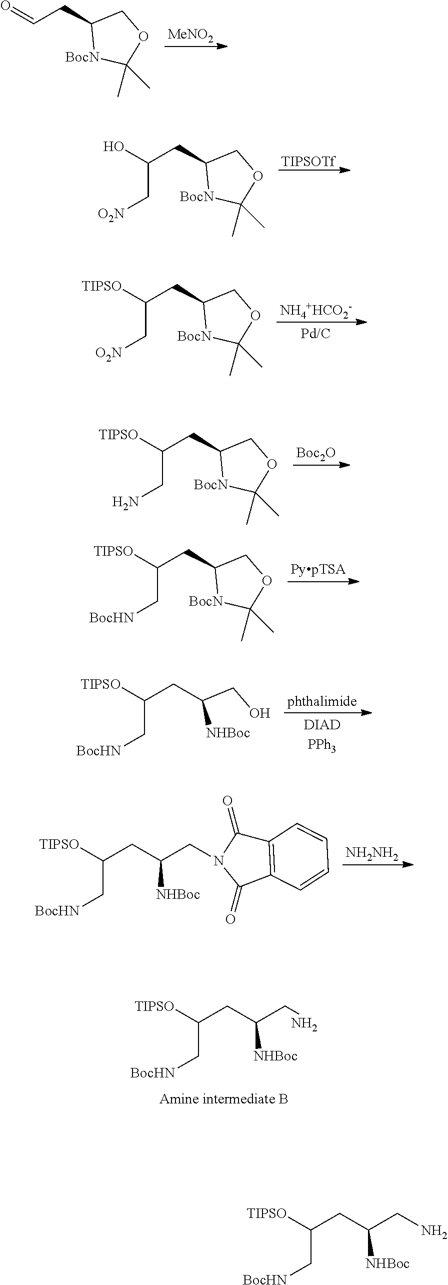

### di-tert-butyl ((4S)-5-amino-2-((triisopropylsilyl)oxy)pentane-1,4-diyl)dicarbamate

To a solution of di-tert-butyl ((4S)-5-hydroxy-2-((triisopropylsilyl)oxy)pentane-1,4-diyl)dicarbamate (3.72 g, 6 mmol) in EtOH (15 mL) was added NH₂NH₂·H₂O (3.0 mL, 62 mmol). It was stirred at 50° C. for 3 hrs and then the white ppt was filtered off and was washed with EtOH. The combined filtrate was concentrated and dissolved in DCM, then washed with water, sat. NaHCO₃ solution and brine. After dried over anhydrous Na₂SO₄, filtered, concentrated it was purified with column chromatography on silica gel to provide the amine as a colorless oil (1.52 g, 52% yield). ¹H NMR (300 MHz, CDCl₃): δ 5.45 (br s, 1H), 5.05 (br s, 1H), 4.79 (br s, 1H), 4.00 (m, 1H), 3.60 (m, 2H), 3.15 (m, 1H), 2.73 (m, 2H), 1.56-1.78 (m, 2H), 1.45 (br, 21H), 1.06 (br, 18H). MS (ESI): Calcd for C₂₄H₅₂N₃O₅Si⁺[M+H]⁺: 490.37, found: 490.25 [M+H]⁺.

The requisite intermediates were prepared as shown in the following steps.

### Step 1)

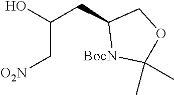 [SMILES: CC(C)(C)OC(=O)N1[C@@H](CC(O)C[N+](=O)[O-])COC1(C)C]

### tert-butyl (4S)-4-(2-hydroxy-3-nitropropyl)-2,2-dimethyloxazolidine-3-carboxylate

To a solution of tert-butyl (S)-2,2-dimethyl-4-(2-oxoethyl)oxazolidine-3-carboxylate (5.0 g, 20.6 mmol) in nitromethane (10 mL, 178.0 mmol) was added TBAF solution (1.0 M in THF, 3 mL, 3 mmol) at 0° C. and the resulting solution was stirred at room temperature overnight. The reaction mixture was diluted with EtOAc, quenched with aq. sat. NH₄Cl solution, washed with water, brine, and then dried over anhydrous Na₂SO₄. The solids were filtered off and solvent was removed in vacuo, the residue was dissolved in DCM and loaded on column chromatography on silica gel. The desired alcohol was collected (6.0 g, 96% yield) as a colorless oil with the eluent of 0-40% of EtOAc in hexane. MS (ESI): Calcd for C₁₃H₂₅N₂O₆⁺305.17 [M+H]⁺, found 305.00 [M+H]⁺.

### Step 2)

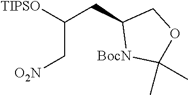

### tert-butyl (4S)-2,2-dim ethyl-4-(3-nitro-2-((triisopropylsilyl)oxy)propyl)oxazolidine-3-carboxylate

To a solution of tert-butyl (4S)-4-(2-hydroxy-3-nitropropyl)-2,2-dimethyloxazolidine-3-carboxylate (6.0 g, 19.7 mmol) in DCM (200 mL) was added 2,6-lutidine (3.26 mL, 28 mmol) and TIPSOTf (7 mL, 26 mmol) dropwise at 0° C. The reaction mixture was stirred at 0° C. to room temperature overnight then quenched with water, extracted with EtOAc, washed with NaHCO₃ solution and NH₄C₁ solution. The combined organic layers were washed with brine, dried over anhydrous Na₂SO₄, filtered off and concentrated in vacuo. The crude product was purified with flash chromatography on silica gel with 0-15% EtOAc in hexane to afford the silyl ether as a colorless oil (7.9 g, 87% yield). ¹H NMR (300 MHz, CDCl₃): δ 4.64-4.87 (m, 1H), 4.42-4.58 (m, 2H), 3.92-4.02 (m, 2H), 3.70-3.88 (m, 1H), 1.96-2.13 (m, 1H), 1.81-1.95 (m, 1H), 1.55 (s, 3H), 1.47 (s, 3H), 1.46 (s, 12H), 1.05 (br, 18H). MS (ESI): Calcd for C₂₂H₄₅N₂O₆Si⁺[M+H]⁺: 461.30, found: 483.20 [M+Na]⁺.

### Step 3)

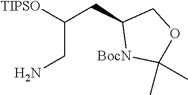

### tert-butyl (4S)-4-(3-amino-2-((triisopropylsilyl)oxy)propyl)-2,2-dimethyloxazolidine-3-carboxylate

To a solution of tert-butyl (4S)-2,2-dimethyl-4-(3-nitro-2-(triisopropylsilyl)oxy)-propyl)oxazolidine-3-carboxylate (7.9 g, 17.16 mmol) in MeOH (90 mL) was added ammonium formate (10.85 g, 171.6 mmol) and palladium on carbon (10%, 2.1 g, 1.97 mmol) under N₂. After stirring the reaction mixture for 1 day at room temperature, the catalyst was removed by filtrating through a pad of Celite and the filtrate was concentrated in vacuo. The residue was purified by silica gel column chromatography using EtOAc as eluent to provide the desired amine as a colorless oil (6.5 g, 88% yield). ¹H NMR (300 MHz, CDCl₃): δ 6.18 (br, 2H), 3.85-4.11 (m, 1H), 3.78-3.83 (m, 2H), 3.42 (dd, J=4.1, 13.0 Hz, 1H), 2.99-3.22 (m, 2H), 1.88-2.10 (m, 2H), 1.53 (s, 3H), 1.46 (br, 15H), 1.05 (br, 18H). MS (ESI): Calcd for C₂₂H₄₇N₂O₄Si⁺[M+H]⁺: 431.33, found: 431.20 [M+H]⁺.

### Step 4)

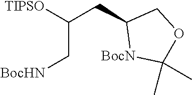

### tert-butyl (4S)-4-(3-((tert-butoxycarbonyl)amino)-2-((triisopropylsilyl)oxy)propyl)-2,2-dimethyloxazolidine-3-carboxylate

To a solution of tert-butyl (4S)-4-(3-amino-2-((triisopropylsilyl)oxy)propyl)-2,2-dimethyloxazolidine-3-carboxylate (6.5 g, 15.1 mmol) in DCM (80 mL) was added Boc₂O (4.94 g, 22.5 mmol) and TEA (4.18 mL, 30 mmol). The reaction mixture was stirred overnight at room temperature Then it was diluted with DCM, washed with water, brine and dried over anhydrous Na₂SO₄. After removing solvent in vacuo, it was purified by silica gel column chromatography with 0-30% EtOAc in hexane as eluents to give a colorless oil as the protected product (7.45 g, 93% yield). ¹H NMR (300 MHz, CDCl₃): δ 5.92 (br s, 1H), 4.06 (m, 1H), 3.94 (m, 2H), 3.83 (m, 2H), 3.03 (m, 1H), 1.78-1.93 (m, 2H), 1.48 (br, 12H), 1.43 (br, 15H), 1.05 (br, 18H).

### Step 5)

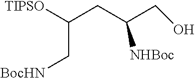

### di-tert-butyl ((4S)-5-hydroxy-2-((triisopropylsilyl)oxy)pentane-1,4-diyl)dicarbamate Pic

To a solution of tert-butyl (4S)-4-(3-((tert-butoxycarbonyl)amino)-2-((triisopropylsilyl)oxy)propyl)-2,2-dimethyloxazolidine-3-carboxylate (7.45 g, 14.1 mmol) in MeOH (50 mL) was added pyridinium p-toluenesulfonate (2.11 g, 8.4 mmol). It was refluxed for 4 hr. MeOH was removed in vacuo and the residue was dissolved in EtOAc, washed with water, brine and dried over anhydrous Na₂SO₄. After concentration, it was purified by column chromatography on silica gel with 0-70% EtOAc in hexane as eluents to give a colorless oil (2.95 g, 43% yield) and 1.15 g starting material was recovered. ¹H NMR (300 MHz, CDCl₃): δ 5.45 (br s, 1H), 5.02 (bs, 1H), 4.09 (m, 1H), 3.75 (m, 1H), 3.59 (m, 2H), 3.31 (m, 1H), 3.15 (m, 1H), 2.80 (br, 1H), 1.62-1.83 (m, 2H), 1.43 (br, 21H), 1.05 (br, 18H). MS (ESI): Calcd for C₂₄H₅₁N₂O₆Si⁺[M+H]⁺: 491.35, found: 491.30 [M+H]⁺.

### Step 6)

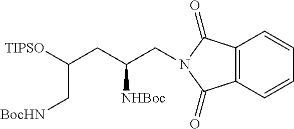

### di-tert-butyl ((4S)-5-(1,3-dioxoisoindolin-2-yl)-2-((triisopropylsilyl)oxy)pentane-1,4-diyl)dicarbamate

To a solution of di-tert-butyl ((4S)-5-hydroxy-2-((triisopropylsilyl)oxy)pentane-1,4-diyl)dicarbamate (2.95 g, 6 mmol) in THF (40 mL) was added triphenylphosphine (2.36 g, 9 mmol), phthalimide (1.33 g, 9 mmol), followed by DIAD (1.65 mL, 8.4 mmol) at 0° C. Then it was stirred at 0° C. to room temperature overnight. The mixture was diluted with EtOAc, washed with water, brine and concentrated under reduced pressure and the residue was purified using column chromatography on silica gel with 0-40% EtOAc in hexane as eluents to give phthalimide product (3.72 g, 99%) as a colorless oil which solidified after standing. ¹H NMR (300 MHz, CDCl₃): δ 7.86 (m, 2H), 7.78 (m, 2H), 6.35 (br, 2H), 4.05 (m, 2H), 3.70 (m, 1H), 3.60 (m, 1H), 3.29 (m, 1H), 3.18 (m, 1H), 1.63-1.82 (m, 2H), 1.43 (s, 3H), 1.27 (br, 9H), 1.24 (br, 9H), 1.06 (br, 18H). MS (ESI): Calcd for C₃₂H₅₄N₃O₇Si⁺[M+H]⁺: 620.37, found: 642.45 [M+Na]⁺.

### Preparation of amine Intermediate C (di-tert-butyl ((4R)-5-amino-2-(((tert-butyldiphenyl silyl)oxy)methyl)pentane-1,4-diyl)dicarbamate)

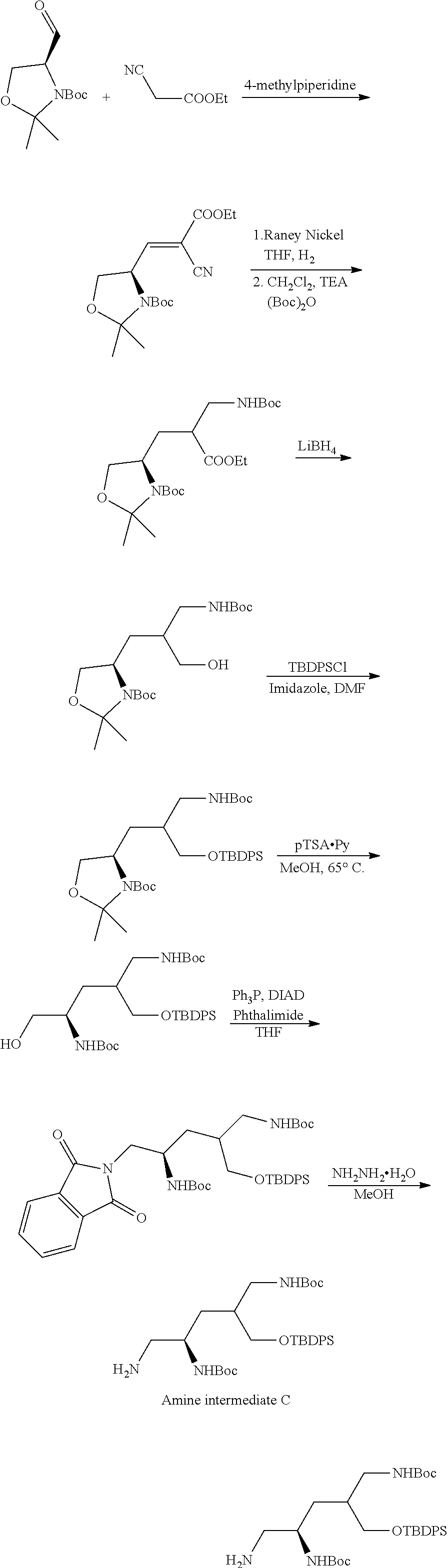

### di-tert-butyl ((4R)-5-amino-2-(((tert-butyldiphenylsilyl)oxy)methyl)pentane-1,4-diyl)dicarbamate

To a solution of di-tert-butyl ((4R)-2-(((tert-butyldiphenylsilyl)oxy)methyl)-5-(1,3-dioxo-2,3-dihydro-1H-inden-2-yl)pentane-1,4-diyl)dicarbamate (2.52 g, 3.52 mmol) in MeOH (50 mL) was added hydrazine monohydrate (0.83 mL, 10.6 mmol). The mixture was stirred at room temperature and the precipitate formed was filtered off and washed with CH₂Cl₂. The filtrate was concentrated and diluted with CH₂Cl₂, washed with saturated NaHCO₃, brine and dried over Na₂SO₄. The organic solution was filtered and concentrated to give a crude product. The crude product was purified on silica gel column chromatography. Elution with EtOAc, then 10% MeOH/CH₂Cl₂ with 1% NH₃·H₂O afforded the product (1.32 g, 54% yield) as a colorless gum. ¹H NMR (300 MHz, CDCl₃) δ 7.64 (m, 4H), 7.41 (m, 6H), 4.65-4.90 (br, 2H), 3.40-3.68 (m, 3H), 3.17 (m, 2H), 2.51-2.70 (m, 2H), 1.75 (br s, 1H), 1.41 (s, 18H), 1.22-1.42 (m, 2H), 1.06 (s, 9H).

The requisite intermediates were prepared as shown in the following steps.

### Step 1)

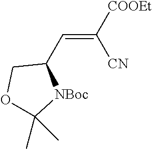

### tert-butyl (4R)-4-(2-cyano-3-ethoxy-3-oxoprop-1-en-1-yl)-2,2-dimethyloxazolidine-3-carboxylate

To a solution of tert-butyl (S)-4-formyl-2,2-dimethyloxazolidine-3-carboxylate (5.0 g, 21.8 mmol), ethyl 2-cyanoacetate (2.47 g, 21.8 mmol) in CH₂Cl₂ (60 mL) at room temperature was added 4-methylpiperidine (216 mg, 2.18 mmol). The reaction mixture was stirred for 2 h then concentrated and purified by column chromatography on silica gel by using 10% EtOAc/hexanes. The mixture of cis- and trans-products (4.95 g, 70% yield) was collected as colorless oil.

### Step 2)

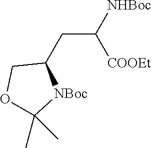

### tert-butyl (4R)-4-(2-(((tert-butoxycarbonyl)amino)methyl)-3-ethoxy-3-oxopropyl)-2,2-dimethyloxazolidine-3-carboxylate

To a solution tert-butyl (4R)-4-(2-cyano-3-ethoxy-3-oxoprop-1-en-1-yl)-2,2-dimethyloxazolidine-3-carboxylate (2.5 g, 7.72 mmol) in THF (40 mL) was added Raney-Ni (2.5 g, H₂O was removed by washing with dry THF). The reaction mixture was hydrogenated under hydrogen (55 psi) at room temperature overnight. The solid was removed by passing through a Celite plug. The Celite was washed with CH₂Cl₂. TEA (1.30 mL, 9.26 mmol), (Boc)₂O (2.02 g, 9.26 mmol) was added to the filtrate and stirred at room temperature for 3 h and then concentrated to afford a residue. The residue was diluted with EtOAc and washed with H₂O, saturated NaHCO₃, and brine. The organic solution was dried over Na₂SO₄, filtered and concentrated to give a crude product. The crude product was purified by column chromatography on silica gel using 10-30% EtOAc/Hexanes to afford the desired product (2.4 g, 72% yield) as a white solid. ¹H NMR (300 MHz, CDCl₃) δ 5.21 (br s, 1H), 4.86 (br s, 1H), 4.08-4.19 (m, 2H), 3.23-4.02 (m, 5H), 2.60 (m, 1H), 1.67 (m, 2H), 1.58 (s, 3H), 1.40-1.55 (m, 21H), 1.21-1.32 (m, 3H).

### Step 3)

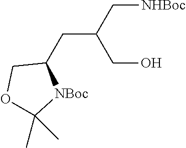

### tert-butyl (4R)-4-(3-((tert-butoxycarbonyl)amino)-2-(hydroxymethyl)propyl)-2,2-dimethyloxazolidine-3-carboxylate

To a solution of tert-butyl (4R)-4-(2-(((tert-butoxycarbonyl)amino)methyl)-3-ethoxy-3-oxopropyl)-2,2-dim ethyloxazolidine-3-carboxylate (4.8 g, 11.2 mmol) in THF (100 mL) at room temperature was added LiBH₄ (730 mg, 33.5 mmol). The reaction mixture was stirred at room temperature overnight then quenched by the addition of acetone in portions and the resulting mixture was concentrated to give a residue. The residue was diluted with EtOAc and washed with H₂O, saturated NaHSO₄, and brine. The organic solution was dried over Na₂SO₄, filtered, and concentrated to give the crude product (4.3 g, 99% yield). It was used directly for next step without further purification. ¹H NMR (300 MHz, CDCl₃) δ 5.65 (br s, 1H), 4.97 (br s, 1H), 3.05-4.22 (m, 7H), 2.06 (m, 1H), 1.33-1.72 (m, 26H).

### Step 4)

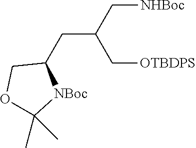

### tert-butyl (4R)-4-(3-((tert-butoxycarbonyl)amino)-2-(((tert-butyldiphenylsilyl)oxy)methyl)propyl)-2,2-dimethyloxazolidine-3-carboxylate

To a solution of tert-butyl (4R)-4-(3-((tert-butoxycarbonyl)amino)-2-(hydroxymethyl)-propyl)-2,2-dim ethyloxazolidine-3-carboxylate (4.3 g, 11.2 mmol) in DMF (50 mL) was added imidazole (1.65 g, 24.2 mmol), and TBDPSCl (3.98 g, 14.5 mmol). The above reaction mixture was stirred at room temperature overnight, then diluted with EtOAc and washed with 15% LiCl solution, saturated NaHCO₃, and brine. The organic layer was dried over Na₂SO₄ and filtered. The filtrate was concentrated and purified using column chromatography on silica gel to give the desired product (6.57 g, 94% yield) as a colorless oil. ¹H NMR (300 MHz, CDCl₃) δ 7.64 (m, 4H), 7.42 (m, 6H), 5.12-5.39 (m, 2H), 2.92-3.90 (m, 7H), 1.29-1.80 (m, 27H), 1.06 (s, 9H).

### Step 5)

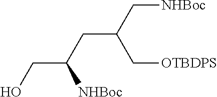

### di-tert-butyl ((4R)-2-(((tert-butyldiphenylsilyl)oxy)methyl)-5-hydroxypentane-1,4-diyl)dicarbamate

To a solution of tert-butyl (4R)-4-(3-((tert-butoxycarbonyl)amino)-2-(((tert-butyldiphenylsilyl)oxy)methyl)propyl)-2,2-dimethyloxazolidine-3-carboxylate (6.57 g, 10.5 mmol) in MeOH (100 mL) was added pyridinium p-toluenesulfonate (1.93 g, 7.68 mmol). The reaction mixture was stirred at 65° C. for 3 h then cooled to room temperature, concentrated, and diluted with EtOAc. The organic solution was washed with water, brine and dried over Na₂SO₄. The crude product was purified by column chromatography on silica gel using 10-50% EtOAc in hexane. A colorless oil was collected (3.10 g, 50% yield). ¹H NMR (300 MHz, CDCl₃) δ 7.64 (m, 4H), 7.43 (m, 6H), 4.64-5.01 (br m, 2H), 3.44-3.70 (m, 5H), 2.97-3.26 (m, 3H), 1.75 (br s, 1H), 1.39-1.42 (m, 20H), 1.07 (s, 9H).

### Step 6)

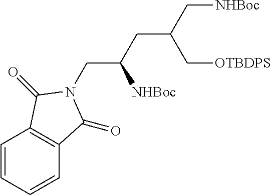

### di-tert-butyl ((4R)-2-(((tert-butyldiphenylsilyl)oxy)methyl)-5-(1,3-dioxoisoindolin-2-yl)pentane-1,4-diyl)dicarbamate

To a solution of di-tert-butyl ((4R)-2-(((tert-butyldiphenylsilyl)oxy)methyl)-5-hydroxypentane-1,4-diyl)dicarbamate (2.52 g, 4.30 mmol), triphenylphosphine (1.24 g, 4.73 mmol) and phthalimide (695 mg, 4.73 mmol) in THE (50 mL) was added DIAD (0.96 mL, 4.73 mmol) at 0° C. The reaction mixture was stirred at 0° C., then room temperature overnight. The reaction mixture was concentrated and purified by column chromatography on silica gel using 5-20% EtOAc/hexanes to give the desired product (2.51 g, 82% yield) as a white solid. ¹H NMR (300 MHz, CDCl₃) δ 7.82 (m, 2H), 7.60-7.71 (m, 6H), 7.35-7.44 (m, 6H), 6.43 (br s, 1H), 4.68-4.84 (m, 1H), 4.00 (m, 1H), 3.65 (m, 3H), 3.13 (m, 2H), 1.59-1.83 (m, 2H), 1.30-1.48 (m, 1H), 1.26 (m, 18H), 1.06 (s, 9H).

### Preparation of amine Intermediate D (benzyl (R)-(1-amino-3-(2-((tert-butoxycarbonyl)amino)ethoxy)propan-2-yl)carbamate)

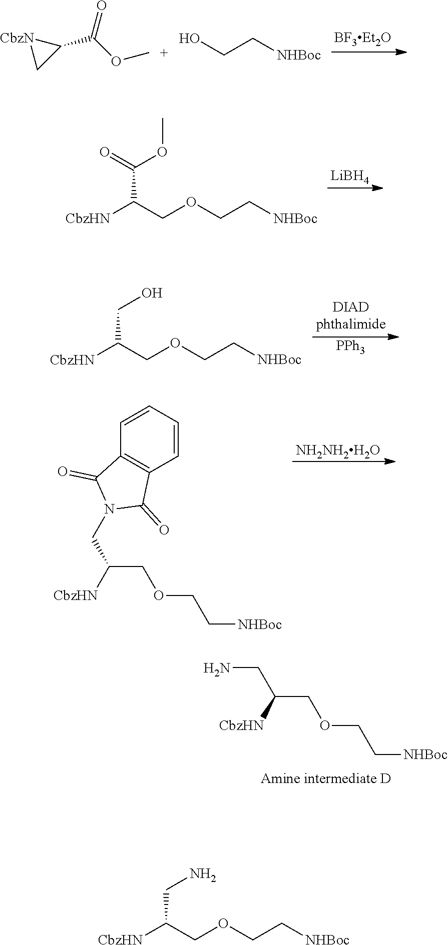

### benzyl (R)-(1-amino-3-(2-((tert-butoxycarbonyl)amino)ethoxy)propan-2-yl)carbamate

To a solution of benzyl (R)-(1-(2-((tert-butoxycarbonyl)amino)ethoxy)-3-(1,3-dioxoisoindolin-2-yl)propan-2-yl)carbamate (525 mg, 1.06 mmol) in EtOH (8 mL) was added NH₂NH₂·H₂O (0.2 mL, 4 mmol). It was stirred at 50° C. for 4 hr, then the white ppt was filtered off and washed with EtOH. The filtrate was concentrated and dissolved in EtOAc, then washed with water, sat. NaHCO₃ solution and brine. After dried over anhydrous Na₂SO₄, it was concentrated and purified by column chromatography on silica gel to provide a colorless oil (330 mg, 85% yield). (300 MHz, CDCl₃) δ 7.27-7.40 (m, 5H), 5.36 (br s, 1H), 5.11 (m, 2H), 4.90 (br, 1H), 3.77 (m, 1H), 3.58 (m, 1H), 3.49 (m, 4H), 3.29 (m, 2H), 2.86 (m, 1H), 1.44 (s, 9H). MS (ESI): Calcd for C₁₈H₃₀N₃O₅⁺368.21 [M+H]⁺, found 368.25 [M+H]⁺.

The requisite intermediates were prepared as shown in the following steps.

### Step 1)

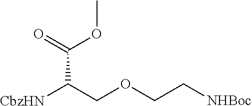

### methyl N-((benzyloxy)carbonyl)-O-(2-((tert-butoxycarbonyl)amino)ethyl)-L-serinate

To a solution of tert-butyl (2-hydroxyethyl)carbamate (3.23 g, 20 mmol) and (S)-1-benzyl 2-methyl aziridine-1,2-dicarboxylate (0.94 g, 4 mmol) in CH₂Cl₂ (20 mL) was added BF₃·Et₂O (0.286 mL, 2.33 mmol) at 0° C. slowly under N₂. The reaction mixture was allowed to warm slowly to room temperature and was stirred overnight. The reaction mixture was partitioned between EtOAc and saturated NaHCO₃. The combined organic phase was washed with brine and dried over Na₂SO₄, filtered, and concentrated. The crude product was purified by column chromatography on silica gel (0-50% EtOAc in hexane) to give methyl N-((benzyloxy)carbonyl)-O-(2-((tert-butoxycarbonyl)amino)ethyl)-L-serinate (0.87 g, 55% yield). ¹H NMR (300 MHz, CDCl₃) δ 7.35 (m, 5H), 5.66 (br s, 1H), 5.13 (s, 2H), 4.79 (br, 1H), 4.51 (m, 1H), 3.77 (s, 3H), 3.74 (m, 2H), 3.48 (m, 2H), 3.27 (m, 2H), 1.45 (s, 3H), 1.43 (s, 6H). MS (ESI): Calcd for C₁₉H₂₉N₂O₇⁺ 397.20 [M+H]⁺, found 397.25 [M+H]⁺.

### Step 2)

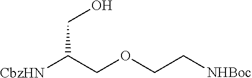

### benzyl (R)-(1-(2-((tert-butoxycarbonyl)amino)ethoxy)-3-hydroxypropan-2-yl)carbamate

To a solution of methyl N-((benzyloxy)carbonyl)-O-(2-((tert-butoxycarbonyl)amino)ethyl)-L-serinate (0.86 g, 2.17 mmol) in THE (10 mL) was added LiBH₄ (95 mg, 4.3 mmol) at room temperature. It was stirred at room temperature overnight and quenched with water, extracted with EtOAc, washed with water, brine and dried over Na₂SO₄. The concentrated crude product was purified by silica gel column chromatography using 0-70% EtOAc in hexane to provide the alcohol (0.54 g, 67% yield) as a colorless oil. MS (ESI): Calcd for C₁₈H₂₉N₂O₆⁺369.20 [M+H]⁺, found 369.20 [M+H]⁺.

### Step 3)

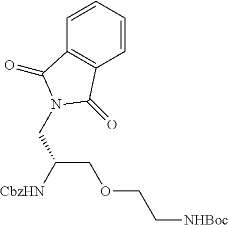 [SMILES: CC(C)(C)OC(=O)NCCOC[C@@H](CN1C(=O)c2ccccc2C1=O)NC(=O)OCc1ccccc1]

### benzyl (R)-(1-(2-((tert-butoxycarbonyl)amino)ethoxy)-3-(1,3-dioxoisoindolin-2-yl)propan-2-yl)carbamate

To a solution of benzyl (R)-(1-(2-((tert-butoxycarbonyl)amino)ethoxy)-3-hydroxypropan-2-yl)carbamate (0.535 g, 1.45 mmol) in THE (15 mL) was added phthalimide (0.255 g, 1.74 mmol) and PPh₃ (0.455 g, 1.74 mmol). Then DIAD (0.34 mL, 1.74 mmol) was added slowly at 0° C. under N₂. It was stirred at 0° C. to room temperature for 5 hrs, then quenched with water, extracted with EtOAc, washed with water and brine, dried and concentrated. The crude product was loaded on a silica gel column and purified with 0-70% EtOAc in hexane to provide the product (0.525 g, 73% yield) as a colorless oil. ¹H NMR (300 MHz, CDCl₃) δ 7.84 (m, 2H), 7.72 (m, 2H), 7.29 (m, 5H), 5.41 (br s, 1H), 4.99 (m, 2H), 4.79 (br, 1H), 4.71 (m, 1H), 3.90 (m, 2H), 3.57 (m, 1H), 3.49 (m, 3H), 3.28 (m, 2H), 1.44 (s, 9H). MS (ESI): Calcd for C₂₆H₃₂N₃O₇ 498.23 [M+H]⁺, found 498.25 [M+H]⁺.

### Preparation of amine Intermediate E (((3aR,6R,6aS)-2,5-dibenzylhexahydro-2H-pyrrolo[3,4-d]isoxazol-6-yl)methanamine)

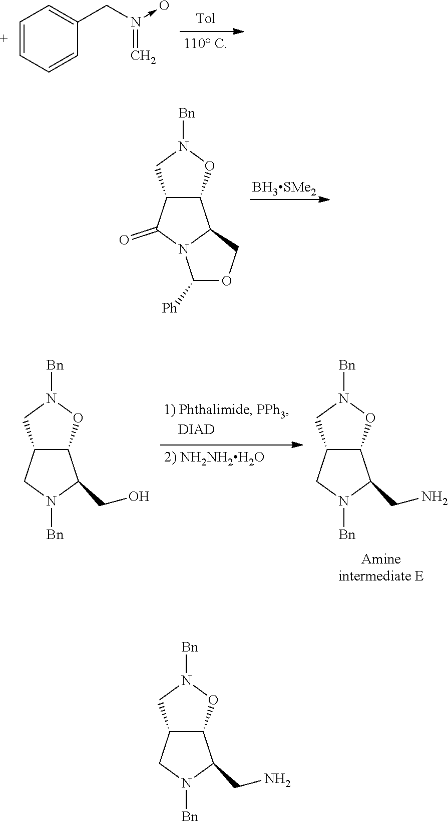

### ((3aR,6R,6aS)-2,5-dibenzylhexahydro-2H-pyrrolo[3,4-d]isoxazol-6-yl)methanamine

To a mixture of ((3aR,6R,6aS)-2,5-dibenzylhexahydro-2H-pyrrolo[3,4-d]isoxazol-6-yl)methanol (1.39 g, 4.3 mmol) in 30 mL of THE was added phthalimide (0.69 g, 4.73 mmol), triphenylphosphine (1.23 g, 4.73 mmol). Then diisopropyl azodicarboxylate (0.96 g, 4.73 mmol) was added at 0° C. Reaction mixture was stirred at room temperature overnight. Solvent was removed and residue was directly purified on column chromatography on silica gel to give 1.37 g of intermediate. To this intermediate (1.37 g, 3 mmol) in methanol (30 mL) was added 1.5 mL of hydrazine monohydrate. Reaction mixture was stirred at 50° C. for 1 hour after which the solvent was removed, residue was diluted with 30 mL of DCM. White solid was formed and filtered off, washed with DCM. The solution was collected and washed with brine and dried over Na₂SO₄. Solvent was removed and residue was purified on column chromatography on silica gel to give the product (0.45 g, 32% yield) as a colorless oil. ¹H NMR (300 MHz, CDCl₃) δ 7.22-7.38 (m, 10H), 4.58 (m, 1H), 4.08 (m, 1H), 3.90 (m, 3H), 3.25 (br, 1H), 3.12 (t, J=8.1 Hz, 1H), 2.78-3.00 (m, 5H), 2.40-2.62 (m, 2H), 2.25 (t, J=8.1 Hz, 1H).

The requisite intermediates were prepared as shown in the following steps.

### Step 1)

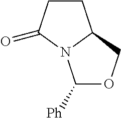 [SMILES: O=C1CC[C@H]2CO[C@H](c3ccccc3)N12]

### (5R,7aR)-5-phenylhexahydro-3H-pyrrolizin-3-one

To a stirred solution of (S)-5-(hydroxymethyl)pyrrolidin-2-one (10.0 g, 87 mmol) in toluene (80 mL) was added benzaldehyde (10.6 g, 100 mmol) and 4-methylbenzenesulfonic acid hydrate (1.0 g, 5 mmol). It was stirred at reflux overnight and water was removed using a Dean-Stark trap. Then solvent was removed in vacuo and the residue was dissolved in DCM and purified by column chromatography on silica gel. Product was collected as a colorless oil (16.6 g, 94%). ¹H NMR (300 MHz, CDCl₃) δ 7.26-7.51 (m, 5H), 6.36 (s, 1H), 4.25 (m, 1H), 4.16 (m, 1H), 3.48 (t, J=7.8 Hz, 1H), 2.82 (m, 1H), 2.58 (m, 1H), 2.39 (m, 1H), 1.98 (m, 1H).

### Step 2)

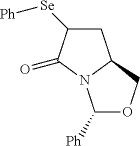 [SMILES: O=C1C([Se]c2ccccc2)C[C@H]2CO[C@H](c3ccccc3)N12]

### (3R,7aS)-3-phenyl-6-(phenyl selanyl)tetrahydro-3H, 5H-pyrrolo[1,2-c]oxazol-5-one

To a stirred solution of LDA (67 mmol, prepared by treatment of diisopropylamine (6.8 g, 67 mmol) in 60 mL of THF with n-Butyllithium (2.5 M in hexanes, 26.8 mL, 67 mmol) at −78° C. under N₂) was added a solution of the (3R,7aS)-3-phenyltetrahydro-3H,5H-pyrrolo[1,2-c]oxazol-5-one (9.7 g, 33 mmol) in 30 mL of THF and it was stirred for 30 min. The resulting enolate was rapidly added to a solution of phenylselenyl bromide (prepared by treatment of diphenyldiselenide (9.85 g, 31.6 mmol) in 30 mL of THF with bromine (4.96 g, 31 mmol) at 0° C. under N₂. After stirring for 15 minutes, reaction mixture was poured into a mixture of HCl (0.5 N, 150 mL, 75 mmol) and extracted with EtOAc. The Combined organic layer was washed with water, Saturated aqueous NaHCO₃ and brine, and dried over Na₂SO₄. After filtration, solvent was removed in vacuo and the residue was purified by column chromatography on silica gel to give the product (4.0 g, 24% yield). ¹H NMR (300 MHz, CDCl₃) δ 7.64 (m, 2H), 7.33 (m, 4H), 7.26 (m, 2H), 7.18 (m, 2H), 6.27 (s, 1H), 3.97 (dd, J₁=4.7, 6.9 Hz, 1H), 3.60 (quint, J=6.5 Hz, 1H), 3.40 (t, J=8.0 Hz, 1H), 2.51 (m, 2H).

### Step 3)

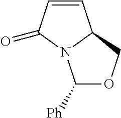 [SMILES: O=C1C=C[C@H]2CO[C@H](c3ccccc3)N12]

### (3R,7aS)-3-phenyl-1,7a-dihydro-3H, 5H-pyrrolo[1,2-c]oxazol-5-one

To a solution of (3R,7aS)-3-phenyl-6-(phenylselanyl)tetrahydro-3H,5H-pyrrolo[1,2-c]oxazol-5-one (4.0 g, 11.1 mmol) in DCM (50 mL) was added pyridine (1.72 mL) and hydrogen peroxide (3.4 mL, 30% in water) at 0° C. Reaction mixture was stirred at room temperature for 2 hours. The mixture was diluted with 50 mL of DCM and 100 mL of 0.5 N HCl, the organic layer was washed with water, saturated aqueous NaHCO₃ and brine, and dried over Na₂SO₄. After filtration, solvent was removed in vacuo and the residue was purified by column chromatography on silica gel to give the product (1.94 g, 86% yield). ¹H NMR (300 MHz, CDCl₃) δ 7.53 (m, 2H), 7.34-7.43 (m, 3H), 7.27 (dd, J=2.0, 5.8 Hz, 1H), 6.19 (m, 1H), 6.17 (dd, J=1.5, 5.8 Hz, 1H), 4.63 (m, 1H), 4.27 (m, 1H), 3.43 (t, J=8.4 Hz, 1H).

### Step 4)

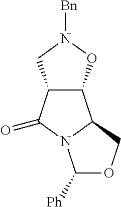 [SMILES: O=C1[C@@H]2CN(Cc3ccccc3)O[C@@H]2[C@H]2CO[C@H](c3ccccc3)N12]

### (3aR,6R,8aR,8bS)-2-benzyl-6-phenylhexahydro-4H,6H-oxazolo[3′,4′:1,5]pyrrolo[3,4-d]isoxazol-4-one

A mixture of (3R,7aS)-3-phenyl-1,7a-dihydro-3H, 5H-pyrrolo[1,2-c]oxazol-5-one (1.6 g, 8 mmol) and N-benzylmethanimine oxide (1.6 g, 12 mmol) (prepared by treating N-benzylhydroxylamine HCl salt with formalin in EtOH in the presence of NaOH) in toluene (10 mL) was stirred at 110° C. overnight. After concentration, the residue was dissolved in small amount of DCM and insoluble solid was filtered off. Solvent was removed and residue was purified by column chromatography on silica gel to give the product as a pale-yellow thick oil (2.4 g, 89% yield). ¹H NMR (300 MHz, CDCl₃) δ 7.20-7.50 (m, 10H), 6.35 (s, 1H), 4.67 (d, J=8 Hz, 1H), 4.24-4.36 (t, J=8 Hz, 1H), 4.02-4.16 (m, 1H), 3.99 (s, 2H), 3.40-3.70 (m, 3H), 2.78-2.96 (m, 1H).

### Step 5)

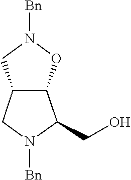 [SMILES: OC[C@@H]1[C@H]2ON(Cc3ccccc3)C[C@H]2CN1Cc1ccccc1]

### ((3aR,6R,6aS)-2,5-dibenzylhexahydro-2H-pyrrolo[3,4-d]isoxazol-6-yl)methanol

To a solution of (3aR,6R,8aR,8bS)-2-benzyl-6-phenylhexahydro-4H,6H-oxazolo[3′,4′:1,5]pyrrolo[3,4-d]isoxazol-4-one (2.4 g, 7.1 mmol) in THF (30 mL) was added borane dimethylsulfide solution (2M in THF, 12 mL, 24 mmol). Reaction mixture was stirred at reflux for 2 hours then cooled to 0° C., to this mixture was slowly added aq. HCl solution (6 M, 12 mL, 72 mmol). Resulting reaction mixture was stirred at 70° C. for 30 minutes then cooled to 0° C. again, 30% aq. NaOH solution was added to adjust the pH to over 10. The mixture was extracted with 3×50 mL of EtOAc. The combined organic layer was washed with brine and dried. Solvent was removed and the residue was purified by column chromatography on silica gel to give the product (1.39 g, 60% yield) as a colorless oil. ¹H NMR (300 MHz, CDCl₃) δ 7.25-7.39 (m, 10H), 4.67 (dd, J=4.8, 8.0 Hz, 1H), 4.09 (d, J=12.9 Hz, H), 3.98 (d, J=12.9 Hz, H), 3.92 (br, 1), 3.84 (dd, J=2.3, 11.3 Hz, 1H), 3.73 (d, J=11.0 Hz, 1H), 3.30 (d, J=12.9 Hz, 1H), 3.18 (t, J=8.7 Hz, 1H), 2.95 (m, 1H), 2.89 (m, 1H), 2.74 (m, 1H), 2.51 (m, 1H), 2.34 (t, J=8.7 Hz, 1H).

### Preparation of amine Intermediate F (tert-butyl (2S)-2-(aminomethyl)-4-((dibenzylamino)methyl)-4-hydroxypyrrolidine-1-carboxylate)

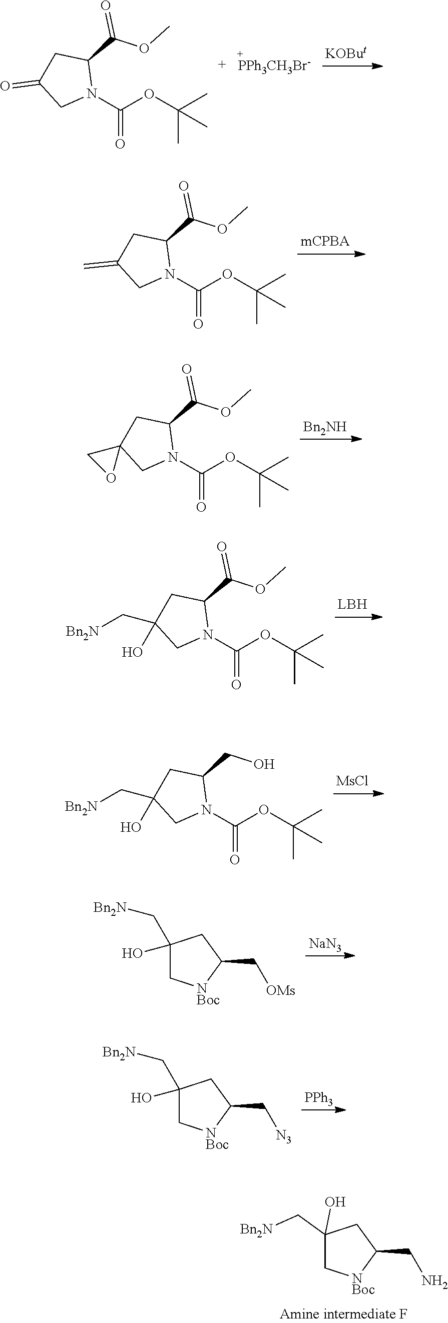

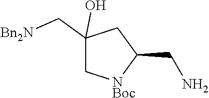

### tert-butyl (2S)-2-(aminomethyl)-4-((dibenzylamino)methyl)-4-hydroxypyrrolidine-1-carboxylate

To a solution of tert-butyl (2S)-2-(azidomethyl)-4-((dibenzylamino)methyl)-4-hydroxypyrrolidine-1-carboxylate (42 mg, 0.1 mmol) in THF (2 mL) was added PPh₃ (50 mg, 0.2 mmol). It was heated at 50° C. for 1 hr. To the reaction mixture was added EtOAc then washed with water, brine and concentrated. It was purified by column chromatography on silica gel (0-100% ethyl acetate in hexane) to give the product as a colorless oil (22 mg, 52% yield). ¹H NMR (300 MHz, CDCl₃) δ 7.36 (m, 4H), 7.31 (m, 4H), 7.25 (m, 2H), 3.92 (d, J=9.4 Hz, 1H), 3.72 (m, 4H), 3.51 (m, 1H), 3.27 (m, 1H), 3.05 (d, J=11.4 Hz, 1H), 2.61 (m, 2H), 2.48 (d, J=13.3 Hz, 1H), 2.42 (br s, 2H), 2.08 (m, 1H), 1.75 (t, J=14.3 Hz, 1H), 1.44 (s, 9H). MS (ESI): Calcd for C₂₆H₃₃N₃O₃⁺426.27 [M+H]⁺, found 426.15 [M+H]⁺.

The requisite intermediates were prepared as shown in the following steps.

### Step 1)

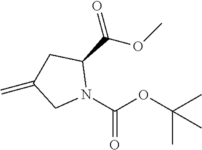 [SMILES: C=C1C[C@@H](C(=O)OC)N(C(=O)OC(C)(C)C)C1]

### 1-(tert-butyl) 2-methyl (S)-4-methylenepyrrolidine-1,2-dicarboxylate

To a suspension of methyltriphenylphosphonium bromide (12 g, 22.6 mmol) in THE (90 mL) was added KOBu^{t} solution in 50 mL THF (3.77 g, 33.6 mmol) at 0° C. under N₂. The reaction mixture was stirred for 2 h at the same temperature and added to a solution of 1-(tert-butyl) 2-methyl (S)-4-oxopyrrolidine-1,2-dicarboxylate (6.05 g, 25.0 mmol) in THE (30 mL) slowly. The reaction was stirred at 0° C. for 1 h and then at room temperature for 3 h. The reaction was quenched with sat. aq. solution of NH₄C₁ and extracted with EtOAc. The combined organics were dried over Na₂SO₄, filtered, concentrated, and purified by column chromatography on silica gel (0-20% EtOAc in hexanes) to provide the product (3.98 g, 66% yield) as a colorless oil. ¹H NMR (300 MHz, CDCl₃) δ 4.97-5.04 (m, 2H), 4.36-4.53 (m, 1H), 4.09 (s, 1H), 4.05 (s, 1H), 3.72 (s, 3H), 2.86-3.05 (m, 1H), 2.56-2.67 (m, 1H), 1.47 (s, 3H), 1.42 (s, 6H). MS (ESI): Calcd for C₁₂H₂₀NO₄⁺242.14 [M+H]⁺, found 242.15 [M+H]⁺.

### Step 2)

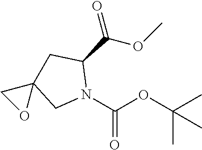 [SMILES: COC(=O)[C@@H]1CC2(CO2)CN1C(=O)OC(C)(C)C]

### 5-(tert-butyl) 6-methyl (6S)-1-oxa-5-azaspiro[2.4]heptane-5,6-dicarboxylate

To a solution of 1-(tert-butyl) 2-methyl (S)-4-methylenepyrrolidine-1,2-dicarboxylate (1.85 g, 7.7 mmol) in DCM (50 mL) was added mCPBA (4.29 g, 77%, 19.1 mmol) at 0° C. The reaction was stirred at 0° C. to room temperature for 8 h. TLC showed starting material was remaining. Another batch of mCPBA (2.00 g, 8.9 mmol) was added. It was stirred at room temperature overnight. The reaction was quenched with 10% aq. Na₂SO₃ solution, extracted with EtOAc. The organic phase was washed with sat. aq. NaHCO₃, water, brine, and dried over Na₂SO₄. After concentration, the residue was purified by column chromatography on silica gel (20-35% EtOAc in hexane) to provide two diastereomers (0.52 g, 26% and 1.39 g, 71% yield) as colorless powder. The relatively polar diastereomer's ¹H NMR was provided. ¹H NMR (300 MHz, CDCl₃) δ 4.42-4.60 (m, 1H), 3.78 (m, 1H), 3.77 (s, 3H), 3.48 (dd, J=12.4, 15.6 Hz, 1H), 2.19 (m, 1H), 2.89 (m, 1H), 2.58-2.73 (m, 1H), 1.91-2.04 (m, 1H), 1.47 (s, 3H), 1.43 (s, 6H). MS (ESI): Calcd for C₁₂H₂₀NO₅⁺258.13 [M+H]⁺, found 257.95[M+H]⁺.

### Step 3)

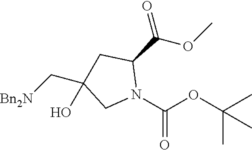

### 1-(tert-butyl) 2-methyl (2S)-4-((dibenzylamino)methyl)-4-hydroxypyrrolidine-1,2-dicarboxylate

To a solution of the epoxide (1.2 g, 4.66 mmol) in ACN (10 mL) was added dibenzylamine (4 mL, 20 mmol) at 0° C. It was stirred at 0° C. to room temperature overnight. Then it was concentrated and loaded on silica gel column chromatography and purified with MeOH in EtOAc to provide the product (0.35 g, 21% yield). ¹H NMR (300 MHz, CDCl₃) δ 7.35 (m, 10H), 4.16 (m, 1H), 3.75 (s, 3H), 3.58 (s, 4H), 3.53 (m, 1H), 3.17 (m, 1H), 2.66 (s, 2H), 2.02 (m, 2H), 1.43 (s, 3H), 1.39 (s, 6H). MS (ESI): Calcd for C₂₆H₃₅N₂O₅⁺455.26 [M+H]⁺, found 455.10 [M+H]⁺.

### Step 4)

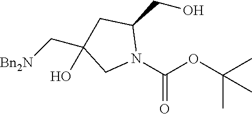

### tert-butyl (2S)-4-((dibenzylamino)methyl)-4-hydroxy-2-(hydroxymethyl)pyrrolidine-1-carboxylate

To a solution of 1-(tert-butyl) 2-methyl (2S)-4-((dibenzylamino)methyl)-4-hydroxypyrrolidine-1,2-dicarboxylate (165 mg, 0.36 mmol) in THE (5 mL) was added LiBH₄ (22 mg, 1 mmol) at room temperature. It was stirred at room temperature overnight and quenched with water, extracted with EtOAc, washed with water, brine and dried over anhydrous Na₂SO₄. The concentrated crude colorless oil was used for next step reaction without further purification (0.15 g, 98% yield). MS (ESI): Calcd for C₂₆H₃₅N₂O₄⁺427.26 [M+H]⁺, found 427.15 [M+H]⁺.

### Step 5)

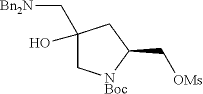

### tert-butyl (2S)-4-((dibenzylamino)methyl)-4-hydroxy-2-(((methyl sulfonyl)oxy)methyl)pyrrolidine-1-carboxylate

To a solution of tert-butyl (2S)-4-((dibenzylamino)methyl)-4-hydroxy-2-(hydroxymethyl)pyrrolidine-1-carboxylate (0.15 g, 0.36 mmol) in DCM (5 mL) was added DIEA (0.12 mL, 0.7 mmol). Then MsCl (0.035 mL, 0.44 mmol) was added at 0° C. It was stirred at 0° C. to room temperature overnight and extracted with EtOAc, washed with water, NaHCO₃, brine and dried over Na₂SO₄. After filtering off, the filtrate was concentrated and loaded on silica gel column with 0-30% EtOAc in hexane as eluents to provide the mesylate (0.15 g, 83% yield). MS (ESI): Calcd for C₂₆H₃₇N₂O₆⁺505.23 [M+H]⁺, found 505.20 [M+H]⁺.

### Step 6)

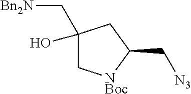

### tert-butyl (2S)-2-(azidomethyl)-4-((dibenzylamino)methyl)-4-hydroxypyrrolidine-1-carboxylate

To a solution of tert-butyl (2S)-4-((dibenzylamino)methyl)-4-hydroxy-2-(((methylsulfonyl)oxy)methyl)pyrrolidine-1-carboxylate (0.15 g, 0.3 mmol) in DMF (2 mL) was added NaN3 under N₂. It was heated at 90° C. overnight. Then the reaction mixture was diluted with EtOAc, washed with water, brine and dried over Na₂SO₄. After concentration it was purified by column chromatography on silica gel to provide the above intermediate (0.045 g, 33% yield). MS (ESI): Calcd for C₂₅H₃₄N₅O₃⁺452.26 [M+H]⁺, found 452.20 [M+H]⁺.

### Preparation of Amine Intermediate G: benzyl (R)-(1-amino-3-(2-(((benzyloxy)carbonyl)amino)ethoxy)propan-2-yl)carbamate

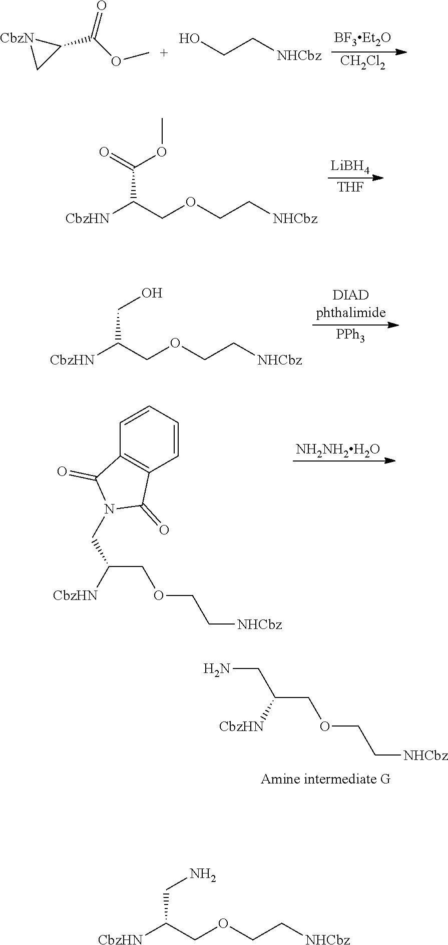

### benzyl (R)-(1-amino-3-(2-(((benzyloxy)carbonyl)amino)ethoxy)propan-2-yl)carbamate

To a solution of benzyl (R)-(1-(2-(((benzyloxy)carbonyl)amino)ethoxy)-3-(1,3-dioxoisoindolin-2-yl)propan-2-yl)carbamate (9.0 g, 16.9 mmol) in EtOH (80 mL) was added NH₂NH₂·H₂O (5 mL, 103 mmol). It was stirred at room temperature overnight, then the white ppt was filtered off and washed with EtOH. The filtrate was concentrated and dissolved in EtOAc, then washed with water, sat. NaHCO₃ solution and brine. After dried over anhydrous Na₂SO₄, it was concentrated and purified by column chromatography on silica gel to provide a colorless oil (5.8 g, 86% yield). (300 MHz, CDCl₃) δ 7.27-7.38 (m, 10H), 5.28 (br s, 1H), 5.20 (br s, 1H), 5.09 (s, 4H), 3.75 (m, 1H), 3.57 (m, 1H), 3.49 (m, 5H), 3.36 (m, 2H). MS (ESI): Calcd for C₂₁H₂₈N₃O₅ 402.21 [M+H]⁺, found 401.95 [M+H]⁺.

The requisite intermediates were prepared as follows:

### Step 1)

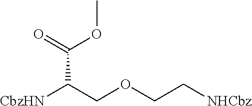

### methyl N-((benzyloxy)carbonyl)-O-(2-(((benzyloxy)carbonyl)amino)ethyl)-L-serinate

To a solution of benzyl (2-hydroxyethyl)carbamate (17.9 g, 92 mmol) and (S)-1-benzyl 2-methyl aziridine-1,2-dicarboxylate (7.2 g, 30.5 mmol) in CH₂Cl₂ (130 mL) was added BF₃·Et₂O (1.92 mL, 15.3 mmol) at 0° C. slowly under N₂. The reaction mixture was allowed to warm slowly to r.t. and was stirred overnight. The reaction mixture was partitioned between DCM and saturated Na₂CO₃. The combined organic phase was washed with brine and dried over anhydrous Na₂SO₄, filtered, and concentrated. The crude product was purified by column chromatography on silica gel (0-50% EtOAc in hexane) to give methyl N-((benzyloxy)carbonyl)-O-(2-(((benzyloxy)carbonyl)amino)ethyl)-L-serinate (10.15 g, 77% yield). ¹H NMR (300 MHz, CDCl₃) δ 7.29-7.43 (m, 10H), 5.63 (br d, 1H), 5.11 (s, 2H), 5.08 (s, 2H), 5.04 (br s, 1H), 4.50 (m, 1H), 3.84 (m, 1H), 3.73 (s, 3H), 3.68 (m, 1H), 3.50 (m, 2H), 3.35 (m, 2H). MS (ESI): Calcd for C₂₂H₂₇N₂O₇ 431.18 [M+H]⁺, found 431.05 [M+H]⁺.

### Step 2)

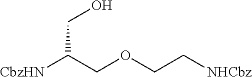

### benzyl (R)-(1-(2-(((benzyloxy)carbonyl)amino)ethoxy)-3-hydroxypropan-2-yl)carbamate

To a solution of methyl N-((benzyloxy)carbonyl)-O-(2-(((benzyloxy)carbonyl)amino)ethyl)-L-serinate (10.15 g, 23.6 mmol) in THE (250 mL) was added LiBH₄ (1.03 g, 47.2 mmol) at r.t. It was stirred at r.t. overnight and quenched with water, extracted with EtOAc, washed with water, brine and dried over anhydrous Na₂SO₄. The concentrated crude product was purified on silica gel column chromatography using 50-80% EtOAc in hexane to provide benzyl (R)-(1-(2-(((benzyloxy)carbonyl)amino)ethoxy)-3-hydroxypropan-2-yl)carbamate (8.25 g, 87% yield) as a colorless oil. MS (ESI): Calcd for C₂₁H₂₇N₂O₆⁺ 403.19 [M+H]⁺, found 403.05 [M+H]⁺.

### Step 3)

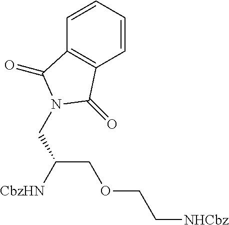 [SMILES: O=C(NCCOC[C@@H](CN1C(=O)c2ccccc2C1=O)NC(=O)OCc1ccccc1)OCc1ccccc1]

### benzyl (R)-(1-(2-(((benzyloxy)carbonyl)amino)ethoxy)-3-(1,3-dioxoisoindolin-2-yl)propan-2-yl)carbamate

To a solution of benzyl (R)-(1-(2-(((benzyloxy)carbonyl)amino)ethoxy)-3-hydroxypropan-2-yl)carbamate (8.23 g, 20.5 mmol) in THE (150 mL) was added phthalimide (4.44 g, 30.2 mmol) and PPh₃ (7.92 g, 30.2 mmol). Then DIAD (5.93 mL, 30.2 mmol) was added slowly at 0° C. under N₂. It was stirred at 0° C. to r.t. overnight, then quenched with water, extracted with EtOAc, washed with water and brine, dried and concentrated. The crude product was loaded on silica gel column chromatography and purified with 0-70% EtOAc in hexane to provide the product (8.0 g, 73% yield) as a colorless oil. MS (ESI): Calcd for C₂₉H₃₀N₃O₇⁺ 532.20 [M+H]⁺, found 532.10 [M+H]⁺.

### Preparation of Amine Intermediate H: benzyl tert-butyl ((S′)-5-((R)-3-amino-2-(((benzyloxy)carbonyl)amino-propoxy)pentane-1,4-diyl)dicarbamate

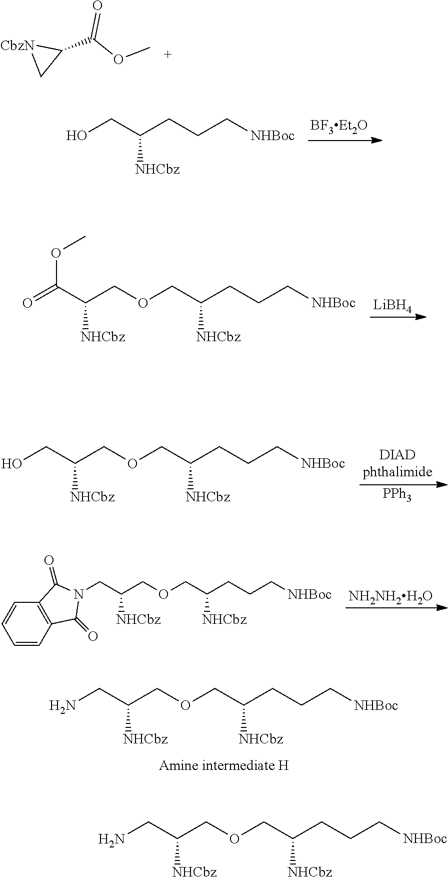

### benzyl tert-butyl ((S)-5-((R)-3-amino-2-(((benzyloxy)carbonyl)amino)propoxy)pentane-1,4-diyl)dicarbamate

To a solution of benzyl tert-butyl ((S)-5-((R)-2-(((benzyloxy)carbonyl)amino)-3-(1,3-dioxoisoindolin-2-yl)propoxy)pentane-1,4-diyl)dicarbamate (140 mg, 0.2 mmol) in EtOH (5 mL) was added NH₂NH₂·H₂O (0.1 mL, 2 mmol). It was stirred at r.t., then the white ppt was filtered off and washed with EtOH. The filtrate was concentrated and dissolved in EtOAc, then washed with water, sat. NaHCO₃ solution and brine. After dried over anhydrous Na₂SO₄, it was concentrated and purified by column chromatography on silica gel to provide a colorless oil (87 mg, 78% yield). MS (ESI): Calcd for C₂₉H₄₃N₄O₇ 559.31 [M+H]⁺, found 559.20 [M+H]⁺

The requisite intermediates were prepared as follows:

### Step 1)

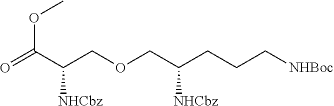 [SMILES: COC(=O)[C@H](COC[C@H](CCCNC(=O)OC(C)(C)C)NC(=O)OCc1ccccc1)NC(=O)OCc1ccccc1]

### methyl N-((benzyloxy)carbonyl)-O—((S)-2-(((benzyloxy)carbonyl)amino)-5-((tert-butoxycarbonyl)amino)pentyl)-L-serinate

To a solution of tert-butyl (2-hydroxyethyl)carbamate (1.4 g, 4 mmol) and (S)-1-benzyl 2-methyl aziridine-1,2-dicarboxylate (0.94 g, 4 mmol) in CH₂Cl₂ (20 mL) was added BF₃·Et₂O (0.13 mL, 1 mmol) at 0° C. slowly under N₂. The reaction mixture was allowed to warm to r.t. and was stirred overnight. The reaction mixture was partitioned between DCM and saturated NaHCO₃. The combined organic phase was washed with brine and dried over anhydrous Na₂SO₄, filtered, and concentrated. The crude product was purified by column chromatography on silica gel (0-50% EtOAc in hexane) to give the product (0.50 g, 21% yield) as a colorless oil. MS (ESI): Calcd for C₃₀H₄₂N₃O₉⁺588.29 [M+H]⁺, found 588.20 [M+H]⁺

### Step 2)

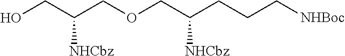 [SMILES: CC(C)(C)OC(=O)NCCC[C@@H](COC[C@@H](CO)NC(=O)OCc1ccccc1)NC(=O)OCc1ccccc1]

### benzyl tert-butyl ((5)-5-((R)-2-(((benzyloxy)carbonyl)amino)-3-hydroxypropoxy)pentane-1,4-diyl)dicarbamate

To a solution of methyl N-((benzyloxy)carbonyl)-O—((S)-2-(((benzyloxy)carbonyl)amino)-5-((tert-butoxycarbonyl)amino)pentyl)-L-serinate (0.46 g, 0.78 mmol) in THE (5 mL) was added LiBH₄ (55 mg, 2.5 mmol) at r.t. It was stirred at r.t. overnight and then quenched with water, extracted with EtOAc, washed with water, brine and dried over Na₂SO₄. The concentrated crude product was purified on a silica gel column chromatography using 0-100% EtOAc in hexane to provide the alcohol (0.155 g, 36% yield) as a colorless oil. ¹H NMR (300 MHz, CDCl₃) δ 7.30-7.40 (m, 10H), 5.64 (br s, 1H), 5.10 (m, 4H), 3.94 (m, 2H), 3.82 (m, 1H), 3.70 (m, 1H), 3.58 (m, 3H), 3.47 (m, 2H), 3.35 (m, 1H), 1.54 (m, 4H), 1.44 (s, 9H). MS (ESI): Calcd for C₂₉H₄₂N₃O₈ 560.30 [M+H]⁺, found 560.20 [M+H]⁺.

### Step 3)

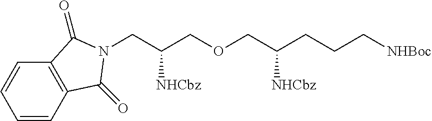 [SMILES: CC(C)(C)OC(=O)NCCC[C@@H](COC[C@@H](CN1C(=O)c2ccccc2C1=O)NC(=O)OCc1ccccc1)NC(=O)OCc1ccccc1]

### benzyl tert-butyl ((S)-5-((R)-2-(((benzyloxy)carbonyl)amino)-3-(1,3-dioxoisoindolin-2-yl)propoxy)pentane-1,4-diyl)dicarbamate

To a solution of benzyl tert-butyl ((S)-5-((R)-2-(((benzyloxy)carbonyl)amino)-3-hydroxypropoxy)pentane-1,4-diyl)dicarbamate (0.155 g, 0.27 mmol) in THE (5 mL) was added phthalimide (0.061 g, 0.4 mmol) and PPh₃ (0.11 g, 0.4 mmol). Then DIAD (0.08 mL, 0.4 mmol) was added slowly at 0° C. under N₂. It was stirred at 0° C. to r.t. for 5 hrs, then quenched with water, extracted with EtOAc, washed with water and brine, dried and concentrated. The crude product was loaded on a silica gel column chromatography and purified with 0-70% EtOAc in hexane to provide the product (0.14 g, 75% yield) as a colorless oil. ¹H NMR (300 MHz, CDCl₃) δ 7.80 (m, 2H), 7.70 (m, 2H), 7.34 (m, 5H), 7.27 (m, 5H), 5.51 (br s, 1H), 5.08 (m, 2H), 4.99 (m, 2H), 4.15 (m, 1H), 3.90 (m, 3H), 3.84 (m, 1H), 3.48 (m, 1H), 3.45 (m, 1H), 3.35 (m, 1H), 3.17 (m, 1H), 3.11 (m, 1H), 1.58 (m, 4H), 1.43 (s, 9H). MS (ESI): Calcd for C₃₇H₄₄N₄NaO₉⁺711.30 [M+Na]⁺, found 711.25 [M+Na]⁺.

### Preparation of amine I: (S)-di-tert-butyl (4-amino-2-((triisopropylsilyl)oxy)butane-1,3-diyl)dicarbamate

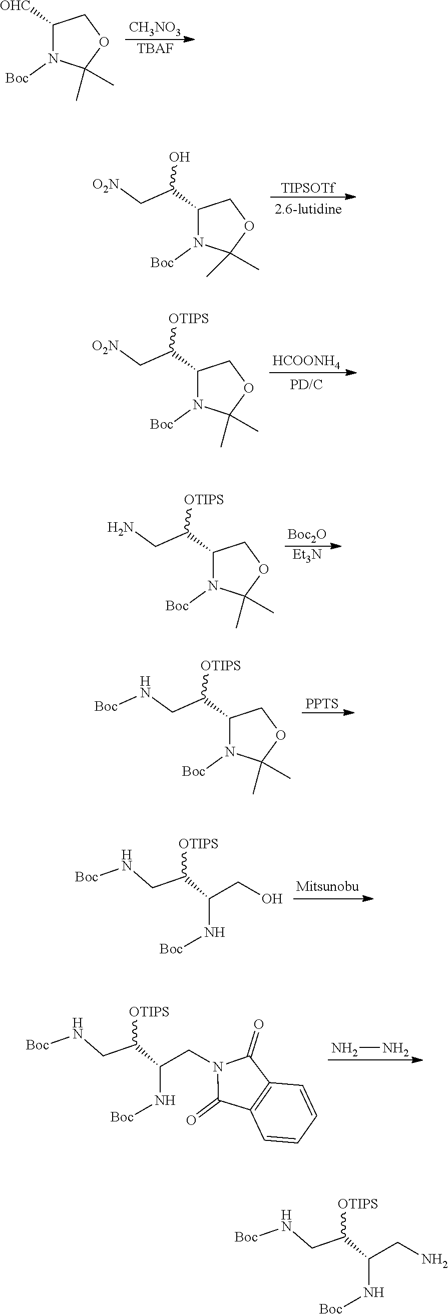

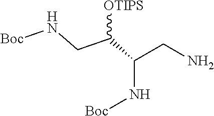

### di-tert-butyl (4-amino-2-((triisopropylsilyl)oxy)butane-1,3-diyl)(S)-dicarbamate

To a solution of 110 mg of (S)-di-tert-butyl (4-(1,3-dioxoisoindolin-2-yl)-2-((triisopropylsilyl) oxy)butane-1,3-diyl)dicarbamate (0.18 mmol) in 2 mL of methanol was added 0.1 mL of hydrazine hydrate. Reaction mixture was stirred at 50° C. for 1 hour then cooled to room temperature. Solvent removed by rotovapor, white solid was filter of and washed with dichloromethane. Combined organic layer washed with water, brine and dried over Na₂SO₄. Solvent removed and residue was purified on column to give 72 mg amine, 83.7%. 1H NMR (300 MHz, CDCl3) δ 5.01-5.18 (br s, 1H), 3.98-4.02 (br s, 1H), 3.52-3.16 (br s, 1H), 3.18-3.26 (m, 1H), 2.73-2.96 (br s, 1H), 1.55-1.93 (br s, 1H), 1.41 (s, 18H), 1.00-1.11 (m, 3H), 1.05 (s, 18H);

The requisite intermediates were prepared as follows:

### Step 1)

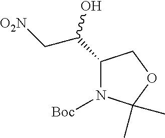 [SMILES: CC(C)(C)OC(=O)N1[C@H](C(O)C[N+](=O)[O-])COC1(C)C]

### tert-butyl (S)-4-(1-hydroxy-2-nitroethyl)-2,2-dimethyloxazolidine-3-carboxylate

To a solution of (S)-tert-butyl 4-formyl-2,2-dimethyloxazolidine-3-carboxylate (5.0 g, 20.6 mmol) in 10 mL of nitromethane (178 mmol) was added 3 mL of TBAF solution (1.0 M/THF, 3 mmol) at 0° C. with stirring. Resulting reaction mixture was stirred at room temperature overnight then diluted with 100 mL of EtOAc. The mixture was continuously washed with 0.5 N HCl, water, aq. Sat. NaHCO₃, brine then dried over Na₂SO₄. Solvent removed and residue was purified on column to give 5.2 g nitro compound as white solid, 82%. 1H NMR (300 MHz, CDCl3) δ 4.78 (br s, 1H), 4.38-4.51 (m, 3H), 3.39-4.19 (m, 3H), 1.58 (s, 3H), 1.56 (s, 3H), 1.49 (s, 9H);

### Step 2)

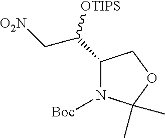

### tert-butyl (S)-2,2-dimethyl-4-(2-nitro-1-((triisopropylsilyl)oxy)ethyl)oxazolidine-3-carboxylate

To solution of (S)-tert-butyl 4-(1-hydroxy-2-nitroethyl)-2,2-dimethyloxazolidine-3-carboxylate (1.2 g, 41.4 mmol) in 30 mL of dichloromethane was added 2,6-lutidine (0.65 mL, 5.6 mmol) and TIPSOTf (1.4 mL, 5.2 mmol) dropwise at 0° C. Reaction mixture was stirred at room temperature overnight then diluted with 30 mL of dichloromethane. The mixture was washed with 0.5 N HCl, water, aq. Sat. NaHCO₃, brine and dried over Na₂SO₄. Solvent removed and residue was purified on column to give 1.6 g product as oil, 86.7%. 1H NMR (300 MHz, CDCl3) δ 4.64 (br s, 1H), 4.39-4.52 (m, 2H), 4.00-4.11 (m, 2H), 3.94 (br s, 1H), 1.48-1.59 (m, 3H), 1.48 (s, 9H), 1.06 (s, 18H).

### Step 3)

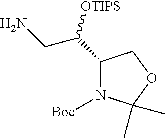

### tert-butyl (S)-4-(2-amino-1-((triisopropylsilyl)oxy)ethyl)-2,2-dimethyloxazolidine-3-carboxylate

To solution of (S)-tert-butyl 2,2-dimethyl-4-(2-nitro-1-((triisopropylsilyl)oxy)ethyl) oxazolidine-3-carboxylate (1.4 g, mmol) in 20 mL of methanol was added 0.5 g of Pd/C (10%) and 2.2 g of HCOONH₄. Reaction mixture was stirred at room temperature overnight then filtered through a peddle of celite, washed with methanol and then concentrated. Residue was purified on column to give 0.66 g product of free amine as oil, 59%. 1H NMR (300 MHz, CDCl3) δ 4.00-4.13 (m, 2H), 3.55-3.90 (m, 2H), 2.60-2.80 (m, 2H), 1.50-1.65 (m, 3H), 1.57 (s, 3H), 1.51 (s, 3H), 1.48 (s, 9H), 1.06 (s, 18H).

### Step 4)

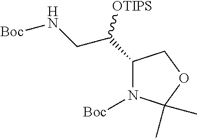

### tert-butyl (S)-4-(3,3-diisopropyl-2,10,10-trimethyl-8-oxo-4,9-dioxa-7-aza-3-silaundecan-5-yl)-2,2-dimethyloxazolidine-3-carboxylate

To a solution of 0.6 g of (S)-tert-butyl 4-(2-amino-1-((triisopropylsilyl)oxy)ethyl)-2,2-dimethyloxazolidine-3-carboxylate (4, 0.6 g, mmol) in 10 mL of dichloromethane was added 0.6 g of triethylamine and 0.35 g of Boc₂O. Reaction mixture was stirred at room temperature overnight then diluted with 20 mL of dichloromethane. The mixture was washed with 0.5 N HCl, water, aq. Sat. NaHCO₃, brine and dried over Na₂SO₄. Solvent removed and residue was purified on column to give 0.42 g protected diamine as colorless oil, 56.4%. 1H NMR (300 MHz, CDCl3) δ 5.22-5.30 (br s, 1H), 4.07-4.32 (m, 1H), 3.89-3.92 (m, 2H), 3.35-3.67 (m, 1H), 3.00-3.18 (m, 1H), 1.51-1.64 (m, 3H), 1.61 (s, 3H), 1.52 (s, 3H), 1.48 (s, 9H), 1.47 (s, 9H), 1.08 (s, 18H).

### Step 5)

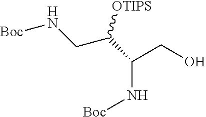

### di-tert-butyl (4-hydroxy-2-((triisopropylsilyl)oxy)butane-1,3-diyl)(S)-dicarbamate

To a solution of 0.42 g of (S)-tert-butyl 4-(3,3-diisopropyl-2,10,10-trimethyl-8-oxo-4,9-dioxa-7-aza-3-silaundecan-5-yl)-2,2-dimethyloxazolidine-3-carboxylate (0.81 mmol) in 10 mL of methanol was added PPTS (121 mg, 0.48 mmol). Reaction mixture was stirred at reflux for 4 hour then cooled to room temperature. The mixture was washed with 0.5 N HCl, water, aq. Sat. NaHCO₃, brine and dried over Na₂SO₄. Solvent removed and residue was purified on column to give 100 mg alcohol as colorless oil, 26.0%. 1H NMR (300 MHz, CDCl3) δ 5.24 (br s, 1H), 4.93 (br s, 1H), 4.03-4.09 (m, 1H), 3.93 (d, J=9 Hz, 1H), 3.60-3.73 (m, 2H), 3.20-3.38 (m, 2H), 3.05 (br s, 1H), 1.41 (s, 9H), 1.39 (s, 9H), 0.99-1.05 (m, 3H), 1.06 (d, J=3 Hz, 18H).

### Step 6)

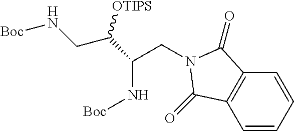

### di-tert-butyl (4-(1,3-dioxoisoindolin-2-yl)-2-((triisopropylsilyl)oxy)butane-1,3-diyl)(S)-dicarbamate

To a solution of 100 mg of (S)-di-tert-butyl (4-hydroxy-2-((triisopropylsilyl)oxy)butane-1,3-diyl)dicarbamate (0.21 mmol) in 10 mL of THE was added isoindoline-1,3-dione (34 mg, 0.23 mmol), triphenylphosphine (30 mg, 0.27 mmol) and (E)-diisopropyl diazene-1,2-dicarboxylate (46 mg, 0.23) at 0° C. Reaction mixture was stirred at room temperature overnight. The mixture was poured into 10 mL of water and extracted with 3×10 mL of EtOAc followed by washing with 0.5 N HCl, water, aq. Sat. NaHCO₃, brine and dried over Na₂SO₄. Solvent removed and residue was purified on column to give 110 mg product as white solid, 86.8%. 1H NMR (300 MHz, CDCl3) δ 7.79-7.85 (m, 2H), 7.65-7.68 (m, 2H), 5.07 (br s, 1H), 4.95 (d, J=9.3 Hz, 1H), 4.05-4.13 (m, 2H), 3.80-3.91 (m, 2H), 3.49 (br s, 1H), 3.22-3.31 (m, 1H), 1.44 (s, 9H, 1.18-1.30 (m, 3H), 1.21 (s, 9H), 1.09 (s, 18H).

### Example 1. Preparation of N-((2S,3R)-2, 5-diamino-3-hydroxypentyl)-3-(4-fluorophenyl)-1H-indole-2-carboxamide hydrogen chloride salt

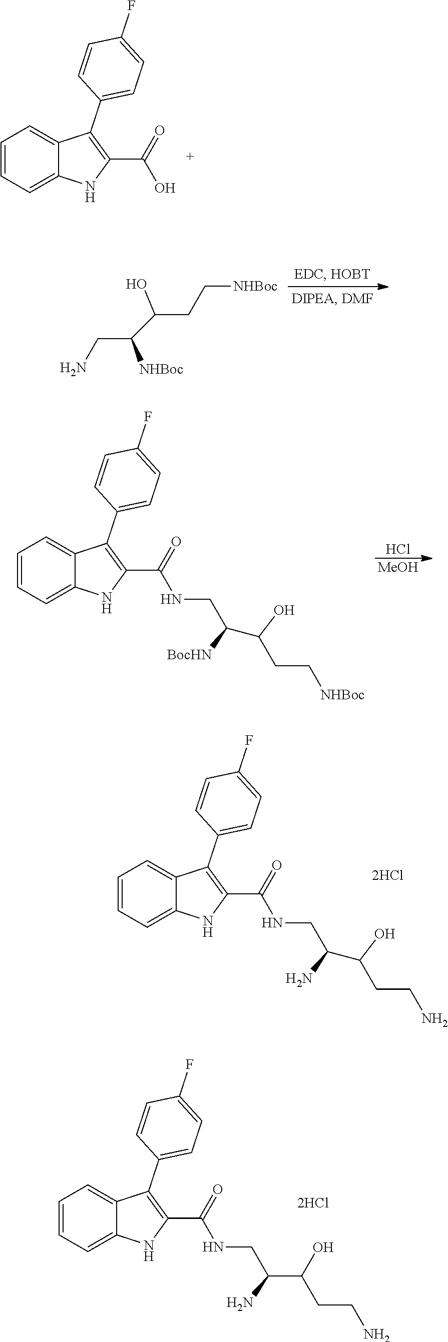

### N-((2S)-2,5-diamino-3-hydroxypentyl)-3-(4-fluorophenyl)-1H-indole-2-carboxamide hydrogen chloride salt

To a solution of di-tert-butyl ((4S)-5-(3-(4-fluorophenyl)-1H-indole-2-carboxamido)-3-hydroxypentane-1,4-diyl)dicarbamate (80 mg, 0.14 mmol) in MeOH (5 mL) was added HCl (4 M in dioxane, 0.5 mL, 2 mmol). The reaction mixture was stirred at 50° C. for 1 h then concentrated to give a residue. The residue was triturated with EtOAc to give the desired product (52 mg, 84% yield) as a yellow solid. ¹H NMR (300 MHz, D₂O) δ 7.51 (m, 4H), 7.33 (m 1H), 7.23 (m, 2H), 7.15 (m, 1H), 3.92 (m, 1H), 3.66 (m, 1H), 3.48 (m, 2H), 3.07 (m, 2H), 1.81 (m, 2H). MS (ESI): Calcd for C₂₀H₂₄FN₄O₂⁺ 371.19 [M+H]⁺, found 371.05 [M+H]⁺.

The requisite intermediate was prepared as shown in the following step.

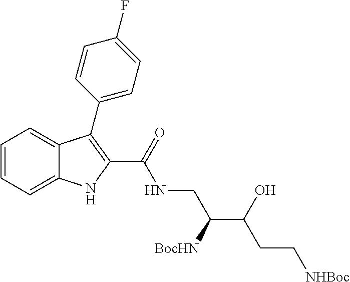 [SMILES: CC(C)(C)OC(=O)NCCC(O)[C@H](CNC(=O)c1[nH]c2ccccc2c1-c1ccc(F)cc1)NC(=O)OC(C)(C)C]

### di-tert-butyl ((4S)-5-(3-(4-fluorophenyl)-1H-indole-2-carboxamido)-3-hydroxypentane-1,4-diyl)dicarbamate

To a solution of 3-(4-fluorophenyl)-1H-indole-2-carboxylic acid (46 mg, 018 mmol) in dry DMF (1 mL) was added DIPEA (0.07 mL, 0.36 mmol), HOBt (15 mg, 0.11 mmol) and EDC (38 mg, 0.21 mmol). The reaction mixture was stirred at room temperature for 10 min then di-tert-butyl ((3R,4S)-5-amino-3-hydroxypentane-1,4-diyl) dicarbamate (A A) (60 mg, 0.18 mmol) was added. The reaction mixture was stirred at room temperature overnight then added H₂O with stirring. A solid was formed and collected by filtration. After air drying the solid was purified by chromatography on silica gel. Elution with 50% EtOAc/hexanes afforded the desired compound (80 mg, 78% yield) as greenish yellow solid. ¹H NMR (300 MHz, CDCl₃) δ 9.70 (br s, 1H), 7.10-7.51 (m, 8H), 6.53 (br s, 1H), 4.94 (br, 2H), 4.50 (br s, 1H), 3.90 (m, 1H), 3.50 (m, 4H), 3.09 (m, 1H), 1.30-1.70 (m, 20H).

### Example 2. Preparation of N-((2S)-2,5-diamino-3-hydroxypentyl)-3-phenyl-1H-indole-2-carboxamide hydrogen chloride salt

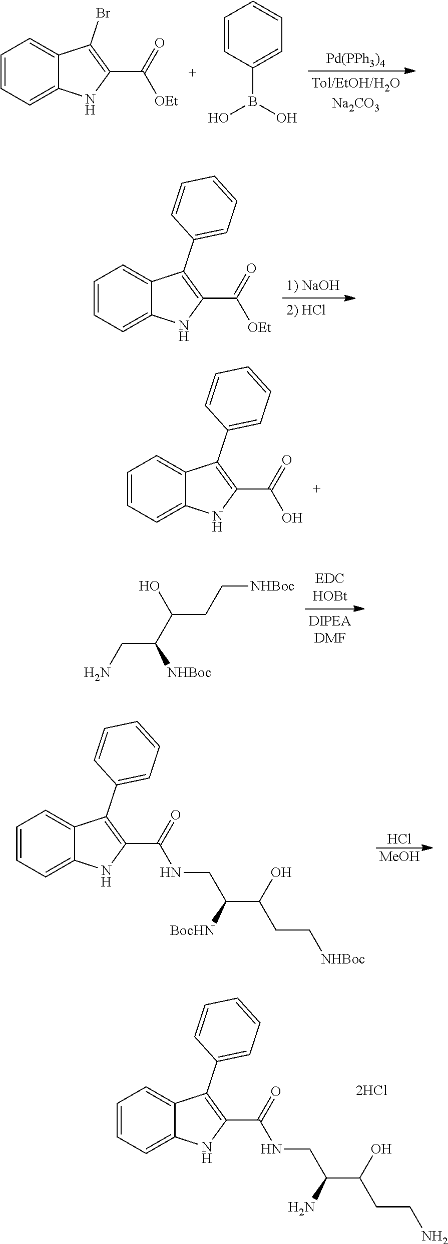

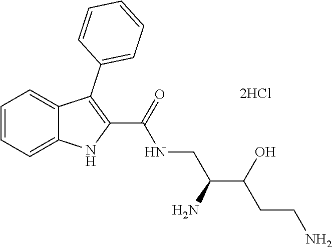 [SMILES: NCCC(O)[C@@H](N)CNC(=O)c1[nH]c2ccccc2c1-c1ccccc1]

### N-((2S)-2,5-diamino-3-hydroxypentyl)-3-phenyl-1H-indole-2-carboxamide hydrogen chloride salt

To a solution of di-tert-butyl ((4S)-3-hydroxy-5-(3-phenyl-1H-indole-2-carboxamido)pentane-1,4-diyl)dicarbamate (73 mg, 0.13 mmol) in MeOH (5 mL) was added HCl (4 M in dioxane, 0.5 mL, 2 mmol). The reaction mixture was stirred at 50° C. for 1 h then concentrated to give a residue. The residue was triturated with EtOAc to give the desired product (38 mg, 68% yield) as off white solid. ¹H NMR (300 MHz, D₂O) δ 7.83 (m, 1H), 7.56 (m, 7H), 7.36 (m, 1H), 7.16 (m, 1H), 3.90 (d, J=9.9 Hz, 1H), 3.63 (m, 1H), 3.50 (m, 2H), 3.05 (m, 2H), 1.82 (m, 2H). MS (ESI): Calcd for C₂₀H₂₅N₄O₂ 353.20 [M+H]⁺, found 353.00 [M+H]⁺.

The requisite intermediates were prepared as shown in the following steps.

### Step 1)

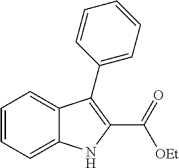 [SMILES: CCOC(=O)c1[nH]c2ccccc2c1-c1ccccc1]

### ethyl 3-phenyl-1H-indole-2-carboxylate

The mixture of ethyl 3-bromo-1H-indole-2-carboxylate (1.35 g, 5 mmol), phenylboronic acid (1.0 g, 8.2 mmol), toluene, ethanol and saturated Na₂CO₃ solution (40/10/10 mL) was degassed and Pd(dppf)Cl₂ (50 mg, 0.068 mmol) was added. The reaction mixture was heated at 100° C. for 1 h, it was extracted with EtOAc and washed with brine and dried over Na₂SO₄, then concentrated. It was purified by column chromatography on silica gel with 0-20% EtOAc in hexane as eluents to give the product (0.63 g, 48% yield) as a white powder.

### Step 2)

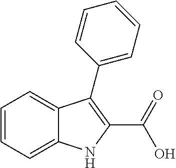 [SMILES: O=C(O)c1[nH]c2ccccc2c1-c1ccccc1]

### 3-phenyl-1H-indole-2-carboxylic acid

To a solution of ethyl 3-phenyl-1H-indole-2-carboxylate (630 mg, 2.37 mmol) in THE (10 mL) was added NaOH solution (2 M, 6 mL). It was heated at 50° C. overnight. THE was removed in vacuo and the residue was acidified with HCl solution. The precipitate was filtered and washed with water. It was dried to provide the product as a pale brown powder (480 mg, 85% yield) which was used for next step reaction without further purification. ¹H NMR (300 MHz, DMSO-d₆) δ 11.55 (br s, 1H), 7.50 (m, 2H), 7.43 (m, 2H), 7.36 (m, 2H), 7.27 (m, 1H), 7.20 (m, 1H), 7.01 (m, 1H). MS (ESI): Calcd for C₁₅H₁₂NO₂⁺ 238.09 [M+H]⁺, found 237.85 [M+H]⁺.

### Step 3)

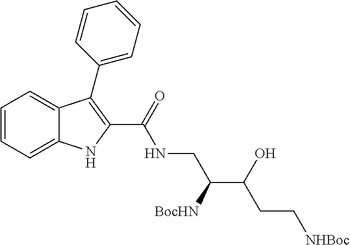 [SMILES: CC(C)(C)OC(=O)NCCC(O)[C@H](CNC(=O)c1[nH]c2ccccc2c1-c1ccccc1)NC(=O)OC(C)(C)C]

### di-tert-butyl ((4S)-3-hydroxy-5-(3-phenyl-1H-indole-2-carboxamido)pentane-1,4-diyl)dicarbamate

To a solution of 3-phenyl-1H-indole-2-carboxylic acid (43 mg, 018 mmol) in dry DMF (1 mL) was added DIPEA (0.07 mL, 0.36 mmol), HOBt (15 mg, 0.11 mmol) and EDC (38 mg, 0.21 mmol). The reaction mixture was stirred at room temperature for 10 min then di-tert-butyl ((3R,4S)-5-amino-3-hydroxypentane-1,4-diyl) dicarbamate (Amine Intermediate A) (60 mg, 0.18 mmol) was added. The reaction mixture was stirred at room temperature overnight then added H₂O with stirring. A solid was formed and was collected by filtration. After air drying the solid was purified by chromatography on silica gel. Elution with 50% EtOAc/hexanes afforded the desired compound (73 mg, 73% yield) as greenish yellow solid. ¹H NMR (300 MHz, CDCl₃) δ 9.73 (br s, 1H), 7.45-7.59 (m, 7H), 7.32 (m, 1H), 7.12 (m, 1H), 6.47 (br s, 1H), 4.99 (br s, 1H), 4.89 (d, J=8.7 Hz, 1H), 4.40 (d, J=4.2 Hz, 1H), 3.31-3.95 (m, 4H), 3.10 (m, 1H), 1.30-1.75 (m, 20H).

### Example 3. Preparation of 3-(4-cyanophenyl)-N-((2S)-2,5-diamino-3-hydroxypentyl)-1H-indole-2-carboxamide hydrogen chloride salt

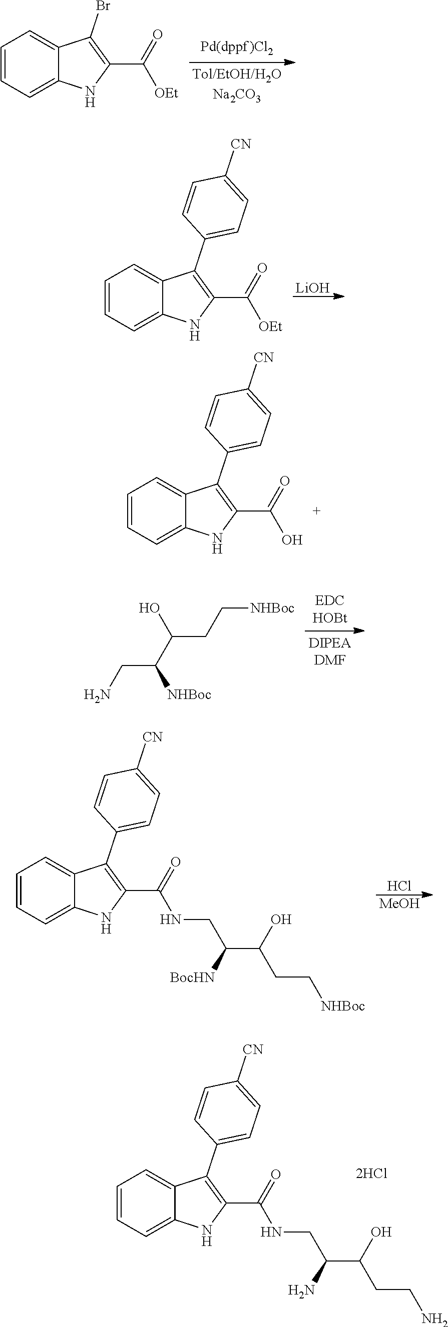

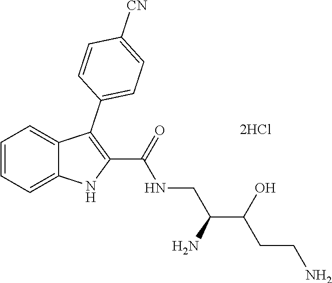 [SMILES: N#Cc1ccc(-c2c(C(=O)NC[C@H](N)C(O)CCN)[nH]c3ccccc23)cc1]

### 3-(4-cyanophenyl)-N-((2S)-2,5-diamino-3-hydroxypentyl)-1H-indole-2-carboxamide hydrogen chloride salt

To a solution of di-tert-butyl ((4S)-5-(3-(4-cyanophenyl)-1H-indole-2-carboxamido)-3-hydroxypentane-1,4-diyl)dicarbamate (72 mg, 0.12 mmol) in MeOH (5 mL) was added HCl (4 M in dioxane, 0.5 mL, 2 mmol). The reaction mixture was stirred at 50° C. for 1 h then concentrated to give a residue. The residue was triturated with EtOAc to give the desired product (35 mg, 63% yield) as yellow solid. ¹H NMR (300 MHz, D₂O) δ 7.80 (d, J 7.5 Hz, 2H), 7.57 (m, 4H), 7.37 (m, 1H), 7.16 (m, 1H), 3.95 (m, 1H), 3.67 (m, 1H), 3.50 (m, 2H), 3.08 (m, 2H), 1.83 (m, 2H). MS (ESI): Calcd for C₂₁H₂₄N₅O₂ 378.19 [M+H]⁺, found 378.05 [M+H]⁺.

The requisite intermediates were prepared as shown in the following steps.

### Step 1)

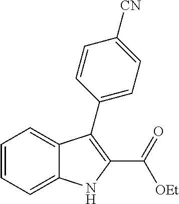 [SMILES: CCOC(=O)c1[nH]c2ccccc2c1-c1ccc(C#N)cc1]

### ethyl 3-(4-cyanophenyl)-1H-indole-2-carboxylate

The mixture of ethyl 3-bromo-5-1H-indole-2-carboxylate (268 mg, 1.0 mmol), (4-cyanophenyl)boronic acid (220 mg, 1.5 mmol), toluene, ethanol and saturated Na₂CO₃ solution (15/4/4 mL) was degassed and Pd(dppf)Cl₂ (50 mg, 0.07 mmol) was added. The reaction mixture was heated at 105° C. overnight, it was extracted with EtOAc and washed with brine and dried over Na₂SO₄, then concentrated. It was purified by column chromatography on silica gel with 0-15% EtOAc in hexane as eluents to give the product (126 mg, 43% yield) as an off-white powder. ¹H NMR (300 MHz, CDCl₃) δ 9.08 (br s, 1H), 7.75 (d, J=6.3 Hz, 2H), 7.68 (d, J=6.3 Hz, 2H), 7.57 (m, 1H), 7.48 (m, 1H), 7.40 (m, 1H), 7.19 (m, 1H), 4.31 (q, J=7.1 Hz, 2H), 1.26 (t, J=7.1 Hz, 3H). MS (ESI): Calcd for C₁₈H₁₉N₂O₂⁺291.11 [M+H]⁺, found 291.20 [M+H]⁺.

### Step 2)

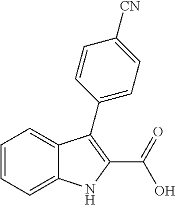 [SMILES: N#Cc1ccc(-c2c(C(=O)O)[nH]c3ccccc23)cc1]

### 3-(4-cyanophenyl)-1H-indole-2-carboxylic acid

To a solution of ethyl 3-(4-cyanophenyl)-1H-indole-2-carboxylate (122 mg, 0.4 mmol) in THE (10 mL) was added LiOH solution (2 M, 2 mL). It was heated at 50° C. until no starting material left. THE was removed in vacuo and the residue was acidified with HCl solution. The precipitate was collected and washed with water. It was dried to provide the acid as a pale brown powder (95 mg, 86% yield) which was used for next step reaction without further purification. Calcd for C₁₆H₁₁N₂O₂⁺ 263.08 [M+H]⁺, found 263.10 [M+H]⁺.

### Step 3)

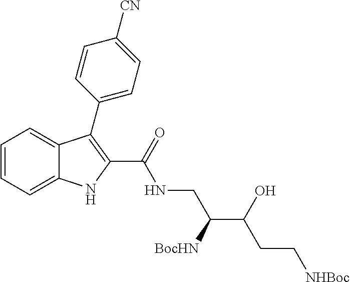 [SMILES: CC(C)(C)OC(=O)NCCC(O)[C@H](CNC(=O)c1[nH]c2ccccc2c1-c1ccc(C#N)cc1)NC(=O)OC(C)(C)C]

### di-tert-butyl ((4S)-5-(3-(4-cyanophenyl)-1H-indole-2-carboxamido)-3-hydroxypentane-1,4-diyl)dicarbamate

To a solution of 3-(4-cyanophenyl)-1H-indole-2-carboxylic acid (47 mg, 018 mmol) in dry DMF (1 mL) was added DIPEA (0.07 mL, 0.36 mmol), HOBt (15 mg, 0.11 mmol) and EDC (38 mg, 0.21 mmol). The reaction mixture was stirred at room temperature for 10 min then di-tert-butyl ((3R,4S)-5-amino-3-hydroxypentane-1,4-diyl) dicarbamate (Amine Intermediate A) (60 mg, 0.18 mmol) was added. The reaction mixture was stirred at room temperature overnight then added H₂O with stirring. A solid was formed and collected by filtration. After air drying the solid was purified by chromatography on silica gel. Elution with 50% EtOAc/hexanes afforded the desired compound (72 mg, 69% yield) as green solid. ¹H NMR (300 MHz, CDCl₃) δ 9.93 (br s, 1H), 7.82 (d, J=7.5 Hz, 2H), 7.65 (d, J=7.5 Hz, 2H), 7.48 (m, 2H), 7.34 (m, 1H), 7.16 (m, 1H), 6.76 (br s, 1H), 5.06 (d, J=8.7 Hz, 1H), 4.86 (br s, 1H), 4.47 (br s, 1H), 3.30-3.73 (m, 4H), 3.03 (m, 1H), 1.30-1.60 (m, 20H).

### Example 4. Preparation of 3-cyclopentyl-N-((2S)-2,5-diamino-3-hydroxypentyl)-1H-indole-2-carboxamide hydrogen chloride salt

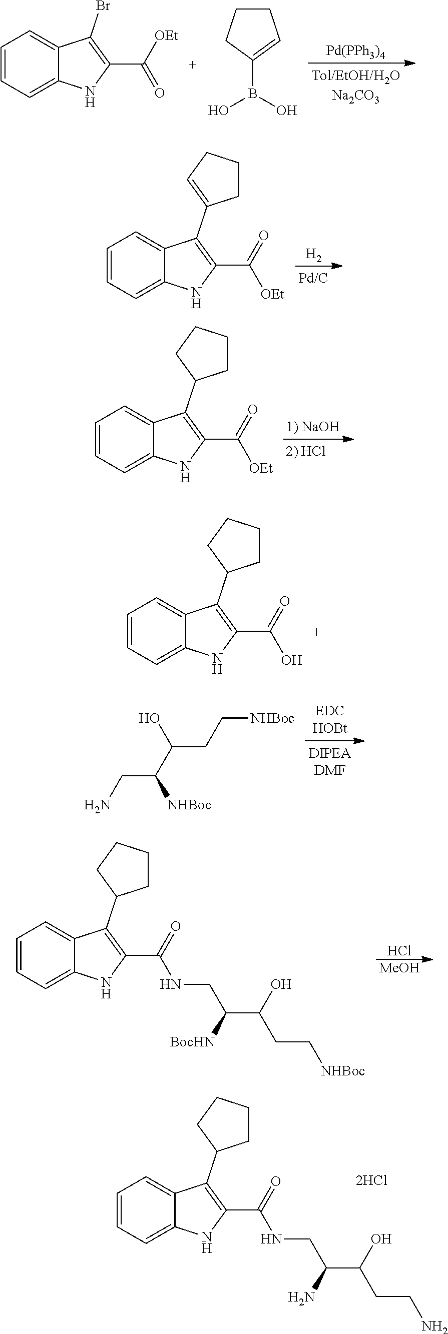

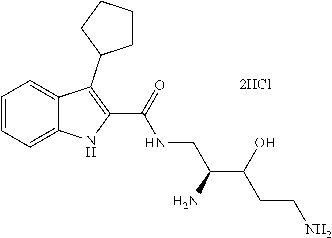 [SMILES: NCCC(O)[C@@H](N)CNC(=O)c1[nH]c2ccccc2c1C1CCCC1]

### 3-cyclopentyl-N-((2S)-2,5-diamino-3-hydroxypentyl)-1H-indole-2-carboxamide hydrogen chloride salt

To a solution of di-tert-butyl ((4S)-5-(3-cyclopentyl-1H-indole-2-carboxamido)-3-hydroxypentane-1,4-diyl)dicarbamate (53 mg, 0.1 mmol) in MeOH (2 mL) was added HCl (4 M in dioxane, 0.5 mL, 2 mmol). The reaction mixture was stirred at 50° C. for 1 h then concentrated to give a residue. The residue was triturated with EtOAc to give the desired product (20 mg, 49% yield) as off white solid. ¹H NMR (300 MHz, D₂O) δ 7.83 (d, J=8.4 Hz, 1H), 7.47 (d, J=8.7 Hz, 1H), 7.30 (m, 1H), 7.11 (m, 1H), 4.06 (m, 2H), 3.78 (m, 1H), 3.59-3.69 (m, 3H), 3.14 (m, 2H), 1.81-2.03 (m, 6H), 1.70 (m, 2H), 1.17 (m, 2H). MS (ESI): Calcd for C₁₉H₂₉N₄O₂ 345.23 [M+H]⁺, found 345.00 [M+H]⁺.

The requisite intermediates were prepared as shown in the following steps.

### Step 1)

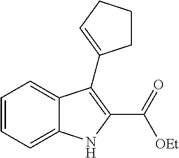 [SMILES: CCOC(=O)c1[nH]c2ccccc2c1C1=CCCC1]

### ethyl 3-(cyclopent-1-en-1-yl)-1H-indole-2-carboxylate

Ethyl 3-bromo-1H-indole-2-carboxylate (1.6 g, 6 mmol) and cyclopent-1-en-1-ylboronic acid (1 g, 8.9 mmol) in a mixture of toluene, ethanol and saturated Na₂CO₃ solution (40/8/8 mL) was degassed and Pd(dppf)Cl₂ (150 mg, 0.2 mmol) was added. The reaction mixture was heated at 100° C. for 4 hours, it was extracted with EtOAc and washed with brine and dried over Na₂SO₄, then concentrated. It was purified by column chromatography on silica gel with 0-20% EtOAc in hexane as eluents to give the product (1.48 g, 97% yield) as a white powder. ¹H NMR (300 MHz, CDCl₃) δ 8.82 (br s, 1H), 7.73 (m, 1H), 7.37 (m, 1H), 7.33 (m, 2H), 7.16 (m, 1H), 5.98 (m, 1H), 4.39 (q, J=7.1 Hz, 2H), 2.81 (m, 2H), 2.62 (m, 2H), 2.08 (m, 2H), 1.41 (t, J=7.1 Hz, 3H).

### Step 2)

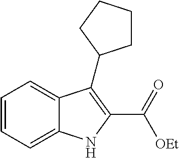 [SMILES: CCOC(=O)c1[nH]c2ccccc2c1C1CCCC1]

### ethyl 3-cyclopentyl-1H-indole-2-carboxylate

To a solution of ethyl 3-(cyclopent-1-en-1-yl)-1H-indole-2-carboxylate (1.48 g, 5.8 mmol) in MeOH (50 mL) was added Pd/C (10 w/w %, 150 mg, 0.14 mmol). It was stirred under H₂ (50 psi) overnight. The catalyst was filter off through a Celite pad and washed with MeOH. The combined filtrate was concentrated and used without further purification (1.40 g, 94%).

### Step 3)

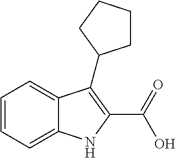 [SMILES: O=C(O)c1[nH]c2ccccc2c1C1CCCC1]

### 3-cyclopentyl-1H-indole-2-carboxylic acid

To a solution of ethyl 3-cyclopentyl-1H-indole-2-carboxylate (1.4 g, 5.4 mmol) in THE (10 mL) was added NaOH solution (2 M, 20 mL). It was heated at 50° C. overnight. THE was removed in vacuo and the residue was acidified with HCl solution. The precipitate was filtered and washed with water. It was dried to provide the acid as a pale brown powder (0.56 g, 45% yield) which was used for next step reaction without further purification. MS (ESI): Calcd for C₁₄H₁₆NO₂⁺ 230.12 [M+H]⁺, found 229.85 [M+H]⁺.

### Step 4)

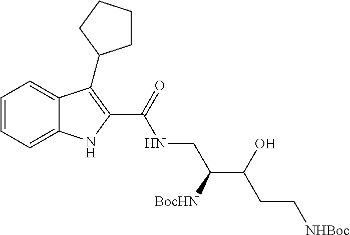 [SMILES: CC(C)(C)OC(=O)NCCC(O)[C@H](CNC(=O)c1[nH]c2ccccc2c1C1CCCC1)NC(=O)OC(C)(C)C]

### di-tert-butyl ((4S)-5-(3-cyclopentyl-1H-indole-2-carboxamido)-3-hydroxypentane-1,4-diyl)dicarbamate

To a solution of 3-cyclopentyl-1H-indole-2-carboxylic acid (39 mg, 018 mmol) in dry DMF (1 mL) was added DIPEA (0.07 mL, 0.36 mmol), HOBt (15 mg, 0.11 mmol) and EDC (38 mg, 0.21 mmol). The reaction mixture was stirred at room temperature for 10 min then di-tert-butyl ((3R,4S)-5-amino-3-hydroxypentane-1,4-diyl) dicarbamate (Amine Intermediate A) (60 mg, 0.18 mmol) was added. The reaction mixture was stirred at room temperature overnight then added H₂O with stirring. A solid was formed and collected by filtration. After air drying the solid was purified by chromatography on silica gel. Elution with 50% EtOAc/hexanes afforded the desired compound (53 mg, 54% yield) as yellow solid. ¹H NMR (300 MHz, CDCl₃) δ 9.15 (br s, 1H), 7.73 (d, J=8.1 Hz, 2H), 7.34 (d, J=8.4 Hz, 1H), 7.23 (m, 1H), 7.06 (m, 1H), 5.39 (d, J=6.3 Hz, 1H), 5.16-5.18 (br, 1H), 4.93 (br, 1H), 4.83 (br s, 1H), 3.40-3.95 (m, 6H), 3.10 (m, 1H), 1.30-2.15 (m, 28H).

### Example 5. Preparation of 3-cyclohexyl-N-((2S)-2,5-diamino-3-hydroxypentyl)-1H-indole-2-carboxamide hydrogen chloride salt

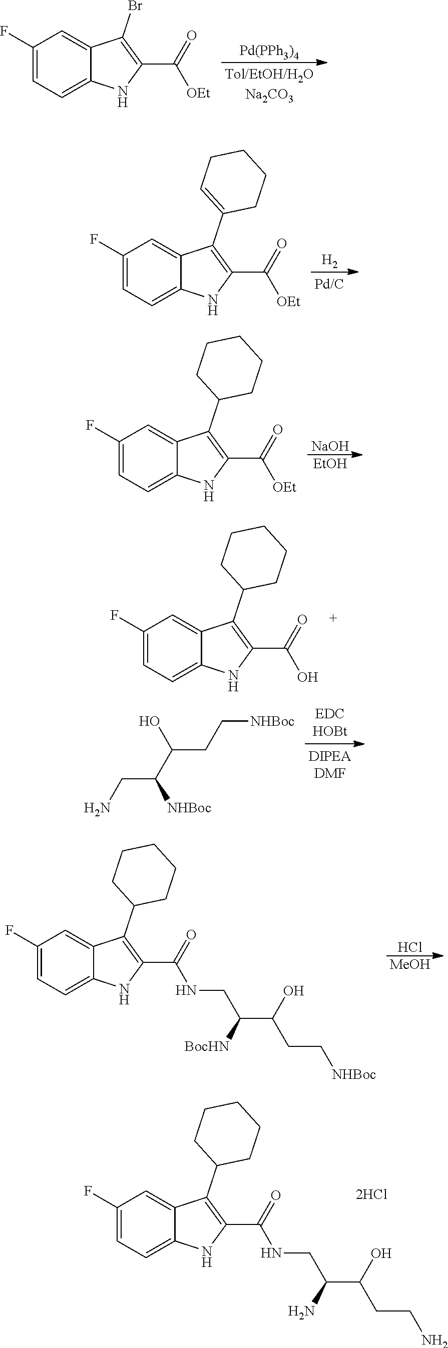

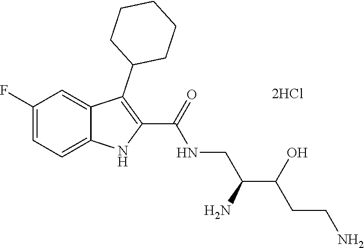 [SMILES: NCCC(O)[C@@H](N)CNC(=O)c1[nH]c2ccc(F)cc2c1C1CCCCC1]

### 3-cyclohexyl-N-((2S)-2,5-diamino-3-hydroxypentyl)-5-fluoro-1H-indole-2-carboxamide hydrogen chloride salt

To a solution of di-tert-butyl ((4S)-5-(3-cyclohexyl-5-fluoro-1H-indole-2-carboxamido)-3-hydroxypentane-1,4-diyl)dicarbamate (30 mg, 0.05 mmol) in MeOH (2 mL) was added HCl (4 M in dioxane, 0.3 mL, 1.2 mmol). The reaction mixture was stirred at 50° C. for 1 h then concentrated to give a residue. The residue was triturated with EtOAc to give the desired product (17 mg, 74% yield) as a brown solid. ¹H NMR (300 MHz, D₂O) δ 7.68 (d, J 10.8 Hz, 1H), 7.43 (dd, J=4.5, 8.7 Hz, 1H), 7.10 (m, 1H), 4.08 (m, 1H), 3.80 (m, 1H), 3.61 (m, 2H), 3.10-3.32 (m, 3H), 1.68-2.06 (m, 8H), 1.35 (m, 4H). MS (ESI): Calcd for C₂₀H₃₀FN₄O₂⁺ 377.23 [M+H]⁺, found 377.05 [M+H]⁺.

The requisite intermediates were prepared as shown in the following steps.

### Step 1)

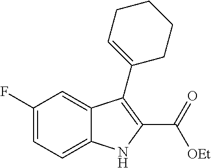 [SMILES: CCOC(=O)c1[nH]c2ccc(F)cc2c1C1=CCCCC1]

### ethyl 3-(cyclohex-1-en-1-yl)-5-fluoro-1H-indole-2-carboxylate

Ethyl 3-bromo-5-fluoro-1H-indole-2-carboxylate (650 mg, 2.3 mmol) and cyclohex-1-en-1-ylboronic acid (350 mg, 2.4 mmol) in a mixture of toluene, ethanol and saturated Na₂CO₃ solution (20/6/6 mL) was degassed and Pd(dppf)Cl₂ (100 mg, 0.14 mmol) was added to the reaction mixture. It was heated at 100° C. overnight and extracted with EtOAc, washed with brine and concentrated. Then it was purified by column chromatography on silica gel to give the product (400 mg, 61% yield) as an off-white powder. ¹H NMR (300 MHz, CDCl₃) δ 8.79 (br s, 1H), 7.30 (m, 1H), 7.27 (m, 1H), 7.09 (dt, J=2.5, 8.9 Hz, 1H), 5.78 (m, 1H), 4.38 (q, J=7.1 Hz, 2H), 2.35 (m, 2H), 2.26 (m, 2H), 1.78 (m, 4H), 1.41 (t, J=7.1 Hz, 3H).

### Step 2)

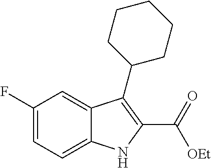 [SMILES: CCOC(=O)c1[nH]c2ccc(F)cc2c1C1CCCCC1]

### ethyl 3-cyclohexyl-1H-indole-2-carboxylate

To a solution of ethyl 3-(cyclohex-1-en-1-yl)-1H-indole-2-carboxylate (0.2 g, 0.7 mmol) in MeOH was added Pd/C (10 w/w %, 50 mg, 0.05 mmol). It was stirred under H₂ (55 psi) overnight. The catalyst was filter off through a Celite pad and washed with MeOH. The combined filtrate was concentrated and used without further purification (0.19 g, 95%). MS (ESI): Calcd for C₁₇H₁₉FNO₂·288.14 [M−H]⁻, found 287.95 [M−H]⁻.

### Step 3)

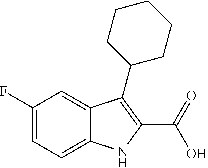 [SMILES: O=C(O)c1[nH]c2ccc(F)cc2c1C1CCCCC1]

### 3-(cyclohex-1-en-1-yl)-5-fluoro-1H-indole-2-carboxylic acid

To a solution of ethyl 3-cyclohexyl-1H-indole-2-carboxylate (200 mg, 0.7 mmol) in THE (10 mL) was added NaOH solution (2 M, 10 mL). It was heated at 50° C. overnight. THE was removed in vacuo and the residue was acidified with HCl solution. The precipitate was filtered and washed with water. It was dried to provide the product as a pale brown powder (116 mg, 63% yield) which was used for next step reaction without further purification. MS (ESI): Calcd for C₁₅H₁₃FNO₂⁺ 260.11 [M+H]⁺, found 260.05 [M+H]⁺.

### Step 4)

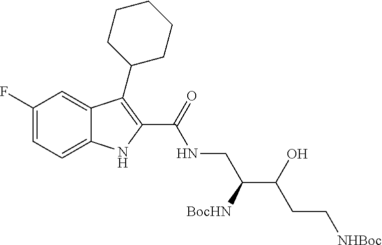 [SMILES: CC(C)(C)OC(=O)NCCC(O)[C@H](CNC(=O)c1[nH]c2ccc(F)cc2c1C1CCCCC1)NC(=O)OC(C)(C)C]

### di-tert-butyl ((4S)-5-(3-cyclohexyl-5-fluoro-1H-indole-2-carboxamido)-3-hydroxypentane-1,4-diyl)dicarbamate

To a solution of 3-cyclohexyl-5-fluoro-1H-indole-2-carboxylic acid (20 mg, 0.08 mmol) in dry DMF (1 mL) was added DIPEA (0.03 mL, 0.15 mmol), HOBt (6.2 mg, 0.05 mmol) and EDC (16 mg, 0.08 mmol). The reaction mixture was stirred at room temperature for 10 min then di-tert-butyl ((3R,4S)-5-amino-3-hydroxypentane-1,4-diyl) dicarbamate (Amine Intermediate A) (26 mg, 0.08 mmol) was added. The reaction mixture was stirred at room temperature overnight then added H₂O with stirring. A solid was formed and collected by filtration. After air drying the solid was purified by chromatography on silica gel. Elution with 50% EtOAc/hexanes afforded the desired compound (30 mg, 54% yield) as yellow solid. ¹H NMR (300 MHz, CDCl₃) δ 9.42 (br s, 1H), 7.49 (m, 1H), 7.23 (m, 1H), 6.96 (m, 1H), 5.43 (d, J=7.5 Hz, 1H), 4.93 (br s, 1H), 3.77 (m, 2H), 3.63 (m, 1H), 3.52 (m, 1H), 3.24 (m, 1H), 3.12 (m, 1H), 1.78-2.00 (m, 8H), 1.61 (m, 2H), 1.36-1.40 (m, 20H).

### Example 6. Preparation of N-((2S)-2,5-diamino-3-hydroxypentyl)-3-(4,4-difluorocyclohexyl)-1H-indole-2-carboxamide hydrogen chloride salt

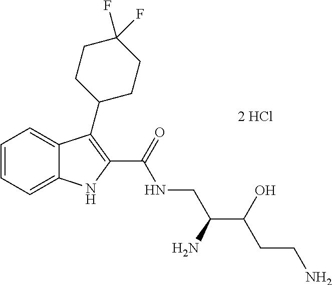 [SMILES: NCCC(O)[C@@H](N)CNC(=O)c1[nH]c2ccccc2c1C1CCC(F)(F)CC1]

### N-((2S)-2,5-diamino-3-hydroxypentyl)-3-(4,4-difluorocyclohexyl)-1H-indole-2-carboxamide hydrogen chloride salt

To a solution of di-tert-butyl ((4S)-5-(3-(4,4-difluorocyclohexyl)-1H-indole-2-carboxamido)-3-hydroxypentane-1,4-diyl)dicarbamate (23 mg, 0.04 mmol) in MeOH (2 mL) was added HCl (4 M in dioxane, 0.3 mL, 1.2 mmol). The reaction mixture was stirred at 50° C. for 1 h then concentrated to give a residue. The residue was triturated with EtOAc to give the product (12 mg, 67% yield) as a brown solid. ¹H NMR (300 MHz, D₂O) δ 7.91 (d, J 8.1 Hz, 1H), 7.50 (d, J=8.1 Hz, 1H), 7.33 (m, 1H), 7.15 (m, 1H), 4.08 (m, 1H), 3.81 (m, 1H), 3.62 (m, 2H), 3.50 (m, 1H), 3.16 (m, 2H), 2.10-2.36 (m, 4H), 1.80-2.08 (m, 6H). MS (ESI): Calcd for C₂₀H₂₉F₂N₄O₂⁺ 395.23 [M+H]⁺, found 395.05 [M+H]⁺.

The requisite intermediates were prepared as shown in the following steps.

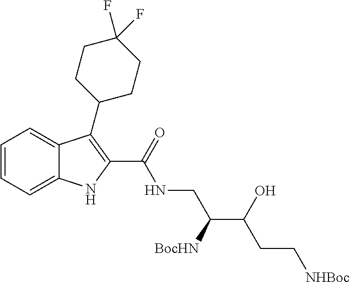 [SMILES: CC(C)(C)OC(=O)NCCC(O)[C@H](CNC(=O)c1[nH]c2ccccc2c1C1CCC(F)(F)CC1)NC(=O)OC(C)(C)C]

### di-tert-butyl ((4S)-5-(3-(4,4-difluorocyclohexyl)-1H-indole-2-carboxamido)-3-hydroxypentane-1,4-diyl)dicarbamate

To a solution of 3-(4,4-difluorocyclohexyl)-1H-indole-2-carboxylic acid (22 mg, 0.08 mmol) (prepared in a similar manner of Example 4) in dry DMF (1 mL) was added DIPEA (0.03 mL, 0.15 mmol), HOBt (6.2 mg, 0.05 mmol) and EDC (16 mg, 0.08 mmol). The reaction mixture was stirred at room temperature for 10 min then di-tert-butyl ((3R,4S)-5-amino-3-hydroxypentane-1,4-diyl) dicarbamate (Amine Intermediate A) (26 mg, 0.08 mmol) was added. The reaction mixture was stirred at room temperature overnight then added H₂O with stirring. A solid was formed and collected by filtration. After air drying the solid was purified by column chromatography on silica gel. Elution with 50% EtOAc/hexanes afforded the desired compound (23 mg, 49% yield) as off-white solid. ¹H NMR (300 MHz, CDCl₃) δ 9.28 (br s, 1H), 7.80 (d, J=8.4 Hz, 1H), 7.34 (m, 2H), 7.23 (m, 1H), 7.09 (m, 1H), 5.44 (d, J=7.5 Hz, 1H), 4.99 (br s, 1H), 4.90 (br, 1H), 3.78 (m, 2H), 3.60 (m, 2H), 3.51 (m, 2H), 3.12 (m, 1H), 2.37 (m, 2H), 2.21 (m, 2H), 1.93 (m, 4H), 161 (m, 2H), 1.36-1.40 (m, 18H).

### Example 7. Preparation of N-((2S)-2,5-diamino-3-hydroxypentyl)-3-(2-fluorophenyl)-1H-indole-2-carboxamide hydrogen chloride salt

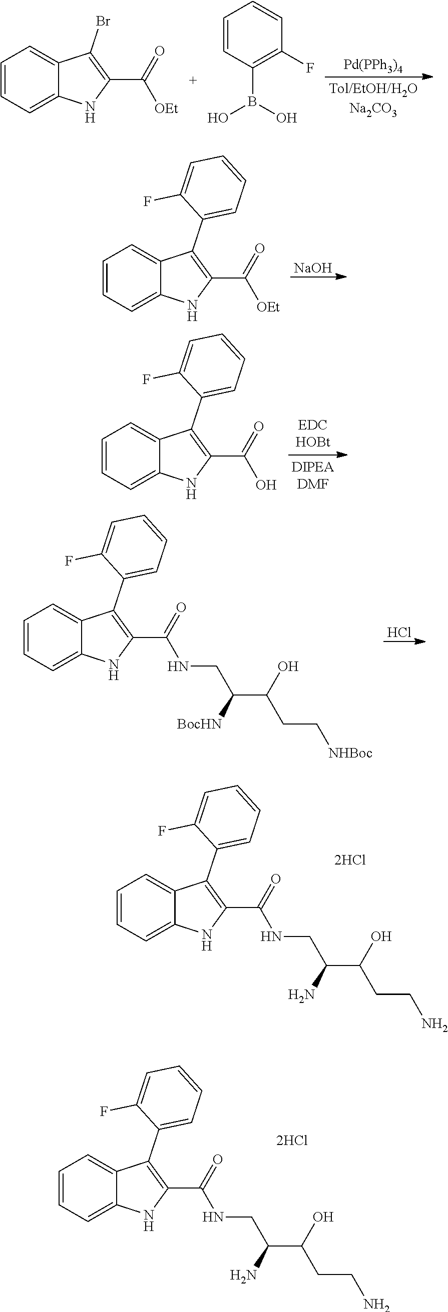

### N-((2S)-2,5-diamino-3-hydroxypentyl)-3-(2-fluorophenyl)-1H-indole-2-carboxamide hydrogen chloride salt

To a solution of di-tert-butyl ((4S)-5-(3-(2-fluorophenyl)-1H-indole-2-carboxamido)-3-hydroxypentane-1,4-diyl)dicarbamate (18 mg, 0.032 mmol) in methanol (1 mL) was added HCl (4 M in dioxane, 0.2 mL, 0.8 mmol). Reaction mixture was stirred at 50° C. for 30 minutes then solvents were removed in vacuo. Residue was washed with small amount of ethyl ether and dried. Product was collected as a gray solid (7.4 mg, 53% yield). ¹H NMR (300 MHz, D₂O) δ 7.15-7.62 (m, 8H), 3.93 (d, J=9.0 Hz, 1H), 3.22-3.79 (m, 4H), 3.11 (t, J=4.5 Hz, 1H), 1.66 (m, 2H). MS (ESI): Calcd for C₂₀H₂₄FN₄O₂⁺ 371.19 [M+H]⁺, found 371.00 [M+H]⁺.

The requisite intermediates were prepared as shown in the following steps.

### Step 1)

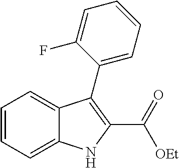 [SMILES: CCOC(=O)c1[nH]c2ccccc2c1-c1ccccc1F]

### ethyl 3-(2-fluorophenyl)-1H-indole-2-carboxylate

Ethyl 3-bromo-1H-indole-2-carboxylate (268 mg, 1 mmol) and (2-fluorophenyl)boronic acid (280 mg, 2 mmol) in a mixture of toluene, ethanol and 2 M Na₂CO₃ solution (10/3/3 mL) was degassed and Pd(dppf)Cl₂ (30 mg, 0.04 mmol) was added. The reaction mixture was heated at 100° C. for 3 hours and it was extracted with EtOAc and washed with water, brine and dried over anhydrous sodium sulfate. Then it was concentrated, and the residue was purified by column chromatography on silica gel to give the product (220 mg, 78% yield) as an off-white powder.

### Step 2)

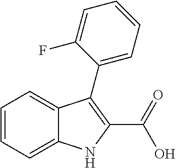 [SMILES: O=C(O)c1[nH]c2ccccc2c1-c1ccccc1F]

### 3-(2-fluorophenyl)-1H-indole-2-carboxylic acid

To a solution of ethyl 3-(2-fluorophenyl)-5-1H-indole-2-carboxylate (220 mg, 1.4 mmol) in THF (10 mL) was added NaOH solution (2 M, 5 mL). It was heated at 50° C. overnight. THF was removed in vacuo and the residue was acidified with HCl solution. The precipitate was filtered and washed with water. It was dried to provide the product as a pale brown powder (230 mg, 56% yield) which was used for next step reaction without further purification. ¹H NMR (300 MHz, DMSO-d₆) δ 11.98 (br s, 1H), 7.38-7.53 (m, 3H), 7.22-7.36 (m, 4H), 7.07 (t, J=7.5 Hz, 1H). MS (ESI): Calcd for C₁₅H₁₁FNO₂⁺ 256.08 [M+H]⁺, found 255.85 [M+H]⁺.

### Step 3)

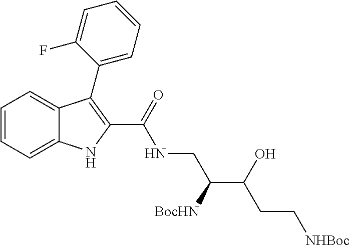 [SMILES: CC(C)(C)OC(=O)NCCC(O)[C@H](CNC(=O)c1[nH]c2ccccc2c1-c1ccccc1F)NC(=O)OC(C)(C)C]

### di-tert-butyl ((4S)-5-(3-(2-fluorophenyl)-1H-indole-2-carboxamido)-3-hydroxypentane-1,4-diyl)dicarbamate

To a solution of di-tert-butyl ((4S)-5-amino-3-hydroxypentane-1,4-diyl)dicarbamate acetate (28 mg, 0.07 mmol) (Amine Intermediate A) in DMF (1 mL) was added 3-(2-fluorophenyl)-1H-indole-2-carboxylic acid (22 mg, 0.085 mmol), EDC (17 mg, 0.11 mmol), HOBt (5 mg, 0.03 mmol) and DIPEA (14 mg, 0.13 mmol). Resulting reaction mixture was stirred at room temperature overnight then diluted with water and stirred for 30 minutes. The precipitate was filtered and washed with small amount of water then dissolved in DCM, washed with brine, and dried. After concentration, the residue was purified on column chromatography on silica gel to give the product as a light yellow solid (23 mg, 74% yield). ¹H NMR (300 MHz, CDCl₃) δ 9.65 (br s, 1H), 7.41-7.56 (m, 4H), 7.26-7.38 (m, 3H), 7.14 (t, J=8.1 Hz, 1H), 6.52 (br s, 1H), 4.80-4.99 (m, 2H), 4.38-4.46 (m, 1H), 3.61-3.78 (m, 2H), 3.32-3.58 (m, 3H), 3.01-3.23 (m, 1H), 1.63 (m, 2H), 1.40 (br, 18H); MS (ESI): Calcd for C₃₀H₄₀FN₄O₆⁺ 571.29 [M+H]⁺, found 571.25 [M+H]⁺.

### Example 8. Preparation of 3-(2-chlorophenyl)-N-((2S)-2,5-diamino-4-hydroxypentyl)-5-fluoro-1H-indole-2-carboxamide hydrogen chloride salt

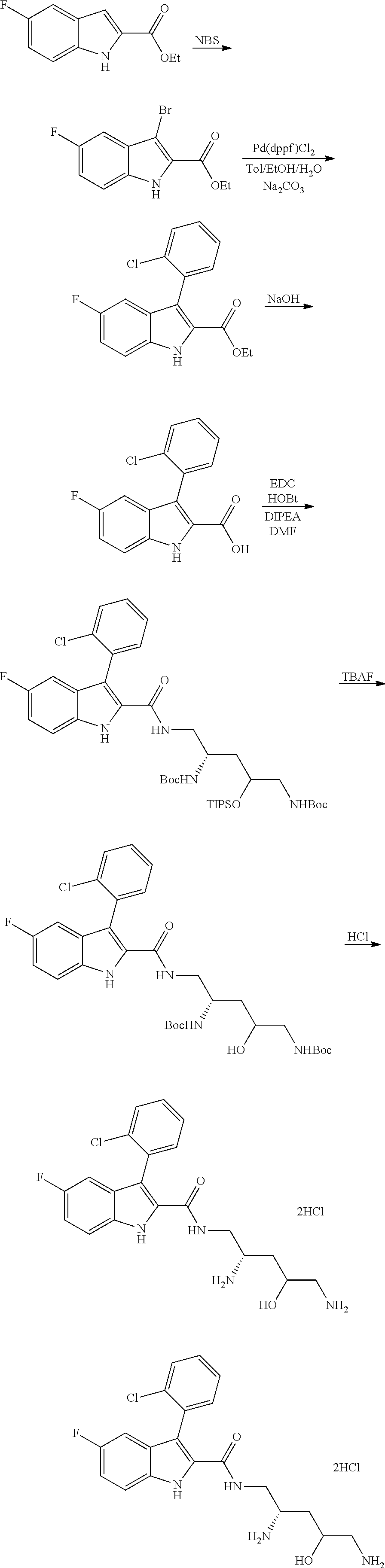

### 3-(2-chlorophenyl)-N-((2S)-2,5-diamino-4-hydroxypentyl)-5-fluoro-1H-indole-2-carboxamide hydrogen chloride salt

To a solution of (S)-di-tert-butyl (5-(6-benzyl-1H-indole-2-carboxamido)pentane-1,4-diyl)dicarbamate (6 mg, 0.01 mmol) in MeOH (2 mL) HCl solution (4 M in dioxane, 0.1 mL, 0.4 mmol) was added. It was stirred at room temperature for 2 hrs, then solvent was removed in vacuo. The residue was triturated with EtOAc and the precipitate was collected as an off-white powder (4 mg, 84% yield). ¹H NMR (300 MHz, D₂O) δ 7.64 (m, 1H), 7.53 (m, 2H), 7.48 (m, 2H), 7.17 (m, 1H), 7.11 (m, 1H), 4.06 (m, 1H), 3.70 (m, 1H), 3.62 (m, 2H), 3.57 (m, 1H), 3.09 (m, 1H), 2.91 (m, 1H), 1.76 (m, 1H), 1.68 (m, 1H). MS (ESI): Calcd for C₂₀H₂₃ClFN₄O₂⁺ 405.15 [M+H]⁺, found 404.95 [M+H]⁺.

The requisite intermediates were prepared as shown in the following steps.

### Step 1)

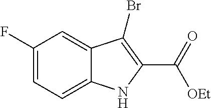 [SMILES: CCOC(=O)c1[nH]c2ccc(F)cc2c1Br]

### ethyl 3-bromo-5-fluoro-1H-indole-2-carboxylate

To a solution of ethyl 5-fluoro-1H-indole-2-carboxylate (5 g, 24.2 mmol) in dry THE (10 mL) was added NBS (4.73 g, 26.6 mmol) at −78° C. Once the temperature reached to room temperature, it was washed with water, sat. NaHCO₃ and brine. After removing solvent in vacuo, the residue was used for next step reaction without purification (6.58 g, 95% yield) as a white powder. ¹H NMR (300 MHz, CDCl₃) δ 9.28 (br s, 1H), 7.37 (m, 1H), 7.32 (m, 1H), 7.14 (m, 1H), 4.46 (q, J=7.2 Hz, 2H), 1.47 (t, J=7.2 Hz, 3H). MS (ESI): Calcd for C₁₁H₁₀BrFNO₂⁻ 285.98 and 287.98 [M−H]⁻, found 285.85 and 287.85 [M−H]⁻.

### Step 2)

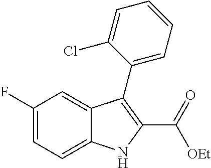 [SMILES: CCOC(=O)c1[nH]c2ccc(F)cc2c1-c1ccccc1Cl]

### ethyl 3-(2-chlorophenyl)-5-fluoro-1H-indole-2-carboxylate

Ethyl 3-bromo-5-fluoro-1H-indole-2-carboxylate (620 mg, 2.1 mmol) and (2-chlorophenyl)boronic acid (495 mg, 3.2 mmol) in a mixture of toluene, ethanol and 2 M Na₂CO₃ solution (20/4/8 mL) was degassed and Pd(dppf)Cl₂ (100 mg, 0.11 mmol) was added. The reaction mixture was heated at 110° C. overnight and it was extracted with EtOAc and washed with brine and dried over anhydrous sodium sulfate. Then it was concentrated, and the residue was purified by column chromatography on silica gel to give the product (450 mg, 68% yield) as an off-white powder. ¹H NMR (300 MHz, CDCl₃) δ 8.97 (br s, 1H), 7.48 (m, 1H), 7.37 (m, 1H), 7.34 (m, 3H), 7.29 (m, 1H), 7.09 (m, 1H), 4.23 (q, J=7.2 Hz, 2H), 1.13 (t, J=7.2 Hz, 3H). MS (ESI): Calcd for C₁₇H₁₄ClFNO₂⁺ 318.07 [M+H]⁺, found 317.90 [M+H]⁺.

### Step 3)

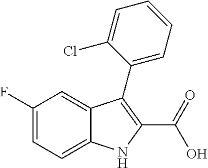 [SMILES: O=C(O)c1[nH]c2ccc(F)cc2c1-c1ccccc1Cl]

### 3-(2-chlorophenyl)-5-fluoro-1H-indole-2-carboxylic acid

To a solution of ethyl 3-(2-chlorophenyl)-5-fluoro-1H-indole-2-carboxylate (450 mg, 1.4 mmol) in THE (10 mL) was added NaOH solution (2 M, 5 mL). It was heated at 50° C. overnight. THE was removed in vacuo and the residue was acidified with HCl solution. The precipitate was filtered and washed with water. It was dried to provide the product as a pale brown powder (230 mg, 56% yield) which was used for next step reaction without further purification. MS (ESI): Calcd for C₁₅H₁₀ClFNO₂⁺ 290.04 [M+H]⁺, found 289.85 [M+H]⁺.

### Step 4)

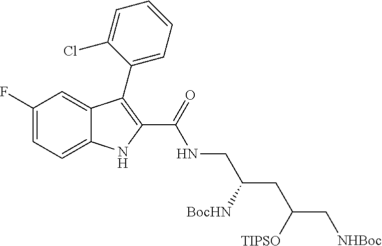

### di-tert-butyl ((4S)-5-(3-(2-chlorophenyl)-5-fluoro-1H-indole-2-carboxamido)-2-((triisopropylsilyl)oxy)pentane-1,4-diyl)dicarbamate

To a solution of 3-(2-chlorophenyl)-5-fluoro-1H-indole-2-carboxylic acid (15 mg, 0.05 mmol) in dry DMF (0.5 mL) was added DIPEA (0.034 mL, 0.2 mmol), HOBt (10 mg, 0.06 mmol) and EDC (12 mg, 0.06 mmol). The reaction mixture was stirred at room temperature and di-tert-butyl ((4S)-5-amino-2-((triisopropylsilyl)oxy)pentane-1,4-diyl)dicarbamate (Amine Intermediate B) (25 mg, 0.05 mmol) was added. The reaction mixture was stirred at room temperature overnight. It was extracted with EtOAc and washed with water and brine. The organic layer was dried over anhydrous sodium sulfate and filtered. The filtrate was concentrated and purified by column chromatography on silica gel using 50-70% EtOAc in hexane to give the product (18 mg, 47% yield) as a pale brown powder. ¹H NMR (300 MHz, CDCl₃) δ 9.75 (br s, 1H), 7.63 (m, 1H), 7.51 (m, 1H), 7.46 (m, 2H), 7.37 (m, 2H), 7.09 (m, 1H), 6.94 (br s, 1H), 5.27 (br s, 1H), 4.82 (br, 1H), 4.00 (m, 1H), 3.68 (m, 1H), 3.42 (m, 2H), 3.26 (m, 1H), 3.13 (m, 1H), 1.66 (m, 2H), 1.43 (s, 9H), 1.39 (s, 12H), 1.04 (s, 18H). MS (ESI): Calcd for C₃₉H₅₉ClFN₄O₆Si⁺761.39 [M+H]⁺, found 761.35 [M+H]⁺.

### Step 5)

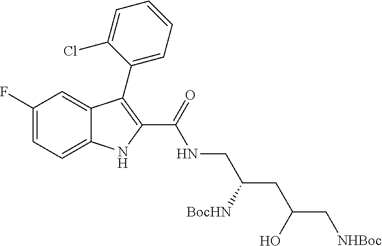 [SMILES: CC(C)(C)OC(=O)NCC(O)C[C@@H](CNC(=O)c1[nH]c2ccc(F)cc2c1-c1ccccc1Cl)NC(=O)OC(C)(C)C]

### di-tert-butyl ((4S)-5-(3-(2-chlorophenyl)-5-fluoro-1H-indole-2-carboxamido)-2-hydroxypentane-1,4-diyl)dicarbamate

To a solution of di-tert-butyl ((4S)-5-(3-(2-chlorophenyl)-5-fluoro-1H-indole-2-carboxamido)-2-((triisopropylsilyl)oxy)pentane-1,4-diyl)dicarbamate (17 mg, 0.022 mmol) in THE (2 mL) was added TBAF solution (1.0 M in THF, 0.4 mL, 0.4 mmol) at room temperature It was stirred for 0.5 hr, then concentrated and loaded on silica gel column chromatography and purified with 50-70% EtOAc in hexane to provide the alcohol as a white powder (7 mg, 53% yield). MS (ESI): Calcd for C₃₀H₃₉ClFN₄O₆⁺ 605.25 [M+H]⁺, found 605.20 [M+H]⁺.

### Example 9. Preparation of 3-(4-cyanophenyl)-N-((2S)-2,5-diamino-4-hydroxypentyl)-5-fluoro-1H-indole-2-carboxamide hydrogen chloride salt

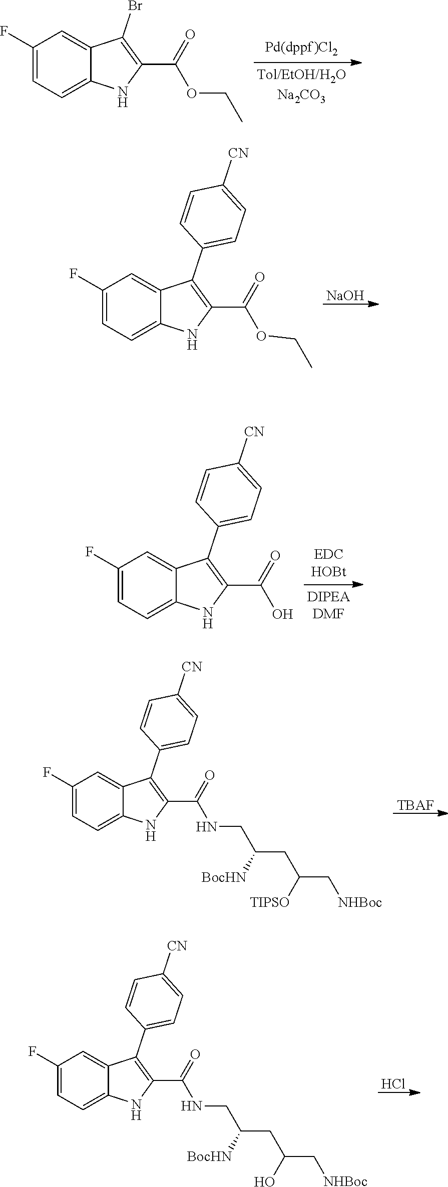

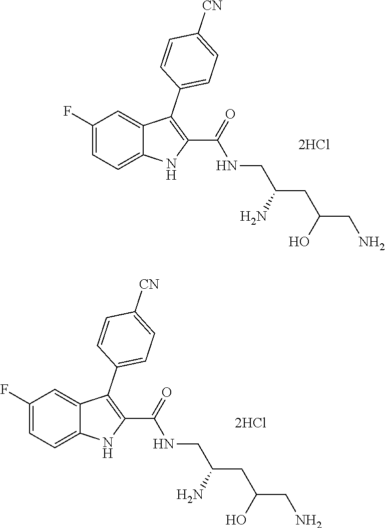 [SMILES: N#Cc1ccc(-c2c(C(=O)NC[C@@H](N)CC(O)CN)[nH]c3ccc(F)cc23)cc1 ; N#Cc1ccc(-c2c(C(=O)NC[C@@H](N)CC(O)CN)[nH]c3ccc(F)cc23)cc1]

### 3-(4-cyanophenyl)-N-((2S)-2,5-diamino-4-hydroxypentyl)-5-fluoro-1H-indole-2-carboxamide hydrogen chloride salt

To a solution of di-tert-butyl ((4S)-5-(3-(4-cyanophenyl)-5-fluoro-1H-indole-2-carboxamido)-2-hydroxypentane-1,4-diyl)dicarbamate (16 mg, 0.027 mmol) in MeOH (2 mL) HCl solution (4 M in dioxane, 0.1 mL, 0.4 mmol) was added. It was stirred at room temperature for 2 hrs, then solvent was removed in vacuo. The residue was triturated with EtOAc and the precipitate was collected as an off-white powder (11 mg, 87% yield). ¹H NMR (300 MHz, D₂O) δ 7.84 (d, J 8.3 Hz, 2H), 7.64 (d, J 8.3 Hz, 2H), 7.50 (dd, J=4.4, 12.0 Hz, 1H), 7.29 (d, J=9.8 Hz, 1H), 7.64 (m, 1H), 4.03 (m, 1H), 3.61 (m, 2H), 3.48 (m, 1H), 3.06 (m, 1H), 2.89 (m, 1H), 1.77 (m, 2H). MS (ESI): Calcd for C₂₁H₂₃FN₅O₂⁺ 396.18 [M+H]⁺, found 396.00 [M+H]⁺.

The requisite intermediates were prepared as shown in the following steps.

### Step 1)

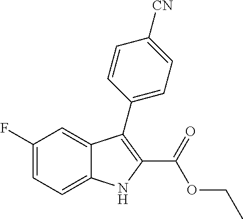 [SMILES: CCOC(=O)c1[nH]c2ccc(F)cc2c1-c1ccc(C#N)cc1]

### ethyl 3-(4-cyanophenyl)-5-fluoro-1H-indole-2-carboxylate

Ethyl 3-bromo-5-fluoro-1H-indole-2-carboxylate (570 mg, 2.0 mmol) and (4-cyanophenyl)boronic acid (350 mg, 2.4 mmol) in a mixture of toluene, ethanol and saturated Na₂CO₃ solution (20/6/6 mL) was degassed and Pd(dppf)Cl₂ (100 mg, 0.14 mmol) was added. The reaction mixture was heated at 110° C. overnight, it was extracted with EtOAc and washed with brine and dried over Na₂SO₄, then concentrated. It was purified by column chromatography on silica gel to give the product (325 mg, 53% yield) as an off-white powder. ¹H NMR (300 MHz, CDCl₃) δ 9.11 (br s, 1H), 7.75 (d, J=6.3 Hz, 2H), 7.69 (d, J=6.3 Hz, 2H), 7.42 (m, 1H), 7.19 (m, 2H), 4.31 (q, J=7.1 Hz, 2H), 1.26 (t, J=7.1 Hz, 3H).

### Step 2)

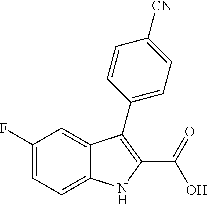 [SMILES: N#Cc1ccc(-c2c(C(=O)O)[nH]c3ccc(F)cc23)cc1]

### 3-(4-cyanophenyl)-5-fluoro-1H-indole-2-carboxylic acid

To a solution of ethyl 3-(4-cyanophenyl)-5-fluoro-1H-indole-2-carboxylate (320 mg, 1.04 mmol) in THF (10 mL) was added NaOH solution (2 M, 10 mL). It was heated at 65° C. for 1 hr. THF was removed in vacuo and the residue was acidified with HCl solution. The precipitate was filtered and washed with water. It was dried to provide the acid as a pale brown powder (235 mg, 81% yield) which was used for next step without further purification.

### Step 3)

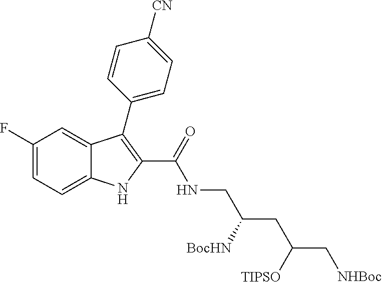

### di-tert-butyl ((4S)-5-(3-(4-cyanophenyl)-5-fluoro-1H-indole-2-carboxamido)-2-((triisopropylsilyl)oxy)pentane-1,4-diyl)dicarbamate

To a solution of 3-(4-cyanophenyl)-5-fluoro-1H-indole-2-carboxylic acid (28 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and di-tert-butyl ((4S)-5-amino-2-((triisopropylsilyl)oxy)pentane-1,4-diyl)dicarbamate (Amine Intermediate B) (37 mg, 0.075 mmol) was added. The reaction mixture was stirred at room temperature overnight. It was extracted with EtOAc and washed with water and brine. The organic layer was dried over anhydrous sodium sulfate and filtered. The filtrate was concentrated and purified by column chromatography on silica gel using 50-70% EtOAc in hexane as eluents to give the amide (33 mg, 59% yield) as a white powder. ¹H NMR (300 MHz, CDCl₃) δ 9.44 (br s, 1H), 7.82 (d, J=8.1 Hz, 2H), 7.69 (d, J=8.1 Hz, 2H), 7.39 (m, 1H), 7.10 (m, 2H), 6.71 (br s, 1H), 5.27 (br s, 1H), 4.77 (br, 1H), 4.01 (m, 1H), 3.71 (m, 1H), 3.41 (m, 2H), 3.25 (m, 1H), 3.16 (m, 1H), 1.63 (m, 2H), 1.41 (s, 9H), 1.25 (s, 12H), 1.06 (s, 18H). MS (ESI): Calcd for C₄₀H₅₉FN₅O₆Si⁺752.42 [M+H]⁺, found 752.60 [M+H]⁺.

### Step 4)

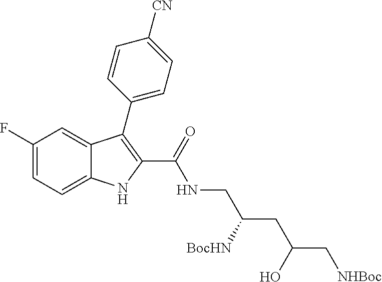 [SMILES: CC(C)(C)OC(=O)NCC(O)C[C@@H](CNC(=O)c1[nH]c2ccc(F)cc2c1-c1ccc(C#N)cc1)NC(=O)OC(C)(C)C]

### di-tert-butyl ((4S)-5-(3-(4-cyanophenyl)-5-fluoro-1H-indole-2-carboxamido)-2-hydroxypentane-1,4-diyl)dicarbamate

To a solution of tert-butyl ((4S)-5-(3-(4-cyanophenyl)-5-fluoro-1H-indole-2-carboxamido)-2-((triisopropylsilyl)oxy)pentane-1,4-diyl)dicarbamate (32 mg, 0.056 mmol) in THE (2 mL) was added TBAF solution (1.0 M in THF, 0.4 mL, 0.4 mmol) at room temperature It was stirred for 0.5 hr, concentrated and loaded on column chromatography on silica gel and purified with 40-65% EtOAc in hexane to provide the alcohol as a white powder (17 mg, 51% yield).

### Example 10. Preparation of 3-(4-fluorophenyl)-N-((2S)-2,5-diamino-4-hydroxypentyl)-1H-indole-2-carboxamide hydrogen chloride salt

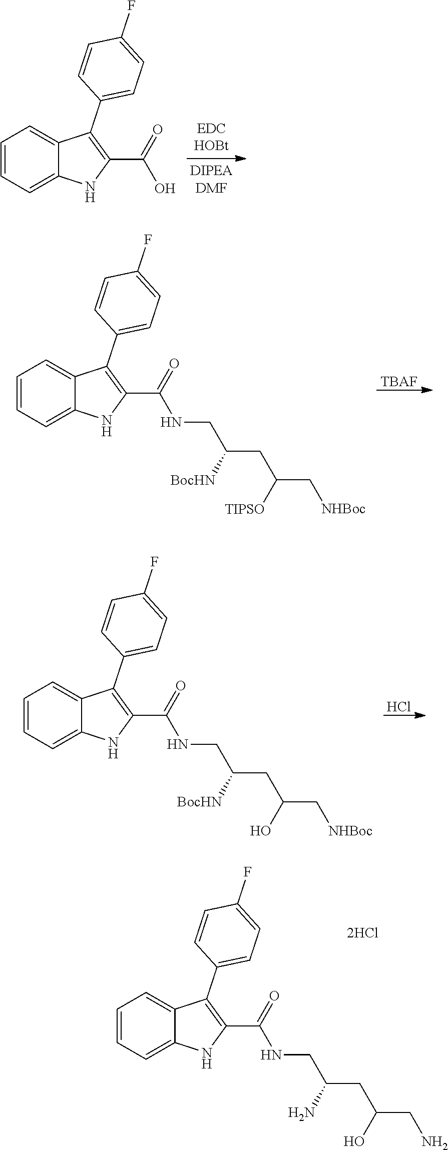

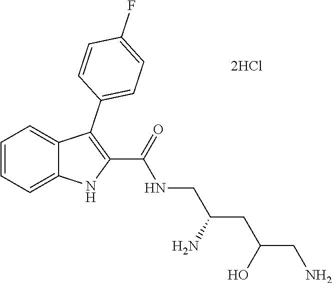 [SMILES: NCC(O)C[C@H](N)CNC(=O)c1[nH]c2ccccc2c1-c1ccc(F)cc1]

### 3-(4-fluorophenyl)-N-((2S)-2,5-diamino-4-hydroxypentyl)-1H-indole-2-carboxamide hydrogen chloride salt

To a solution of di-tert-butyl ((4S)-5-(3-(4-fluorophenyl)-1H-indole-2-carboxamido)-2-hydroxypentane-1,4-diyl)dicarbamate (16 mg, 0.027 mmol) in MeOH (2 mL) HCl solution (4 M in dioxane, 0.1 mL, 0.4 mmol) was added. It was stirred at room temperature for 2 hrs, then solvent was removed in vacuo. The residue was triturated with EtOAc and the precipitate was collected as a white powder (11 mg, 87% yield). ¹H NMR (300 MHz, D₂O) δ 7.50-7.63 (m, 5H), 7.38 (m, 1H), 7.21 (m, 2H), 7.18 (m, 1H), 4.03 (m, 1H), 3.48-3.75 (m, 3H), 3.05 (m, 1H), 2.90 (m, 1H), 1.72 (m, 2H). MS (ESI): Calcd for C₂₀H₂₄FN₄O₂⁺ 371.18 [M+H]⁺, found 371.00 [M+H]⁺.

The requisite intermediates were prepared as shown in the following steps.

### Step 1)

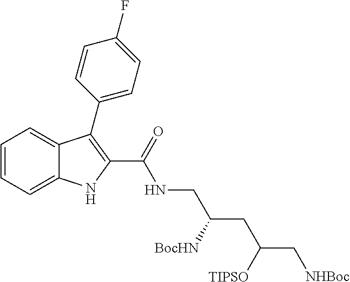

### di-tert-butyl ((4S)-5-(3-(4-fluorophenyl)-1H-indole-2-carboxamido)-2-((triisopropylsilyl)oxy)pentane-1,4-diyl)dicarbamate

To a solution of 3-(4-fluorophenyl)-1H-indole-2-carboxylic acid (26 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and di-tert-butyl ((4S)-5-amino-2-((triisopropylsilyl)oxy)pentane-1,4-diyl)dicarbamate (Amine Intermediate B) (49 mg, 0.1 mmol) was added. The reaction was continued at room temperature overnight. It was extracted with EtOAc and washed with water and brine. The organic layer was dried over anhydrous sodium sulfate and filtered. The filtrate was concentrated and purified by column chromatography on silica gel using 50-70% EtOAc in hexane as eluents to give the amide (52 mg, 72% yield) as a white powder. MS (ESI): Calcd for C₃₉H₆₀FN₄O₆Si⁺727.43 [M+H]⁺, found 727.55 [M+H]⁺.

### Step 2)

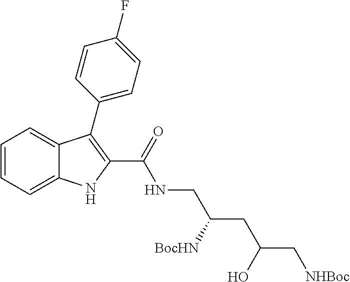 [SMILES: CC(C)(C)OC(=O)NCC(O)C[C@@H](CNC(=O)c1[nH]c2ccccc2c1-c1ccc(F)cc1)NC(=O)OC(C)(C)C]

### di-tert-butyl ((4S)-5-(3-(4-fluorophenyl)-1H-indole-2-carboxamido)-2-hydroxypentane-1,4-diyl)dicarbamate

To a solution of di-tert-butyl ((4S)-5-(3-(4-fluorophenyl)-1H-indole-2-carboxamido)-2-((triisopropylsilyl)oxy)pentane-1,4-diyl)dicarbamate (30 mg, 0.041 mmol) in THE (5 mL) was added TBAF solution (1.0 M in THF, 0.4 mL, 0.4 mmol) at room temperature. It was stirred for 1 hr and concentrated, then loaded on silica gel column chromatography and purified with 50-80% EtOAc in hexane to provide the alcohol as a white powder (15 mg, 64% yield). ¹H NMR (300 MHz, CDCl₃) δ 9.34 (br s, 1H), 7.41-7.53 (m, 4H), 7.34 (m, 1H), 7.25 (m, 2H), 7.15 (m, 2H), 6.10 (m, 1H) 5.21 (br s, 1H), 5.03 (br s, 1H), 4.37 (br, 1H), 4.01 (m, 1H), 3.77 (m, 1H), 3.65 (m, 1H), 3.24-3.53 (m, 3H), 2.95 (m, 1H), 1.62 (m, 2H), 1.41 (br, 18H). MS (ESI): Calcd for C₃₀H₄₀FN₄O₆⁺ 571.29 [M+H]⁺, found 571.25 [M+H]⁺.

### Example 11. Preparation of 3-(4-cyanophenyl)-N-((2S)-2,5-diamino-4-hydroxypentyl)-1H-indole-2-carboxamide hydrogen chloride salt

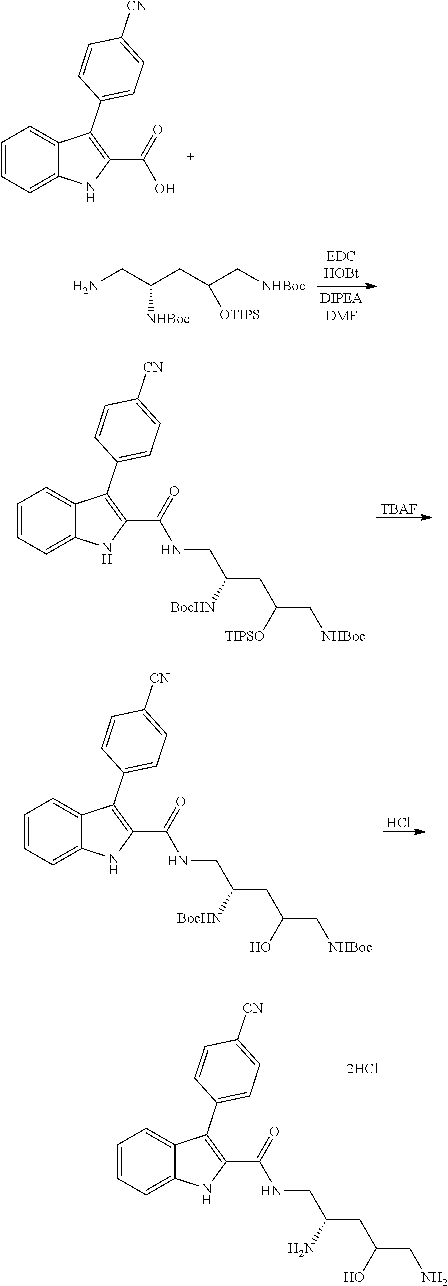

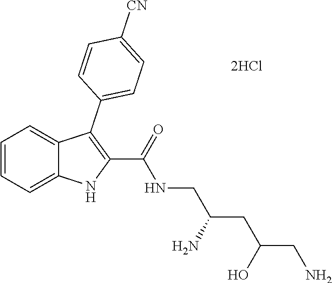 [SMILES: N#Cc1ccc(-c2c(C(=O)NC[C@@H](N)CC(O)CN)[nH]c3ccccc23)cc1]

### 3-(4-cyanophenyl)-N-((2S)-2,5-diamino-4-hydroxypentyl)-1H-indole-2-carboxamide hydrogen chloride salt

To a solution of di-tert-butyl ((4S)-5-(3-(4-cyanophenyl)-1H-indole-2-carboxamido)-2-hydroxypentane-1,4-diyl)dicarbamate (18 mg, 0.03 mmol) in MeOH (2 mL) HCl solution (4 M in dioxane, 0.1 mL, 0.4 mmol) was added. It was stirred at room temperature for 2 hrs, then solvent was removed in vacuo. The residue was triturated with EtOAc and the precipitate was collected as an off-white powder (10 mg, 72% yield). ¹H NMR (300 MHz, D₂O) δ 7.77 (m, 2H), 7.71 (m, 2H), 7.50 (m, 2H), 7.27 (m, 1H), 7.10 (m, 1H), 4.04 (m, 1H), 3.48-3.72 (m, 3H), 3.31 (m, 2H), 3.01 (m, 1H), 2.83 (m, 1H), 1.85 (m, 1H), 1.67 (m, 1H). MS (ESI): Calcd for C₂₁H₂₄FN₅O₂⁺ 378.19 [M+H]⁺, found 378.95 [M+H]⁺.

The requisite intermediates were prepared as shown in the following steps.

### Step 1)

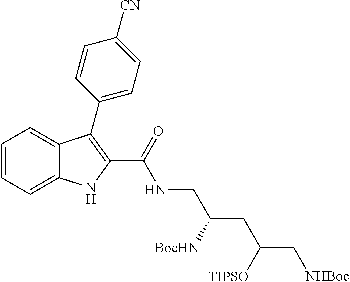

### di-tert-butyl ((4S)-5-(3-(4-cyanophenyl)-1H-indole-2-carboxamido)-2-((triisopropylsilyl)oxy)pentane-1,4-diyl)dicarbamate

To a solution of 3-(4-cyanophenyl)-1H-indole-2-carboxylic acid (26 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and di-tert-butyl ((4S)-5-amino-2-((triisopropylsilyl)oxy)pentane-1,4-diyl)dicarbamate (Amine Intermediate B) (49 mg, 0.1 mmol) was added. The reaction was continued at room temperature overnight. It was extracted with EtOAc and washed with water and brine. The organic layer was dried over anhydrous sodium sulfate and filtered. The filtrate was concentrated and purified by column chromatography on silica gel using 30-60% EtOAc in hexane as eluents to give two diastereomers (55 mg, 73% yield) as a white powder. MS (ESI): Calcd for C₄₀H₆₀N₅O₆Si⁺734.43 [M+H]⁺, found 756.55 [M+Na]⁺.

### Step 2)

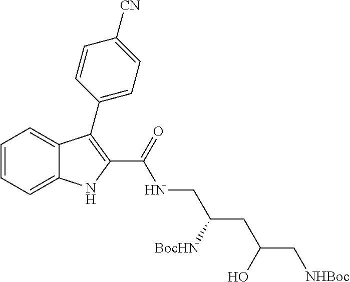 [SMILES: CC(C)(C)OC(=O)NCC(O)C[C@@H](CNC(=O)c1[nH]c2ccccc2c1-c1ccc(C#N)cc1)NC(=O)OC(C)(C)C]

### di-tert-butyl ((4S)-5-(3-(4-cyanophenyl)-1H-indole-2-carboxamido)-2-hydroxypentane-1,4-diyl)dicarbamate

To a solution of the less polar one of the two diastereomers of di-tert-butyl ((4S)-5-(3-(4 cyanophenyl)-1H-indole-2-carboxamido)-2-((triisopropylsilyl)oxy)pentane-1,4-diyl)dicarbamate (35 mg, 0.046 mmol) in THE (5 mL) was added TBAF solution (1.0 M in THF, 0.4 mL, 0.4 mmol) at room temperature It was stirred for 1 hr and concentrated, then loaded on column chromatography on silica gel and purified with 40-80% EtOAc in hexane to provide the alcohol as a white powder (18 mg, 67% yield). MS (ESI): Calcd for C₃₁H₄₀N₅O₆⁺ 578.29 [M+H]⁺, found 578.25 [M+H]⁺.

### Example 12. Preparation of N-((2S)-2,5-diamino-4-hydroxypentyl)-3-phenyl-1H-indole-2-carboxamide hydrogen chloride salt

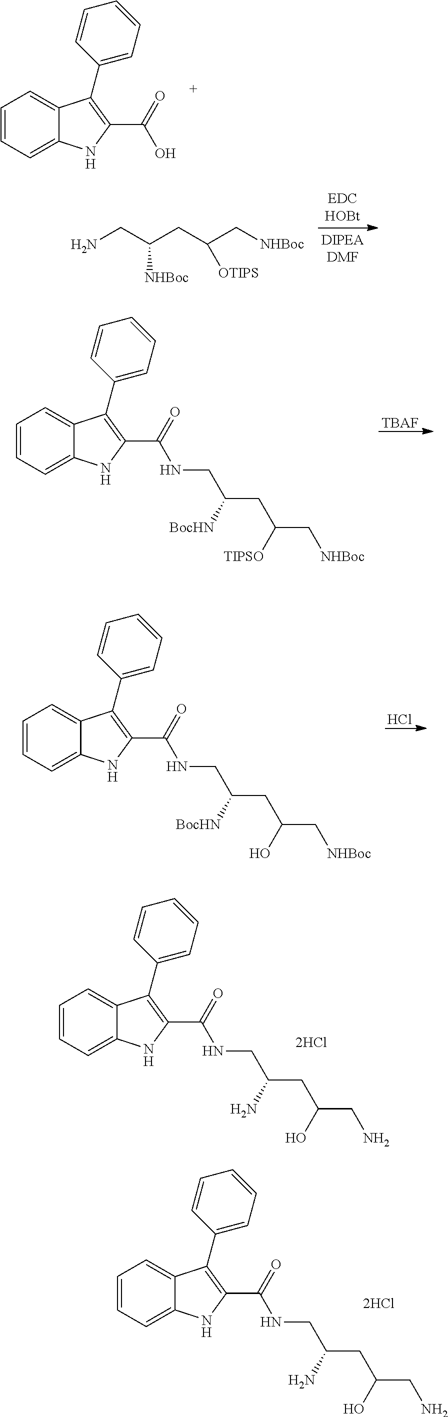

### N-((2S)-2,5-diamino-4-hydroxypentyl)-3-phenyl-1H-indole-2-carboxamide hydrogen chloride salt

To a solution of di-tert-butyl ((4S)-5-(3-phenyl)-1H-indole-2-carboxamido)-2-hydroxypentane-1,4-diyl)dicarbamate (18 mg, 0.032 mmol) in MeOH (2 mL) HCl solution (4 M in dioxane, 0.1 mL, 0.4 mmol) was added. It was stirred at room temperature for 2 hrs, then solvent was removed in vacuo. The residue was triturated with EtOAc and the precipitate was collected as an off-white powder (10 mg, 74% yield). ¹H NMR (300 MHz, D₂O) δ 7.73 (m, 1H), 7.64-7.72 (m, 5H), 7.61 (m, 1H), 7.50 (m, 1H), 7.31 (m, 1H), 4.11 (m, 1H), 3.56-3.76 (m, 3H), 3.17 (m, 1H), 3.00 (m, 1H), 1.81 (m, 2H). MS (ESI): Calcd for C₂₀H₂₅N₄O₂⁺ 353.18 [M+H]⁺, found 353.00 [M+H]⁺.

The requisite intermediates were prepared as shown in the following steps.

### Step 1)

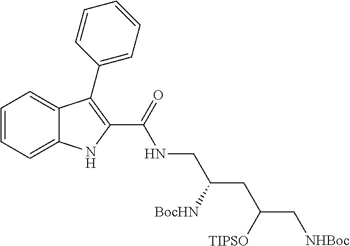

### di-tert-butyl ((4S)-5-(3-phenyl-1H-indole-2-carboxamido)-2-((triisopropylsilyl)oxy)pentane-1,4-diyl)dicarbamate

To a solution of 3-phenyl-1H-indole-2-carboxylic acid (24 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and di-tert-butyl ((4S)-5-amino-2-((triisopropylsilyl)oxy)pentane-1,4-diyl)dicarbamate (Amine Intermediate B) (49 mg, 0.1 mmol) was added. The reaction was continued at room temperature overnight. It was extracted with EtOAc and washed with water and brine. The organic layer was dried over anhydrous sodium sulfate and filtered. The filtrate was concentrated and purified by column chromatography on silica gel using 20-60% EtOAc in hexane as eluents to give two diastereomers (60 mg, 85% yield) as a white powder. ¹H NMR (300 MHz, CDCl₃) δ 9.26 (br s, 1H), 7.57 (m, 1H), 7.54 (m, 4H), 7.27 (t, J=8.1 Hz, 2H), 7.69 (d, J=8.1 Hz, 2H), 7.33 (m, 1H), 7.13 (m, 1H), 6.16 (br s, 1H), 5.13 (br s, 1H), 4.87 (br, 1H), 3.99 (m, 1H), 3.61 (m, 2H), 3.54 (m, 1H), 3.26 (m, 1H), 3.10 (m, 1H), 1.60 (m, 2H), 1.39 (s, 9H), 1.38 (s, 12H), 1.04 (s, 18H). MS (ESI): Calcd for C₃₉H₆₁N₄O₆Si⁺709.43 [M+H]⁺, found 709.45 [M+H]⁺.

### Step 2)

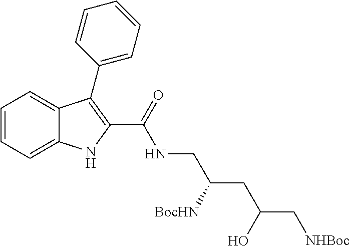 [SMILES: CC(C)(C)OC(=O)NCC(O)C[C@@H](CNC(=O)c1[nH]c2ccccc2c1-c1ccccc1)NC(=O)OC(C)(C)C]

### di-tert-butyl ((4S)-2-hydroxy-5-(3-phenyl-1H-indole-2-carboxamido)pentane-1,4-diyl)dicarbamate

To a solution of the less polar one of the two diastereomers of di-tert-butyl ((4S)-5-(3-phenyl)-1H-indole-2-carboxamido)-2-((triisopropylsilyl)oxy)pentane-1,4-diyl)dicarbamate (40 mg, 0.055 mmol) in THE (5 mL) was added TBAF solution (1.0 M in THF, 0.4 mL, 0.4 mmol) at room temperature It was stirred for 1 hr and concentrated, then loaded on column chromatography on silica gel and purified with 50-80% EtOAc in hexane to provide the alcohol as a white powder (18 mg, 58% yield). MS (ESI): Calcd for C₃₀H₄₁N₄O₆⁺ 553.30 [M+H]⁺, found 553.15 [M+H]⁺.

### Example 13. Preparation of N-((2S)-2,5-diamino-4-hydroxypentyl)-5-fluoro-3-phenyl-1H-indole-2-carboxamide hydrogen chloride salt

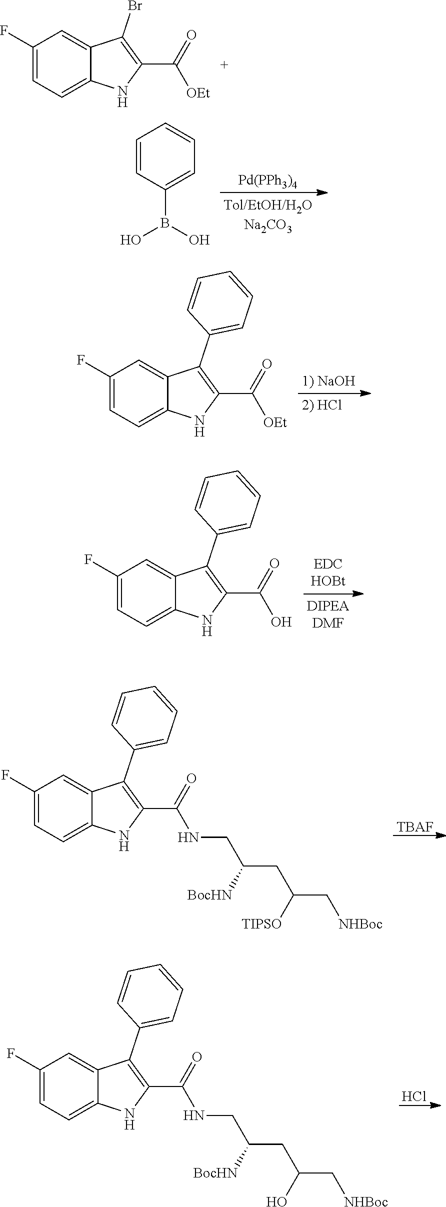

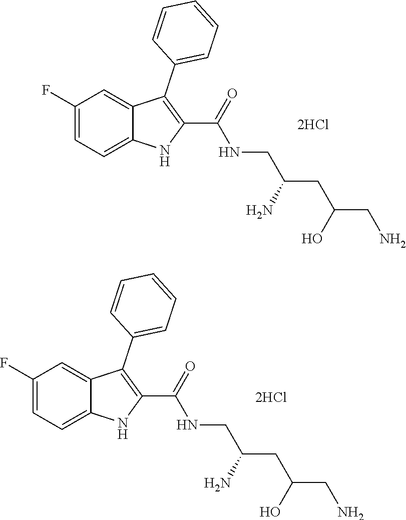 [SMILES: NCC(O)C[C@H](N)CNC(=O)c1[nH]c2ccc(F)cc2c1-c1ccccc1 ; NCC(O)C[C@H](N)CNC(=O)c1[nH]c2ccc(F)cc2c1-c1ccccc1]

### N-((2S)-2,5-diamino-4-hydroxypentyl)-5-fluoro-3-phenyl-1H-indole-2-carboxamide hydrogen chloride salt

To a solution of di-tert-butyl ((4S)-5-(5-fluoro-3-phenyl-1H-indole-2-carboxamido)-2-hydroxypentane-1,4-diyl)dicarbamate (17 mg, 0.03 mmol) in MeOH (2 mL) HCl solution (4 M in dioxane, 0.1 mL, 0.4 mmol) was added. It was stirred at room temperature for 2 hrs, then solvent was removed in vacuo. The residue was triturated with EtOAc and the precipitate was collected as an off-white powder (10 mg, 76% yield). ¹H NMR (300 MHz, CD₃OD) δ 7.73 (m, 4H), 7.0 (m, 2H), 7.36 (m, 1H), 7.26 (m, 1H), 4.08 (m, 1H), 3.50-3.74 (m, 6H), 3.13 (m, 1H), 2.98 (m, 1H), 1.78 (m, 2H). MS (ESI): Calcd for C₂₀H₂₅FN₄O₂⁺ 370.19 [M+H]⁺, found 371.00 [M+H]⁺.

The requisite intermediates were prepared as shown in the following steps.

### Step 1)

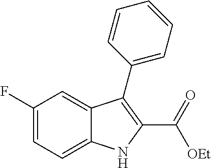 [SMILES: CCOC(=O)c1[nH]c2ccc(F)cc2c1-c1ccccc1]

### ethyl 5-fluoro-3-phenyl-1H-indole-2-carboxylate

Ethyl 5-fluoro-3-bromo-1H-indole-2-carboxylate (0.86 g, 3 mmol) and phenylboronic acid (0.5 g, 4.1 mmol) in a mixture of toluene, ethanol and saturated Na₂CO₃ solution (20/4/4 mL) was degassed and Pd(dppf)Cl₂ (50 mg, 0.07 mmol) was added. The reaction mixture was heated at 100° C. for 2 h, it was extracted with EtOAc and washed with brine and dried over Na₂SO₄, then concentrated. It was purified by column chromatography on silica gel with 0-20% EtOAc in hexane as eluents to give the product (0.51 g, 61% yield) as a white powder.

### Step 2)

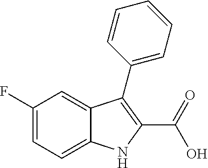 [SMILES: O=C(O)c1[nH]c2ccc(F)cc2c1-c1ccccc1]

### 5-fluoro-3-phenyl-1H-indole-2-carboxylic acid

To a solution of ethyl 5-fluoro-3-phenyl-1H-indole-2-carboxylate (500 mg, 1.78 mmol) in THF (10 mL) was added NaOH solution (2 M, 5 mL). It was heated at 50° C. overnight. THF was removed in vacuo and the residue was acidified with HCl solution. The precipitate was filtered and washed with water. It was dried to provide the product as a pale brown powder (390 mg, 86% yield) which was used for next step reaction without further purification. ¹H NMR (300 MHz, DMSO-d₆) δ 13.0 (br s, 1H), 11.95 (br s, 1H), 7.50 (m, 2H), 7.45 (m, 2H), 7.38-7.43 (m, 2H), 7.33 (m, 1H), 7.09-7.18 (m, 2H). MS (ESI): Calcd for C₁₅H₁₁FNO₂⁺ 256.08 [M+H]⁺, found 255.80 [M+H]⁺.

### Step 3)

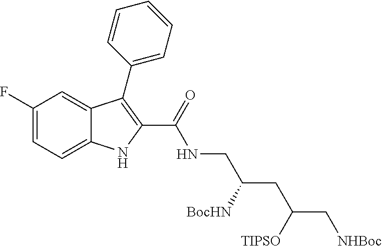

### di-tert-butyl ((4S)-5-(5-fluoro-3-phenyl-1H-indole-2-carboxamido)-2-((triisopropylsilyl)oxy)pentane-1,4-diyl)dicarbamate

To a solution of 5-fluoro-3-phenyl-1H-indole-2-carboxylic acid (26 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and di-tert-butyl ((4S)-5-amino-2-((triisopropylsilyl)oxy)pentane-1,4-diyl)dicarbamate (Amine Intermediate B) (49 mg, 0.1 mmol) was added. The reaction mixture was stirred at room temperature overnight. It was extracted with EtOAc and washed with water and brine. The organic layer was dried over anhydrous sodium sulfate and filtered. The filtrate was concentrated and purified by column chromatography on silica gel using 20-60% EtOAc in hexane as eluents to give two diastereomers (54 mg, 74% yield) as a white powder. ¹H NMR (300 MHz, CDCl₃) δ 9.22 (br s, 1H), 7.57 (m, 1H), 7.51 (m, 4H), 7.37 (m, 1H), 7.09 (m, 2H), 6.17 (br s, 1H), 5.07 (br s, 1H), 4.82 (br, 1H), 3.99 (m, 1H), 3.62 (m, 2H), 3.54 (m, 1H), 3.29 (m, 1H), 3.09 (m, 1H), 1.60 (m, 2H), 1.39 (s, 9H), 1.38 (s, 12H), 1.04 (s, 18H). MS (ESI): Calcd for C₃₉H₆₀FN₄O₆Si⁺727.43 [M+H]⁺, found 727.40 [M+H]⁺.

### Step 4)

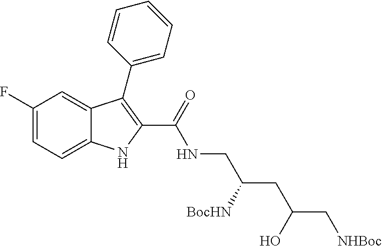 [SMILES: CC(C)(C)OC(=O)NCC(O)C[C@@H](CNC(=O)c1[nH]c2ccc(F)cc2c1-c1ccccc1)NC(=O)OC(C)(C)C]

### di-tert-butyl ((4S)-5-(5-fluoro-3-phenyl-1H-indole-2-carboxamido)-2-hydroxypentane-1,4-diyl)dicarbamate

To a solution of the less polar one of the two diastereomers of di-tert-butyl ((4S)-5-(5-fluoro-3-phenyl-1H-indole-2-carboxamido)-2-((triisopropylsilyl)oxy)pentane-1,4-diyl)dicarbamate (33 mg, 0.045 mmol) in THE (5 mL) was added TBAF solution (1.0 M in THF, 0.4 mL, 0.4 mmol) at room temperature It was stirred for 1 hr and concentrated, then loaded on silica gel column chromatography and purified with 50-80% EtOAc in hexane to provide the alcohol as a white powder (17 mg, 65% yield). MS (ESI): Calcd for C₃₀H₄₀FN₄O₆⁺571.29 [M+H]⁺, found 571.15 [M+H]⁺.

### Example 14. Preparation of 3-(4-cyanophenyl)-N-((2S)-2,5-diamino-4-hydroxypentyl)-7-fluoro-1H-indole-2-carboxamide hydrogen chloride salt

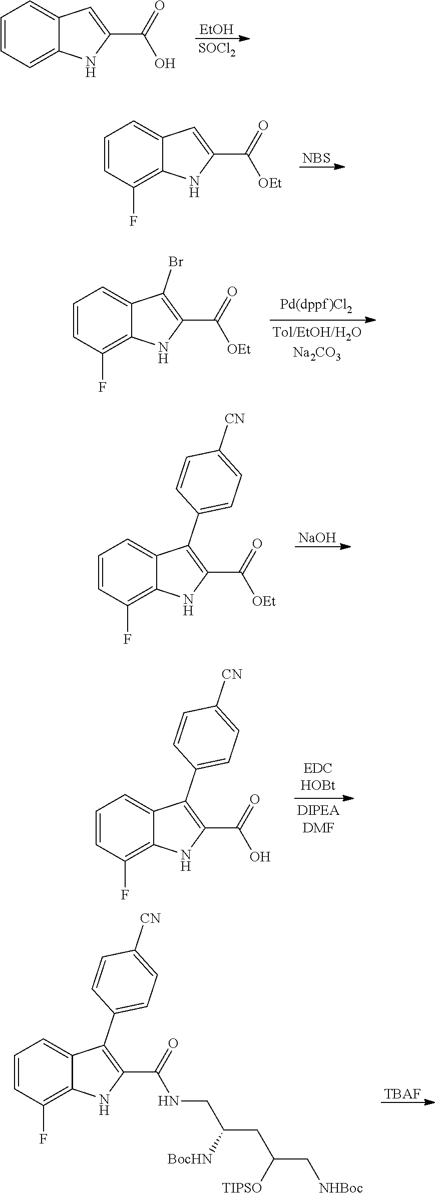

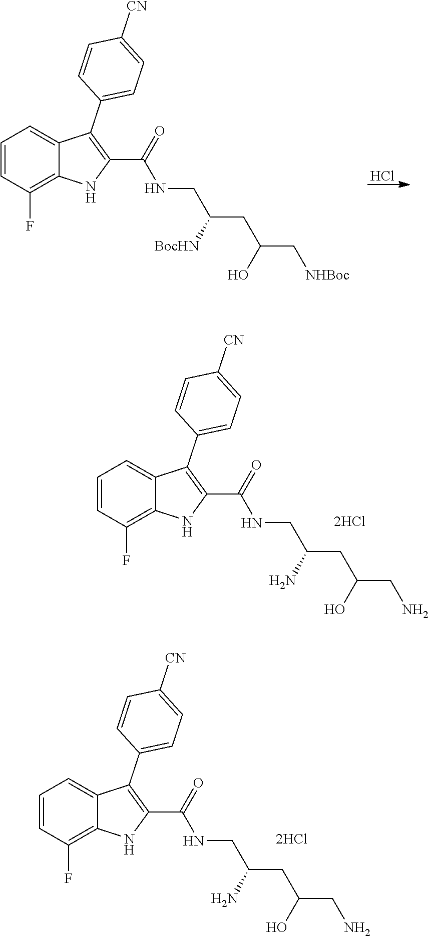 [SMILES: CC(C)(C)OC(=O)NCC(O)C[C@@H](CNC(=O)c1[nH]c2c(F)cccc2c1-c1ccc(C#N)cc1)NC(=O)OC(C)(C)C ; Cl ; N#Cc1ccc(-c2c(C(=O)NC[C@@H](N)CC(O)CN)[nH]c3c(F)cccc23)cc1 ; N#Cc1ccc(-c2c(C(=O)NC[C@@H](N)CC(O)CN)[nH]c3c(F)cccc23)cc1]

### 3-(4-cyanophenyl)-N-((2S)-2,5-diamino-4-hydroxypentyl)-7-fluoro-1H-indole-2-carboxamide hydrogen chloride salt

To a solution of di-tert-butyl ((4S)-5-(3-(4-cyanophenyl)-7-fluoro-1H-indole-2-carboxamido)-2-hydroxypentane-1,4-diyl)dicarbamate (11 mg, 0.018 mmol) in MeOH (2 mL) HCl solution (4 M in dioxane, 0.1 mL, 0.4 mmol) was added. It was stirred at room temperature for 2 hrs, then solvent was removed in vacuo. The residue was triturated with EtOAc and the precipitate was collected as an off-white powder (7 mg, 81% yield). ¹H NMR (300 MHz, D₂O) δ 7.99 (d, J=8.3 Hz, 2H), 7.80 (d, J=8.3 Hz, 2H), 7.54 (m, 1H), 7.26 (m, 1H), 7.64 (m, 1H), 4.17 (m, 1H), 3.80 (m, 1H), 3.76 (m, 2H), 3.66 (m, 1H), 3.22 (m, 1H), 3.03 (m, 1H), 1.91 (m, 2H). MS (ESI): Calcd for C₂₁H₂₃FN₅O₂⁺ 396.18 [M+H]⁺, found 396.00 [M+H]⁺.

The requisite intermediates were prepared as shown in the following steps.

### Step 1)

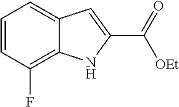 [SMILES: CCOC(=O)c1cc2cccc(F)c2[nH]1]

### ethyl 7-fluoro-1H-indole-2-carboxylate

To a suspension of 7-fluoro-1H-indole-2-carboxylic acid (0.8 g, 4.4 mmol) in EtOH (50 mL) was added SOCl₂ (0.66 mL, 9 mmol) very slowly. The mixture was heated under reflux until TLC showed no starting material left. Solvent was removed in vacuo and the crude product was dissolved in EtOAc, washed with sat. NaHCO₃ solution, brine and dried over Na₂SO₄. After filtering off, the organic phase was removed in vacuo to provide the ester as a brown powder (0.88 g, 95% yield) after drying. It was used for next step reaction without purification.

### Step 2)

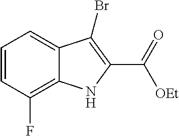 [SMILES: CCOC(=O)c1[nH]c2c(F)cccc2c1Br]

### ethyl 3-bromo-7-fluoro-1H-indole-2-carboxylate

To a solution of ethyl 7-fluoro-1H-indole-2-carboxylate (0.45 g, 2.2 mmol) in dry THE (10 mL) was added NBS (0.39 g, 2.2 mmol) at −78° C. Once the temperature reached to room temperature it was washed with water, sat. NaHCO₃ and brine. After removing solvent in vacuo, the residue was used for next step reaction without purification (0.60 g, 96% yield) as a white powder. ¹H NMR (300 MHz, CDCl₃) δ 9.25 (br s, 1H), 7.45 (m, 1H), 7.16 (m, 1H), 7.10 (m, 1H), 4.48 (q, J=7.2 Hz, 2H), 1.47 (t, J=7.2 Hz, 3H).

### Step 3)

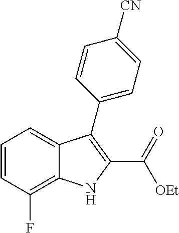 [SMILES: CCOC(=O)c1[nH]c2c(F)cccc2c1-c1ccc(C#N)cc1]

### ethyl 3-(4-cyanophenyl)-7-fluoro-1H-indole-2-carboxylate

Ethyl 3-bromo-7-fluoro-1H-indole-2-carboxylate (200 mg, 0.7 mmol) and (4-cyanophenyl)boronic acid (147 mg, 1 mmol) in a mixture of toluene, ethanol and saturated Na₂CO₃ solution (10/3/3 mL) was degassed and Pd(dppf)Cl₂ (30 mg, 0.04 mmol) was added. The reaction mixture was heated at 100° C. overnight, it was extracted with EtOAc and washed with brine and dried over Na₂SO₄, then concentrated. It was purified by column chromatography on silica gel with 0-25% EtOAc in hexane as eluents to give the product (93 mg, 43% yield) as an off-white powder. ¹H NMR (300 MHz, CDCl₃) δ 9.22 (br s, 1H), 7.75 (d, J=8.4 Hz, 2H), 7.67 (d, J=8.4 Hz, 2H), 7.33 (m, 1H), 7.10 (m, 2H), 4.33 (q, J=7.1 Hz, 2H), 1.23 (t, J=7.1 Hz, 3H).

### Step 4)

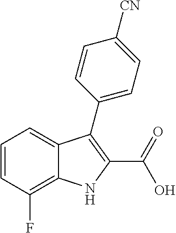 [SMILES: N#Cc1ccc(-c2c(C(=O)O)[nH]c3c(F)cccc23)cc1]

### 3-(4-cyanophenyl)-7-fluoro-1H-indole-2-carboxylic acid

To a solution of ethyl 3-(4-cyanophenyl)-7-fluoro-1H-indole-2-carboxylate (93 mg, 0.3 mmol) in THF (4 mL) was added LiOH solution (2 M, 2 mL). It was heated at 45° C. for 2 hrs. THF was removed in vacuo and the residue was acidified with HCl solution. The precipitate was filtered and washed with water. It was dried to provide the acid as a pale brown powder (60 mg, 71% yield) which was used for next step reaction without further purification. MS (ESI): Calcd for C₁₆H₈FN₂O₂⁻ 279.06 [M−H]⁻, found 279.00 [M−H]⁻.

### Step 5)

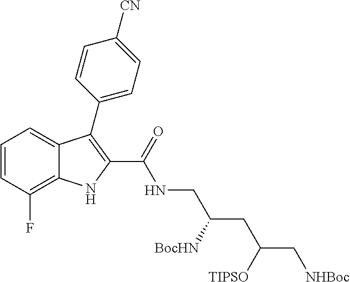

### di-tert-butyl ((4S)-5-(3-(4-cyanophenyl)-7-fluoro-1H-indole-2-carboxamido)-2-((triisopropylsilyl)oxy)pentane-1,4-diyl)dicarbamate

To a solution of 3-(4-cyanophenyl)-7-fluoro-1H-indole-2-carboxylic acid (28 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and di-tert-butyl ((4S)-5-amino-2-((triisopropylsilyl)oxy)pentane-1,4-diyl)dicarbamate (Amine Intermediate B) (49 mg, 0.1 mmol) was added. The reaction mixture was stirred at room temperature overnight. It was extracted with EtOAc and washed with water and brine. The organic layer was dried over anhydrous sodium sulfate and filtered. The filtrate was concentrated and purified by column chromatography on silica gel using 40-70% EtOAc in hexane as eluents to give two diastereomers (51 mg, 68% yield) as a white powder. ¹H NMR (300 MHz, CDCl₃) δ 9.44 (br s, 1H), 7.82 (d, J=8.1 Hz, 2H), 7.69 (d, J=8.1 Hz, 2H), 7.39 (m, 1H), 7.10 (m, 2H), 6.71 (br s, 1H), 5.27 (br s, 1H), 4.77 (br, 1H), 4.01 (m, 1H), 3.71 (m, 1H), 3.41 (m, 2H), 3.25 (m, 1H), 3.16 (m, 1H), 1.63 (m, 2H), 1.41 (s, 9H), 1.25 (s, 12H), 1.06 (s, 18H). MS (ESI): Calcd for C₄₀H₅₉FN₅O₆Si⁺752.42 [M+H]⁺, found 752.40 [M+H]⁺.

### Step 6)

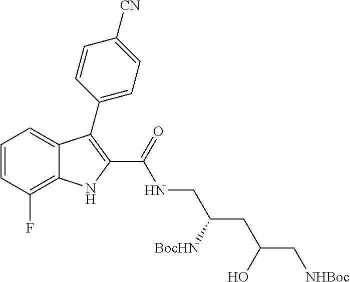 [SMILES: CC(C)(C)OC(=O)NCC(O)C[C@@H](CNC(=O)c1[nH]c2c(F)cccc2c1-c1ccc(C#N)cc1)NC(=O)OC(C)(C)C]

### di-tert-butyl ((4S)-5-(3-(4-cyanophenyl)-7-fluoro-1H-indole-2-carboxamido)-2-hydroxypentane-1,4-diyl)dicarbamate

To a solution of the relatively polar one of the two diastereomers of tert-butyl ((4S)-5-(3-(4-cyanophenyl)-7-fluoro-1H-indole-2-carboxamido)-2-((triisopropylsilyl)oxy)pentane-1,4-diyl)dicarbamate (20 mg, 0.026 mmol) in THE (2 mL) was added TBAF solution (1.0 M in THF, 0.4 mL, 0.4 mmol) at room temperature It was stirred for 0.5 hr, concentrated and loaded on silica gel column chromatography and purified with 40-65% EtOAc in hexane to provide the alcohol as a white powder (11 mg, 69% yield). MS (ESI): Calcd for C₃₁H₃₉FN₅O₆⁺ 596.29 [M+H]⁺, found 596.25 [M+H]⁺.

### Example 15. Preparation of 3-cyclohexyl-N-((2S)-2,5-diamino-4-hydroxypentyl)-1H-indole-2-carboxamide hydrogen chloride salt

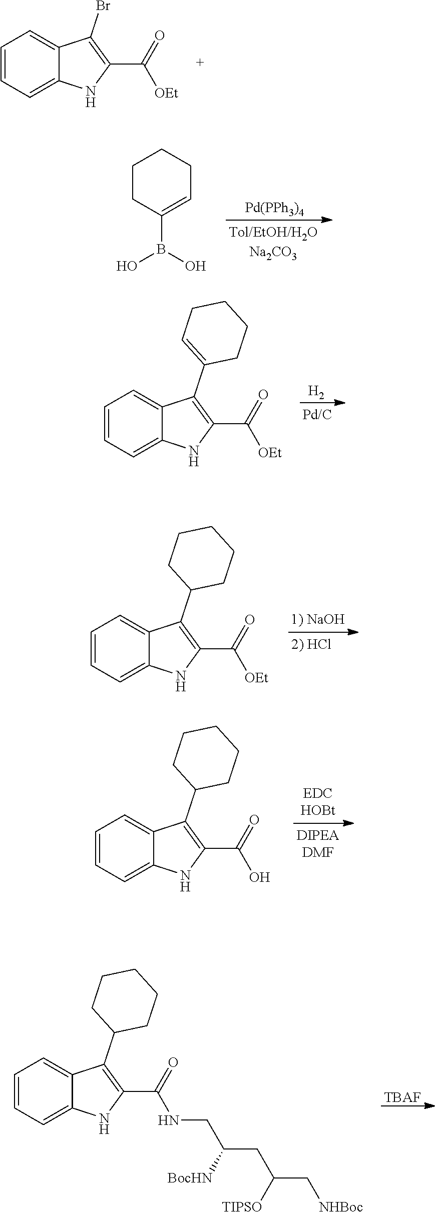

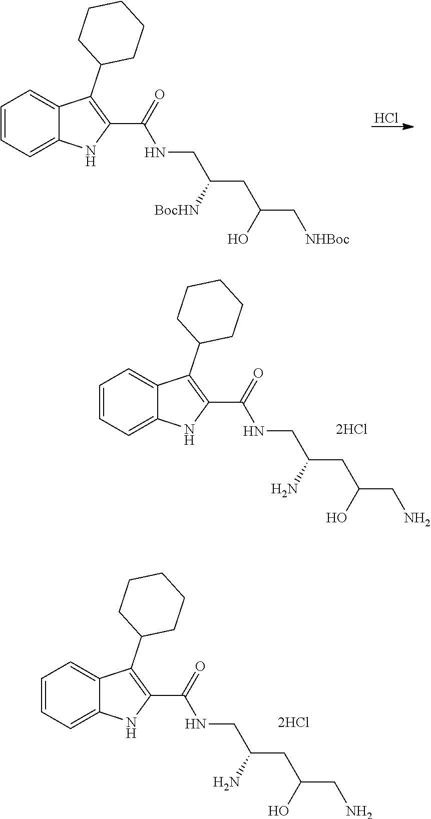 [SMILES: CC(C)(C)OC(=O)NCC(O)C[C@@H](CNC(=O)c1[nH]c2ccccc2c1C1CCCCC1)NC(=O)OC(C)(C)C ; Cl ; NCC(O)C[C@H](N)CNC(=O)c1[nH]c2ccccc2c1C1CCCCC1 ; NCC(O)C[C@H](N)CNC(=O)c1[nH]c2ccccc2c1C1CCCCC1]

### 3-cyclohexyl-N-((2S)-2,5-diamino-4-hydroxypentyl)-1H-indole-2-carboxamide hydrogen chloride salt

To a solution of di-tert-butyl ((4S)-5-(3-cyclohexyl-1H-indole-2-carboxamido)-2-hydroxypentane-1,4-diyl)dicarbamate (20 mg, 0.036 mmol) in MeOH (2 mL) HCl solution (4 M in dioxane, 0.1 mL, 0.4 mmol) was added. It was stirred at room temperature for 2 hrs, then solvent was removed in vacuo. The residue was loaded on a C18 column and purified with EtOH in water to provide the product as a colorless powder (7 mg, 45% yield). ¹H NMR (300 MHz, CD₃OD) δ 7.81 (d, J=8.2 Hz, 1H), 7.37 (d, J=8.2 Hz, 1H), 7.18 (m, 1H), 7.00 (m, 1H), 3.92 (m, 1H), 3.54 (m, 1H), 3.48 (m, 1H), 3.23 (m, 1H), 2.85 (m, 1H), 2.73 (m, 1H), 2.05 (m, 1H), 1.73-1.95 (m, 6H), 1.26-1.54 (m, 4H). MS (ESI): Calcd for C₂₀H₃₁N₄O₂⁺ 359.24 [M+H]⁺, found 359.05 [M+H]⁺.

The requisite intermediates were prepared as shown in the following steps.

### Step 1)

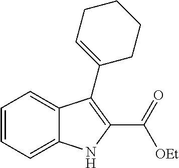 [SMILES: CCOC(=O)c1[nH]c2ccccc2c1C1=CCCCC1]

### ethyl 3-(cyclohex-1-en-1-yl)-1H-indole-2-carboxylate

Ethyl 3-bromo-1H-indole-2-carboxylate (1.0 g, 3.73 mmol) and cyclohex-1-en-1-ylboronic acid (0.7 g, 5.56 mmol) in a mixture of toluene, ethanol and saturated Na₂CO₃ solution (30/6/6 mL) was degassed and Pd(dppf)Cl₂ (70 mg, 0.1 mmol) was added. The reaction mixture was heated at 100° C. for 4 hours, it was extracted with EtOAc and washed with brine and dried over Na₂SO₄, then concentrated. It was purified by column chromatography on silica gel with 0-20% EtOAc in hexane as eluents to give the product (0.98 g, 99% yield) as an off-white powder. ¹H NMR (300 MHz, CDCl₃) δ 8.74 (br s, 1H), 7.37 (d, J=7.6 Hz, 1H), 7.34 (m, 1H), 7.32 (m, 1H), 7.13 (m, 1H), 5.80 (m, 1H), 4.38 (q, J=7.1 Hz, 2H), 2.37 (m, 2H), 2.27 (m, 2H), 1.78 (m, 4H), 1.41 (t, J=7.1 Hz, 3H).

### Step 2)

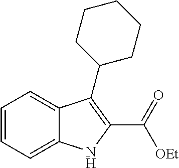 [SMILES: CCOC(=O)c1[nH]c2ccccc2c1C1CCCCC1]

### ethyl 3-cyclohexyl-1H-indole-2-carboxylate

To a solution of ethyl 3-(cyclohex-1-en-1-yl)-1H-indole-2-carboxylate (0.98 g, 3.6 mmol) in MeOH (50 mL) was added Pd/C (10 w/w %, 100 mg, 0.09 mmol). It was stirred under H₂ (50 psi) overnight. The catalyst was filter off through a Celite pad and washed with MeOH. The combined filtrate was concentrated and used without further purification (0.77 g, 78%). ¹H NMR (300 MHz, CDCl₃) δ 8.67 (br s, 1H), 7.93 (d, J=8.2 Hz, 1H), 7.36 (m, 1H), 7.29 (m, 1H), 7.10 (m, 1H), 4.40 (q, J=7.1 Hz, 2H), 3.74 (m, 1H), 1.99 (m, 2H), 1.87 (m, 6H), 1.78 (m, 4H), 1.46 (m, 2H), 1.43 (t, J=7.1 Hz, 3H).

### Step 3)

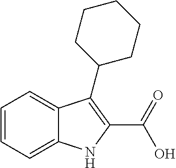 [SMILES: O=C(O)c1[nH]c2ccccc2c1C1CCCCC1]

### 3-cyclohexyl-1H-indole-2-carboxylic acid

To a solution of ethyl 3-cyclohexyl-1H-indole-2-carboxylate (770 mg, 2.8 mmol) in THE (4 mL) was added NaOH solution (2 M, 10 mL). It was heated at 50° C. overnight. THE was removed in vacuo and the residue was acidified with HCl solution. The precipitate was filtered and washed with water. It was dried to provide the acid as a pale brown powder (250 mg, 36% yield) which was used for next step reaction without further purification. MS (ESI): Calcd for C₁₅H₁₈NO₂⁺ 244.14 [M+H]⁺, found 243.90 [M+H]⁺.

### Step 4)

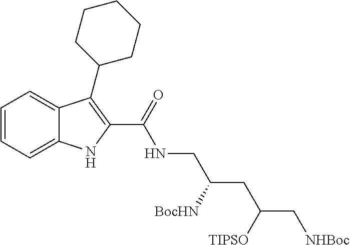

### di-tert-butyl ((4S)-5-(3-cyclohexyl-1H-indole-2-carboxamido)-2-((triisopropylsilyl)oxy)pentane-1,4-diyl)dicarbamate

To a solution of 3-cyclohexyl-1H-indole-2-carboxylic acid (24 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and di-tert-butyl ((4S)-5-amino-2-((triisopropylsilyl)oxy)pentane-1,4-diyl)dicarbamate (Amine Intermediate B) (49 mg, 0.1 mmol) was added. The reaction mixture was stirred at room temperature overnight. It was extracted with EtOAc and washed with water and brine. The organic layer was dried over anhydrous sodium sulfate and filtered. The filtrate was concentrated and purified by column chromatography on silica gel using 50-70% EtOAc in hexane as eluents to give the amide (42 mg, 59% yield) as a white powder. ¹H NMR (300 MHz, CDCl₃) δ 8.66 (br s, 1H), 7.93 (m, 1H), 7.36 (m, 1H), 7.29 (m, 1H), 7.09 (m, 1H), 6.84 (br s, 1H), 5.11 (br s, 1H), 4.95 (br, 1H), 4.03 (m, 1H), 3.73 (m, 2H), 3.47 (m, 1H), 3.35 (m, 1H), 3.18 (m, 1H), 2.00 (m, 1H), 1.71-1.98 (m, 4H), 1.60 (m, 2H), 1.33-1.50 (m, 27H), 1.07 (s, 18H). MS (ESI): Calcd for C₃₉H₆₇N₄O₆Si⁺ 715.48 [M+H]⁺, found 715.40 [M+H]⁺.

### Step 5)

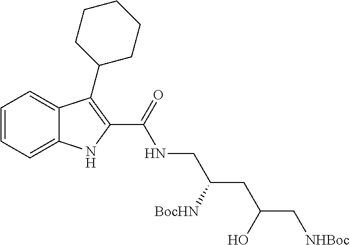 [SMILES: CC(C)(C)OC(=O)NCC(O)C[C@@H](CNC(=O)c1[nH]c2ccccc2c1C1CCCCC1)NC(=O)OC(C)(C)C]

### di-tert-butyl ((4S)-5-(3-cyclohexyl-1H-indole-2-carboxamido)-2-hydroxypentane-1,4-diyl)dicarbamate

To a solution of di-tert-butyl ((4S)-5-(3-cyclohexyl-1H-indole-2-carboxamido)-2-((triisopropylsilyl)oxy)pentane-1,4-diyl)dicarbamate (42 mg, 0.059 mmol) in THE (5 mL) was added TBAF solution (1.0 M in THF, 0.4 mL, 0.4 mmol) at room temperature It was stirred for 1 hr and concentrated, then loaded on silica gel column chromatography and purified with 50-80% EtOAc in hexane to provide the alcohol as a white powder (20 mg, 61% yield). MS (ESI): Calcd for C₃₀H₄₇N₄O₆ 559.35 [M+H]⁺, found 559.05 [M+H]⁺.

### Example 16. Preparation of N-((2R)-2,5-diamino-4-(hydroxymethyl)pentyl)-3-(4-fluorophenyl)-1H-indole-2-carboxamide hydrogen chloride salt

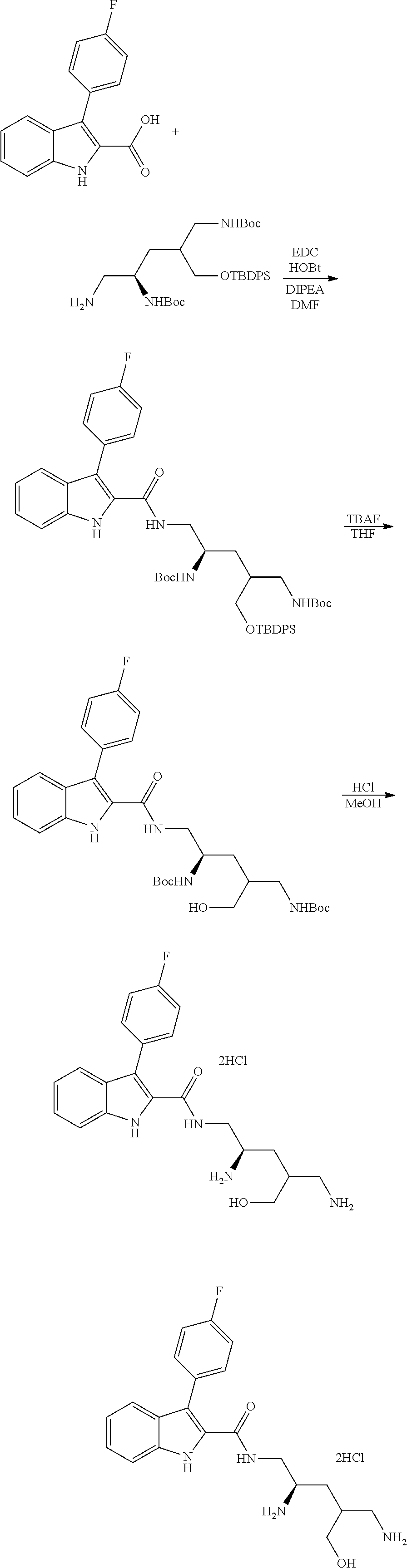

### N-((2R)-2,5-diamino-4-(hydroxymethyl)pentyl)-3-(4-fluorophenyl)-1H-indole-2-carboxamide hydrogen chloride salt

To a solution of di-tert-butyl ((4R)-5-(3-(4-fluorophenyl)-1H-indole-2-carboxamido)-2-(hydroxymethyl)pentane-1,4-diyl)dicarbamate (136 mg, 0.23 mmol) in MeOH (5 mL) was added HCl (0.5 mL, 4 N in dioxane). The reaction mixture was stirred at 50° C. for 1 h and then concentrated to give a residue. The residue was triturated with EtOAc to give the desired product (75 mg, 71% yield) as a white solid. ¹H NMR (300 MHz, D₂O) δ 7.55 (m, 4H), 7.15-7.39 (m, 4H), 3.54-3.68 (m, 3H), 3.44-3.52 (m 2H), 3.00 (m, 2H), 2.00 (m, 1H), 1.57 (m, 2H). MS (EPI): Calcd for C₂₁H₂₆FN₄O₂⁺ 385.20 [M+H]⁺, found 385.00 [M+H]⁺.

The requisite intermediates were prepared as shown in the following steps.

### Step 1)

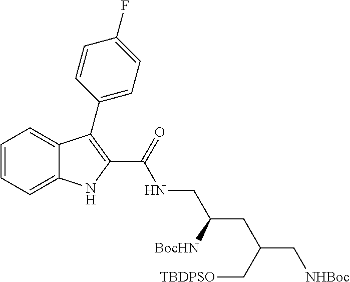

### di-tert-butyl ((4R)-2-(((tert-butyldiphenylsilyl)oxy)methyl)-5-(3-(4-fluorophenyl)-1H-indole-2-carboxamido)pentane-1,4-diyl)dicarbamate

To a solution of 3-(4-fluorophenyl)-1H-indole-2-carboxylic acid (106 mg, 041 mmol) in dry DMF (3 mL) was added DIPEA (0.14 mL, 0.75 mmol), HOBt (30 mg, 0.23 mmol) and EDC (80 mg, 0.41 mmol). The reaction mixture was stirred at room temperature for 10 min and then di-tert-butyl ((3R,4S)-5-amino-3-hydroxypentane-1,4-diyl) dicarbamate (Amine Intermediate C) (223 mg, 0.38 mmol) was added. The reaction mixture was stirred at room temperature overnight then added H₂O with stirring. A solid was formed and collected by filtration. After air drying the solid was purified by chromatography on silica gel. Elution with 10-20% EtOAc/hexanes afforded the desired compound (270 mg, 86% yield) as a white solid. ¹H NMR (300 MHz, CDCl₃) δ 9.22 (br s, 1H), 7.63 (m, 4H), 7.31-7.52 (m, 11H), 7.23 (m, 2H), 7.13 (m, 1H), 6.14 (br s, 1H), 4.80 (br s, 1H), 4.57 (d, J=9.0 Hz), 1H), 3.42-3.65 (m, 4H), 3.05-3.25 (m, 3H), 1.73 (m, 1H), 1.22-1.44 (m, 20H), 1.04 (s, 9H).

### Step 2)

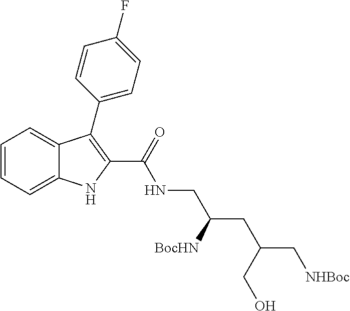 [SMILES: CC(C)(C)OC(=O)NCC(CO)C[C@H](CNC(=O)c1[nH]c2ccccc2c1-c1ccc(F)cc1)NC(=O)OC(C)(C)C]

### di-tert-butyl ((4R)-5-(3-(4-fluorophenyl)-1H-indole-2-carboxamido)-2-(hydroxymethyl)pentane-1,4-diyl)dicarbamate

To a solution of di-tert-butyl ((4R)-2-(((tert-butyldiphenylsilyl)oxy)methyl)-5-(3-(4-fluorophenyl)-1H-indole-2-carboxamido)pentane-1,4-diyl)dicarbamate (0.27 g, 0.33 mmol) in THF (15 mL) was added TBAF solution in THF (1.0 M, 1.0 mL, 1.0 mmol). It was stirred at room temperature until no starting material remained in the reaction mixtures. The crude product was purified by column chromatography on silica gel using 50% EtOAc in hexane as eluents. A white solid was collected (0.136 g, 71% yield). ¹H NMR (300 MHz, CDCl₃) δ 9.86 (br s, 1H), 7.40-7.51 (m, 4H), 7.22-7.34 (m, 3H), 7.12 (m, 1H), 6.16 (br, 1H), 5.07-5.19 (m, 1H), 4.69-4.86 (m, 1H), 3.06-3.86 (m, 7H), 1.63 (m, 1H), 1.20-1.44 (m, 20H).

### Example 17. Preparation of N-((2R)-2,5-diamino-4-(hydroxymethyl)pentyl)-5-fluoro-3-phenyl-1H-indole-2-carboxamide hydrogen chloride salt

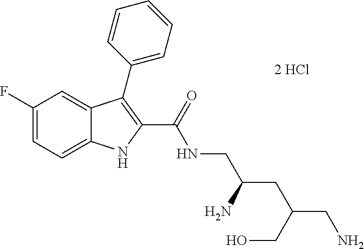 [SMILES: NCC(CO)C[C@@H](N)CNC(=O)c1[nH]c2ccc(F)cc2c1-c1ccccc1]

### N-((2R)-2,5-diamino-4-(hydroxymethyl)pentyl)-5-fluoro-3-phenyl-1H-indole-2-carboxamide hydrogen chloride salt

To a solution of di-tert-butyl ((4R)-5-(5-fluoro-3-phenyl-1H-indole-2-carboxamido)-2-(hydroxymethyl)pentane-1,4-diyl)dicarbamate (52 mg, 0.09 mmol) in MeOH (3 mL) was added HCl (0.5 mL, 4 N in dioxane, 2 mmol). The reaction mixture was stirred at 50° C. for 1 h and then concentrated to give a residue. The residue was triturated with EtOAc to give the desired product (27 mg, 66% yield) as white solid. ¹H NMR (300 MHz, D₂O) δ 7.44-7.58 (m, 5H), 7.27 (m, 1H), 7.16 (m, 1H), 3.54-3.67 (m, 3H), 3.44-3.50 (m 2H), 2.99 (m, 2H), 2.00 (m, 1H), 1.56 (m, 2H). MS (EPI): Calcd for C₂₁H₂₆FN₄O₂⁺ 385.20 [M+H]⁺, found 385.00 [M+H]⁺.

The requisite intermediates were prepared as shown in the following steps.

### Step 1)

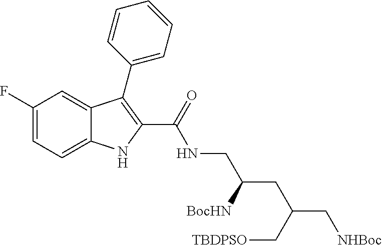

### di-tert-butyl ((4R)-2-(((tert-butyldiphenylsilyl)oxy)methyl)-5-(5-fluoro-3-phenyl-1H-indole-2-carboxamido)pentane-1,4-diyl)dicarbamate

To a solution of 5-fluoro-3-phenyl-1H-indole-2-carboxylic acid (45 mg, 018 mmol) in dry DMF (2 mL) was added DIPEA (0.06 mL, 0.32 mmol), HOBt (13 mg, 0.10 mmol) and EDC (34 mg, 0.18 mmol). The reaction mixture was stirred at room temperature for 10 min and then di-tert-butyl ((3R,4S)-5-amino-3-hydroxypentane-1,4-diyl) dicarbamate (Amine Intermediate C) (96 mg, 0.16 mmol) was added. The reaction mixture was stirred at room temperature overnight then added H₂O with stirring. A solid was formed and collected by filtration. After air drying the solid was purified by chromatography on silica gel. Elution with 10-20% EtOAc/hexanes afforded the desired compound (130 mg, 98% yield) as a white solid. ¹H NMR (300 MHz, CDCl₃) δ 9.45 (br s, 1H), 7.63 (m, 4H), 7.31-7.52 (m, 12H), 6.09 (br s, 1H), 4.82 (br s, 1H), 4.48-4.64 (m, 1H), 3.32-3.65 (m, 4H), 3.05-3.28 (m, 3H), 1.71 (m, 1H), 1.22-1.41 (m, 20H), 1.04 (s, 9H).

### Step 2)

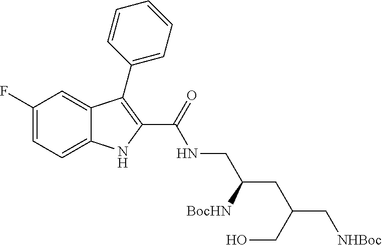 [SMILES: CC(C)(C)OC(=O)NCC(CO)C[C@H](CNC(=O)c1[nH]c2ccc(F)cc2c1-c1ccccc1)NC(=O)OC(C)(C)C]

### di-tert-butyl ((4R)-5-(5-fluoro-3-phenyl-1H-indole-2-carboxamido)-2-(hydroxymethyl)pentane-1,4-diyl)dicarbamate

To a solution of di-tert-butyl ((4R)-2-(((tert-butyldiphenylsilyl)oxy)methyl)-5-(5-fluoro-3-phenyl-1H-indole-2-carboxamido)pentane-1,4-diyl)dicarbamate (130 mg, 0.16 mmol) in THE (10 mL) was added TBAF solution in THE (1.0 M, 0.5 mL, 0.5 mmol). It was stirred at room temperature until no starting material remained in the reaction mixtures. The crude product was purified by column chromatography on silica gel using 50% EtOAc in hexane to afford the desired product as a white solid (53 mg, 57% yield). ¹H NMR (300 MHz, CDCl₃) δ 9.76 (br s, 1H), 7.45-7.60 (m, 5H), 7.40 (m, 1H), 7.07 (m, 2H), 6.18 (br s, 1H), 4.98-5.19 (m, 1H), 4.56-4.80 (m, 1H), 3.02-3.81 (m, 7H), 1.62 (m, 1H), 1.20-1.44 (m, 20H).

### Example 18. Preparation of 3-cyclohexyl-N-((2R)-2,5-diamino-4-(hydroxymethyl)pentyl)-5-fluoro-1H-indole-2-carboxamide hydrogen chloride salt

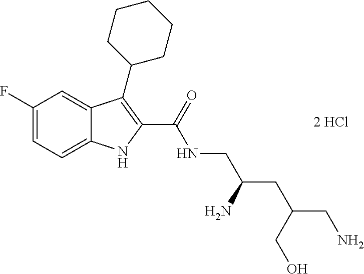 [SMILES: NCC(CO)C[C@@H](N)CNC(=O)c1[nH]c2ccc(F)cc2c1C1CCCCC1]

### 3-cyclohexyl-N-((2R)-2,5-diamino-4-(hydroxymethyl)pentyl)-5-fluoro-1H-indole-2-carboxamide hydrogen chloride salt

To a solution of di-tert-butyl ((4R)-5-(3-cyclohexyl-5-fluoro-1H-indole-2-carboxamido)-2-(hydroxymethyl)pentane-1,4-diyl)dicarbamate (16 mg, 0.03 mmol) in MeOH (1 mL) was added HCl (0.2 mL, 4 M in dioxane, 0.8 mmol). The reaction mixture was stirred at 50° C. for 1 h then concentrated to give a residue. The residue was triturated with EtOAc to afford the desired product (7 mg, 54% yield) as white solid. ¹H NMR (300 MHz, D₂O) δ 7.68 (dd, J=2.4, 10.5 Hz, 1H), 7.44 (dd, J=4.5, 8.7 Hz, 1H), 7.10 (m, 1H), 3.59-3.80 (m, 5H), 3.27 (m, 1H), 3.08 (m, 1H), 2.20 (m, 1H), 1.66-1.98 (m, 10H), 1.35 (m, 3H). MS (EPI): Calcd for C₂₁H₃₂FN₄O₂⁺ 391.25 [M+H]⁺, found 391.05 [M+H]⁺.

The requisite intermediates were prepared as shown in the following steps.

### Step 1)

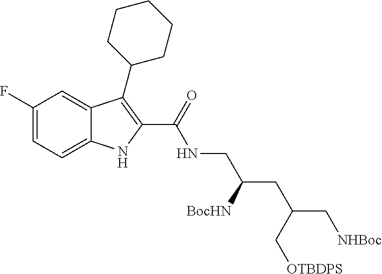

### di-tert-butyl ((4R)-2-(((tert-butyldiphenylsilyl)oxy)methyl)-5-(3-cyclohexyl-5-fluoro-1H-indole-2-carboxamido)pentane-1,4-diyl)dicarbamate

To a solution of 3-cyclohexyl-5-fluoro-1H-indole-2-carboxylic acid (24 mg, 0.09 mmol) in dry DMF (1 mL) was added DIPEA (0.03 mL, 0.16 mmol), HOBt (7 mg, 0.05 mmol) and EDC (17 mg, 0.09 mmol). The reaction mixture was stirred at room temperature for 10 min then di-tert-butyl ((3R,4S)-5-amino-3-hydroxypentane-1,4-diyl) dicarbamate (Amine Intermediate C) (48 mg, 0.08 mmol) was added. The reaction mixture was stirred at room temperature overnight then added H₂O with stirring. A solid was formed and collected by filtration. After air drying the solid was purified by chromatography on silica gel. Elution with 10-20% EtOAc/hexanes afforded the desired compound (26 mg, 34% yield) as a white solid. ¹H NMR (300 MHz, CDCl₃) δ 9.22 (br s, 1H), 7.65 (m, 4H), 7.50 (m, 1H), 7.36-7.45 (m, 5H), 7.23 (m, 1H), 7.04 (br, 1H), 6.90-7.01 (m, 1H), 4.99 (d, J=8.4 Hz, 1H), 4.48-4.64 (m, 1H), 4.69 (br s, 1H), 3.80 (m, 1H), 3.61 (m, 2H), 3.47 (m, 2H), 3.15 (m, 2H), 1.85 (m, 9H), 1.22-1.54 (m, 23H), 1.07 (m, 9H).

### Step 2)

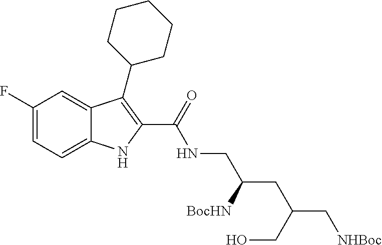 [SMILES: CC(C)(C)OC(=O)NCC(CO)C[C@H](CNC(=O)c1[nH]c2ccc(F)cc2c1C1CCCCC1)NC(=O)OC(C)(C)C]

### di-tert-butyl ((4R)-5-(3-cyclohexyl-5-fluoro-1H-indole-2-carboxamido)-2-(hydroxymethyl)pentane-1,4-diyl)dicarbamate

To a solution of di-tert-butyl ((4R)-2-(((tert-butyldiphenylsilyl)oxy)methyl)-5-(3-cyclohexyl-5-fluoro-1H-indole-2-carboxamido)pentane-1,4-diyl)dicarbamate (25 mg, 0.03 mmol) in THE (1 mL) was added TBAF solution in THE (1.0 M, 0.2 mL, 0.2 mmol). It was stirred at room temperature until no starting material remained in the reaction mixtures. The crude product was purified by column chromatography on silica gel using 50% EtOAc in hexane to afford the desired product as white solid (16 mg, 88% yield). ¹H NMR (300 MHz, CDCl₃) δ 9.24 (br s, 1H), 7.50 (dd, J=2.1, 10.5 Hz, 1H), 7.22 (m, 1H), 6.98 (m, 1H), 6.89 (br s, 1H), 4.95 (m, 2H), 3.85 (m, 1H), 3.54 (m, 4H), 3.20 (m, 3H), 1.85 (m, 9H), 1.22-1.54 (m, 23H).

### Example 19. Preparation of (R)—N-(2-amino-3-(2-aminoethoxy)propyl)-3-(4-fluorophenyl)-1H-indole-2-carboxamide hydrogen chloride salt

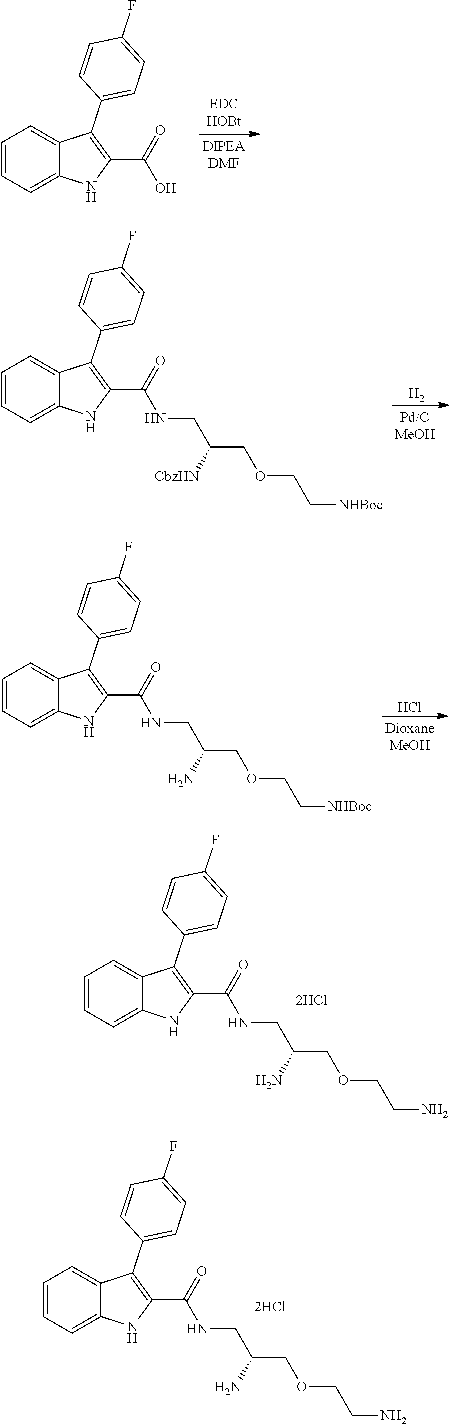

### (R)—N-(2-amino-3-(2-aminoethoxy)propyl)-3-(4-fluorophenyl)-1H-indole-2-carboxamide hydrogen chloride salt

To a solution of tert-butyl (R)-(2-(2-amino-3-(3-(4-fluorophenyl)-1H-indole-2-carboxamido)propoxy)ethyl)carbamate (25 mg, 0.05 mmol) in MeOH (5 mL) HCl solution (4 M in dioxane, 0.15 mL, 0.6 mmol) was added. It was stirred at room temperature for 2 hrs, then solvent was removed in vacuo. The residue was triturated with EtOAc and the precipitate was collected as a pale brown powder (16 mg, 65% yield). ¹H NMR (300 MHz, D₂O) δ 7.59 (m, 2H), 7.55 (m, 2H), 7.38 (m, 1H), 7.28 (d, J=9.0 Hz, 2H), 7.20 (t, J=7.5 Hz, 1H), 3.72 (m, 2H), 3.69 (m, 2H), 3.56 (m, 3H), 3.18 (m, 2H). MS (ESI): Calcd for C₂₀H₂₄FN₄O₂⁺ 371.19 [M+H]⁺, found 371.00 [M+H]⁺.

The requisite intermediates were prepared as shown in the following steps.

### Step 1)

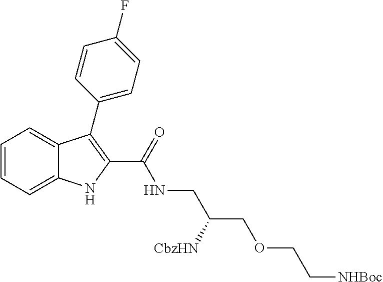 [SMILES: CC(C)(C)OC(=O)NCCOC[C@@H](CNC(=O)c1[nH]c2ccccc2c1-c1ccc(F)cc1)NC(=O)OCc1ccccc1]

### tert-butyl (R)-(2-(2-(((benzyloxy)carbonyl)amino)-3-(3-(4-fluorophenyl)-1H-indole-2-carboxamido)propoxy)ethyl)carbamate

To a solution of 3-(4-fluorophenyl)-1H-indole-2-carboxylic acid (26 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and benzyl (R)-(1-amino-3-(2-((tert-butoxycarbonyl)amino)ethoxy)propan-2-yl)carbamate (Amine Intermediate D) (37 mg, 0.1 mmol) was added. The reaction mixture was stirred at room temperature overnight. It was extracted with EtOAc and washed with water and brine. The organic layer was dried over anhydrous sodium sulfate and filtered. The filtrate was concentrated and purified by column chromatography on silica gel to give the product (50 mg, 83% yield) as a white powder. ¹H NMR (300 MHz, CDCl₃) δ 9.31 (br s, 1H), 7.44 (m, 4H), 7.31 (m, 5H), 7.21 (m, 3H), 7.14 (m, 1H), 6.17 (br s, 1H), 5.29 (br s, 1H), 5.06 (m, 2H), 3.80 (m, 1H), 3.57 (m, 2H), 3.48 (m, 2H), 3.39 (m, 2H), 3.26 (m, 2H), 1.44 (s, 9H). MS (ESI): Calcd for C₃₃H₃₈FN₄O₆⁺ 605.28 [M+H]⁺, found 605.20 [M+H]⁺.

### Step 2)

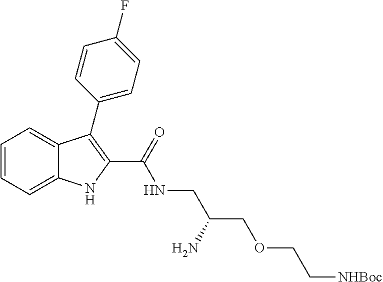 [SMILES: CC(C)(C)OC(=O)NCCOC[C@H](N)CNC(=O)c1[nH]c2ccccc2c1-c1ccc(F)cc1]

### tert-butyl (R)-(2-(2-amino-3-(3-(4-fluorophenyl)-1H-indole-2-carboxamido)propoxy)ethyl)carbamate

To a solution of tert-butyl (R)-(2-(2-(((benzyloxy)carbonyl)amino)-3-(3-(4-fluorophenyl)-1H-indole-2-carboxamido)propoxy)ethyl)carbamate (50 mg, 0.08 mmol) in MeOH (5 mL) was added Pd/C (10%, 20 mg, 0.02 mmol). Then it was stirred under H₂ (50 psi) overnight. It was filtered through a Celite pad and washed with methanol, then concentrated to provide the crude product which was purified by column chromatography on silica gel to give the amine as an off-white powder (25 mg, 64% yield). MS (ESI): Calcd for C₂₅H₃₂FN₄O₄⁺ 471.24 [M+H]⁺, found 471.15 [M+H]⁺.

### Example 20. Preparation of (R)—N-(2-amino-3-(2-aminoethoxy)propyl)-3-(4-cyanophenyl)-5-fluoro-1H-indole-2-carboxamide hydrogen chloride salt

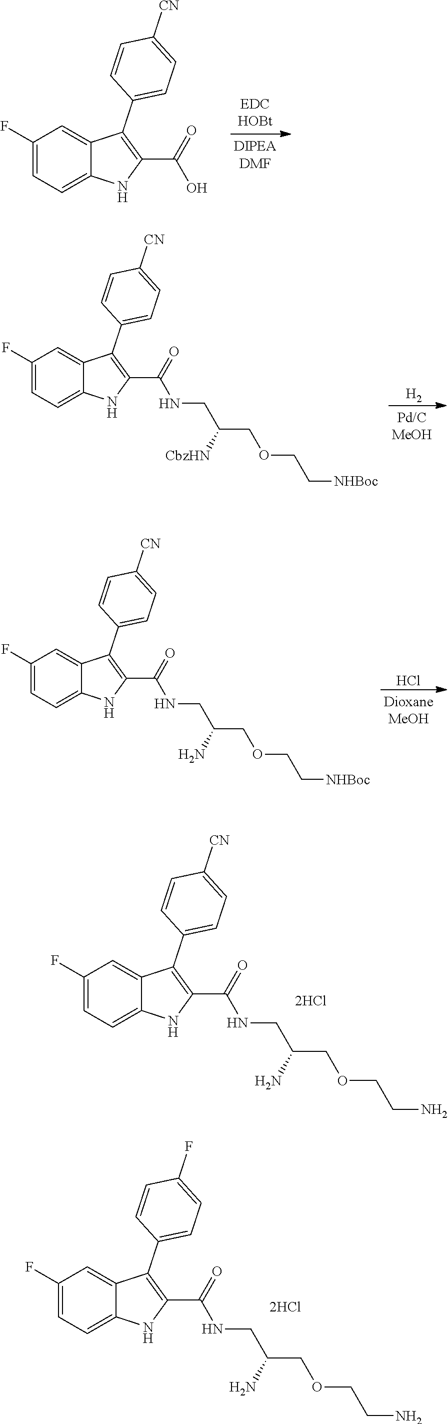

### (R)—N-(2-amino-3-(2-aminoethoxy)propyl)-3-(4-cyanophenyl)-5-fluoro-1H-indole-2-carboxamide hydrogen chloride salt

To a solution tert-butyl (R)-(2-(2-amino-3-(3-(4-cyanophenyl)-5-fluoro-1H-indole-2-carboxamido)propoxy)ethyl)carbamate (16 mg, 0.03 mmol) in MeOH (5 mL) HCl solution (4 M in dioxane, 0.15 mL, 0.6 mmol) was added. It was stirred at room temperature for 1 hour then solvent was removed in vacuo at 50° C. The residue was dissolved in water and loaded on C₁₈ column chromatography and purified with 0-15% EtOH in water to give the desired product as a white powder (8 mg, 52% yield). ¹H NMR (300 MHz, D₂O) δ 7.86 (d, J=8.2 Hz, 2H), 7.65 (d, J=8.2 Hz, 2H), 7.52 (t, J=4.4 Hz, 1H), 7.31 (d, J=9.4 Hz, 1H), 6.87 (t, J=9.4 Hz, 1H), 3.72 (m, 2H), 3.68 (m, 2H), 3.58 (m, 3H), 3.19 (m, 2H). MS (ESI): Calcd for C₂₁H₂₃FN₅O₂⁺ 396.18 [M+H]⁺, found 396.05 [M+H]⁺.

The requisite intermediates were prepared as shown in the following steps.

### Step 1)

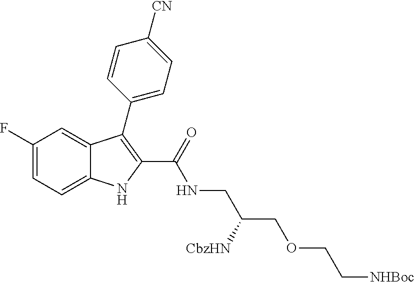 [SMILES: CC(C)(C)OC(=O)NCCOC[C@@H](CNC(=O)c1[nH]c2ccc(F)cc2c1-c1ccc(C#N)cc1)NC(=O)OCc1ccccc1]

### tert-butyl (R)-(2-(2-(((benzyloxy)carbonyl)amino)-3-(3-(4-cyanophenyl)-5-fluoro-1H-indole-2-carboxamido)propoxy)ethyl)carbamate

To a solution of 3-(4-cyanophenyl)-5-fluoro-1H-indole-2-carboxylic acid (28 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and benzyl (R)-(1-amino-3-(2-((tert-butoxycarbonyl)amino)ethoxy)propan-2-yl)carbamate (Amine Intermediate D) (37 mg, 0.1 mmol) was added. The reaction mixture was stirred at room temperature overnight. It was extracted with EtOAc and washed with water and brine. The organic layer was dried over Na₂SO₄ and filtered. The filtrate was concentrated and purified by column chromatography on silica gel to give the product (43 mg, 68% yield) as a white powder. ¹H NMR (300 MHz, CDCl₃) δ 9.66 (br s, 1H), 7.77 (m, 2H), 7.61 (m, 2H), 7.42 (m, 2H), 7.28 (m, 4H), 7.11 (m, 2H), 6.45 (br s, 1H), 5.39 (br s, 1H), 5.06 (m, 2H), 3.85 (m, 1H), 3.56 (m, 3H), 3.47 (m, 3H), 3.28 (m, 2H), 1.44 (s, 9H). MS (ESI): Calcd for C₃₄H₃₇FN₅O₆⁺ 630.27 [M+H]⁺, found 630.25 [M+H]⁺.

### Step 2)

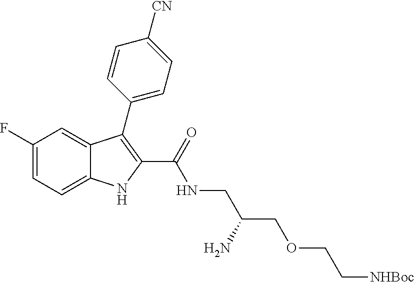 [SMILES: CC(C)(C)OC(=O)NCCOC[C@H](N)CNC(=O)c1[nH]c2ccc(F)cc2c1-c1ccc(C#N)cc1]

### tert-butyl (R)-(2-(2-amino-3-(3-(4-cyanophenyl)-5-fluoro-1H-indole-2-carboxamido)propoxy)ethyl)carbamate

To a solution of tert-butyl (R)-(2-(2-(((benzyloxy)carbonyl)amino)-3-(3-(4-cyanophenyl)-5-fluoro-1H-indole-2-carboxamido)propoxy)ethyl)carbamate (43 mg, 0.068 mmol) in MeOH (5 mL) was added Pd/C (10%, 35 mg, 0.03 mmol). Then it was stirred under H₂ (50 psi) overnight. It was filtered through a Celite pad and washed with methanol, then concentrated to provide the crude product which was purified on column chromatography on silica gel with MeOH in EtOAc as eluents to give the amine as an off-white powder (16 mg, 47% yield). MS (ESI): Calcd for C₂₆H₃₁N₅O₄⁺496.23 [M+H]⁺, found 496.15 [M+H]⁺.

### Example 21. Preparation of (R)—N-(2-amino-3-(2-aminoethoxy)propyl)-3-phenyl-1H-indole-2-carboxamide hydrogen chloride salt

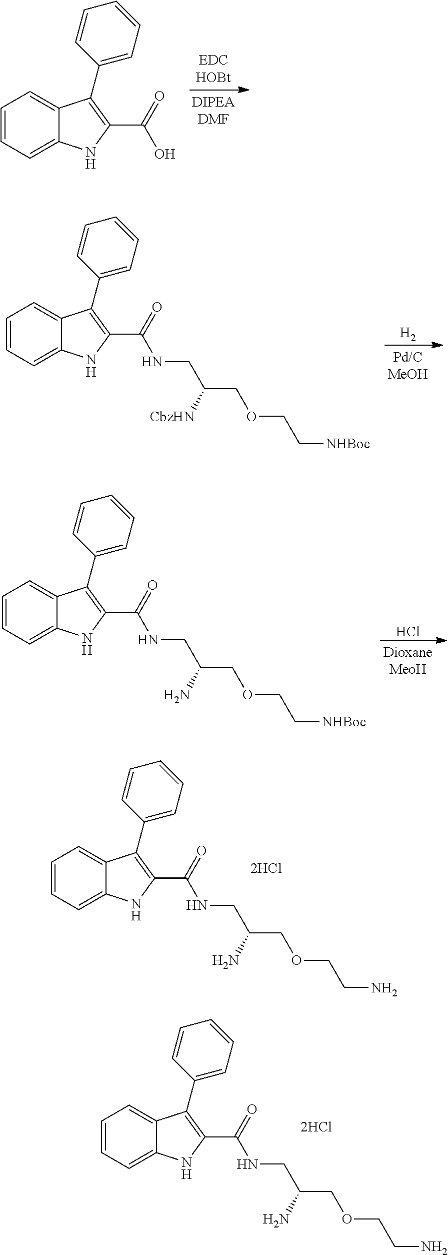

### (R)—N-(2-amino-3-(2-aminoethoxy)propyl)-3-phenyl-1H-indole-2-carboxamide hydrogen chloride salt

To a solution of tert-butyl (R)-(2-(2-amino-3-(3-phenyl-1H-indole-2-carboxamido)propoxy)ethyl)carbamate (20 mg, 0.044 mmol) in MeOH (5 mL) HCl solution (4 M in dioxane, 0.15 mL, 0.6 mmol) was added. It was stirred at room temperature until no starting material left, then solvent was removed in vacuo. The residue was dissolved in water and loaded on C18 column chromatography and purified with 0-15% EtOH in water to give the desired product as a white powder (11 mg, 59% yield). ¹H NMR (300 MHz, CD₃OD) δ 7.56 (m, 2H), 7.54 (m, 2H), 7.51 (m, 1H), 7.48 (m, 1H), 7.45 (m, 1H), 7.30 (t, J=7.5 Hz, 1H), 7.10 (t, J=7.5 Hz, 1H), 3.72 (m, 2H), 3.67 (m, 2H), 3.60 (m, 1H), 3.52 (m, 2H), 3.18 (m, 2H). MS (ESI): Calcd for C₂₀H₂₅N₄O₂⁺ 353.20 [M+H]⁺, found 353.00 [M+H]⁺.

The requisite intermediates were prepared as shown in the following steps.

### Step 1)

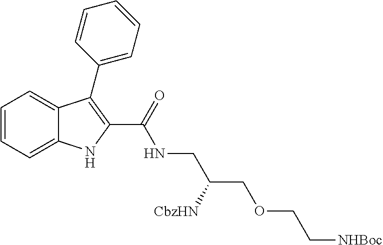 [SMILES: CC(C)(C)OC(=O)NCCOC[C@@H](CNC(=O)c1[nH]c2ccccc2c1-c1ccccc1)NC(=O)OCc1ccccc1]

### tert-butyl (R)-(2-(2-(((benzyloxy)carbonyl)amino)-3-(3-phenyl-1H-indole-2-carboxamido)propoxy)ethyl)carbamate

To a solution of 3-phenyl-1H-indole-2-carboxylic acid (24 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and benzyl (R)-(1-amino-3-(2-((tert-butoxycarbonyl)amino)ethoxy)propan-2-yl)carbamate (Amine Intermediate D) (37 mg, 0.1 mmol) was added. The reaction mixture was stirred at room temperature overnight. It was extracted with EtOAc and washed with water and brine. The organic layer was dried over anhydrous sodium sulfate and filtered. The filtrate was concentrated and purified by column chromatography on silica gel to give the product (30 mg, 51% yield) as a white powder. ¹H NMR (300 MHz, CDCl₃) δ 9.25 (br s, 1H), 7.51 (m, 1H), 7.45 (m, 3H), 7.44 (m, 2H), 7.32 (m, 5H), 7.27 (m, 2H), 7.13 (m, 1H), 6.19 (br s, 1H), 5.28 (br s, 1H), 5.13 (br, 1H), 5.06 (s, 2H), 3.77 (m, 1H), 3.56 (m, 2H), 3.44 (m, 3H), 3.32 (m, 1H), 3.23 (m, 2H), 1.44 (s, 9H). MS (ESI): Calcd for C₃₃H₃₉N₄O₆⁺587.29 [M+H]⁺, found 587.15 [M+H]⁺.

### Step 2)

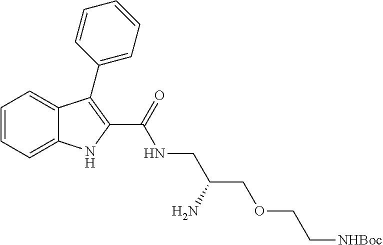 [SMILES: CC(C)(C)OC(=O)NCCOC[C@H](N)CNC(=O)c1[nH]c2ccccc2c1-c1ccccc1]

### tert-butyl (R)-(2-(2-amino-3-(3-phenyl-1H-indole-2-carboxamido)propoxy)ethyl)carbamate

To a solution tert-butyl (R)-(2-(2-(((benzyloxy)carbonyl)amino)-3-(3-phenyl-1H-indole-2-carboxamido)propoxy)ethyl)carbamate (30 mg, 0.05 mmol) in MeOH (5 mL) was added Pd/C (10%, 20 mg, 0.02 mmol). Then it was stirred under H₂ (55 psi) overnight. It was filtered through a Celite pad and washed with methanol, then concentrated to provide the crude product as an off-white powder (20 mg, 87% yield). MS (ESI): Calcd for C₂₅H₃₃N₄O₄⁺453.25 [M+H]⁺, found 453.10 [M+H]⁺.

### Example 22. Preparation of (R)—N-(2-amino-3-(2-aminoethoxy)propyl)-3-cyclohexyl-5-fluoro-1H-indole-2-carboxamide hydrogen chloride salt

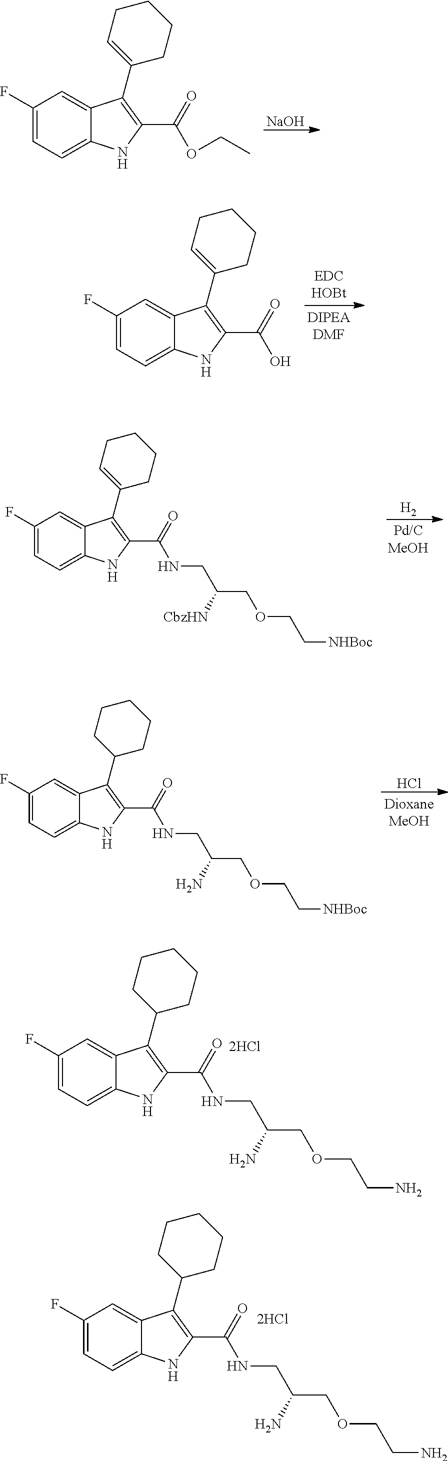

### (R)—N-(2-amino-3-(2-aminoethoxy)propyl)-3-cyclohexyl-5-fluoro-1H-indole-2-carboxamide hydrogen chloride salt

To a solution tert-butyl (R)-(2-(2-amino-3-(3-cyclohexyl-5-fluoro-1H-indole-2-carboxamido)propoxy)ethyl)carbamate (20 mg, 0.035 mmol) in MeOH (5 mL) HCl solution (4 M in dioxane, 0.15 mL, 0.6 mmol) was added. It was stirred at room temperature until no starting material left, then solvent was removed in vacuo. The residue was triturated with EtOAc and the precipitate was collected as a pale brown powder (9 mg, 57% yield). ¹H NMR (300 MHz, CD₃OD) δ 7.86 (dd, J=2.3, 10.2 Hz, 1H), 7.38 (dd, J=4.6, 9.1 Hz, 1H), 7.01 (m, 1H), 3.76 (m, 4H), 3.64 (m, 2H), 3.58 (m, 1H), 3.21 (m, 2H), 1.91 (m, 1H), 1.87 (m, 4H), 1.82 (m, 4H), 1.44 (m, 2H). MS (ESI): Calcd for C₂₀H₃₀FN₄O₂⁺ 377.23 [M+H]⁺, found 377.00 [M+H]⁺.

The requisite intermediates were prepared as shown in the following steps.

### Step 1)

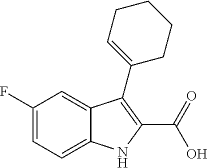 [SMILES: O=C(O)c1[nH]c2ccc(F)cc2c1C1=CCCCC1]

### 3-(cyclohex-1-en-1-yl)-5-fluoro-1H-indole-2-carboxylic acid

To a solution of ethyl 3-(cyclohex-1-en-1-yl)-5-fluoro-1H-indole-2-carboxylate (200 mg, 0.7 mmol) in THF (10 mL) was added NaOH solution (2 M, 10 mL). It was heated at 50° C. overnight. THF was removed in vacuo and the residue was acidified with HCl solution. The precipitate was filtered and washed with water. It was dried to provide the product as a pale brown powder (110 mg, 61% yield) which was used for next step reaction without further purification. MS (ESI): Calcd for C₁₅H₁₃FNO₂⁻ 258.10 [M−H]⁻, found 258.05 [M−H]⁻.

### Step 2)

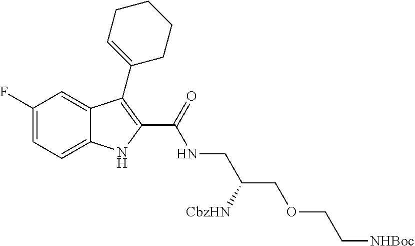 [SMILES: CC(C)(C)OC(=O)NCCOC[C@@H](CNC(=O)c1[nH]c2ccc(F)cc2c1C1=CCCCC1)NC(=O)OCc1ccccc1]

### tert-butyl (R)-(2-(2-(((benzyloxy)carbonyl)amino)-3-(3-cyclohexyl-1H-indole-2-carboxamido)propoxy)ethyl)carbamate

To a solution of 3-(cyclohex-1-en-1-yl)-5-fluoro-1H-indole-2-carboxylic acid (29 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at room temperature and benzyl (R)-(1-amino-3-(2-((tert-butoxycarbonyl)amino)ethoxy)propan-2-yl)carbamate (Amine Intermediate D) (37 mg, 0.1 mmol) was added. The reaction mixture was stirred at room temperature overnight. It was extracted with EtOAc and washed with water and brine. The organic layer was dried over anhydrous sodium sulfate and filtered. The filtrate was concentrated and purified by column chromatography on silica gel to give the product (43 mg, 75% yield) as a white powder. MS (ESI): Calcd for C₃₃H₄₂FN₄O₆⁺609.31 [M+H]⁺, found 609.20 [M+H]⁺.

### Step 3)

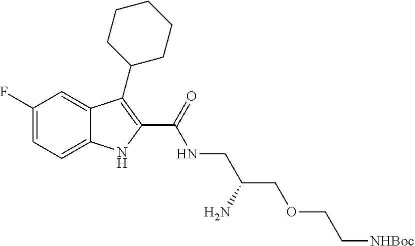 [SMILES: CC(C)(C)OC(=O)NCCOC[C@H](N)CNC(=O)c1[nH]c2ccc(F)cc2c1C1CCCCC1]

### tert-butyl (R)-(2-(2-amino-3-(3-cyclohexyl-5-fluoro-1H-indole-2-carboxamido)propoxy)ethyl)carbamate

To a solution of tert-butyl (R)-(2-(2-(((benzyloxy)carbonyl)amino)-3-(3-cyclohexyl-5-fluoro-1H-indole-2-carboxamido)propoxy)ethyl)carbamate (40 mg, 0.7 mmol) in MeOH (5 mL) was added Pd/C (10%, 20 mg, 0.02 mmol). Then it was stirred under H₂ (50 psi) overnight. It was filtered through a Celite pad and washed with methanol, then concentrated to provide the crude product which was purified on column chromatography on silica gel to give the amine as an off-white powder (20 mg, 50% yield). MS (ESI): Calcd for C₂₅H₃₈N₄O₄⁺477.29 [M+H]⁺, found 477.15 [M+H]⁺.

### Example 23. Preparation of N-(((2R,3S,4S)-4-(aminomethyl)-3-hydroxypyrrolidin-2-yl)methyl)-3-(4-fluorophenyl)-1H-indole-2-carboxamide hydrogen chloride salt

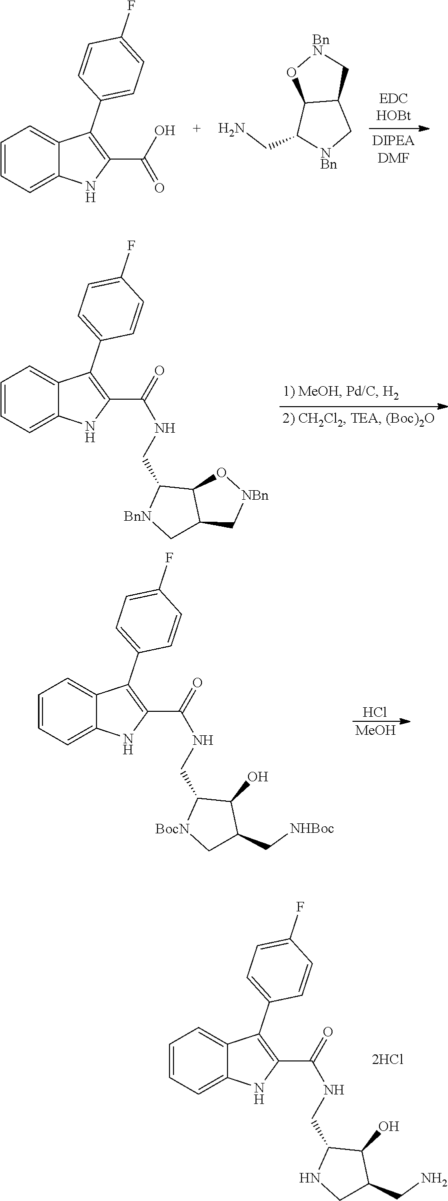

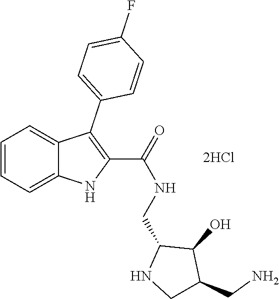 [SMILES: NC[C@H]1CN[C@H](CNC(=O)c2[nH]c3ccccc3c2-c2ccc(F)cc2)[C@H]1O]

### N-(((2R,3S,4S)-4-(aminomethyl)-3-hydroxypyrrolidin-2-yl)methyl)-3-(4-fluorophenyl)-1H-indole-2-carboxamide hydrogen chloride salt

To a solution of tert-butyl (2R,3S,4S)-4-(((tert-butoxycarbonyl)amino)methyl)-2-((3-(4-fluorophenyl)-1H-indole-2-carboxamido)methyl)-3-hydroxypyrrolidine-1-carboxylate (30 mg, 0.05 mmol) in MeOH (2 mL) was added HCl (4 M in dioxane, 0.5 mL, 2 mmol). The reaction mixture was stirred at 50° C. for 1 h and then concentrated to give a residue. The residue was triturated with EtOAc to give the desired product (17 mg, 73% yield) as yellow solid. ¹H NMR (300 MHz, D₂O) δ 7.52-7.63 (m, 4H), 7.39 (m, 1H), 7.29 (m, 2H), 7.20 (m, 1H), 4.38 (m, 1H), 3.49-3.77 (m, 4H), 3.11-3.34 (m, 3H), 2.76 (m, 1H). MS (ESI): Calcd for C₂₁H₂₄FN₄O₂ 383.19 [M+H⁺], found 382.95 [M+H]⁺.

The requisite intermediates were prepared as shown in the following steps.

### Step 1)

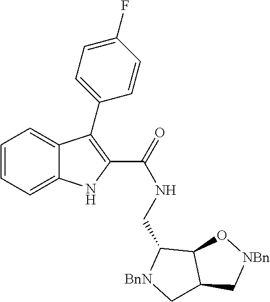 [SMILES: O=C(NC[C@@H]1[C@H]2ON(Cc3ccccc3)C[C@H]2CN1Cc1ccccc1)c1[nH]c2ccccc2c1-c1ccc(F)cc1]

### N-(((3aR,6R,6aS)-2,5-dibenzylhexahydro-2H-pyrrolo[3,4-d]isoxazol-6-yl)methyl)-3-(4-fluorophenyl)-1H-indole-2-carboxamide

To a solution of 3-(4-fluorophenyl)-1H-indole-2-carboxylic acid (356 mg, 1.40 mmol) in dry DMF (5 mL) was added DIPEA (0.41 mL, 2.33 mmol), HOBt (95 mg, 0.70 mmol) and EDC (267 mg, 1.40 mmol). The reaction mixture was stirred at room temperature for 10 min and then a solution of ((3aR,6R,6aS)-2,5-dibenzylhexahydro-2H-pyrrolo[3,4-d]isoxazol-6-yl)methanamine (Amine Intermediate E) (377 mg, 1.17 mmol) in DMF (5 mL) was added. The reaction mixture was stirred at room temperature overnight then added H₂O with stirring. A solid was formed and collected by filtration. After air drying the solid was purified by chromatography on silica gel. Elution with 50-100% EtOAc/hexanes afforded the desired compound (625 mg, 95% yield) as white solid. ¹H NMR (300 MHz, CDCl₃) δ 9.34 (br s, 1H), 7.00-7.50 (m, 14H), 6.41 (d, J=4.8 Hz, 1H), 4.13 (m, 1H), 4.04 (m, 1H), 3.86 (m, 2H), 3.73 (d, J=12.9 Hz, 1H), 3.36 (d, J=12.9 Hz, 1H), 3.10 (d, J=12.3 Hz, 1H), 2.90 (m, 1H), 2.83 (m, 1H), 2.68 (m, 1H), 2.61 (m, 1H), 2.44 (m, 1H), 2.11 (m, 1H).

### Step 2)

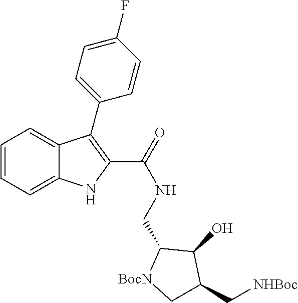 [SMILES: CC(C)(C)OC(=O)NC[C@H]1CN(C(=O)OC(C)(C)C)[C@H](CNC(=O)c2[nH]c3ccccc3c2-c2ccc(F)cc2)[C@H]1O]

### tert-butyl (2R,3S,4S)-4-(((tert-butoxycarbonyl)amino)methyl)-2-((3-(4-fluorophenyl)-1H-indole-2-carboxamido)methyl)-3-hydroxypyrrolidine-1-carboxylate

To a solution of N-(((3aR,6R,6aS)-2,5-dibenzylhexahydro-2H-pyrrolo[3,4-d]isoxazol-6-yl)methyl)-3-(4-fluorophenyl)-1H-indole-2-carboxamide (625 mg, 1.11 mmol) in MeOH (50 mL) was added Pd/C (10%, 200 mg, 0.2 mmol). The reaction mixture was stirred under H₂ at 55 psi at room temperature overnight. The completion of the reaction was monitored by checking the disappearance of the staring material by LC-MS. The solid was removed by passing through Celite pad, and was washed with MeOH. The filtrate was concentrated, then dissolved in CH₂Cl₂. To this solution was added TEA (0.39 mL, 2.79 mmol) and (Boc)₂O (535 mg, 2.45 mmol). The resulting reaction mixture was stirred at room temperature for 2 h, then diluted with CH₂Cl₂, washed with saturated NaHCO₃ solution, brine, and dried over Na₂SO₄. After removal of solid, the organic solution was concentrated and purified by chromatography on silica gel. Elution with 50-100% EtOAc/hexanes afforded the desired compound (365 mg, 56% yield) as white solid. ¹H NMR (300 MHz, CDCl₃) δ 9.30 (br s, 1H), 7.46 (m, 4H), 7.10-7.36 (m, 4H), 6.64 (br s, 1H), 5.95 (br s, 1H), 4.89 (br s, 1H), 4.60 (br, 1H), 3.94 (br s, 1H), 3.75 (m, 1H), 3.34-3.60 (m, 3H), 3.24 (m, 1H), 3.08 (m, 1H), 2.93 (m, 1H), 2.19 (m, 1H), 1.36-1.44 (m, 18H).

### Example 24. Preparation of N-(((2R,3S,4S)-4-(aminomethyl)-3 hydroxypyrrolidin-2-yl)methyl)-5-fluoro-3-phenyl-1H-indole-2-carboxamide hydrogen chloride salt

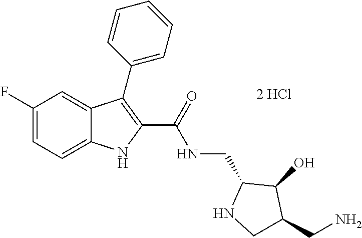 [SMILES: NC[C@H]1CN[C@H](CNC(=O)c2[nH]c3ccc(F)cc3c2-c2ccccc2)[C@H]1O]

### N-(((2R,3S,4S)-4-(aminomethyl)-3-hydroxypyrrolidin-2-yl)methyl)-5-fluoro-3-phenyl-1H-indole-2-carboxamide hydrogen chloride salt

To a solution of tert-butyl (2R,3S,4S)-4-(((tert-butoxycarbonyl)amino)methyl)-2-((5-fluoro-3-phenyl-1H-indole-2-carboxamido)methyl)-3-hydroxypyrrolidine-1-carboxylate (18 mg, 0.03 mmol) in MeOH (1 mL) was added HCl (4 M in dioxane, 0.2 mL, 0.4 mmol). The reaction mixture was stirred at 50° C. for 1 h and then concentrated to give a residue. The residue was triturated with EtOAc to give the desired product (10 mg, 71% yield) as a white solid. ¹H NMR (300 MHz, D₂O) δ 7.48-7.66 (m, 6H), 7.30 (dd, J=2.1, 9.9 Hz, 1H), 7.20 (m, 1H), 4.40 (m, 1H), 3.52-3.81 (m, 4H), 3.34 (m, 2H), 3.19 (m, 1H), 2.81 (m, 1H). MS (ESI): Calcd for C₂₁H₂₄FN₄O₂ 383.19 [M+H⁺], found 382.95 [M+H]⁺.

The requisite intermediates were prepared as shown in the following steps.

### Step 1)

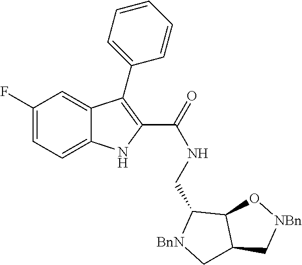 [SMILES: O=C(NC[C@@H]1[C@H]2ON(Cc3ccccc3)C[C@H]2CN1Cc1ccccc1)c1[nH]c2ccc(F)cc2c1-c1ccccc1]

### N-(((3aR,6R,6aS)-2,5-dibenzylhexahydro-2H-pyrrolo[3,4-d]isoxazol-6-yl)methyl)-5-fluoro-3-phenyl-1H-indole-2-carboxamide

To a solution of 5-fluoro-3-phenyl-1H-indole-2-carboxylic acid (36 mg, 0.14 mmol) in dry DMF (1 mL) was added DIPEA (0.04 mL, 0.23 mmol), HOBt (9.5 mg, 0.07 mmol) and EDC (27 mg, 0.14 mmol). The reaction mixture was stirred at room temperature for 10 min and then a solution of ((3aR,6R,6aS)-2,5-dibenzylhexahydro-2H-pyrrolo[3,4-d]isoxazol-6-yl)methanamine (Amine Intermediate E) (38 mg, 0.12 mmol) in DMF (5 mL) was added. The reaction mixture was stirred at room temperature overnight after which H₂O was added with stirring. A solid was formed which was collected by filtration. After air drying, the solid was purified by chromatography on silica gel. Elution with 50-100% EtOAc/hexanes afforded the desired compound (35 mg, 53% yield) as white solid. ¹H NMR (300 MHz, CDCl₃) δ 9.73 (br s, 1H), 7.22-7.52 (m, 10H), 7.07 (m, 2H), 6.99 (m, 2H), 6.53 (d, J=4.5 Hz, 1H), 4.16 (m, 1H), 4.05 (m, 1H), 3.87 (m, 2H), 3.72 (d, J=12.9 Hz, 1H), 3.36 (d, J=15 Hz, 1H), 3.08 (d, J=12.3 Hz, 1H), 2.74-2.88 (m, 2H), 2.69 (m, 1H), 2.60 (m, 1H), 2.42 (m, 1H), 2.08 (m, 1H).

### Step 2)

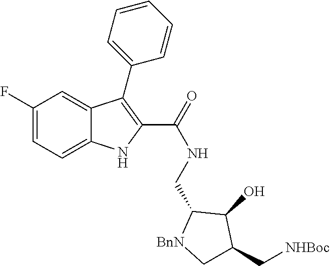 [SMILES: CC(C)(C)OC(=O)NC[C@H]1CN(Cc2ccccc2)[C@H](CNC(=O)c2[nH]c3ccc(F)cc3c2-c2ccccc2)[C@H]1O]

### tert-butyl (2R,3S,4S)-4-(((tert-butoxycarbonyl)amino)methyl)-2-((5-fluoro-3-phenyl-1H-indole-2-carboxamido)methyl)-3-hydroxypyrrolidine-1-carboxylate

To a solution of N-(((3aR,6R,6aS)-2,5-dibenzylhexahydro-2H-pyrrolo[3,4-d]isoxazol-6-yl)methyl)-5-fluoro-3-phenyl-1H-indole-2-carboxamide (22 mg, 0.04 mmol) in MeOH (10 mL) was added Pd/C (10%, 30 mg, 0.03 mmol). The reaction mixture was stirred under H₂ at 55 psi at room temperature overnight. The completion of the reaction was monitored by checking the disappearance of the staring material by LC-MS. The solid was removed by passing through a celite pad, and was washed with MeOH. The filtrate was concentrated and then dissolved in CH₂Cl₂. To this solution was added TEA (0.014 mL, 0.1 mmol), (Boc)₂O (19 mg, 0.09 mmol). The resulting reaction mixture was stirred at room temperature for 2 h, then diluted with CH₂Cl₂, washed with saturated NaHCO₃ solution, brine, and dried over Na₂SO₄. After removal of solid the organic solution was concentrated and purified by chromatography on silica gel. Elution with 50-100% EtOAc/hexanes afforded the desired compound (18 mg, 78% yield) as white solid. ¹H NMR (300 MHz, CDCl₃) δ 9.65 (br s, 1H), 7.35-7.60 (m, 6H), 7.09 (m, 2H), 6.49 (br s, 1H), 6.08 (br s, 1H), 4.93 (br s, 1H), 4.58 (br, 1H), 3.93 (s, 1H), 3.73 (m, 1H), 3.36-3.62 (m, 3H), 3.22 (m, 1H), 3.07 (m, 1H), 2.92 (m, 1H), 2.19 (m, 1H), 1.36-1.44 (m, 18H).

### Example 25. Preparation of N-(((2R,4S)-4-(aminomethyl)-3-oxopyrrolidin-2-yl)methyl)-3-(4-fluorophenyl)-1H-indole-2-carboxamide hydrogen chloride salt

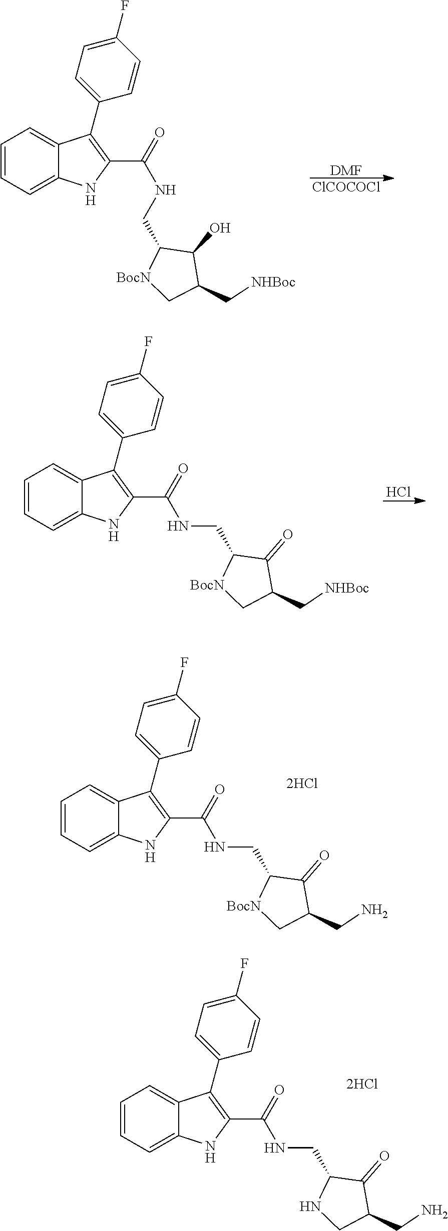

### N-(((2R,4S)-4-(aminomethyl)-3-oxopyrrolidin-2-yl)methyl)-3-(4-fluorophenyl)-1H-indole-2-carboxamide hydrogen chloride salt

To a solution of tert-butyl (2R,4S)-4-(((tert-butoxycarbonyl)amino)methyl)-2-((3-(4-fluorophenyl)-1H-indole-2-carboxamido)methyl)-3-oxopyrrolidine-1-carboxylate (25 mg, 0.043 mmol) in 0.5 mL of methanol was added 0.2 mL of HCl (4N in dioxane, 0.8 mmol). Reaction mixture was stirred at 50° C. for 30 minutes and then solvents were removed in vacuo. Residue was washed with ethyl ether and dried. The product was collected as a beige solid (11 mg, 56% yield). ¹H NMR (300 MHz, D₂O) δ 7.49 (m, 2H), 7.38 (m 2H), 7.31 (m, 1H), 7.91 (m, 2H), 7.11 (m, 1H), 4.17 (m, 1H), 3.58-3.69 (m, 2H), 3.37-3.49 (m, 2H), 3.04-3.33 (m, 2H), 2.62 (m, 1H). MS (ESI): Calcd for C₂₁H₂₂FN₄O₂⁺ 381.17 [M+H]⁺, found 380.95 [M+H]⁺.

The requisite intermediate was prepared as shown in the following step.

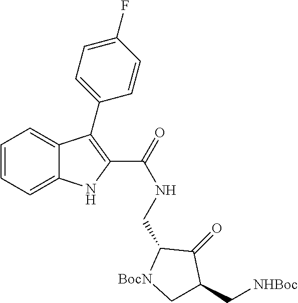 [SMILES: CC(C)(C)OC(=O)NC[C@H]1CN(C(=O)OC(C)(C)C)[C@H](CNC(=O)c2[nH]c3ccccc3c2-c2ccc(F)cc2)C1=O]

### tert-butyl (2R,4S)-4-(((tert-butoxycarbonyl)amino)methyl)-2-((3-(4-fluorophenyl)-1H-indole-2-carboxamido)methyl)-3-oxopyrrolidine-1-carboxylate

To a stirred solution of DMSO (11.6 mg, 0.15 mmol) in DCM (2 mL) was added oxalyl chloride (9.45 mg, 0.08 mmol) at −78° C. under nitrogen. After 15 minutes a solution of (2R,3S,4S)-tert-butyl 4-(((tert-butoxycarbonyl)amino)methyl)-2-((3-(4-fluorophenyl)-1H-indole-2-carboxamido)methyl)-3-hydroxypyrrolidine-1-carboxylate (29 mg, 0.05 mmol) (prepared as in Example 23, step 2) in DCM (1 mL) was added. After 10 minutes, triethylamine (15 mg, 0.15 mmol) was added and it was stirred for additional 15 minutes, then warmed to room temperature slowly and stirred for 1 hour. Solvent was removed and the residue was purified on column chromatography on silica gel to give the product (25 mg, 86% yield). MS (ESI): Calcd for C₃₁H₃₈FN₄O₆⁺ 581.28 [M+H]⁺, found 581.15 [M+H]⁺.

### Example 26. Preparation of N-(((2R,3R,4S)-4-(aminomethyl)-3-hydroxypyrrolidin-2-yl)methyl)-3-(4-fluorophenyl)-1H-indole-2-carboxamide hydrogen chloride salt

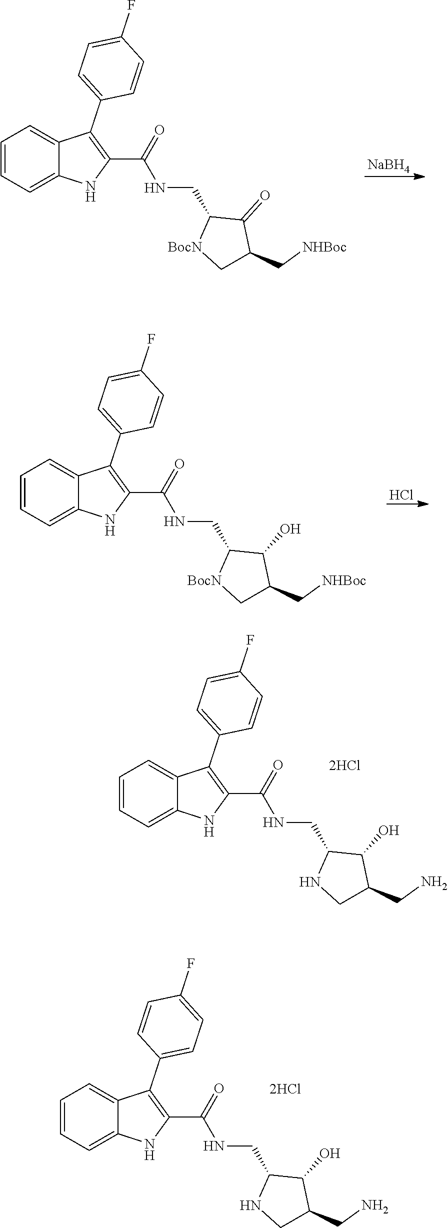

### N-(((2R,3R,4S)-4-(aminomethyl)-3-hydroxypyrrolidin-2-yl)methyl)-3-(4-fluorophenyl)-1H-indole-2-carboxamide hydrogen chloride salt

To a solution of tert-butyl (2R,3R,4S)-4-(((tert-butoxycarbonyl)amino)methyl)-2-((3-(4-fluorophenyl)-1H-indole-2-carboxamido)methyl)-3-hydroxypyrrolidine-1-carboxylate (6 mg, 0.01 mmol) in 0.5 mL of methanol was added 0.1 mL of HCl (4N in dioxane, 0.4 mmol). Reaction mixture was stirred at 50° C. for 30 minutes then solvents were removed in vacuo. The residue was washed with small amount of ethyl ether and dried to provide the product as a beige solid (2.3 mg, 49% yield). MS (ESI): Calcd for C₂₁H₂₄FN₄O₂⁺ 383.19 [M+H]⁺, found 383.10 [M+H]⁺.

The requisite intermediate was prepared as shown in the following step.

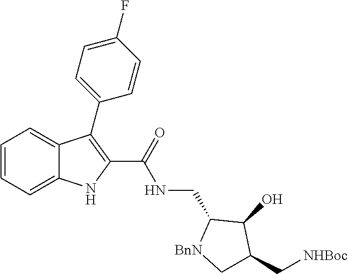 [SMILES: CC(C)(C)OC(=O)NC[C@H]1CN(Cc2ccccc2)[C@H](CNC(=O)c2[nH]c3ccccc3c2-c2ccc(F)cc2)[C@H]1O]

### tert-butyl (2R,3R,4S)-4-(((tert-butoxycarbonyl)amino)methyl)-2-((3-(4-fluorophenyl)-1H-indole-2-carboxamido)methyl)-3-hydroxypyrrolidine-1-carboxylate

To a stirred solution of tert-butyl (2R,4S)-4-(((tert-butoxycarbonyl)amino)methyl)-2-((3-(4-fluorophenyl)-1H-indole-2-carboxamido)methyl)-3-oxopyrrolidine-1-carboxylate (obtained as described in Example 25) (20 mg, 0.034 mmol) in 1 mL of methanol was added NaBH₄ (30 mg, 0.8 mmol) by portions at 0° C. Reaction mixture was stirred at room temperature for 20 minutes, TLC indicated two products were formed. The first product's R_{f}=0.52 and the second product's R_{f}=0.48 (EtOAc/Hexanes=1/1), ratio was around ½. Reaction was quenched by adding sat. aq. NH₄C₁ and extracted by ethyl acetate, washed with brine, and dried over Na₂SO₄. After filtration, solvent was removed in vacuo, residue was purified on column chromatography on silica gel to give the major product (6 mg, 30% yield). NMR (300 MHz, CDCl₃) δ 9.40 (br s, 1H), 7.08-7.55 (m, 8H), 6.68 (br s, 1H), 5.02 (br, 1H), 3.80-4.06 (m, 2H), 3.44-3.78 (m, 3H), 3.20-3.40 (m, 1H), 2.73-3.18 (m, 3H), 1.39 (br, 18H). MS (ESI): Calcd for C₃₁H₄₀FN₄O₆⁺ 583.29 [M+H]⁺, found 583.20 [M+H]⁺

### Example 27. Preparation of N-(((2S)-4-(aminomethyl)-4-hydroxypyrrolidin-2-yl)methyl)-3-(2-chlorophenyl)-5-fluoro-1H-indole-2-carboxamide hydrogen chloride salt

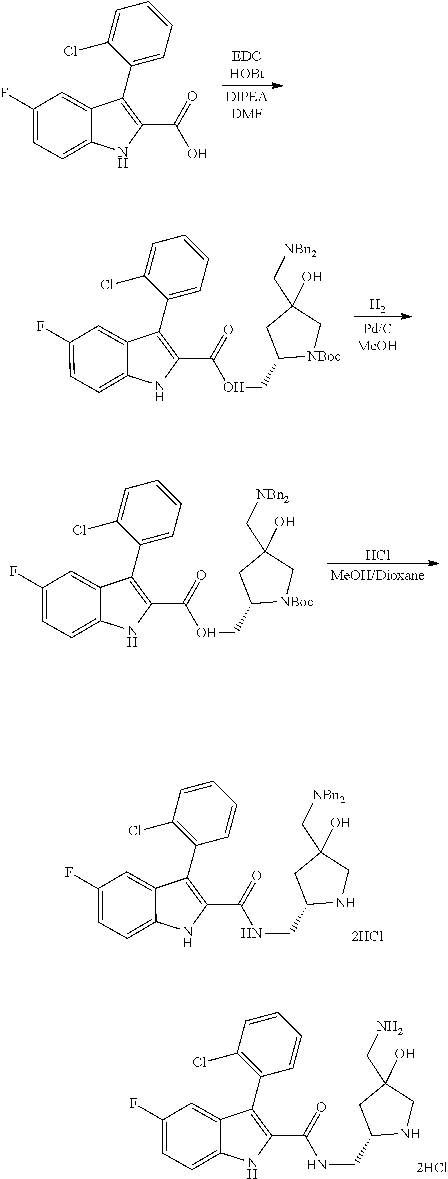

### N-(((2S)-4-(aminomethyl)-4-hydroxypyrrolidin-2-yl)methyl)-3-(2-chlorophenyl)-5-fluoro-1H-indole-2-carboxamide hydrogen chloride salt

To a solution of tert-butyl (2S)-4-(aminomethyl)-2-((3-(2-chlorophenyl)-5-fluoro-1H-indole-2-carboxamido)methyl)-4-hydroxypyrrolidine-1-carboxylate (17 mg, 0.032 mmol) in MeOH (5 mL) HCl solution (4 M in dioxane, 0.2 mL, 0.8 mmol) was added. It was stirred at room temperature for 1 hour, then solvent was removed in vacuo at 55° C. The residue was loaded on a C18 column chromatography and purified with EtOH in water to give the product as a white powder (6 mg, 34% yield). ¹H NMR (300 MHz, D₂O) δ 7.49 (m, 2H), 7.43 (m, 3H), 7.12 (m, 2H), 4.04 (m, 1H), 3.66 (m, 2H), 3.50 (m, 2H), 3.32 (m, 2H), 3.09 (m, 1H), 2.35 (m, 1H), 1.98 (m, 1H).

The requisite intermediates were prepared as shown in the following steps.

### Step 1)

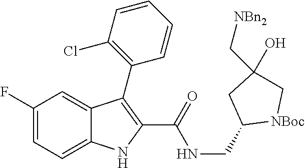

### tert-butyl (2S)-2-((3-(2-chlorophenyl)-5-fluoro-1H-indole-2-carboxamido)methyl)-4-((dibenzylamino)methyl)-4-hydroxypyrrolidine-1-carboxylate

To a solution of 3-(2-chlorophenyl)-5-fluoro-1H-indole-2-carboxylic acid (29 mg, 0.1 mmol) in dry DMF (0.5 mL) was added DIPEA (0.035 mL, 0.2 mmol), HOBt (10 mg, 0.06 mmol) and EDC (23 mg, 0.12 mmol). The reaction mixture was stirred at room temperature and tert-butyl (2S)-2-(aminomethyl)-4-((dibenzylamino)methyl)-4-hydroxypyrrolidine-1-carboxylate (Amine Intermediate F) (33 mg, 0.077 mmol) was added. The reaction mixture was stirred at room temperature overnight. It was extracted with EtOAc and washed with water and brine. The organic layer was dried over anhydrous Na₂SO₄ and filtered. The filtrate was concentrated and purified by column chromatography on silica gel to give the product (22 mg, 42% yield) as a colorless powder. MS (ESI): Calcd for C₄₀H₄₃ClFN₄O₄⁺697.29 [M+H]⁺, found 697.25 [M+H]⁺.

### Step 2)

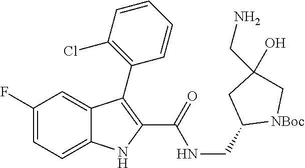 [SMILES: CC(C)(C)OC(=O)N1CC(O)(CN)C[C@H]1CNC(=O)c1[nH]c2ccc(F)cc2c1-c1ccccc1Cl]

### tert-butyl (2S)-4-(aminomethyl)-2-((3-(2-chlorophenyl)-5-fluoro-1H-indole-2-carboxamido)methyl)-4-hydroxypyrrolidine-1-carboxylate

To a solution of tert-butyl (2S)-2-((3-(2-chlorophenyl)-5-fluoro-1H-indole-2-carboxamido)methyl)-4-((dibenzylamino)methyl)-4-hydroxypyrrolidine-1-carboxylate (22 mg, 0.032 mmol) in MeOH (5 mL) was added Pd/C (10%, 20 mg, 0.02 mmol). The reaction mixture was stirred under H₂ (55 psi) overnight. It was filtered through a Celite pad and washed with methanol, then concentrated to provide the crude product which was used for next step reaction without further purification.

### Example 28. Preparation of N-((2S)-2,5-diamino-3-hydroxypentyl)-7-(4-fluorophenyl)-1H-indole-2-carboxamide hydrogen chloride salt

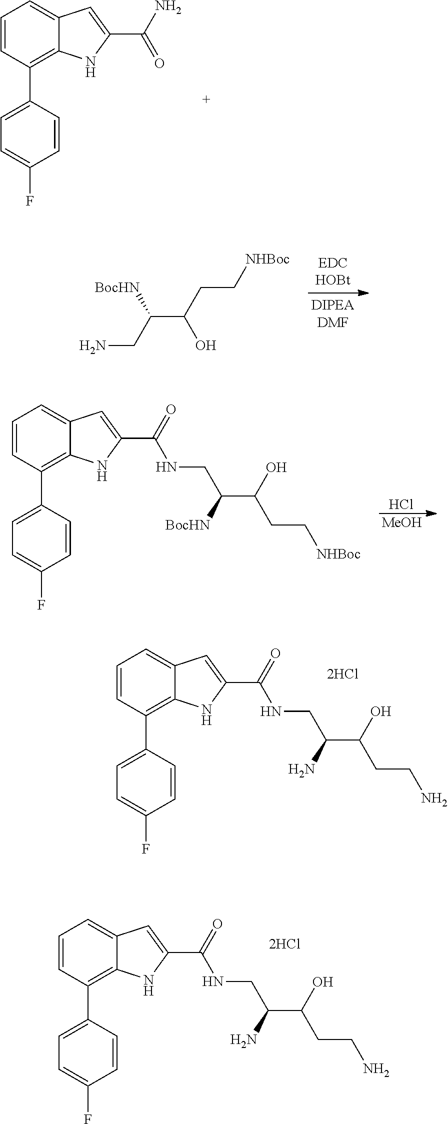

### N-((2S)-2,5-diamino-3-hydroxypentyl)-7-(4-fluorophenyl)-1H-indole-2-carboxamide hydrogen chloride salt

To a solution of di-tert-butyl ((4S)-5-(7-(4-fluorophenyl)-1H-indole-2-carboxamido)-3-hydroxypentane-1,4-diyl)dicarbamate (35 mg, 0.063 mmol) in methanol (1 mL) was added HCl (4 M in dioxane, 0.2 mL, 0.8 mmol). Reaction mixture was stirred at 50° C. for 30 minutes then solvents were removed in vacuo. Residue was triturated with small amount of ethyl acetate and dried. Product was collected as an off white solid (16 mg, 59% yield). MS (ESI): Calcd for C₂₀H₂₄FN₄O₂⁺371.19 [M+H]⁺, found 371.05 [M+H]⁺.

The requisite intermediate was prepared as follows:

### Step 1)

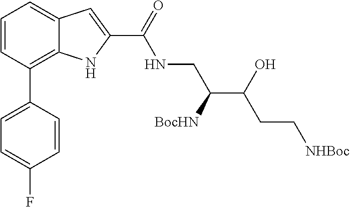 [SMILES: CC(C)(C)OC(=O)NCCC(O)[C@H](CNC(=O)c1cc2cccc(-c3ccc(F)cc3)c2[nH]1)NC(=O)OC(C)(C)C]

### di-tert-butyl ((4S)-5-(7-(4-fluorophenyl)-1H-indole-2-carboxamido)-3-hydroxypentane-1,4-diyl)dicarbamate

To a solution of di-tert-butyl ((4S)-5-amino-3-hydroxypentane-1,4-diyl)dicarbamate acetate (Amine Intermediate A) (56 mg, 0.17 mmol) in DMF (2.0 mL) was added 7-(4-fluorophenyl)-1H-indole-2-carboxylic acid (44 mg, 0.17 mmol), EDC (39 mg, 0.21 mmol), HOBt (14 mg, 0.10 mmol) and DIPEA (44 mg, 0.34 mmol). The resulting reaction mixture was stirred at room temperature overnight then diluted with water and stirred for 30 minutes. The precipitate was filtered and washed with small amount of water then dissolved in DCM, washed with brine, and dried. After concentration, the residue was purified by column chromatography on silica gel to give the product as a light-yellow solid (45 mg, 45% yield). ¹H NMR (300 MHz, CDCl₃) δ 9.15 (br s, 1H), 7.63 (m, 1H), 7.58 (m, 2H), 7.25 (m, 2H), 7.22 (m, 2H), 6.95 (d, J=2.1 Hz, 1H), 5.31 (br s, 1H), 4.86 (br s, 1H), 4.81 (br s, 1H), 3.61-3.78 (m, 2H), 3.32-3.58 (m, 3H), 3.01-3.23 (m, 1H), 1.65 (m, 2H), 1.42 (br, 18H).

### Example 29. Preparation of N-((2S)-2,5-diamino-4-hydroxypentyl)-3-((3-fluorophenyl)thio)-1H-indole-2-carboxamide hydrogen chloride salt

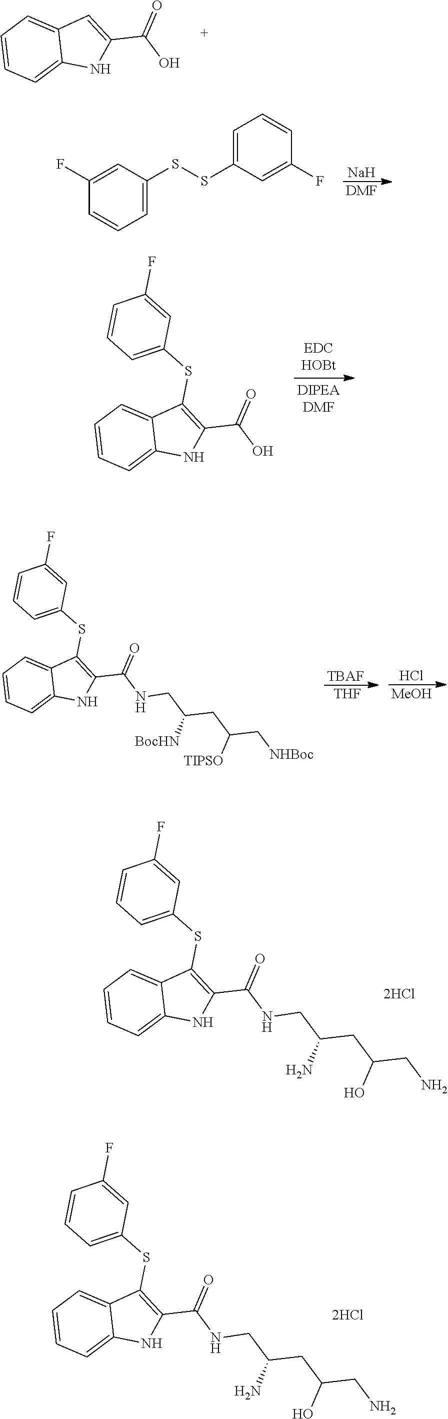

### N-((2S)-2,5-diamino-4-hydroxypentyl)-3-((3-fluorophenyl)thio)-1H-indole-2-carboxamide hydrogen chloride salt

To a solution of di-tert-butyl ((4S)-5-(3-((3-fluorophenyl)thio)-1H-indole-2-carboxamido)-2-((triisopropylsilyl)oxy)pentane-1,4-diyl)dicarbamate (52 mg, 0.07 mmol) in THE (2.0 mL) was added TBAF solution (1.0 M in THF, 0.5 mL, 0.5 mmol) at r.t. It was stirred for 1 hr and concentrated, then loaded on silica gel column chromatography and purified with 50-80% EtOAc in hexane to provide the alcohol as a white powder (36 mg, 87% yield). Then to a solution in MeOH (2 mL) HCl solution (4 M in dioxane, 1 mL, 4 mmol) was added. It was stirred at r.t. for 2 hrs, then solvent was removed in vacuo. The residue was dissolved in water and purified on a C18 column chromatography using 0-15% ethanol in water as eluents to provide the product as a white powder (21 mg, 74% yield). ¹H NMR (300 MHz, CD₃OD) b 7.56 (t, J=8.4 Hz, 2H), 7.35 (m, 1H), 7.27 (m, 1H), 7.17 (m, 1H), 6.92 (m, 1H), 6.87 (m, 1H), 6.77 (m, 1H), 4.02 (m, 1H), 3.88 (m, 1H), 3.75 (m, 1H), 3.67 (m, 2H), 2.97 (m, 1H), 2.76 (m, 1H), 1.75 (t, J=6.0 Hz, 1H). MS (ESI): Calcd for C₂₀H₂₄FN₄O₂S⁺ 403.15 [M+H]⁺, found 403.00 [M+H]⁺.

The requisite intermediates were prepared as follows:

### Step 1)

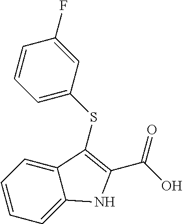 [SMILES: O=C(O)c1[nH]c2ccccc2c1Sc1cccc(F)c1]

### 3-((3-fluorophenyl)thio)-1H-indole-2-carboxylic acid

To a solution of DMF (20 mL) was added NaH (60% in mineral oil, 0.92 g, 23 mmol), then 1H-indole-2-carboxylic acid (1.61 g, 10 mmol) was added. After stirring at r.t. for 0.5 hr., 1,2-bis(3-fluorophenyl)disulfane (2.8 g, 11 mmol) was added. The mixture was heat at 110° C. for 6 hrs. under nitrogen. After cooling to r.t., it was diluted with EtOAc, treated with aqueous HCl solution, then the organic phase was washed with water, brine, and dried over anhydrous Na₂SO₄. The solvent was removed under vacuo and the residual brown oil was used for the next step of reaction without purification (2.0 g, 70% yield). ¹H NMR (300 MHz, CDCl₃) b 9.82 (br s, 1H), 7.58 (d, J=8.4 Hz, 1H), 7.48 (m, 1H), 7.36 (m, 1H), 7.15 (m, 2H), 7.02 (m, 1H), 6.95 (m, 1H), 6.79 (m, 1H). MS (ESI): Calcd for C₁₅H₁₆FNO₂S⁺ 287.05 [M+H]⁺, found 287.00 [M+H]⁺.

### Step 2)

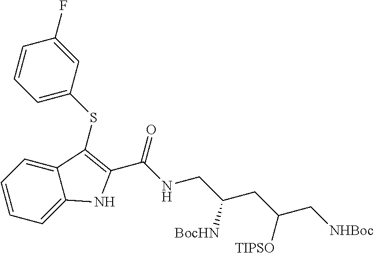

### di-tert-butyl ((4S)-5-(3-((3-fluorophenyl)thio)-1H-indole-2-carboxamido)-2-((triisopropylsilyl)oxy)pentane-1,4-diyl)dicarbamate

To a solution of 3-((3-fluorophenyl)thio)-1H-indole-2-carboxylic acid (29 mg, 0.1 mmol) in dry DMF (1.0 mL) was added DIPEA (0.035 mL, 0.2 mmol), HOBt (10 mg, 0.06 mmol) and EDC (24 mg, 0.12 mmol). The reaction mixture was stirred at r.t. and di-tert-butyl ((4S)-5-amino-2-((triisopropylsilyl)oxy)pentane-1,4-diyl)dicarbamate (Amine Intermediate B) (50 mg, 0.1 mmol) was added. The reaction mixture was stirred at r.t. overnight. It was extracted with EtOAc and washed with water and brine. The organic layer was dried over anhydrous sodium sulfate and filtered. The filtrate was concentrated and purified by column chromatography on silica gel using 50-70% EtOAc in hexane as eluents to give the amide (56 mg, 74% yield) as a pale yellow powder.

### Example 30. Preparation of 6-(4-fluorophenyl)-N-((2S)-2,5-diamino-4-hydroxypentyl)-1H-indole-2-carboxamide hydrogen chloride salt

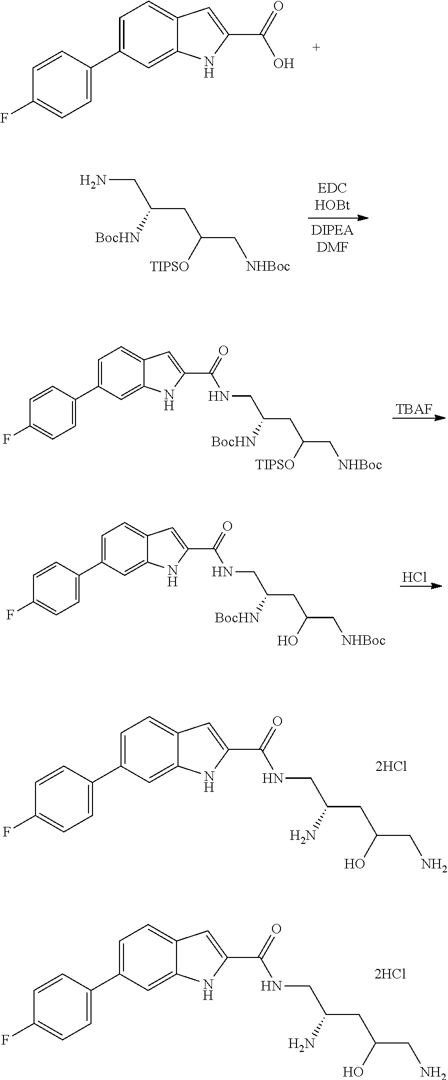

### 6-(4-fluorophenyl)-N-((2S)-2,5-diamino-4-hydroxypentyl)-1H-indole-2-carboxamide hydrogen chloride salt

To a solution of di-tert-butyl ((4S)-5-(6-(4-fluorophenyl)-1H-indole-2-carboxamido)-2-hydroxypentane-1,4-diyl)dicarbamate (470 mg, 0.82 mmol) in MeOH (5 mL) HCl solution (4 M in dioxane, 1 mL, 4 mmol) was added. It was stirred at r.t. for 2 hrs, then solvent was removed in vacuo. The residue was dissolved in water and purified on a C18 column chromatography using 0-20% ethanol in water as eluents to provide the product as a white powder (336 mg, 92% yield). ¹H NMR (300 MHz, CD₃OD) δ 7.48 (m, 1H), 7.32 (m, 4H), 7.10 (m, 1H), 6.96 (m, 1H), 6.88 (m, 2H), 4.00 (m, 1H), 3.58 (m, 1H), 3.47 (m, 2H), 3.01 (m, 1H), 2.83 (m, 1H), 1.78 (m, 1H), 1.66 (m, 1H). ¹³C NMR (75 MHz, D₂O) δ 164.1, 160.2, 137.2, 136.7, 136.3, 129.8, 128.2, 128.1, 126.3, 122.2, 119.9, 115.2, 114.9, 109.6, 105.2, 65.7, 50.7, 44.7, 41.2, 33.9. MS (ESI): Calcd for C₂₀H₂₄FN₄O₂⁺ 371.18 [M+H]⁺, found 371.00 [M+H]⁺.

The requisite intermediates were prepared as follows:

### Step 1)

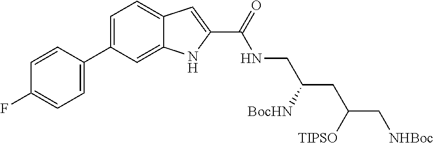

### di-tert-butyl ((4S)-5-(6-(4-fluorophenyl)-1H-indole-2-carboxamido)-2-((triisopropylsilyl)oxy)pentane-1,4-diyl)dicarbamate

To a solution of 6-(4-fluorophenyl)-1H-indole-2-carboxylic acid (385 mg, 1.5 mmol) in dry DMF (5 mL) was added DIPEA (0.51 mL, 3 mmol), HOBt (130 mg, 0.9 mmol) and EDC (380 mg, 2 mmol). The reaction mixture was stirred at r.t. and di-tert-butyl ((4S)-5-amino-2-((triisopropylsilyl)oxy)pentane-1,4-diyl)dicarbamate (Amine Intermediate B) (750 mg, 1.5 mmol) was added. The reaction mixture was stirred at r.t. overnight. It was extracted with EtOAc and washed with water and brine. The organic layer was dried over anhydrous sodium sulfate and filtered. The filtrate was concentrated and purified by column chromatography on silica gel using 50-70% EtOAc in hexane as eluents to give the amide (950 mg, 87% yield) as a white powder. MS (ESI): Calcd for C₃₉H₅₉FN₄O₆NaSi⁺749.42 [M+H]⁺, found 749.35 [M+Na]⁺.

### Step 2)

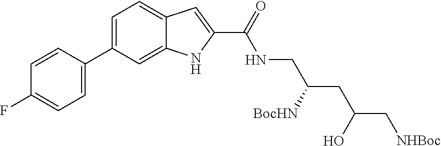 [SMILES: CC(C)(C)OC(=O)NCC(O)C[C@@H](CNC(=O)c1cc2ccc(-c3ccc(F)cc3)cc2[nH]1)NC(=O)OC(C)(C)C]

### di-tert-butyl ((4S)-5-(6-(4-fluorophenyl)-1H-indole-2-carboxamido)-2-hydroxypentane-1,4-diyl)dicarbamate

To a solution of di-tert-butyl ((4S)-5-(3-(4-fluorophenyl)-1H-indole-2-carboxamido)-2-((triisopropylsilyl)oxy)pentane-1,4-diyl)dicarbamate (930 mg, 1.28 mmol) in THE (20 mL) was added TBAF solution (1.0 M in THF, 4 mL, 4 mmol) at r.t. It was stirred for 1 hr and concentrated, then loaded on a silica gel column chromatography and purified with 50-80% EtOAc in hexane to provide the alcohol as a white powder (490 mg, 67% yield). ¹H NMR (300 MHz, DMSO-d₆) δ 11.13 (br s, 1H), 8.43 (br s, 1H), 8.14 (m, 1H), 8.08 (m, 3H), 7.75 (dd, J=8.1 Hz, 1.5 Hz, 1H), 7.57 (t, J=8.7 Hz, 2H), 7.48 (s, 1H), 6.56 (br d, 1H), 6.29 (br s, 1H), 4.72 (br, 1H), 4.41 (m, 1H), 4.28 (m, 1H), 3.98 (m, 2H), 3.62 (m, 1H), 3.54 (m, 1H), 3.46 (m, 1H), 2.12 (m, 1H), 2.06 (m, 1H), 1.81 (s, 18H). MS (ESI): Calcd for C₃₀H₄₀FN₄O₆⁺ 571.29 [M+H]⁺, found 571.25 [M+H]⁺.

### Example 31. Preparation of (R)—N-(2-amino-3-(2-aminoethoxy)propyl)-6-(4-fluorophenyl)-1H-indole-2-carboxamide hydrogen chloride salt

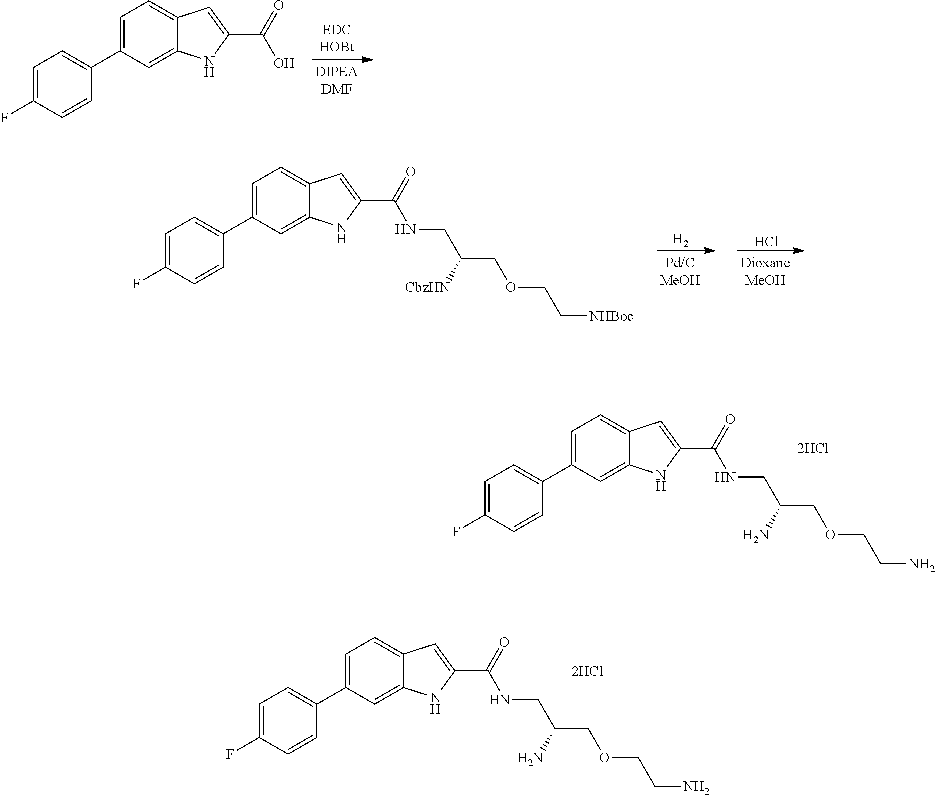 [SMILES: O=C(O)c1cc2ccc(-c3ccc(F)cc3)cc2[nH]1 ; CC(C)(C)OC(=O)NCCOC[C@@H](CNC(=O)c1cc2ccc(-c3ccc(F)cc3)cc2[nH]1)NC(=O)OCc1ccccc1 ; "H2" ; Cl ; NCCOC[C@H](N)CNC(=O)c1cc2ccc(-c3ccc(F)cc3)cc2[nH]1 ; NCCOC[C@H](N)CNC(=O)c1cc2ccc(-c3ccc(F)cc3)cc2[nH]1]

### (R)—N-(2-amino-3-(2-aminoethoxy)propyl)-6-(4-fluorophenyl)-1H-indole-2-carboxamide hydrogen chloride salt

To a solution tert-butyl (R)-(2-(2-(((benzyloxy)carbonyl)amino)-3-(6-(4-fluorophenyl)-1H-indole-2-carboxamido)propoxy)ethyl)carbamate (130 mg, 0.22 mmol) in MeOH (20 mL) was added Pd/C (10%, 50 mg, 0.05 mmol). Then it was stirred under H₂ (55 psi) overnight. It was filtered through a Celite pad and washed with methanol, then concentrated to provide the crude product as an off-white powder. MS (ESI): Calcd for C₂₅H₃₂FN₄O₄⁺ 471.23 [M+H]⁺, found 471.15 [M+H]⁺. To a solution of tert-butyl (R)-(2-(2-amino-3-(6-(4-fliorophenyl)-1H-indole-2-carboxamido)propoxy)ethyl)carbamate in MeOH (5 mL) HCl solution (4 M in dioxane, 0.2 mL, 0.8 mmol) was added. It was stirred at r.t. until no starting material left, then solvent was removed in vacuo. The residue was dissolved in water and loaded on a C18 column chromatography and purified with 0-15% EtOH in water to give the desired product as a white powder (82 mg, 86% yield). ¹H NMR (300 MHz, CD₃OD) δ 8.77 (br 1H), 7.66 (m, 4H), 7.33 (d, J=8.4 Hz, 1H), 7.18 (m, 3H), 3.80 (m, 3H), 3.77 (m, 2H), 3.70 (m, 2H), 3.22 (m, 2H). MS (ESI): Calcd for C₂₀H₂₄FN₄O₂⁺371.18 [M+H]⁺, found 371.05 [M+H]⁺.

The requisite intermediate was prepared as follow:

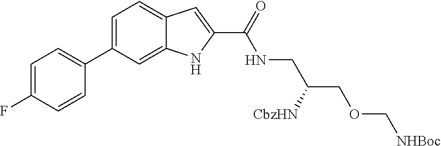 [SMILES: CC(C)(C)OC(=O)NCOC[C@@H](CNC(=O)c1cc2ccc(-c3ccc(F)cc3)cc2[nH]1)NC(=O)OCc1ccccc1]

### tert-butyl (R)-(2-(2-(((benzyloxy)carbonyl)amino)-3-(6-(4-fluorophenyl)-1H-indole-2-carboxamido)propoxy)ethyl)carbamate

To a solution of 6-(4-fluorophenyl)-1H-indole-2-carboxylic acid (124 mg, 0.4 mmol) in dry DMF (2 mL) was added DIPEA (0.17 mL, 1 mmol), HOBt (42 mg, 0.3 mmol) and EDC (100 mg, 0.5 mmol). The reaction mixture was stirred at r.t. and benzyl (R)-(1-amino-3-(2-((tert-butoxycarbonyl)amino)ethoxy)propan-2-yl)carbamate (Amine Intermediate D) (148 mg, 0.4 mmol) was added. The reaction mixture was stirred at r.t. overnight. It was extracted with EtOAc and washed with water and brine. The organic layer was dried over anhydrous sodium sulfate and filtered. The filtrate was concentrated and purified by column chromatography on silica gel to give the product (136 mg, 56% yield) as a white powder. ¹H NMR (300 MHz, CDCl₃) δ 9.27 (br s, 1H), 7.71 (m, 1H), 7.57 (m, 2H), 7.55 (m, 1H), 7.24-7.36 (m, 5H), 7.14 (m, 3H), 7.05 (m, 1H), 6.19 (br s, 1H), 5.80 (br s, 1H), 5.12 (s, 2H), 4.93 (br, 1H), 3.64 (m, 1H), 3.47-3.61 (m, 4H), 3.45 (m, 2H), 3.29 (m, 2H), 1.47 (s, 9H). MS (ESI): Calcd for C₃₃H₃₈FN₄O₆⁺ 605.28 [M+H]⁺, found 605.20 [M+H]⁺.

### Example 32. Preparation of (R)—N-(2-amino-3-(2-aminoethoxy)propyl)-6-phenyl-1H-indole-2-carboxamide hydrogen chloride salt

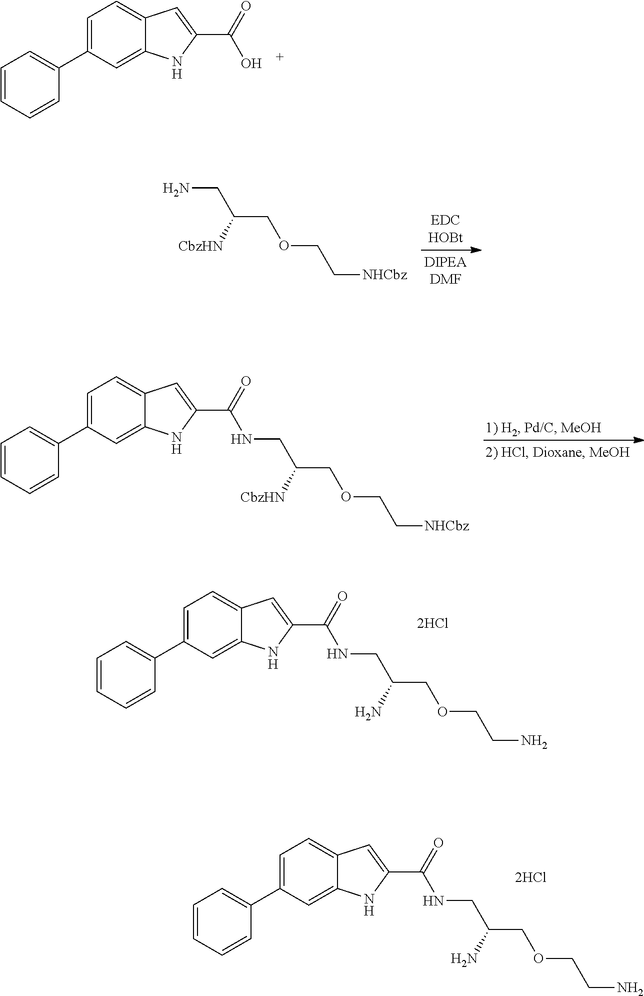

### (R)—N-(2-amino-3-(2-aminoethoxy)propyl)-6-phenyl-1H-indole-2-carboxamide hydrogen chloride salt

To a solution of benzyl (R)-(2-(2-(((benzyloxy)carbonyl)amino)-3-(6-phenyl-1H-indole-2-carboxamido)propoxy)ethyl)carbamate (245 mg, 0.48 mmol) in MeOH (20 mL) was added Pd/C (10%, 50 mg, 0.05 mmol). Then it was stirred under H₂ (50 psi) overnight. It was filtered through a Celite pad and washed with methanol, then concentrated. To the solution was added HCl solution (4 M in dioxane, 0.5 mL, 2.0 mmol), then solvent was removed in vacuo. The residue was dissolved in water and purified on C18 column with 0-15% acetonitrile in water to give a white powder (135 mg, 80% yield). ¹H NMR (300 MHz, D₂O) δ 7.79 (m, 2H), 7.74 (m, 2H), 7.51 (m, 3H), 7.41 (t, J=7.2 Hz, 1H), 7.14 (m, 1H), 3.83 (m, 1H), 3.78 (m, 3H), 3.71 (m, 3H), 3.21 (t, J=5.1 Hz, 2H). MS (ESI): Calcd for C₂₀H₂₅N₄O₂⁺ 353.20 [M+H]⁺, found 352.95 [M+H]⁺.

The requisite intermediate was prepared as follow:

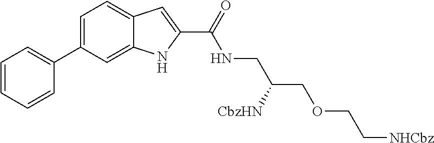 [SMILES: O=C(NCCOC[C@@H](CNC(=O)c1cc2ccc(-c3ccccc3)cc2[nH]1)NC(=O)OCc1ccccc1)OCc1ccccc1]

### benzyl (R)-(2-(2-(((benzyloxy)carbonyl)amino)-3-(6-phenyl-1H-indole-2-carboxamido)propoxy)ethyl)carbamate

To a solution of 6-phenyl-1H-indole-2-carboxylic acid (119 mg, 0.5 mmol) in dry DMF (3 mL) was added EDC (120 mg, 0.6 mmol) and HOBt (50 mg, 0.3 mmol). The reaction mixture was stirred at r.t. and DIPEA (0.17 mL, 1 mmol) was added followed by benzyl (R)-(1-amino-3-(2-(((benzyloxy)carbonyl)amino)ethoxy)propan-2-yl)carbamate (Amine Intermediate G) (201 mg, 0.5 mmol). The reaction mixture was stirred at r.t. overnight. It was extracted with EtOAc and washed with water and brine. The organic layer was dried over anhydrous sodium sulfate and filtered. The filtrate was concentrated and purified by column chromatography on silica gel to give the product (251 mg, 81% yield) as a white powder. ¹H NMR (300 MHz, CDCl₃) δ 9.23 (br d, 1H), 7.71 (d, J=8.4 Hz, 1H), 7.64 (m, 2H), 7.48 (m, 2H), 7.42 (m, 2H), 7.35 (m, 1H) 7.27-7.33 (m, 10H), 6.98 (s, 1H), 5.74 (br s, 1H), 5.46 (br t, 1H), 5.12 (m, 2H), 5.11 (m, 2H), 3.97 (m, 1H), 3.76 (m, 1H), 3.64 (m, 1H), 3.55 (m, 4H), 3.42 (m, 2H). MS (ESI): Calcd for C₃₆H₃₇N₄O₆⁺621.27 [M+H]⁺, found 621.20 [M+H]⁺.

### Example 33. Preparation of (R)—N-(2-amino-3-(2-aminoethoxy)propyl)-4-(4-fluorophenyl)-3-methyl-1H-indole-2-carb oxamide hydrogen chloride salt

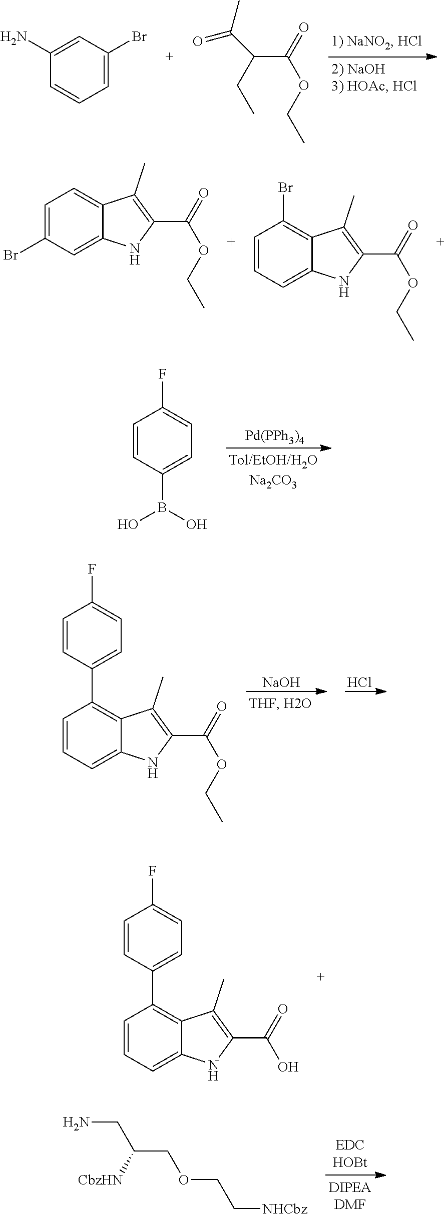

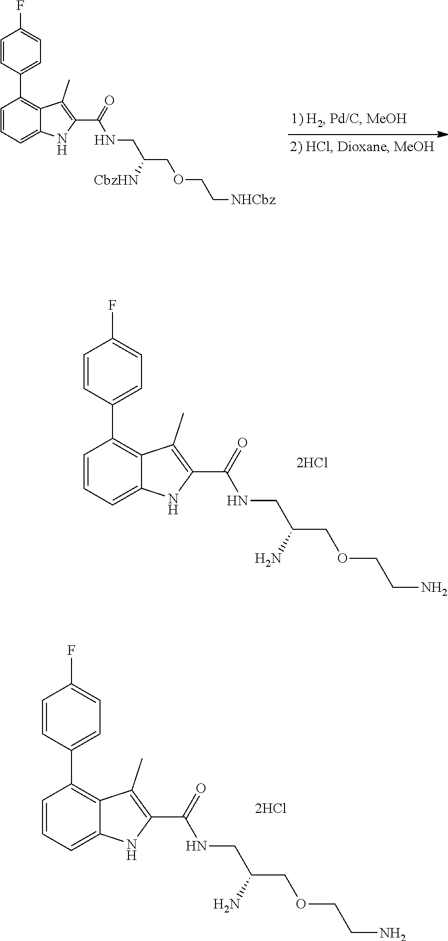 [SMILES: Cc1c(C(=O)NC[C@H](COCCNC(=O)OCc2ccccc2)NC(=O)OCc2ccccc2)[nH]c2cccc(-c3ccc(F)cc3)c12 ; Cc1c(C(=O)NC[C@@H](N)COCCN)[nH]c2cccc(-c3ccc(F)cc3)c12 ; Cc1c(C(=O)NC[C@@H](N)COCCN)[nH]c2cccc(-c3ccc(F)cc3)c12]

### (R)—N-(2-amino-3-(2-aminoethoxy)propyl)-4-(4-fluorophenyl)-3-methyl-1H-indole-2-carboxamide hydrogen chloride salt

To a solution of benzyl (R)-(2-(2-(((benzyloxy)carbonyl)amino)-3-(4-(4-fluorophenyl)-3-methyl-1H-indole-2-carboxamido)propoxy)ethyl)carbamate (310 mg, 0.48 mmol) in MeOH (20 mL) was added Pd/C (10%, 50 mg, 0.05 mmol). Then it was stirred under H₂ (50 psi) overnight. It was filtered through a Celite pad and washed with methanol, then concentrated. To the solution was added HCl solution (4 M in dioxane, 0.5 mL, 2.0 mmol), then solvent was removed in vacuo. The residue was dissolved in water and purified on C18 column with 0-15% acetonitrile in water to give a white powder (153 mg, 79% yield). ¹H NMR (300 MHz, CD₃OD) δ 7.41 (m, 1H), 7.38 (m, 2H), 7.27 (dd, J 6.9 Hz, 8.4 Hz, 1H), 7.16 (m, 2H), 6.88 (dd, J=0.9 Hz, 6.9 Hz, 1H), 3.80 (m, 3H), 3.78 (m, 2H), 3.66 (m, 2H), 3.20 (t, J=4.8 Hz, 2H), 2.09 (s, 3H). MS (ESI): Calcd for C₂₁H₂₆FN₄O₂⁺ 385.20 [M+H]⁺, found 385.00 [M+H]⁺.

The requisite intermediates were prepared as follows:

### Step 1)

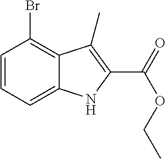 [SMILES: CCOC(=O)c1[nH]c2cccc(Br)c2c1C]

### ethyl 4-bromo-3-methyl-1H-indole-2-carboxylate

To a suspension of 3-bromoaniline (11.8 g, 62.8 mmol) in water (28 mL) was added conc. HCl (35% in H₂O, 15.4 mL) at 0° C., then NaNO₂ (4.8 g, 69.2 mmol) was added by portions. The reaction mixture was stirred at 0° C. for 20 minutes, then ethyl-2-ethylacetate (12 g, 76 mmol) in EtOAc (60 mL) was added dropwise at same temperature. After 30 minutes, the pH of the reaction mixture was adjusted to 7-8 by adding 4.4 g of NaOAc and 7.3 g of NaOH, then it was continued to stir for 3 hours at room temperature. It was stirred at 50° C. overnight. After cooled to room temperature, the reaction mixture was separated, and the aqueous layer was extracted with EtOAc (3×40 mL). The combined organic layer was washed with water, brine and dried over Na₂SO₄. Solvent was removed and the residue was purified by column chromatography on silica gel to give two isomers. The first isomer ethyl 6-bromo-3-methyl-1H-indole-2-carboxylate was got as a pale brown powder (1.4 g, 7.9%)¹H NMR (300 MHz, CDCl₃) δ 8.96 (br s, 1H), 7.50-7.73 (m, 2H), 7.22-7.26 (m, 1H), 4.42 (q, J=7.2 Hz, 2H), 2.56 (s, 3H), 1.43 (t, J=7.2 Hz, 3H). The second isomer ethyl 4-bromo-3-methyl-1H-indole-2-carboxylate was got as a pale brown powder (1.1 g, 6.2%). ¹H NMR (300 MHz, CDCl₃) δ 8.75 (br s, 1H), 7.26-7.32 (m, 2H), 7.20 (t, J=7.5 Hz, 1H), 4.42 (q, J=7.2 Hz, 2H), 2.92 (s, 3H), 1.44 (t, J=7.2 Hz, 3H).

### Step 2)

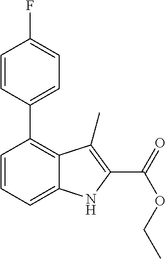 [SMILES: CCOC(=O)c1[nH]c2cccc(-c3ccc(F)cc3)c2c1C]

### ethyl 4-(4-fluorophenyl)-3-methyl-1H-indole-2-carboxylate

The mixture of ethyl 4-bromo-3-methyl-1H-indole-2-carboxylate (565 mg, 2.0 mmol) and (4-fluorophenyl)boronic acid (350 mg, 2.5 mmol) in a mixture of toluene, ethanol and saturated Na₂CO₃ solution (20/5/5 mL) was degassed and Pd(dppf)Cl₂ (70 mg, 0.1 mmol) was added. The reaction mixture was heated at 100° C. overnight, it was extracted with EtOAc and washed with brine and dried over Na₂SO₄, then concentrated. It was purified by column chromatography on silica gel with 0-20% EtOAc in hexane as eluents to give the product (510 mg, 86% yield) as an off-white powder. ¹H NMR (300 MHz, CDCl₃) δ 8.79 (br s, 1H), 7.36 (m, 3H), 7.29 (m, 1H), 7.11 (t, J=8.7 Hz, 2H), 6.94 (dd, J=1.2 Hz, 6.9 Hz, 1H), 4.40 (q, J=7.2 Hz, 2H), 2.14 (s, 3H), 1.40 (t, J=7.2 Hz, 3H). MS (ESI): Calcd for C₁₈H₁₇FNO₂⁺298.12 [M+H]⁺, found 297.95 [M+H]⁺.

### Step 3)

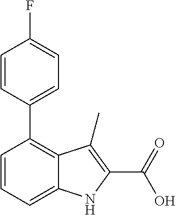 [SMILES: Cc1c(C(=O)O)[nH]c2cccc(-c3ccc(F)cc3)c12]

### 4-(4-fluorophenyl)-3-methyl-1H-indole-2-carboxylic acid

To a solution of ethyl 4-(4-fluorophenyl)-3-methyl-1H-indole-2-carboxylate (350 mg, 1.18 mmol) in THE (10 mL) and EtOH (5 mL) was added NaOH solution (1 M, 10 mL). It was heated at 50° C. until no starting material left. Solvents were removed in vacuo and the residue was diluted with water, then acidified with HCl solution. The precipitate was collected and washed with water. It was dried to provide the acid as a pale brown powder (250 mg, 79% yield) which was used for next step reaction without further purification. Calcd for C₁₆H₁₃FNO₂⁺ 270.09 [M+H]⁺, found 269.90 [M+H]⁺.

### Step 4)

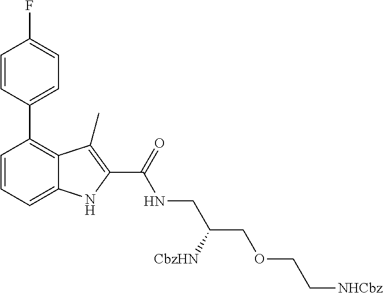 [SMILES: Cc1c(C(=O)NC[C@H](COCCNC(=O)OCc2ccccc2)NC(=O)OCc2ccccc2)[nH]c2cccc(-c3ccc(F)cc3)c12]

### benzyl (R)-(2-(2-(((benzyloxy)carbonyl)amino)-3-(4-(4-fluorophenyl)-3-methyl-1H-indole-2-carboxamido)propoxy)ethyl)carbamate

To a solution of 3-methyl-4-(4-fluoro-phenyl)-1H-indole-2-carboxylic acid (135 mg, 0.5 mmol) in dry DMF (3 mL) was added EDC (120 mg, 0.6 mmol) and HOBt (50 mg, 0.3 mmol). The reaction mixture was stirred at r.t. and DIPEA (0.17 mL, 1 mmol) was added followed by benzyl (R)-(1-amino-3-(2-(((benzyloxy)carbonyl)amino)ethoxy)propan-2-yl)carbamate (Amine Intermediate G) (201 mg, 0.5 mmol). The reaction mixture was stirred at r.t. overnight. It was extracted with EtOAc and washed with water and brine. The organic layer was dried over anhydrous sodium sulfate and filtered. The filtrate was concentrated and purified by column chromatography on silica gel to give the product (314 mg, 96% yield) as a colorless oil. ¹H NMR (300 MHz, CDCl₃) δ 9.37 (br s, 1H), 7.35 (m, 2H), 7.31 (m, 6H), 7.27 (m, 4H), 7.24 (m, 2H), 7.11 (t, J=8.7 Hz, 2H), 6.91 (m, 1H), 6.71 (br, s, 1H), 5.72 (br s, 1H), 5.57 (br s, 1H), 5.09 (m, 2H), 5.06 (m, 2H), 3.96 (m, 1H), 3.80 (m, 1H), 3.62 (m, 1H), 3.54 (m, 4H), 3.38 (m, 2H), 2.05 (s, 3H). MS (ESI): Calcd for C₃₇H₃₈FN₄O₆⁺ 653.27 [M+H]⁺, found 653.25 [M+H]⁺.

### Example 34. Preparation of N—((R)-2-amino-3-(((S)-2,5-diaminopentyl)oxy)propyl)-3-(4-fluorophenyl)-1H-indole-2-carboxamide hydrogen chloride salt

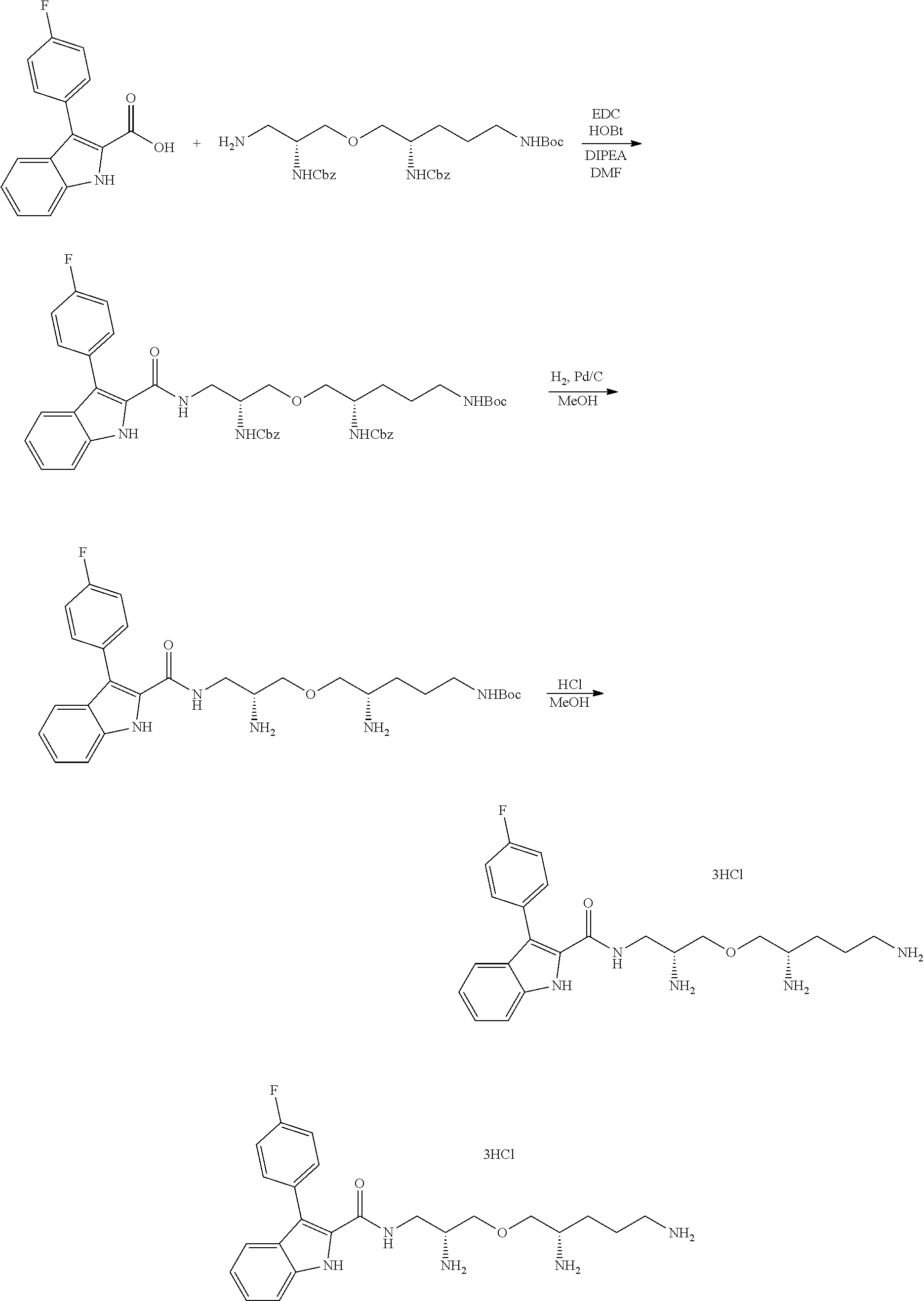

### N—((R)-2-amino-3-(((S)-2,5-diaminopentyl)oxy)propyl)-3-(4-fluorophenyl)-1H-indole-2-carboxamide hydrogen chloride salt

To a solution of tert-butyl ((S)-4-amino-5-((R)-2-amino-3-(3-(4-fluorophenyl)-1H indole-2-carboxamido)propoxy)pentyl)carbamate (15 mg, 0.03 mmol) in MeOH (2 mL) HCl solution (4 M in dioxane, 0.1 mL, 0.4 mmol) was added. It was stirred at r.t. for 1 hrs, then solvent was removed in vacuo. The residue was dissolved in water and purified on a C18 column chromatography using water as eluent to give the product as a white powder (4 mg, 27% yield). ¹H NMR (300 MHz, CD₃OD) δ 7.67 (m, 1H), 7.56 (m, 2H), 7.59 (m, 1H), 7.32 (m, 3H), 7.13 (m, 1H), 3.77 (m, 3H), 3.74 (m, 2H), 3.52 (m, 3H), 2.97 (m, 2H), 1.78 (m, 4H). MS (ESI): Calcd for C₂₃H₃₁FN₅O₂⁺428.25 [M+H]⁺, found 428.15 [M+H]⁺.

The requisite intermediates were prepared as follows:

### Step 1)

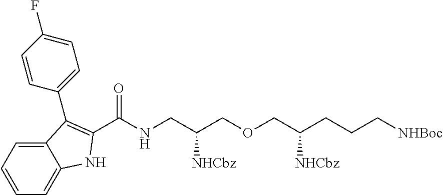 [SMILES: CC(C)(C)OC(=O)NCCC[C@@H](COC[C@@H](CNC(=O)c1[nH]c2ccccc2c1-c1ccc(F)cc1)NC(=O)OCc1ccccc1)NC(=O)OCc1ccccc1]

### benzyl tert-butyl ((S)-5-((R)-2-(((benzyloxy)carbonyl)amino)-3-(3-(4-fluorophenyl)-1H-indole-2-carboxamido)propoxy)pentane-1,4-diyl)dicarbamate

To a solution of 3-(4-fluorophenyl)-1H-indole-2-carboxylic acid (26 mg, 0.1 mmol) in dry DMF (1 mL) was added DIPEA (0.034 mL, 0.2 mmol), HOBt (10 mg, 0.06 mmol) and EDC (24 mg, 0.12 mmol). The reaction mixture was stirred at r.t. and benzyl tert-butyl ((S)-5-((R)-3-amino-2-(((benzyloxy)carbonyl)amino)propoxy)pentane-1,4-diyl)dicarbamate (Amine Intermediate H) (56 mg, 0.1 mmol) was added. The reaction mixture was stirred at r.t. overnight. It was extracted with EtOAc and washed with water and brine. The organic layer was dried over anhydrous sodium sulfate and filtered. The filtrate was concentrated and purified by column chromatography on silica gel to give the product (48 mg, 60% yield) as a white powder. MS (ESI): Calcd for C₄₄H₅₁FN₅O₈⁺ 796.36 [M+H]⁺, found 796.35 [M+H]⁺

### Step 2)

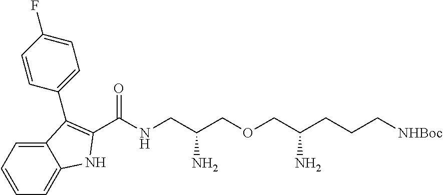 [SMILES: CC(C)(C)OC(=O)NCCC[C@H](N)COC[C@H](N)CNC(=O)c1[nH]c2ccccc2c1-c1ccc(F)cc1]

### tert-butyl ((S)-4-amino-5-((R)-2-amino-3-(3-(4-fluorophenyl)-1H-indole-2-carboxamido)propoxy)pentyl)carbamate

To a solution benzyl tert-butyl ((5)-5-((R)-2-(((benzyloxy)carbonyl)amino)-3-(3-(4-fluorophenyl)-1H-indole-2-carboxamido)propoxy)pentane-1,4-diyl)dicarbamate (46 mg, 0.058 mmol) in MeOH (10 mL) was added Pd/C (10%, 10 mg, 0.01 mmol). Then it was stirred under H₂ (50 psi) overnight. It was filtered through a Celite pad and washed with methanol, then concentrated to provide the crude product which was purified by column chromatography on silica gel to give the amine as an off-white powder (16 mg, 52% yield). MS (ESI): Calcd for C₂₈H₃₉FN₅O₄⁺ 528.30 [M+H]⁺, found 528.20 [M+H]⁺.

### Example 35. Preparation of N-((2R)-2,4-diamino-3-hydroxybutyl)-3-(4-fluorophenyl)-1H-indole-2-carboxamide

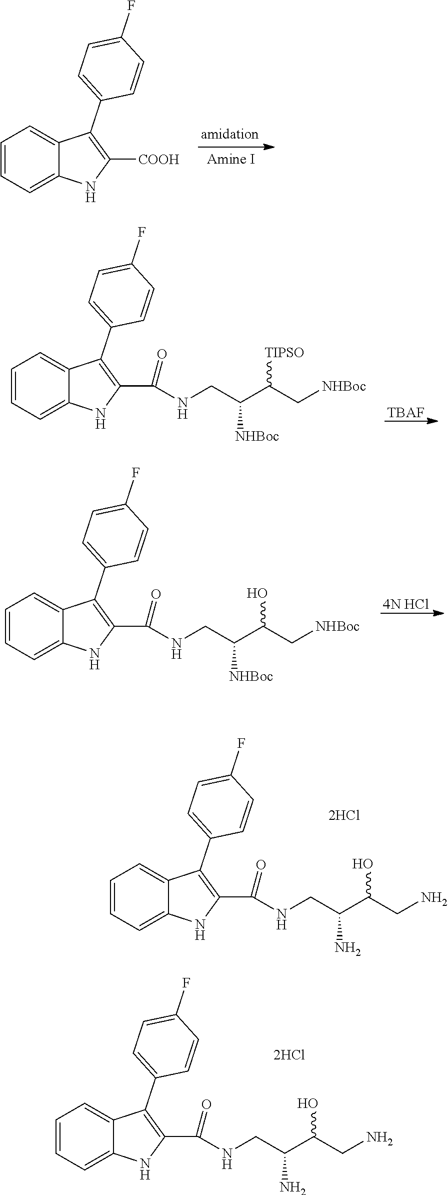

### N-((2R)-2,4-diamino-3-hydroxybutyl)-3-(4-fluorophenyl)-1H-indole-2-carboxamide

To a solution of di-tert-butyl ((3R)-4-(3-(4-fluorophenyl)-1H-indole-2-carboxamido)-2-hydroxybutane-1,3-diyl)dicarbamate (18 mg, 0.03 mmol) in 1 mL of methanol was added 0.2 mL of HCl (4N in dioxane, 0.8 mmol). Reaction mixture was stirred at 50° C. for 30 minutes then solvents were removed by rotovapor. Residue was washed with small amount of ethyl ether and dried. Product was collected as gray solid powder, 7.4 mg, 53%. ¹H NMR (300 MHz, D₂O) δ 7.36-7.46 (m, 3H), 7.24 (t, J=7.8 Hz, 1H), 7.16 (t, J=9.0 Hz, 1H), 7.05 (t, J=7.2 Hz, 1H), 3.99-4.03 (m, 1H), 3.37-3.54 (m, 3H), 3.05 (dd, J₁=3.0 Hz, J₂=13.5 Hz, 1H), 2.89 (dd, J₁=11.1 Hz, J₂=12.9 Hz, 1H); MS: Calcd for C₁₉H₂₂FN₄O₂⁺ 357.17 [M+H⁺], found 357.00 [M+H]

The requisite intermediates were prepared as follows:

### Step 1)

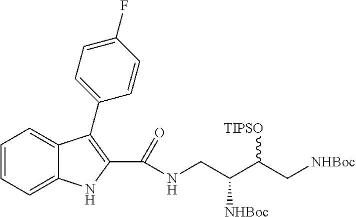

### di-tert-butyl ((3R)-4-(3-(4-fluorophenyl)-1H-indole-2-carboxamido)-2-((triisopropylsilyl)oxy)butane-1,3-diyl)dicarbamate

To a solution of (S)-di-tert-butyl (4-amino-2-((triisopropylsilyl)oxy)butane-1,3-diyl)dicarbamate (Amine intermediate I) (34 mg, 0.07 mmol) in 1 mL of DMF was added 3-(4-fluorophenyl)-1H-indole-2-carboxylic acid (35 mg, 0.08 mmol, 1.2 eq), 1-ethyl-3-(3-dimethylaminopropyl) carbodiimide (EDCI, 16.5 mg, 0.1 mmol, 1.5 eq), hydroxybenzotriazole (HOBt, 1.0 mg, 10 mol % cat.) and N,N-diisopropylethylamine (DIPEA, 13.8 mg, 0.13 umol, 1.5 eq). Resulting reaction mixture was stirred at room temperature overnight then diluted with 5 mL of water and stirred for 30 minutes. Solid was filtered and washed with small amount of water then dissolved in 3 mL of dichloromethane, washed with small amount of brine and dried. After solvent removed, crude product was used to next step without further purification.

### Step 2)

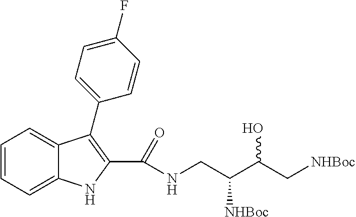 [SMILES: CC(C)(C)OC(=O)NCC(O)[C@@H](CNC(=O)c1[nH]c2ccccc2c1-c1ccc(F)cc1)NC(=O)OC(C)(C)C]

### di-tert-butyl ((3R)-4-(3-(4-fluorophenyl)-1H-indole-2-carboxamido)-2-hydroxybutane-1,3-diyl)dicarbamate

To a solution of 60 mg of di-tert-butyl ((3R)-4-(3-(4-fluorophenyl)-1H-indole-2-carboxamido)-2-((triisopropylsilyl)oxy)butane-1,3-diyl)dicarbamate (0.08 mmol) in 2 mL of THE was added 0.2 mL of TBAF solution (1.0 M/THF, 0.2 mmol) at 0° C. with stirring. Resulting reaction mixture was stirred at room temperature for 1 hour then diluted with 10 mL of EtOAc. The mixture was continuously washed with 0.5 N HCl, water, aq. Sat. NaHCO₃, brine then dried over Na₂SO₄. Solvent removed and residue was purified on column to give 38.5 mg product as white solid, 82%. 1H NMR (300 MHz, CDCl3) δ 9.58 (s, 1H), 7.42-7.51 (m, 4H), 7.23-7.36 (m, 3H), 7.16 (t, J=8.1 Hz, 1H), 6.35 (br s, 1H), 5.46 (br s, 1H), 5.20 (d, J=7.2 Hz, 1H), 4.56 (br s, 1H), 3.72-3.84 (m, 1H), 3.46-3.63 (m, 2H), 3.32-3.46 (m, 2H), 2.91-3.03 (m, 1H), 1.42 (s, 9H), 1.40 (s, 9H); MS: Calcd for C₂₉H₃₈FN₄O₆⁺557.27 [M+H⁺], found 557.00 [M+H]⁺.

### Example 36. Preparation of N-(((2R,4S)-4-(aminomethyl)-3-oxopyrrolidin-2-yl)methyl)-3-(4-fluorophenyl)-1H-indole-2-carboxamide

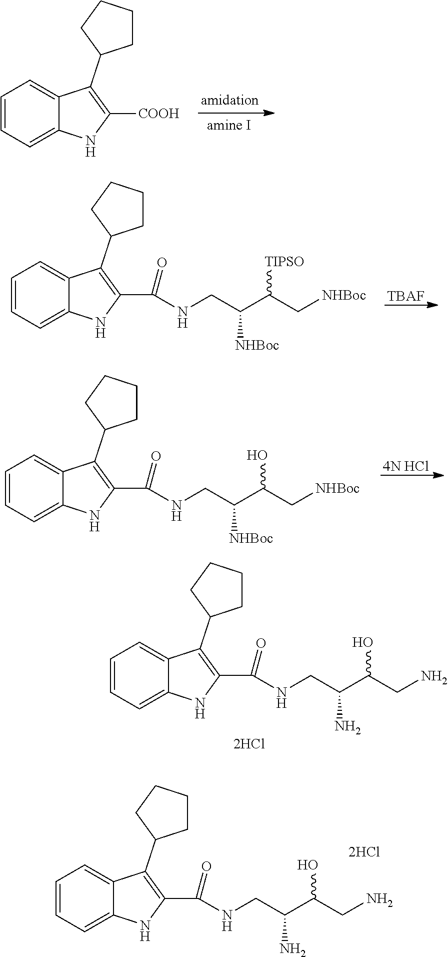

### 3-cyclopentyl-N-((2R)-2,4-diamino-3-hydroxybutyl)-1H-indole-2-carboxamide

To a solution of di-tert-butyl ((3R)-4-(3-cyclopentyl-1H-indole-2-carboxamido)-2-hydroxybutane-1,3-diyl)dicarbamate (11 mg, 0.02 mmol) in 1 mL of methanol was added 0.2 mL of HCl (4N in dioxane, 0.8 mmol). Reaction mixture was stirred at 50° C. for 30 minutes then solvents were removed by rotovapor. Residue was washed with small amount of ethyl ether and dried. Product was collected as gray solid powder, 6.9 mg, 82%. ¹H NMR (300 MHz, D₂O) δ 7.75 (d, J=8.1 Hz, 1H), 7.39 (d, J=9.0 Hz, 1H), 7.23 (t, J=8.4 Hz, 1H), 7.05 (t, J=7.2 Hz, 1H), 4.13 (d, J=11.1 Hz, 1H), 3.53-3.71 (m, 4H), 3.19-3.24 (m, 1H), 3.03-3.09 (m, 1H) 1.83-1.92 (m, 6H), 1.62 (br s, 2H); MS: Calcd for C₁₈H₂₆N₄O₂⁺ 331.21 [M+H⁺], found 331.00 [M+H]⁺

The requisite intermediates were prepared as follows:

### Step 1)

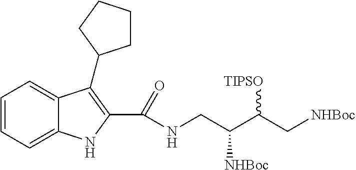

### di-tert-butyl ((3R)-4-(3-cyclopentyl-1H-indole-2-carboxamido)-2-((triisopropylsilyl)oxy)butane-1,3-diyl)dicarbamate

To a solution of di-tert-butyl ((3R)-4-(3-cyclopentyl-1H-indole-2-carboxamido)-2-((triisopropylsilyl)oxy)butane-1,3-diyl)dicarbamate (Amine intermediate I) (40 mg, 0.175 mmol) in 1 mL of DMF was added 3-(4-fluorophenyl)-1H-indole-2-carboxylic acid (86 mg, 0.2 mmol, 1.2 eq), 1-ethyl-3-(3-dimethylaminopropyl) carbodiimide (EDCI, 41 mg, 0.26 mmol, 1.5 eq), hydroxybenzotriazole (HOBt, 2.5 mg, 10 mol % cat.) and N,N-diisopropylethylamine (DIPEA, 28 mg, 0.26 mmol, 1.5 eq). Resulting reaction mixture was stirred at room temperature overnight then diluted with 5 mL of water and stirred for 30 minutes. Solid was filtered and washed with small amount of water then dissolved in 3 mL of dichloromethane, washed with small amount of brine and dried. After solvent removed, residue was used to next step without further purification.

### Step 2)

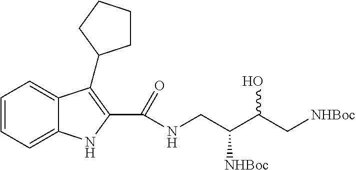 [SMILES: CC(C)(C)OC(=O)NCC(O)[C@@H](CNC(=O)c1[nH]c2ccccc2c1C1CCCC1)NC(=O)OC(C)(C)C]

### di-tert-butyl ((3R)-4-(3-cyclopentyl-1H-indole-2-carboxamido)-2-hydroxybutane-1,3-diyl)dicarbamate

To a solution of 42 mg of di-tert-butyl ((3R)-4-(3-cyclopentyl-1H-indole-2-carboxamido)-2-hydroxybutane-1,3-diyl)dicarbamate (0.06 mmol) in 2 mL of THE was added 0.2 mL of TBAF solution (1.0 M/THF, 0.2 mmol) at 0° C. with stirring. Resulting reaction mixture was stirred at room temperature for 1 hour then diluted with 10 mL of EtOAc. The mixture was continuously washed with 0.5 N HCl, water, aq. Sat. NaHCO₃, brine then dried over Na₂SO₄. Solvent removed and residue was purified on column to give 12 mg product as white solid, 37%. 1H NMR (300 MHz, CDCl₃) δ 9.37 (br s, 1H), 7.23 (d, J=8.1 Hz, 1H), 7.34 (d, J=8.1 Hz, 1H), 7.26 (t, J=14.1 Hz, 1H), 7.07 (t, J=7.5 Hz, 1H), 5.80 (br s, 1H), 5.51 (br s, 1H), 3.66-3.92 (m, 3H), 3.54-3.66 (m, 2H), 3.02-3.08 (m, 1H), 2.01-2.14 (m, 3H), 1.87-2.01 (m, 3H), 1.66-1.87 (m, 3H), 1.44 (s, 9H), 1.35 (s, 9H); MS: Calcd for C₂₈H₄₃N₄O₆⁺ 543.31 [M+H⁺], found 543.00 [M+H]⁺.

### Example 37. Description of General Test Methods

### Intrinsic MIC Assays

MIC assays were conducted in accordance with Clinical and Laboratory Standards Institute (CLSI) guidelines for broth microdilution. A 96-well plate containing cation-adjusted Mueller-Hinton (CAMH broth with 2-fold serial dilution of compounds was inoculated with log-phase bacterial at 5×10⁵ CFU/mL. The final volume in each well was 100 μL. Each compound was tested in duplicate. The microtiter plates were incubated in an aerobic environment for 18 hours at 37° C. Then the bacterial growth was tested by reading the plate with a VersaMax plate reader (Molecular Devices, Inc.) at 600 nm. The MIC was defined as the lowest compound concentration that inhibited 90% of bacteria growth.

The intrinsic MIC of the experimental EPIs was tested with the method described. The 2-fold serial dilution begins with 100 μg/mL of tested compound in the first column of the 96-well plates. The following Gram-negative bacterial strain was included in these assays: Pseudomonas aeruginosa ATCC 27853.

### Bacterial EPI Assays

The EPI assay for the purposes of these studies represents a MIC assay in which the MIC of the antibiotic against the bacteria is tested in the presence of an experimental efflux pump inhibitor (EPI). The highest concentration of the EPI present in the assay typically is ½ of the intrinsic MIC of the compound. If the intrinsic MIC of the EPI is greater than 100 μg/mL, the EPI assay was tested with 50 μg/mL. Using serial dilutions of the EPI, its enhancement of antibiotic activity was then evaluated. The relative EPI activity was decided by comparing the MIC of the antibiotic in the presence of the EPI compound with the intrinsic MIC of the antibiotic alone.

### Example 38. Standard EPI Assays

The impact of the EPIs on the MIC values of three test antibiotics (levofloxacin, ceftazidime and doxycycline) against P. aeruginosa ATCC 27853 were evaluated using our standard EPI assay. All three antibiotics levofloxacin, ceftazidime and doxycycline are known substrates of efflux pumps in P. aeruginosa, and are thus well-suited to be test antibiotics to assay for EPI activity.

In our standard EPI assay, the MIC of the test antibiotic is determined in the absence and presence of sub-inhibitory concentrations of the EPI. For an example, as the intrinsic MIC of Example 4 against P. aeruginosa ATCC 27853 is greater than 100 μg/mL, we used 6.25 μg/mL of the Example 4 in the standard EPI assay. The MIC of levofloxacin against P. aeruginosa ATCC 27853 in the absence of EPI is 1 μg/mL. In the presence of 6.25 μg/mL of the Example 4, the MIC of levofloxacin was markedly reduced to 0.032 μg/mL, a 32-fold reduction relative to the MIC of levofloxacin in the absence of EPI (1 μg/mL). Similarly, the MIC of ceftazidime against P. aeruginosa ATCC 27853 in the absence of EPI is 2 μg/mL. In the presence of 6.25 μg/mL of the Example 4, the MIC of ceftazidime was reduced to 0.5 μg/mL, a 4-fold reduction. For doxycycline, in the presence of 6.25 μg/mL of Example 4, the MIC is reduced 32-fold (1 μg/mL vs. 32 μg/mL without the EPI). For all the examples in Table 1, the potentiation activities are determined at 6.25 μg/mL.

### Example 39. Fluorescent-Based Cellular Assay for Efflux Inhibition

The impact of potential EPI compounds on the activity of efflux pumps was also evaluated with a fluorescence-based cellular assay that measures the efflux of Ethidium Bromide (EtBr), a known substrate of Gram-negative bacterial efflux pumps. When bound to intracellular bacterial DNA, EtBr fluoresces brightly, while the unbound fluorophore outside the bacterial cell exhibits little or no fluorescence. Thus, the efflux of EtBr from inside to outside the bacterial cell is associated with a substantive decrease in fluorescence.

Specifically, for this cellular assay, P. aeruginosa ATCC 27853 bacterial cells are grown overnight in CAMH broth. Bacteria are harvested from the overnight culture by centrifugation, and the cell pellet washed with phosphate-buffered containing 1 mM MgCl₂ (PBSM). The washed cell pellets are resuspended in PBSM to achieve a final OD at 600 nm of 1.0. Carbonyl cyanide 3-chlorophenylhydrazone (CCCP) is added to the bacterial suspension at a final concentration 50 μM, along with the addition of EtBr at a final concentration of 200 μM. The cells are then incubated in the dark at 37° C. for 50 minutes to allow for the depletion of ATP by the CCCP, which negatively impacts cellular efflux pump activity and thus results in the concomitant accumulation of ethidium bromide inside the cells. After the 50-minute incubation, the bacteria are spun down, and the supernatant discarded to remove extracellular CCCP and EtBr. The bacterial pellet is resuspended in an equal volume of PBSM, and 200 μL of the bacterial suspension added to wells of a black, flat-bottom 96-well plate containing test EPI compounds at concentrations ranging from 0.031-0.25-fold MIC, or an equivalent volume of the vehicle (DMSO) alone. The plates are pre-incubated at 37° C. for 5 minutes. EtBr efflux is initiated by addition of glucose (100 mM) to reenergizes the efflux pumps. A Spectramax iD5 fluorescent plate reader (Molecular Devices, Inc., Sunnyvale, CA) is used to monitor the fluorescence of each well at 37° C. once per minute for 240 minutes. The excitation and emission wavelengths were set at 510 and 610 nm, respectively.

In our EtBr efflux assay, our EPIs increase the level of accumulation of ethidium bromide inside the cell in a concentration-dependent manner suggesting that efflux pump inhibition is their mechanism of action.

Example 40. The following can illustrate representative pharmaceutical dosage forms, containing a compound of formula I (‘Compound X’) or a pharmaceutically acceptable salt thereof, for therapeutic or prophylactic use in humans. The tablets can optionally comprise an enteric coating.

| | mg/tablet |
|---|---|
| (i) Tablet 1 | |
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | |
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| | mg/mL |
|---|---|
| (iv) Injection 1 (1 mg/mL) | |
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | |
| Compound X = (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula I: 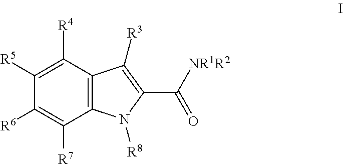
wherein:
R¹ is:
(a) (C₁-C₁₄)alkyl wherein the (C₁-C₁₄)alkyl is substituted with one or more —NR^{a1}R^{b1} groups, and wherein the (C₁-C₁₄)alkyl is substituted with one or more groups independently selected from the group consisting of —OH, —OR^{c}, —CN, NO₂, —B(OH)₂, —SO₂R^{d}, —CO₂R^{r}, —OC(═O)—NR^{a2}R^{b2}, and —O(C═O)—(C₁-C₆)alkyl-NR^{a2}R^{b2}, and wherein the (C₁-C₁₄)alkyl is optionally substituted independently with one or more halo, (C₁-C₄)alkyl or (C₃-C₇)carbocyclyl; or
(b) 4-7 membered monocyclic heterocyclyl-(C₁-C₆)alkyl- wherein the 4-7 membered monocyclic heterocyclyl-(C₁-C₆)alkyl- is substituted independently with one or more Z¹ or Z², and wherein the 4-7 membered monocyclic heterocyclyl-(C₁-C₆)alkyl- is substituted independently with one or more oxo, Z³, or Z⁴, and wherein the 4-7 membered monocyclic heterocyclyl-(C₁-C₆)alkyl- is optionally substituted independently with one or more halo, (C₁-C₄)alkyl, or (C₃-C₇)carbocyclyl;
R² is hydrogen, (C₁-C₄)alkyl, or (C₃-C₇)carbocyclyl;
R³ is hydrogen, halo, (C₁-C₄)alkyl, (C₁-C₄)haloalkyl, (C₁-C₄)alkoxy, (C₁-C₄)haloalkoxy, —OH, —X—(C₃-C₇)carbocyclyl, —X-aryl, —X-heterocyclyl, or —X-heteroaryl, wherein the —X—(C₃-C₇)carbocyclyl, —X-aryl, —X-heterocyclyl, or —X-heteroaryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁴ is hydrogen, halo, (C₁-C₄)alkyl, (C₁-C₄)haloalkyl, (C₁-C₄)alkoxy, (C₁-C₄)haloalkoxy, —OH, X-aryl, —X-heterocyclyl, or —X-heteroaryl, wherein the —X—(C₃-C₇)carbocyclyl, —X-aryl, —X-heterocyclyl, or —X-heteroaryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁵ is hydrogen, halo, (C₁-C₄)alkyl, (C₁-C₄)haloalkyl, (C₁-C₄)alkoxy, (C₁-C₄)haloalkoxy, —OH, X-aryl, —X-heterocyclyl, or —X-heteroaryl, wherein the —X—(C₃-C₇)carbocyclyl, —X-aryl, —X— heterocyclyl, or —X-heteroaryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁶ is hydrogen, halo, (C₁-C₄)alkyl, (C₁-C₄)haloalkyl, (C₁-C₄)alkoxy, (C₁-C₄)haloalkoxy, —OH, X-aryl, —X-heterocyclyl, or —X-heteroaryl, wherein the —X—(C₃-C₇)carbocyclyl, —X-aryl, —X— heterocyclyl, or —X-heteroaryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁷ is hydrogen, halo, (C₁-C₄)alkyl, (C₁-C₄)haloalkyl, (C₁-C₄)alkoxy, (C₁-C₄)haloalkoxy, —OH, X-aryl, —X-heterocyclyl, or —X-heteroaryl, wherein the —X—(C₃-C₇)carbocyclyl, —X-aryl, —X— heterocyclyl, or —X-heteroaryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁸ is hydrogen, (C₁-C₄)alkyl, or (C₃-C₇)carbocyclyl;
each X is independently absent, —(C₁-C₆)alkyl-, —O—, —S—, —S(═O)—, —S(═O)₂—, or —NR^{f}—;
Z¹ is —NR^{a1}R^{b1};
each Z² is independently —(C₁-C₆)alkyl substituted with one or more Z¹, wherein the —(C₁-C₆)alkyl is optionally substituted independently with one or more halo or (C₃-C₇)carbocyclyl;
each Z³ is independently —OH, —OR^{c}, —CN, NO₂, —B(OH)₂, —SO₂R^{d}, —CO₂R, —OC(═O)—NR^{a2}R^{b2} and —O(C═O)—(C₁-C₆)alkyl-NR^{a2}R^{b2},
each Z⁴ is independently —(C₁-C₆)alkyl substituted with one or more Z³, wherein the —(C₁-C₆)alkyl is optionally substituted independently with one or more halo or (C₃-C₇)carbocyclyl;
each R^{a1} and R^{b1} is independently hydrogen, (C₁-C₆)alkyl or (C₃-C₇)carbocyclyl:
each R^{a2} and R^{b2} is independently hydrogen, (C₁-C₆)alkyl, or (C₃-C₇)carbocyclyl; and
each R^{a3} and R^{b3} is independently hydrogen, (C₁-C₆)alkyl, or (C₃-C₇)carbocyclyl;
each R is independently (C₁-C₆)alkyl, wherein the (C₁-C₆)alkyl is optionally substituted independently with one or more —NR^{a3}R^{b3}, OH, (C₁-C₆)alkoxy, or (C₁-C₆)haloalkoxy;
each R^{d} is independently (C₁-C₆)alkyl or (C₃-C₇)carbocyclyl;
each R^{e} is independently hydrogen, (C₁-C₆)alkyl, or (C₃-C₇)carbocyclyl; and
each R^{f} is independently hydrogen, (C₁-C₆)alkyl, or (C₃-C₇)carbocyclyl;
or a salt thereof.

2. The compound of claim 1 wherein:
R¹ is:
(a) (C₁-C₁₄)alkyl wherein the (C₁-C₁₄)alkyl is substituted with one or more —NR^{a1}R^{b1} groups, and wherein the (C₁-C₁₄)alkyl is substituted with one or more groups independently selected from the group consisting of —OH, —OR^{c}, —CN, —B(OH)₂, —SO₂R^{d}, —CO₂R^{e}, —OC(═O)—NR^{a2}R^{b2}, and —O(C═O)—(C₁-C₆)alkyl-NR^{a2}R^{b2}, and wherein the (C₁-C₁₄)alkyl is optionally substituted independently with one or more halo, (C₁-C₄)alkyl or (C₃-C₇)carbocyclyl;
or
(b) 4-7 membered monocyclic heterocyclyl-(C₁-C₆)alkyl- wherein the 4-7 membered monocyclic heterocyclyl-(C₁-C₆)alkyl- is substituted independently with one or more Z¹ or Z², and wherein the 4-7 membered monocyclic heterocyclyl-(C₁-C₆)alkyl- is substituted independently with one or more oxo, Z³, or Z⁴, and wherein the 4-7 membered monocyclic heterocyclyl-(C₁-C₆)alkyl- is optionally substituted independently with one or more halo, (C₁-C₄)alkyl, or (C₃-C₇)carbocyclyl;
R² is hydrogen, (C₁-C₄)alkyl, or (C₃-C₇)carbocyclyl;
R³ is —X—(C₃-C₇)carbocyclyl, —X-aryl, —X-heterocyclyl, or —X-heteroaryl wherein the —X—(C₃-C₇)carbocyclyl, —X-aryl, —X-heterocyclyl, or —X-heteroaryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R⁴ is hydrogen, halo, (C₁-C₄)alkyl, (C₁-C₄)haloalkyl, (C₁-C₄)alkoxy, (C₁-C₄)haloalkoxy, or —OH;
R⁵ is hydrogen, halo, (C₁-C₄)alkyl, (C₁-C₄)haloalkyl, (C₁-C₄)alkoxy, (C₁-C₄)haloalkoxy, or —OH;
R⁶ is hydrogen, halo, (C₁-C₄)alkyl, (C₁-C₄)haloalkyl, (C₁-C₄)alkoxy, (C₁-C₄)haloalkoxy, or —OH;
R⁷ is hydrogen, halo, (C₁-C₄)alkyl, (C₁-C₄)haloalkyl, (C₁-C₄)alkoxy, (C₁-C₄)haloalkoxy, or —OH;
R⁸ is hydrogen, (C₁-C₄)alkyl, or (C₃-C₇)carbocyclyl;
each X is independently absent, —(C₁-C₆)alkyl-, —O—, —S—, —S(═O)—, —S(═O)₂—, or —NR^{f}—;
Z¹ is —NR^{a1}R^{b1};
each Z² is independently —(C₁-C₆)alkyl substituted with one or more Z¹, wherein the —(C₁-C₆)alkyl is optionally substituted independently with one or more halo or (C₃-C₇)carbocyclyl;
each Z³ is independently —OH, —OR, —CN, —B(OH)₂, —SO₂R^{d}, —CO₂R, —OC(═O)—NR^{a2}R^{b2} and —O(C═O)—(C₁-C₆)alkyl-NR^{a2}R^{b2},
each Z⁴ is independently —(C₁-C₆)alkyl substituted with one or more Z³, wherein the —(C₁-C₆)alkyl is optionally substituted independently with one or more halo or (C₃-C₇)carbocyclyl;
each R^{a1} and R^{b1} is independently hydrogen, (C₁-C₆)alkyl or (C₃-C₇)carbocyclyl;
each R^{a2} and R^{b2} is independently hydrogen, (C₁-C₆)alkyl, or (C₃-C₇)carbocyclyl; and
each R^{a3} and R^{b3} is independently hydrogen, (C₁-C₆)alkyl, or (C₃-C₇)carbocyclyl;
each R^{c} is independently (C₁-C₆)alkyl, wherein the (C₁-C₆)alkyl is optionally substituted independently with one or more —NR^{a3}R^{b3}, OH, (C₁-C₆)alkoxy, or (C₁-C₆)haloalkoxy;
each R^{d} is independently (C₁-C₆)alkyl or (C₃-C₇)carbocyclyl;
each R^{e} is independently hydrogen, (C₁-C₆)alkyl, or (C₃-C₇)carbocyclyl; and
each R^{f} is independently hydrogen, (C₁-C₆)alkyl, or (C₃-C₇)carbocyclyl:
or a salt thereof.

3. The compound or salt of claim 1, wherein R² is hydrogen.

4. The compound or salt of claim 1, wherein R⁸ is hydrogen.

5. The compound or salt of claim 1, wherein the compound of formula I is a compound of formula Ia: 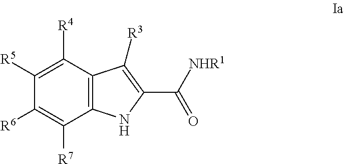
or a salt thereof.

6. The compound or salt of claim 1, wherein R⁴ is hydrogen, halo, or aryl, wherein the aryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

7-8. (canceled)

9. The compound or salt of claim 1, wherein R⁶ is hydrogen, halo, or aryl wherein the aryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

10-12. (canceled)

13. The compound or salt of claim 1, wherein R⁵ is hydrogen or halo.

14. (canceled)

15. The compound or salt of claim 1, wherein R⁷ is hydrogen, halo, or aryl wherein the aryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

16-17. (canceled)

18. The compound or salt of claim 1, wherein R³ is hydrogen, (C₁-C₄)alkyl, —X—(C₃-C₇)carbocyclyl or —X-aryl, wherein the —X—(C₃-C₇)carbocyclyl or —X-aryl is independently optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

19-22. (canceled)

23. The compound or salt of claim 1, wherein R³ is hydrogen, (C₁-C₄)alkyl, (C₄-C₇)carbocyclyl or phenyl, wherein the (C₃-C₇)carbocyclyl or phenyl is independently optionally substituted with one or more groups independently selected from the group consisting of halo and —CN.

24. (canceled)

25. The compound or salt of claim 1, wherein
R₁ is:
(a) (C₂-C₇)alkyl wherein the (C₂-C₇)alkyl is substituted with one or more —NR^{a1}R^{b1} groups, and wherein the (C₂-C₇)alkyl is substituted with one or more groups independently selected from the group consisting of —OH, —OR^{c}, —CN, —B(OH)₂, —SO₂R^{d}, —CO₂R^{e}, —OC(═O)—NR^{a2}R^{b2}, and —O(C═O)—(C₁-C₆)alkyl-NR^{a2}R^{b2}, and wherein the (C₂-C₇)alkyl is optionally substituted independently with one or more halo, (C₁-C₄)alkyl or (C₃-C₇)carbocyclyl;
or
(b) 4-7 membered monocyclic heterocyclyl-(C₁-C₂)alkyl- wherein the 4-7 membered monocyclic heterocyclyl-(C₁-C₂)alkyl- is substituted independently with one or more Z¹ or Z², and wherein the 4-7 membered monocyclic heterocyclyl-(C₁-C₁)alkyl- is substituted independently with one or more oxo, Z³, or Z⁴, and wherein the 4-7 membered monocyclic heterocyclyl-(C₁-C₆)alkyl- is optionally substituted independently with one or more halo, (C₁-C₄)alkyl, or (C₃-C₇)carbocyclyl.

26-28. (canceled)

29. The compound or salt of claim 1, wherein R¹ is (C₂-C₇)alkyl wherein the (C₂-C₇)alkyl is substituted with one or more —NR^{a1}R^{b1} groups, and wherein the (C₂-C₇)alkyl is substituted with one or more groups independently selected from the group consisting of —OH, —OR, —CN, —B(OH)₂, —SO₂R^{d}, —CO₂R^{e}, —OC(═O)—NR^{a2}R^{b2}, and —O(C═O)—(C₁-C₆)alkyl-NR^{a2}R^{b2}, and wherein the (C₂-C₇)alkyl is optionally substituted independently with one or more halo, (C₁-C₄)alkyl or (C₃-C₇)carbocyclyl.

30-32. (canceled)

33. The compound or salt of claim 1, wherein R¹ is 4-7 membered monocyclic heterocyclyl-(C₁-C₂)alkyl- wherein the 4-7 membered monocyclic heterocyclyl-(C₁-C₂)alkyl- is substituted independently with one or more Z¹ or Z₂, and wherein the 4-7 membered monocyclic heterocyclyl-(C₁-C₁)alkyl- is substituted independently with one or more oxo, Z, or Z⁴, and wherein the 4-7 membered monocyclic heterocyclyl-(C₁-C₆)alkyl- is optionally substituted independently with one or more halo, (C₁-C₄)alkyl, or (C₃-C₇)carbocyclyl.

34-36. (canceled)

37. The compound or salt of claim 1, wherein R¹ is:

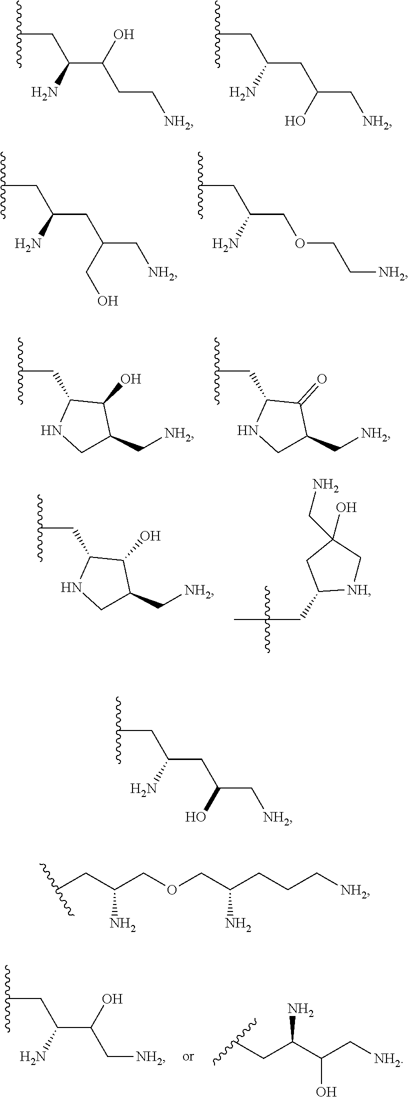

38. The compound of claim 1 that is: 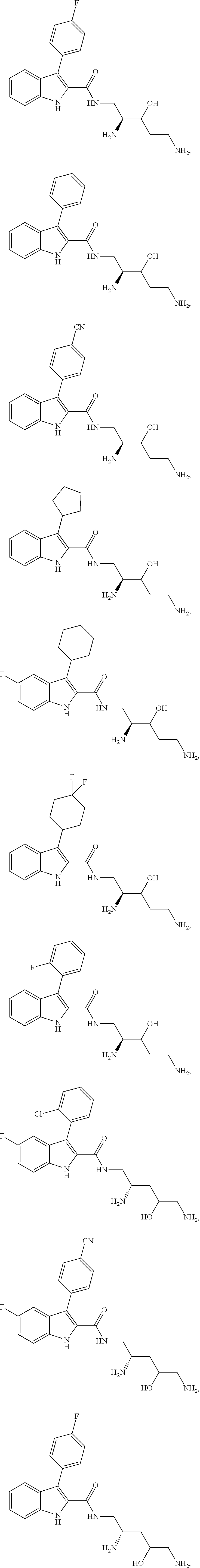 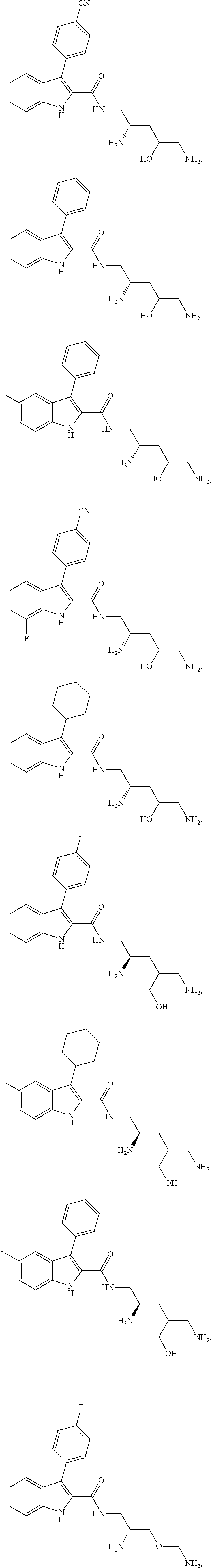 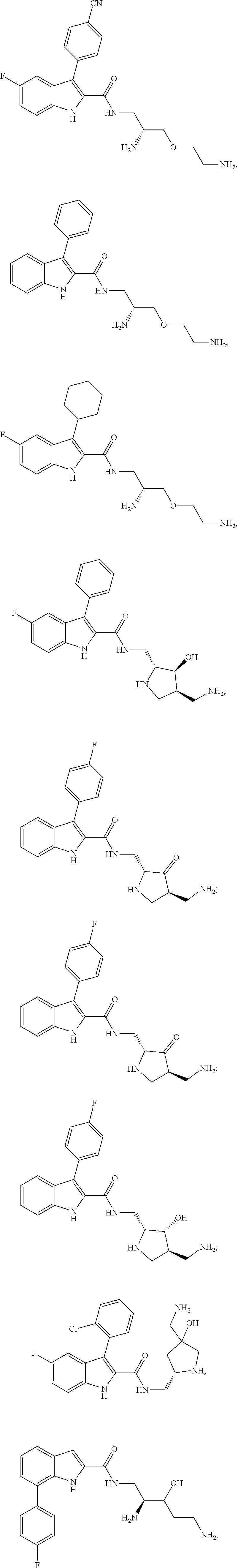 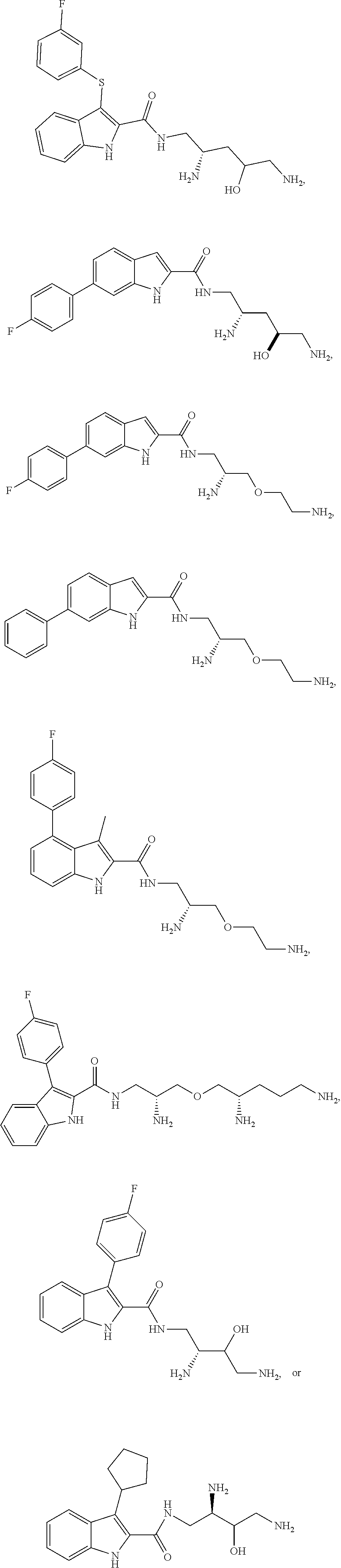
or a salt thereof.

39. A pharmaceutical composition comprising a compound as described in claim 1 or a pharmaceutically acceptable salt thereof, and a pharmaceutically acceptable excipient.

40. A pharmaceutical composition comprising a compound as described in claim 1 or a pharmaceutically acceptable salt thereof, one or more antibacterial agents and a pharmaceutically acceptable excipient.

41. A method of inhibiting a bacterial efflux pump in an animal comprising administering to the animal a compound as described in claim 1 or a pharmaceutically acceptable salt thereof.

42. A method of treating or preventing a bacterial infection in an animal comprising co-administering to the animal a compound as described in claim 1 or a pharmaceutically acceptable salt thereof and one or more antibacterial agents.

43-52. (canceled)
